(19) United States
(12) Patent Application Publication
(10) Pub. No.: US 2022/0136038 A1
(43) Pub. Date: May 5, 2022

(54) COMPOSITIONS AND METHODS FOR DETECTING MODIFIED NUCLEIC ACIDS AND AMPLIFYING ssDNA

(71) Applicant: MAMMOTH BIOSCIENCES, INC., Birsbane, CA (US)

(72) Inventors: Lucas Benjamin HARRINGTON, San Francisco, CA (US); Janice Sha CHEN, San Francisco, CA (US); James Paul BROUGHTON, San Francisco, CA (US); Isaac Paterson WITTE, Brisbane, CA (US)

(21) Appl. No.: 17/365,967

(22) Filed: Jul. 1, 2021

Related U.S. Application Data
(63) Continuation of application No. PCT/US20/12257, filed on Jan. 3, 2020.
(60) Provisional application No. 62/788,703, filed on Jan. 4, 2019.
(60) Provisional application No. 62/788,708, filed on Jan. 4, 2019.

(51) Int. Cl. C12Q 1/6823; C12Q 1/6809; C12Q 1/6876; C12Q 1/683; C12Q 1/6844

(52) U.S. Cl. CPC C12Q 1/6823; C12Q 1/6809; C12Q 1/6876; C12Q 2600/166; C12Q 1/6844; C12Q 2600/154; C12Q 1/683

(57) ABSTRACT

Described herein are methods to detect nucleic acid modifications, such as methylated DNA or methylated RNA, using a programmable nuclease. The present disclosure further provides compositions and methods for generating and amplifying ssDNA from target nucleic acid sequences, or templates, for activation of programmable nucleases. Further provided herein are methods of coupling ssDNA amplification and activation of programmable nucleases with programmable nuclease systems, such as DETECTR systems, including trans cleavage of detector nucleic acids to produce a signal indicating the presence of the target nucleic acid sequence.

### CROSS-REFRENCE

The present application is a continuation of International Patent Application No. PCT/US2020/012257 filed Jan. 3, 2020, which claims priority to and the benefit from U.S. Provisional Application Nos. 62/788,703 filed Jan. 4, 2019 and 62/788,708 filed Jan. 4, 2019, the entire contents of each of which are herein incorporated by reference.

### BACKGROUND

Nucleic acid modifications can impact gene expression. Deregulation or aberrant nucleic acid modifications are implicated in a variety of diseases, such as cancer. For example, hypermethylation of CpG islands in DNA in promoter regions for tumor-suppressor genes is common in several types of cancer, such as colon cancers, bladder cancers, and stomach cancers. Deregulation of methylation of RNA has been associated with aberrant gene expression and the potential activation of oncogenes in cancers, such as breast cancer, non-small-cell lung cancer, and acute myeloid leukemia. However, the presence of nucleic acid modifications on target nucleic acids is difficult to assess, and current approaches are not suitable to rapidly examine nucleic acid modifications for target nucleic acid sequences.

Certain programmable nucleases (e.g., Cas14a1) exhibit indiscriminate trans cleavage of detector nucleic acids when activated by a target single stranded DNA (ssDNA), enabling their use for detection of the target ssDNA in samples. However, there is a need for amplification of target ssDNA from nucleic acid templates (e.g., RNA, ssDNA, double stranded DNA (dsDNA)) to enhance detection of a target nucleic acid using programmable nucleases.

### SUMMARY

The methods and compositions disclosed herein relate to the detection of a modified nucleic acid. The methods of detection as described herein comprise contacting a modification sensitive programmable nuclease to a sample comprising a modified nucleic acid. The methods of detection described herein comprise contacting a sample comprising a modified nucleic acid to an enzyme composition comprising a programmable nuclease, wherein the enzyme composition exhibits modification sensitive cleavage. The methods of detection described herein comprise contacting a sample comprising a modified nucleic acid to a reagent that differentially reacts to modified bases and to a programmable nuclease. The programmable nuclease can be, but is not limited to, a CRISPR enzyme. Furthermore, these methods can be used to assess modification status (also referred to as a “modification state”) of a nucleic acid. For example, the methods as disclosed herein are used to determine if a target nucleic acid molecule has a modification or does not have a modification.

The present disclosure provides methods of generating and amplifying ssDNA from a template target nucleic acid, such as cDNA, dsDNA, ssDNA, or RNA. Amplified ssDNA can be incubated with programmable nucleases (e.g., Cas nucleases) to activate transcleavage. Activated programmable nucleases can cleave detector nucleic acids, which produce a signal indicating the presence of the target nucleic acid.

In various aspects, the present disclosure provides a method of assaying for a target nucleic acid in a sample, comprising: selectively producing a target single stranded DNA (ssDNA) using amplification of the target nucleic acid of the sample; contacting the target ssDNA to a complex comprising a guide nucleic acid comprising a segment that is reverse complementary to a segment of the target ssDNA and a programmable nuclease that exhibits sequence independent cleavage upon forming a complex comprising the segment of the guide nucleic acid binding to the segment of the target ssDNA; and assaying for cleavage of at least one detector nucleic acid molecules of a population of detector nucleic acid molecules, wherein the cleavage indicates a presence of the target nucleic acid in the sample and wherein the absence of the cleavage indicates an absence of the target nucleic acid in the sample. Often, selectively producing the target ssDNA comprises amplifying a target double stranded DNA having a target ssDNA and a nontarget ssDNA and selectively degrading the nontarget ssDNA. Sometimes, selectively producing the target ssDNA comprises amplifying the target ssDNA comprises amplifying a target double stranded DNA having a target ssDNA and a nontarget ssDNA and selectively producing an amplified target ssDNA.

In various aspects, the present disclosure provides a method of assaying for a target nucleic acid in a sample comprising: selectively amplifying a target single stranded DNA (ssDNA); contacting the target ssDNA to a complex comprising a guide nucleic acid comprising a segment that is reverse complementary to a segment of the target ssDNA and a programmable nuclease that exhibits sequence independent cleavage upon forming a complex comprising the segment of the guide nucleic acid binding to the segment of the target ssDNA; and assaying for cleavage of at least some detector nucleic acid molecules of a population of detector nucleic acid molecules, wherein the cleavage indicates a presence of the target nucleic acid in the sample and wherein the absence of the cleavage indicates an absence of the target nucleic acid in the sample. Sometimes, selectively amplifying the target ssDNA comprises amplifying a target double stranded DNA having a target ssDNA and a nontarget ssDNA and selectively degrading the nontarget ssDNA. Often, selectively amplifying the target ssDNA comprises amplifying a target double stranded DNA having a target ssDNA and a nontarget ssDNA and selectively producing an amplified target ssDNA.

Additionally, the present disclosure provides methods of generating and amplifying ssDNA from a template target nucleic acid, such as cDNA, dsDNA, ssDNA, or RNA. Amplified ssDNA can be incubated with programmable nucleases (e.g., Cas nucleases) to activate trans cleavage. Activated programmable nucleases can cleave detector nucleic acids, which produce a signal indicating the presence of the target nucleic acid. Methods of generating and amplifying ssDNA from a template target nucleic acid may comprise generating and amplifying a modified nucleic acid.

In various aspects, the present disclosure provides a method of assaying for a modification state of a segment of a target nucleic acid, the method comprising: contacting a sample comprising the target nucleic acid to: a guide nucleic acid that hybridizes to the segment of the target nucleic acid; a detector nucleic acid; and a programmable nuclease that cleaves the detector nucleic acid upon hybridization of the guide nucleic acid to the segment of the target nucleic acid; and assaying for a signal produced by cleavage of the detector nucleic acid to determine the modification state of the segment of the target nucleic acid.

In some aspects, the target nucleic acid is target RNA. In other aspects, the target nucleic acid is target DNA.

In various aspects, the present disclosure provide a method of assaying for a modification state of a segment of a target RNA, the method comprising: contacting a sample comprising the target RNA to: a guide nucleic acid that hybridizes to the segment of the target RNA; a detector nucleic acid; and a programmable nuclease that cleaves the detector nucleic acid upon hybridization of the guide nucleic acid to the segment of the target RNA; and contacting a second sample comprising an RNA having an unmodified segment comprising the same sequence as the segment of the target RNA to: the guide nucleic acid; the detector nucleic acid; and the programmable nuclease that cleaves the detector nucleic acid upon hybridization of the guide nucleic acid to the segment of the unmodified RNA; assaying for a first signal produced by cleavage of the detector nucleic acid in the sample; assaying for a second signal produced by cleavage of the detector nucleic acid in the second sample; and determining the modification state of the target RNA based on a comparison of the first signal to the second signal.

In some aspects, the modification state of the segment is modified when the first signal is less than the second signal. In other aspects, the modification state of the segment is unmodified when the first signal is substantially the same as the second signal. In some aspects, the modification state is modified when the segment of the target RNA comprises at least one base with a modification. In some aspects, the at least one base with the modification is present on a nucleic acid in a region 5′ to 3′ from nucleic acid 1 to nucleic acid 16 of the segment. In some aspects, the at least one base with the modification is present on a nucleic acid in the region 5′ to 3′ from nucleic acid 1 to nucleic acid 8 of the segment.

In some aspects, the method further comprises reverse transcribing the target RNA into DNA, amplifying the DNA, and in vitro transcribing the DNA into the target RNA.

In various aspects, the present disclosure provides a method of assaying for a modification state of a segment of a target DNA, the method comprising: contacting a sample comprising the target DNA to: a DNA modification reagent; a guide nucleic acid that hybridizes to the segment of the target DNA; a detector nucleic acid; and a programmable nuclease that cleaves the detector nucleic acid upon hybridization of the guide nucleic acid to the segment of the target DNA; and assaying for a signal produced by cleavage of the detector nucleic acid to determine the modification state of the segment of the target DNA.

In some aspects, the DNA modification reagent is a modification-specific restriction enzyme or sodium bisulfite.

In various aspects, the present disclosure provides a method of assaying for a modification state of a segment of a target DNA, the method comprising: contacting a sample comprising the target DNA to: a modification-specific restriction enzyme that cleaves the segment of the target DNA when the segment of the target DNA is unmodified; a guide nucleic acid that hybridizes to the segment of the target DNA; a detector nucleic acid; and a programmable nuclease that cleaves the detector nucleic acid upon hybridization of the guide nucleic acid to the segment of the target DNA; and assaying for a signal produced by cleavage of the detector nucleic acid to determine the modification state of the segment of the target DNA.

In some aspects, detection of the signal indicates the segment of the target DNA is modified. In some aspects, the contacting the sample to the guide nucleic acid, the detector nucleic acid, the programmable nuclease, or any combination thereof occurs after the contacting the sample to the modification-specific restriction enzyme.

In further aspects, the modification-specific restriction enzyme is Dpnl, DpnII, MspI, MspJIAat II, Acc II, Aor13H I, Aor51H I, BspT104 I, BssH II, Cfr10 I, Cla I, Cpo I, Eco52, I, Hae II, Hha I, Mlu I, Nae I, Not I, Nru I, Nsb I, PmaC I, Psp1406 I, Pvu I, Sac II, Sal I, Sma I, SnaB I, or Epi HpaII. In still further aspects, the modification-specific restriction enzyme is Epi HpaII.

In various aspects, the present disclosure provides a method of assaying for a modification state of a segment of a target DNA, the method comprising: contacting the sample to: sodium bisulfite; a guide nucleic acid that hybridizes to the segment of the target DNA; a detector nucleic acid; and a programmable nuclease that cleaves the detector nucleic acid upon hybridization of the guide nucleic acid to the segment of the target DNA; and assaying for a signal produced by cleavage of the detector nucleic acid to determine the modification state of the segment of the target DNA.

In some aspects, detection of the signal indicates the modification state of the segment of the target DNA is unmodified.

In various aspects, the present disclosure provides a method of assaying for a modification state of a segment of a target DNA, the method comprising: contacting a sample comprising the target DNA to: sodium bisulfite; a guide nucleic acid that hybridizes to a sodium bisulfite converted segment of the target DNA; a detector nucleic acid; a programmable nuclease that cleaves the detector nucleic acid upon hybridization of the guide nucleic acid to the sodium bisulfite converted segment of the target DNA; and assaying for a signal produced by cleavage of the detector nucleic acid to determine the modification state of the segment of the target DNA.

In some aspects, the detection of the signal indicates the modification state of the segment of the target DNA is modified. In some aspects, the method further comprising deaminating an unmethylated cytosine into a uracil in the segment of the target DNA upon contacting the sample to the sodium bisulfate, thereby producing a sodium bisulfate converted segment of the target DNA.

In some aspects, the method further comprises amplifying the target DNA. In some aspects, the modification state is modified when the segment of the target DNA comprises at least one base with a modification. In some aspects, the at least one base with the modification is present on a nucleic acid in a region 5′ to 3′ from nucleic acid 1 to nucleic acid 16 of the segment. In some aspects, the at least one base with the modification is present on a nucleic acid in the region 5′ to 3′ from nucleic acid 1 to nucleic acid 8 of the segment. In some aspects, the modification comprises methylation. In some aspects, the methylation comprises methylation of CpG sites. In some aspects, the methylation comprises an N6-methyladenosine. In some aspects, the modification comprises an 5-hydroxymethylcytosine (5hmC), 5-formylcytosine (5fC), 5-carboxylcytosine (5caC), 5-hydroxymethyluracil (5hmU), 5-methylcytosine (5mC), 3 -methylcytosine (3mC), N6-methyladenosine (m6A), N6, 2′-O-dimethyladenosine (m6Am), N1-methyladenosine (m1A), N1-methylguanosine (m1G), 5-methylcytosine (m5C), or 5-hydroxymethylcytosine (hm5C).

In some aspects, the modification comprises acetylation. In some aspects, the programmable nuclease is a Type VI programmable nuclease. In further aspects, Type VI programmable nuclease is a Cas13 protein. In still further aspects, the Cas13 protein is Cas13a, Cas13b, Cas13c, Cas13d, or Cas13e.

In some aspects, the programmable nuclease is a Type V programmable nuclease. In further aspects, the Type V programmable nuclease is a Cas12 protein or a Cas14 protein. In still further aspects, the Cas12 protein is Cas12a, Cas12b, Cas12c, Cas12d, or Cas12e.

In some aspects, the Cas14 protein is Cas14a, Cas14b, Cas14c, Cas14d, Cas14e, Cas14f, Cas14g, Cas14h, Cas14i, Cas14j, or Cas14k.

In some aspects, the amplifying comprises thermal cycling amplification or isothermal amplification. In some aspects, the thermal cycling amplification comprises polymerase chain reaction amplification. In some aspects, the isothermal amplification comprises isothermal recombinase polymerase amplification (RPA), transcription mediated amplification (TMA), strand displacement amplification (SDA), helicase dependent amplification (HDA), loop mediated amplification (LAMP), rolling circle amplification (RCA), single primer isothermal amplification (SPIA), ligase chain reaction (LCR), simple method amplifying RNA targets (SMART), or improved multiple In some aspects, the human subject has cancer. In some aspects, the modification is an epigenetic modification. In some aspects, the modification is indicative of a disease. In some aspects, the disease is cancer. In some aspects, the cancer is breast cancer or non-small cell lung cancer.

In various aspects, the present disclosure provides a method of assaying for a target nucleic acid in a sample, the method comprising: selectively producing a target single stranded DNA (ssDNA) by isothermal amplification of the target nucleic acid of the sample with a forward primer and a reverse primer to produce a target double stranded DNA having the target ssDNA and a nontarget ssDNA; and contacting the sample to: an exonuclease that selectively degrades the nontarget ssDNA; a guide nucleic acid that hybridizes to a segment of the target ssDNA; a detector nucleic acid; and a programmable nuclease that cleaves the detector nucleic acid upon hybridization of the guide nucleic acid to the segment of the target ssDNA; and assaying for a signal produced by cleavage of the detector nucleic acid to determine a presence of the target nucleic acid.

In some aspects, the forward primer comprises at least 2, at least 3, at least 4, at least 5, at least 6, at least, 7, at least 8, at least 9, or at least 10 phosphorothioated nucleotides at the 5′ end.

In some aspects, the exonuclease is T7 exonuclease. In some aspects, the exonuclease is from 1 U per 3 μl final volume to 1 U per 9 μl final volume in the contacting step.

In various aspects, the present disclosure provides a method of assaying for a target nucleic acid in a sample, the method comprising: selectively producing a target single stranded DNA (ssDNA) by isothermal amplification of the target nucleic acid of the sample with a forward primer and a reverse primer, wherein the forward primer is added in excess of the reverse primer or the reverse primer is added in excess of the forward primer; and contacting the sample to: a guide nucleic acid that hybridizes to a segment of the target ssDNA; a detector nucleic acid; and a programmable nuclease that cleaves the detector nucleic acid upon hybridization of the guide nucleic acid to the segment of the target ssDNA; and assaying for a signal produced by cleavage of the detector nucleic acid to determine a presence of the target nucleic acid.

In some aspects, the isothermal amplification is selected from the group consisting of isothermal recombinase polymerase amplification (RPA), transcription mediated amplification (TMA), strand displacement amplification (SDA), helicase dependent amplification (HDA), loop mediated amplification (LAMP), rolling circle amplification (RCA), single primer isothermal amplification (SPIA), ligase chain reaction (LCR), simple method amplifying RNA targets (SMART), improved multiple displacement amplification (IMDA), and nucleic acid sequence-based amplification (NASBA). In some aspects, the forward primer is added in an excess of 30:1, 40:1, 50:1, 60:1, or 70:1 over the reverse primer. In some aspects, the reverse primer is added in an excess of 30:1, 40:1, 50: 1, 60:1, or 70:1 over the forward primer.

In various aspects, the present disclosure provides a method of assaying for a target nucleic acid in a sample, the method comprising: selectively producing a target single stranded DNA (ssDNA) by amplifying the target nucleic acid lacking a PAM sequence with: a strand displacing polymerase; and an outer forward primer, an inner forward primer, and a reverse primer or an outer reverse primer, an inner reverse primer, and a forward primer; and contacting the target ssDNA to: a guide nucleic acid that hybridizes to a segment of the target ssDNA; a detector nucleic acid; and a programmable nuclease that cleaves the detector nucleic acid upon hybridization of the guide nucleic acid to the segment of the target ssDNA; and assaying for a signal produced by cleavage of the detector nucleic acid to determine a presence of the target nucleic acid.

In some aspects, the selectively producing, the contacting, and the assaying are performed in a common reaction volume. In some aspects, the target nucleic acid sequence comprises cDNA, ssDNA, dsDNA, or RNA. In some aspects, the target nucleic acid sequence is RNA and wherein the method further comprises reverse transcribing the RNA prior to the selectively producing. In some aspects, the programmable nuclease is a Type V programmable nuclease. In some aspects, the Type V programmable nuclease is a Cas12 protein or a Cas14 protein. In further aspects, the Cas12 protein is Cas12a, Cas12b, Cas12c, Cas12d, or Cas12e. In some aspects, the Cas14 protein is Cas14a, Cas14b, Cas14c, Cas14d, Cas14e, Cas14f, Cas14g, Cas14h, Cas14i, Cas14j, or Cas14k. In some aspects, cleaving by the programmable nuclease comprises PAM-independent cleavage.

In some aspects, the detector nucleic acid comprises a nucleic acid comprising at least two nucleotides, a fluorophore, and a fluorescence quencher, wherein the fluorophore and the fluorescence quencher are linked by the nucleic acid. In some aspects, the target nucleic acid comprises a sequence encoding a single nucleotide polymorphism (SNP).

In some aspects, the target nucleic acid comprises a sequence encoding a wild type sequence. In some aspects, the sample comprises blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue.

In various aspects, the present disclosure provides a programmable nuclease for use in diagnosis, wherein the programmable nuclease detects the modification according to any of the method described above.

In various aspects, the present disclosure provides a programmable nuclease for use in diagnosis, wherein the programmable nuclease detects the target nucleic acid according to any of the methods described above.

In various aspects, the present disclosure provides a programmable nuclease for use in diagnosis, wherein the programmable nuclease detects the SNP according to any of the methods described above.

In some aspects, the use of a programmable nuclease to assay for a modification state is according to any of the above described methods. In some aspects, the use of a programmable nuclease to assay for a target nucleic acid in a sample is according to any of the methods described above.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a 2% agarose gel confirming restriction digestion of unmethylated or methylated pUC19 with Thermo Scientific EpiJET DNA Methylation Analysis kit. Lane 1: Unmethylated pUC19+no enzyme. Lane 2: Unmethylated pUC19+Epi HpaII. Lane 3: Unmethylated pUC19+Epi Mpsl. Lane 4: Methylated pUC19+no enzyme. Lane 5: Methylated pUC19+Epi HpaII. Lane 6: Methylated pUC19+Epi Mpsl.

FIG. 2A depicts detection of Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) without intermediate amplification.

FIG. 2B depicts detection of Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) after 10 cycles of PCR amplification.

FIG. 2C depicts detection of Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) after 25 cycles of PCR amplification.

FIG. 3A depicts detection of helicase-dependent isothermal amplified Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) after 30 minutes of incubation.

FIG. 3B depicts detection of helicase-dependent isothermal amplified Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) after 60 minutes of incubation.

FIG. 4A depicts a schematic of various positions of adenosines (A) in target RNAs (SEQ ID NO: 141-SEQ ID NO: 145, respectively, in order of appearance), wherein each target RNA contains identical surrounding sequence context (N). The adenosines can either be unmodified or modified (N6-methyladenosine).

FIG. 4B depicts the normalized fluorescence readings from Cas13a detection assay using a LbuCas13a programmable nuclease (SEQ ID NO: 124) with unmodified adenosine or modified adenosine (N6-methyladenosine) target RNAs of FIG. 4A.

FIG. 4C depicts raw fluorescent results of four different crRNAs using a LbuCas13a programmable nuclease (SEQ ID NO: 124) along either an unmodified or modified (N6-methyladenosine) target RNA derived from a natural sequence.

FIG. 5A depicts 10 nM DNA amplified by HDA with standard dNTPs (A/G/C/T) and with a dA/G/C/UTP mix (no thymines). LbCas12a (SEQ ID NO: 21) detection of 2 μL of these reactions is shown alongside a no amplification control, demonstrating that Cas12a can detect uracil-containing amplicons at a rate similar to that of thymine-containing amplicons.

FIG. 5B shows the sequences of the crRNA (pUC19 Cas12a gRNA, GGGTAATTTCTACTAAGTGTAGATTGCTTCCGGCTCGTATGTTG, SEQ ID NO: 146), forward and reverse HDA/PCR primers (SEQ ID NO: 18 and SEQ ID NO: 19, respectively, in order of appearance), and the pUC19 amplicon (SEQ ID NO: 20) used in FIG. 2, FIG. 3, and FIG. 5A.

FIG. 6A illustrates a schematic outlining amplification with a phosphorothioated (PT′d) primer followed by treatment with a T7 exonuclease to generate ssDNA amplicons from ssDNA, dsDNA, or RNA.

FIG. 6B illustrates a schematic exemplifying ssDNA amplification with an asymmetric concentration of primers.

FIG. 6C illustrates a schematic demonstrating ssDNA amplification with a strand displacing polymerase and nested forward primers.

FIG. 7A-FIG. 7C illustrates gel electrophoresis of amplified products following treatment by T7 exonuclease in various conditions to assess a one-pot amplification and T7 digestion process performed in a common reaction volume.

FIG. 7A illustrates that T7 exonuclease activity is inhibited when added directly to unpurified PCR products (lanes 3, 4), but the T7 exonuclease remains active when used with PCR products purified using Zymo DNA Clean and Concentrator kit followed by treatment with 10 U of T7 exonuclease and yields primarily ssDNA amplicons (lanes 7, 8).

FIG. 7B illustrates that treatment of unpurified PCR products with 20 U of T7 exonuclease does not yield ssDNA activator (lane 7), and that addition of 5 μL of 10× Cutsmart buffer to 50 μL unpurified PCR product followed by addition of T7 exonuclease also does not yield ssDNA activator (lane 3, 4)

FIG. 7C illustrates the titration of the amount of T7 exonuclease and NEB Cutsmart buffer added to unpurified PCR product to optimize a one-pot amplification/degradation step performed in a common reaction volume. Following FIG. 7A-FIG. 7C, a simultaneous degradation/detection step was optimized as opposed to a one-pot amplification/degradation step performed in a common reaction volume.

FIG. 8A-FIG. 8B illustrate various amounts of NEB T7 exonuclease added to a 20 DETECTR reaction for achieving a viable fluorescent signal.

FIG. 8A illustrates the minimum amount of NEB T7 exonuclease added to a 20 DETECTR reaction required to achieve a viable fluorescent signal with LbCas12a (SEQ ID NO: 21) DETECTR.

FIG. 8B illustrates the minimum amount of NEB T7 exonuclease added to a 20 DETECTR reaction required to achieve a viable fluorescent signal with Cas14a1 (SEQ ID NO: 33) DETECTR.

FIG. 9 illustrates SNP ssDNA detection using Cas14a-DETECTR with a blue-eye targeting guide for saliva samples from blue-eyed and brown-eyed individuals compared with ssDNA detection using Cas12a. Amplification of the HERC2 gene from human genomic DNA was conducted with a PT′d primer followed by T7 exonuclease treatment, enabling Cas14a1 (SEQ ID NO: 33) detection of an originally dsDNA target and PAM-independent detection by LbCas12a (SEQ ID NO: 21).

FIG. 10 illustrates gel electrophoresis of helicase-dependent amplification (HDA) products demonstrating that HDA prefers amplicons less than 120 bp (lane 3, 4), and that HDA tolerates amplification with PT′d primers making it a compatible amplification platform for use with the PT′d primer/exonuclease ssDNA amplification strategy.

FIG. 11 illustrates Cas14a1 (SEQ ID NO: 33) DETECTR detection of ssDNA amplicons generated by HDA with a PT′d primer followed by treatment with an exonuclease compared to Cas14a1 DETECTR detection of ssDNA oligonucleotides without HDA.

FIG. 12 illustrates LbCas12a (SEQ ID NO: 21) DETECTR detection of HDA amplified M13 ssDNA plasmid following treatment with a T7 exonuclease compared to detection of M13 ssDNA without HDA.

FIG. 13 illustrates gel electrophoresis of amplicons generated by PCR with an asymmetric concentration of primers, demonstrating the effect of varying primer ratios and starting DNA concentration to maximize ssDNA amplification.

FIG. 14A-FIG. 14B illustrate Cas14a1 (SEQ ID NO: 33) DETECTR assays on PCR amplified oligonucleotides. NTS (non-target strand) ssDNA oligonucleotides were the template for the PCR reaction, and TS (target strand) ssDNA amplicons were generated by an asymmetric concentration of primers.

FIG. 14A illustrates the effect of wide range of forward:reverse primer concentration ratios.

FIG. 14B illustrates refining forward:reverse primer concentration ratio to optimize ssDNA amplification. ssDNA amplification is maximal when the forward primer is in 50:1 excess over the reverse primer.

FIG. 15A illustrates Cas12a (SEQ ID NO: 21) trans cleavage of phosphorothioated (PT) FQ Reporter (8 nt+PT) or unmodified FQ Reporter (8 nt−PT) in the presence of unmodified target ssDNA. Full phosphorothioation of the FQ reporter appears to inhibit trans cleavage by SEQ ID NO: 21. FQ Reporter=ssDNA fluorescence-quenching (FQ) reporter molecule

FIG. 15B illustrates Cas14a1 (SEQ ID NO: 33) trans cleavage of phosphorothioated FQ Reporter (12 nt+PT) or unmodified FQ Reporter (12 nt−PT) in the presence of unmodified target ssDNA. Full phosphorothioation of the FQ reporter appears to inhibit trans cleavage by SEQ ID NO: 33. FQ Reporter=ssDNA fluorescence-quenching (FQ) reporter molecule

### DETAILED DESCRIPTION

Disclosed herein are methods of assaying for and detecting a modification state of a segment of a target nucleic acid. The modification state of a segment of a target nucleic acid can be modified or an unmodified. For example, a modification state can be the presence (modified) or absence (unmodified) of any modification disclosed herein on a nucleic acid base. The segment of the target nucleic acid can be a region of bases. Assaying for the modification state can be detection of at least one or more than one bases comprising the modification, indicating the segment of the target nucleic acid is modified. Assaying for the modification state can be detection of at least one or more bases comprising the unmodified nucleic acids, indicating the segment of the target nucleic acid is unmodified. The particular methods disclosed herein, using programmable nucleases, can be tailored to sensitively and specifically assay for the modification state (modified or unmodified). Disclosed herein are methods of detecting a nucleic acid modification. Modified nucleic acids can be modified DNA or modified RNA. Nucleic acid modifications can comprise any functionally relevant changes to genomic expression that do not involve altering the nucleic acid sequence. Nucleic acids can be modified by acetylation of a base. Nucleic acids can be modified by methylation or deamination. For example, a DNA modification is methylation and an RNA modification is methylation. Modified nucleic acids (e.g., methylated or deaminated nucleic acids) may alter genomic expression, for example, by altering interactions of the nucleic acid with histones, thereby altering the chromatin state of the nucleic acid. Detection of nucleic acids with modifications can be used to diagnose or identify diseases associated with modifications (e.g., methylation) of target nucleic acid sequences. The methods described herein use a programmable nuclease, such as the CRISPR/Cas system, to detect modified nucleic acids. For example, a method of detection comprises contacting a nucleic acid modification sensitive programmable nuclease to a sample comprising a modified nucleic acid. A method of detection can comprise contacting a sample comprising a modified nucleic acid to a nucleic acid modification sensitive programmable nuclease composition comprising a programmable nuclease, wherein the programmable nuclease exhibits nucleic acid modification sensitive cleavage. A method of detection can comprise contacting a sample comprising a modified nucleic acid to a reagent that differentially reacts to modified nucleic acid bases and to a programmable nuclease. As a further example, a method of detection comprises contacting a methylation sensitive CRISPR enzyme to a sample comprising a methylated nucleic acid. A method of detection can comprise contacting a sample comprising a methylated nucleic acid to an enzyme composition comprising a CRISPR enzyme, wherein the enzyme composition exhibits methylation sensitive cleavage. A method of detection can comprise contacting a sample comprising a methylated nucleic acid to a reagent that differentially reacts to methylated bases and to a CRISPR enzyme.

A modification state can be a modified state when the segment of the target nucleic acid comprises a modified nucleic acid. A modified nucleic acid can comprise a nucleic acid with an epigenetic modification. An epigenetic modification may comprise methylation. A modified nucleic acid can comprise nucleic acid that is modified to induce a chromatin state. In some embodiments, a nucleic acid that is modified to induce a chromatin state may alter an interaction between the nucleic acid and a histone. In some embodiments, an altered interaction between the nucleic acid and the histone may have downstream epigenetic effects. For example, the nucleic acid in the induced chromatin state may have increased or decreased accessibility for polymerases, thereby increasing or decreasing transcription of the nucleic acid. A modified nucleic acid can be an adenosine-to-inosine (A-to-I) edited nucleic acid. Nucleic acids can be modified by methylation. Nucleic acids can be modified by acetylation. A nucleic acid modification can be 5-hydroxymethylcytidine or hydroxymethyl deoxycytidine in DNA, 5-formylcytidine, 5-carboxylcytidine, 5 -hydroxymethyluridine, 5-methyl cytidine, 3-methylcytidine, N6-methyladenosine, N6, 2′-O-dimethyladenine, N1-methyladenine, N1-methylguanine, 5-methylcytidine in RNA, or 5-hydroxymethylcytidine in RNA. A modified nucleic acid (e.g., a modified ribonucleic acid or a modified deoxyribonucleic acid) may comprise a modified nitrogenous base. A modified nitrogenous base can be an adenine to hypoxanthine edited nitrogenous base. Nucleic acids may be modified by methylation of the nitrogenous base. A modified nitrogenous base may be 5-hydroxymethylcytosine (5hmC), 5-formylcytosine (5fC), 5-carboxylcytosine (5caC), 5-hydroxymethyluracil (5hmU), 5-methylcytosine (5mC), 3-methylcytosine (3mC), N6-methyladenine (m6A), N6, 2′-O-dimethyl adenine (m6Am), N1-methyladenine (m1A), N1-methylguanine (m1G), 5-methylcytosine (m5C) in RNA, or 5-hydroxymethylcytosine (hm5C).

DNA modifications, such as DNA methylation, are involved in genomic imprinting, reprogramming, genomic stability, cellular differentiation, X-chromosome inactivation, transposon silencing, RNA splicing, and DNA repair. DNA methylation has also been associated with aging and carcinogenesis. DNA methylation occurs at approximately 70%-80% of CpG dinucleotides. Methylation of CpG sites in the human genome can stably silence gene expression. Hypermethylation of CpG islands, which are stretches of DNA with a higher frequency of CG sequences than other regions, in promoter regions for tumor-suppressor genes is common in several types of cancer, such as colon cancers, bladder cancers, and stomach cancers. Due to the high frequency of CpG methylation in specific promoter sequences, a nucleic acid based diagnostic test that is also sensitive to DNA methylation can enable simple, reliable early detection of many cancer types and other CpG methylation-related diseases.

RNA modifications, such as methylation, impact RNA structure, RNA function, and the ability of proteins to bind RNA. N6-methyladenosine (e.g., comprising an N6-methyladenine (m6A) nitrogenous base) is a common RNA modification in messenger RNAs (mRNAs). m6A-modifications are generally found near start of the 3′ untranslated region (3′UTR) of mRNAs and at canonical DRACH motifs (D=A, G, or U; R=G or A; A=A; C═C; H=A, C, or U). One of the primary functions of m6A is to mark mRNAs for degradation. m6A-reader proteins, such as YTHDF2, can bind m6A and recruit deadenylases to initiate transcript destabilization. The identification of m6A-writer, -reader, and -eraser proteins suggests that m6A is a dynamic RNA modification that regulates post-transcriptional gene expression. Deregulation of m6A-pathway genes has been implicated in variety of cancers, including breast cancer, non-small-cell lung cancer, and acute myeloid leukemia. Increases or decreases in m6A-levels transcriptome-wide leads to aberrant gene expression and the potential activation of oncogenes.

Despite the importance of m6A and m6A-pathway genes, the presence of m6A on specific oncogenic targets is difficult to assess. Current approaches to determine m6A levels and other RNA modification levels on nucleic acids encoding specific genes require time-consuming, radioactive methods, such as SCARLET. Other approaches, such as miCLIP, require complicated next-generation sequencing library preparations and bioinformatic analyses. Overall, these approaches are not suitable to rapidly examine the m6A-methylation state on a single gene.

The methods described herein can rapidly and specifically determine if a nucleic acid is modified. The methods described herein can therefore be used in a nucleic acid based diagnostic test that can be used for simple, reliable detection of many cancer types and other diseases marked by the modification state (e.g., presence or absence of nucleic acid modifications on nucleic acids in a segment of a target nucleic acid) comprising a target sequence. The methods described herein can also be used in a nucleic acid based diagnostic research tool for simple, reliable detection of a target nucleic acid in laboratory research or field research. Additionally, the methods described herein can be used in a nucleic acid based agricultural diagnostic test for simple, reliable detection of a target nucleic acid.

The present disclosure further provides methods and compositions, which enable ssDNA amplification for detection by programmable nuclease platforms, such as the DNA Endonuclease Targeted CRISPR TransReporter (DETECTR) platform. ssDNA amplification methods that are compatible with the DETECTR technology were developed to enable ssDNA-activated programmable nucleases (e.g., CRISPR/Cas effector proteins like Cas14a1), which can function as viable effector proteins for detection of nucleic acids from biological samples. Moreover, amplification of ssDNA instead of dsDNA enables PAM-independent detection of nucleic acids by proteins with PAM requirements for dsDNA-activated trans cleavage such as LbCas12a. A ssDNA may be selectively amplified by amplifying ssDNA in a sample. A ssDNA may be selectively amplified by amplifying ssDNA in a sample comprising both ssDNA and dsDNA. A ssDNA may be selectively produced by amplifying ssDNA in a sample. A ssDNA may be selectively produced by amplifying ssDNA in a sample comprising both ssDNA and dsDNA. Selectively producing an ssDNA can comprise adding amplification reagents to a sample that target a dsDNA or ssDNA in the sample and selectively amplify a target ssDNA segment. This can be achieved through the amplification strategies described herein including, for example, the use of phosphorothioated (PT′d) primers with an exonuclease that specifically degrades non-PT′d amplicons, the use of asymmetric ratios of forward to reverse primers to drive amplification of thetarget ssDNA, or the use of strand displacement amplification with a set of primers and a strand displacing polymerase.

Certain programmable nucleases (e.g., Cas14a1) exhibit ssDNA-activated indiscriminate trans cleavage of ssDNA, enabling their use for detection of DNA in samples. However, for these programmable nucleases to have realistic applications in nucleic acid detection, ssDNA activators must be generated from many nucleic acid templates (RNA, ss/dsDNA) in order to achieve cleavage of the ssDNA fluorescence-quenching (FQ) reporter molecule in the DETECTR platform. Current amplification strategies focus on dsDNA, and not ssDNA. Thus, there is a need to develop methods that enable full compatibility for ssDNA activated programmable nucleases (e.g., Cas14a1). Furthermore, a DETECTR-compatible amplification strategy of ssDNA would alleviate the PAM requirement for PAM-dependent programmable nucleases (e.g., LbCas12a).

DNA Endonuclease Targeted CRISPR TransReporter (DETECTR) utilizes the trans cleavage abilities of some programmable nucleases (e.g., CRISPR-Cas effector proteins) to achieve fast and high-fidelity detection of DNA samples. Following DNA extraction from a biological sample and an amplification step, crRNA that is complementary to the target DNA sequence of interest binds to the target DNA, initiating indiscriminate ssDNase activity by the effector protein. The protein can then cleave an ssDNA fluorescence-quenching (FQ) reporter molecule, providing a fluorescent readout of target DNA detection.

Certain programmable nucleases (e.g., Cas14a1) are activated by ssDNA, upon which they can exhibit trans cleavage of ssDNA and can, thereby, be used to cleave ssDNA FQ reporter molecules in the DETECTR system. However, these programmable nucleases would need ssDNA to be present in the sample, or generated and/or amplified from any number of nucleic acid templates (RNA, ssDNA, or dsDNA). Provided herein are compositions and methods for generation and amplification of ssDNA from various nucleic acid templates, including RNA, ssDNA, and dsDNA.

LbCas12a can display both ssDNA and dsDNA-activated indiscriminate trans cleavage of ssDNA. The current DETECTR platform with LbCas12a relies on dsDNA activation of trans cleavage (as a result of the above-mentioned dsDNA-generating amplification techniques). One caveat to dsDNA-activation of trans cleavage by LbCas12a is the requirement of a protospacer adjacent motif (PAM), a TTTN sequence immediately flanking the 5′ end of the protospacer on the non-target strand. ssDNA-activated trans cleavage by LbCas12a does not require a PAM, thus a DETECTR-compatible amplification strategy of ssDNA instead of dsDNA would alleviate this PAM requirement and expand the range of detectable nucleic acid sequences by LbCas12a and other PAM-dependent effector proteins.

The present disclosure provides methods of ssDNA amplification from a variety of nucleic acid inputs (e.g., RNA, ssDNA, and dsDNA) to enable nucleic acid detection by ssDNA-activated transcleaving proteins and to free dsDNA-activated transcleaving proteins from PAM requirements. The present disclosure provides three non-limiting, exemplary methods of ssDNA amplification: amplification with one phosphorothioated (which can be referred to as “PT′d”) primer followed by treatment with a T7 exonuclease, amplification with an asymmetric concentration of primers, and amplification with a strand displacing polymerase.

### Programmable Nucleases

Described herein are reagents comprising a programmable nuclease capable of being activated when complexed with the guide nucleic acid and the target nucleic acid sequence. Target nucleic acid sequence may be ssDNA, dsDNA, ssRNA, or dsRNA. In some embodiments, the target nucleic acid sequence may be a modified nucleic acid sequence, for example a modified DNA or a modified RNA. Methods disclosed herein using programmable nucleases (e.g., CRISPR Cas systems) assay for assaying for a modification state of a segment of a target nucleic acid. The modification state of a segment of a target nucleic acid can be modified or an unmodified. For example, a modification state can be the presence (modified) or absence (unmodified) of any modification disclosed herein on a nucleic acid base. The segment of the target nucleic acid can be a region of bases. Assaying for the modification state can be detection of at least one or more than one bases comprising the modification, indicating the segment of the target nucleic acid is modified. Assaying for the modification state can be detection of at least one or more bases comprising the unmodified nucleic acids, indicating the segment of the target nucleic acid is unmodified. The particular methods disclosed herein, using programmable nucleases, can be tailored to sensitively and specifically assay for the modification state (modified or unmodified). In some embodiments, the target nucleic acid sequence may be an amplified ssDNA. The programmable nuclease can be activated upon binding of the guide nucleic acid to its target nucleic acid and non-specifically degrades nucleic acids in its environment. The programmable nuclease, thus, exhibits collateral cleavage (trans cleavage) activity once activated. A programmable nuclease can be a Cas protein. A guide nucleic acid, sometimes referred to as a crRNA, and Cas protein can form a CRISPR enzyme. Sometimes, the programmable nuclease is a type V CRISPR-Cas nuclease. In some embodiments, the programmable nuclease is a type VI CRISPR-Cas nuclease. The programmable nuclease can be activated upon binding of the guide nucleic acid to its target ssDNA nucleic acid and non-specifically degrades (trans cleavage activity) nucleic acid (e.g., an ssDNA FQ reporter molecule) in its environment. The programmable nuclease can have trans cleavage activity once activated.

Sometimes, the programmable nuclease is a Cas protein (also referred to as a Cas nuclease herein). In some embodiments, the programmable nuclease is any DNA guided nuclease. In some embodiments, the programmable nuclease is any RNA guided nuclease. In some embodiments, the programmable nuclease is any guided DNA nuclease. Sometimes, the programmable nuclease can be a type V CRISPR-Cas nuclease. In some embodiments, the Type V CRISPR/Cas nuclease is a programmable Cas12 nuclease. Type V CRISPR/Cas enzymes (e.g., Cas12 or Cas14) lack an HNH domain. A Cas12 nuclease of the present disclosure cleaves a nucleic acid via a single catalytic RuvC domain. This single catalytic RuvC domain includes 3 partial RuvC domains (RuvC-I, RuvC-II, and RuvC-III, also referred to herein as subdomains) that are not contiguous with respect to the primary amino acid sequence of the Cas12 protein, but form an RuvC domain once the protein is produced and folds. The RuvC domain is within a nuclease, or “NUC” lobe of the protein, and the Cas12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain. (Murugan et al., Mol Cell. 2017 Oct. 5; 68(1): 15-25). Alternatively, the Type V CRISPR/Cas enzyme is a programmable Cas14 nuclease. A Cas14 protein of the present disclosure includes 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds. In some cases, the programmable nuclease can be Mad7 or Mad2. In some cases, the programmable nuclease can be Cas12. Sometimes the Cas12 can be Cas12a, Cas12b, Cas12c, Cas12d, or Cas12e. The programmable nuclease can be Mad7 or Mad2. In some cases, the programmable nuclease can be Csm1, Cas9, C2c4, C2c8, C2c5, C2c10, C2c9, or CasZ. Sometimes, the Csml can be also called smCmsl, miCmsl, obCmsl, or suCmsl. Sometimes CasZ can be also called Cas14a, Cas14b, Cas14c, Cas14d, Cas14e, Cas14f, Cas14g, or Cas14h. In some embodiments, the programmable nuclease is any guided RNA nuclease. In some cases, the programmable nuclease can be a type VI CRISPR-Cas nuclease. For example, a type VI CRISPR-Cas nuclease can be a Cas13 nuclease. The general architecture of a Cas13 protein includes an N-terminal domain and two HEPN (higher eukaryotes and prokaryotes nucleotide-binding) domains separated by two helical domains (Liu et al., Cell 2017 Jan. 12; 168(1-2):121-134.e12). The HEPN domains each comprise aR-X4-H motif. Shared features across Cas13 proteins include that upon binding of the crRNA of the guide nucleic acid to a target nucleic acid, the protein undergoes a conformational change to bring together the HEPN domains and form a catalytically active RNase. (Tambe et al., Cell Rep. 2018 Jul .24; 24(4): 1025-1036.). Thus, two activatable HEPN domains are characteristic of a programmable Cas13 nuclease of the present disclosure. However, programmable Cas13 nucleases also consistent with the present disclosure include Cas13 nucleases comprising mutations in the HEPN domain that enhance the Cas13 proteins cleavage efficiency or mutations that catalytically inactivate the HEPN domains. The Cas13 nuclease can be Cas13a, Cas13b, Cas13c, Cas13d, or Cas13e. Sometimes the programmable nuclease can be a type III CRISPR-Cas system. In some cases, the programmable nuclease can be from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pint), Capnocytophaga canimorsus (Cca), Porphyromonas gulae (Pgu), Prevotella sp. (Psp), Porphyromonas gingivalis (Pig), Prevotella intermedia (Pini), Enterococcus italicus (E1), Lactobacillus salivarius (Ls), or Thermus thermophilus (Tt).

In some cases, a suitable Cas12 programmable nuclease comprises an amino acid sequence having at least 80%, at least 85%, at least 90%, at least 95%, at least 98%, at least 99%, or 100%, amino acid sequence identity to any one of SEQ ID NO: 21-SEQ ID NO: 30 or SEQ ID NO: 147-SEQ ID NO: SEQ ID NO: 179. In some embodiments a suitable Cas12 programmable nuclease comprises an amino acid sequence having at least 80%, at least 85%, at least 90%, at least 95%, at least 98%, at least 99%, or 100%, amino acid sequence identity to SEQ ID NO: 147.

TABLE 1
Cas12 Sequences

| SEQ ID NO | Description | Sequence |
|---|---|---|
| SEQ ID NO: 21 | Lachnospiraceae | MSKLEKFTNCYSLSKTLRFKAIPVGKTQENIDNKRLLVEDEKRAEDYKG |
| | bacterium | VKKLLDRYYLSFINDVLHSIKLKNLNNYISLFRKKTRTEKENKELENLE |
| | ND2006 | INLRKEIAKAFKGNEGYKSLFKKDIIETILPEFLDDKDEIALVNSFNGF |
| | (LbCas12a) | TTAFTGFFDNRENMFSEEAKSTSIAFRCINENLTRYISNMDIFEKVDAI |
| | | FDKHEVQEIKEKILNSDYDVEDFFEGEFFNFVLTQEGIDVYNAIIGGFV |
| | | TESGEKIKGLNEYINLYNQKTKQKLPKFKPLYKQVLSDRESLSFYGEGY |
| | | TSDEEVLEVFRNTLNKNSEIFSSIKKLEKLFKNFDEYSSAGIFVKNGPA |
| | | ISTISKDIFGEWNVIRDKWNAEYDDIHLKKKAVVTEKYEDDRRKSFKKI |
| | | GSFSLEQLQEYADADLSVVEKLKEIIIQKVDEIYKVYGSSEKLFDADFV |
| | | LEKSLKKNDAVVAIMKDLLDSVKSFENYIKAFFGEGKETNRDESFYGDF |
| | | VLAYDILLKVDHIYDAIRNYVTQKPYSKDKFKLYFQNPQFMGGWDKDKE |
| | | TDYRATILRYGSKYYLAIMDKKYAKCLQKIDKDDVNGNYEKINYKLLPG |
| | | PNKMLPKVFFSKKWMAYYNPSEDIQKIYKNGTFKKGDMFNLNDCHKLID |
| | | FFKDSISRYPKWSNAYDFNFSETEKYKDIAGFYREVEEQGYKVSFESAS |
| | | KKEVDKLVEEGKLYMFQIYNKDFSDKSHGTPNLHTMYFKLLFDENNHGQ |
| | | IRLSGGAELFMRRASLKKEELVVHPANSPIANKNPDNPKKTTTLSYDVY |
| | | KDKRFSEDQYELHIPIAINKCPKNIFKINTEVRVLLKHDDNPYVIGIDR |
| | | GERNLLYIVVVDGKGNIVEQYSLNEIINNFNGIRIKTDYHSLLDKKEKE |
| | | RFEARQNWTSIENIKELKAGYISQVVHKICELVEKYDAVIALEDLNSGF |
| | | KNSRVKVEKQVYQKFEKMLIDKLNYMVDKKSNPCATGGALKGYQITNKF |
| | | ESFKSMSTQNGFIFYIPAWLTSKIDPSTGFVNLLKTKYTSIADSKKFIS |
| | | SFDRIMYVPEEDLFEFALDYKNFSRTDADYIKKWKLYSYGNRIRIFRNP |
| | | KKNNVFDWEEVCLTSAYKELFNKYGINYQQGDIRALLCEQSDKAFYSSF |
| | | MALMSLMLQMRNSITGRTDVDFLISPVKNSDGIFYDSRNYEAQENAILP |
| | | KNADANGAYNIARKVLWAIGQFKKAEDEKLDKVKIAISNKEWLEYAQTS |
| | | VKH |
| SEQ ID NO: 22 | Acidaminococcus sp. | MTQFEGFTNLYQVSKTLRFELIPQGKTLKHIQEQGFIEEDKARNDHYKE |
| | BV316 | LKPIIDRIYKTYADQCLQLVQLDWENLSAAIDSYRKEKTEETRNALIEE |
| | (AsCas12a) | QATYRNAIHDYFIGRTDNLTDAINKRHAEIYKGLFKAELFNGKVLKQLG |
| | | TVTTTEHENALLRSFDKFTTYFSGFYENRKNVFSAEDISTAIPHRIVQD |
| | | NFPKFKENCHIFTRLITAVPSLREHFENVKKAIGIFVSTSIEEVFSFPF |
| | | YNQLLTQTQIDLYNQLLGGISREAGTEKIKGLNEVLNLAIQKNDETAHI |
| | | IASLPHRFIPLFKQILSDRNTLSFILEEFKSDEEVIQSFCKYKTLLRNE |
| | | NVLETAEALFNELNSIDLTHIFISHKKLETISSALCDHWDTLRNALYER |
| | | RISELTGKITKSAKEKVQRSLKHEDINLQEIISAAGKELSEAFKQKTSE |
| | | ILSHAHAALDQPLPTTLKKQEEKEILKSQLDSLLGLYHLLDWFAVDESN |
| | | EVDPEFSARLTGIKLEMEPSLSFYNKARNYATKKPYSVEKFKLNFQMPT |
| | | LASGWDVNKEKNNGAILFVKNGLYYLGIMPKQKGRYKALSFEPTEKTSE |
| | | GFDKMYYDYFPDAAKMIPKCSTQLKAVTAHFQTHTTPILLSNNFIEPLE |
| | | ITKEIYDLNNPEKEPKKFQTAYAKKTGDQKGYREALCKWIDFTRDFLSK |
| | | YTKTTSIDLSSLRPSSQYKDLGEYYAELNPLLYHISFQRIAEKEIMDAV |
| | | ETGKLYLFQIYNKDFAKGHHGKPNLHTLYWTGLFSPENLAKTSIKLNGQ |
| | | AELFYRPKSRMKRMAHRLGEKMLNKKLKDQKTPIPDTLYQELYDYVNHR |
| | | LSHDLSDEARALLPNVITKEVSHEIIKDRRFTSDKFFFHVPITLNYQAA |
| | | NSPSKFNQRVNAYLKEHPETPIIGIDRGERNLIYITVIDSTGKILEQRS |
| | | LNTIQQFDYQKKLDNREKERVAARQAWSVVGTIKDLKQGYLSQVIHEIV |
| | | DLMIHYQAVVVLENLNFGFKSKRTGIAEKAVYQQFEKMLIDKLNCLVLK |
| | | DYPAEKVGGVLNPYQLTDQFTSFAKMGTQSGFLFYVPAPYTSKIDPLTG |
| | | FVDPFVWKTIKNHESRKHFLEGFDFLHYDVKTGDFILHFKMNRNLSFQR |
| | | GLPGFMPAWDIVFEKNETQFDAKGTPFIAGKRIVPVIENHRFTGRYRDL |
| | | YPANELIALLEEKGIVFRDGSNILPKLLENDDSHAIDTMVALIRSVLQM |
| | | RNSNAATGEDYINSPVRDLNGVCFDSRFQNPEWPMDADANGAYHIALKG |
| | | QLLLNHLKESKDLKLQNGISNQDWLAYIQELRN |
| SEQ ID NO: 23 | Francisella | MSIYQEFVNKYSLSKTLRFELIPQGKTLENIKARGLILDDEKRAKDYKK |
| | novicida | AKQIIDKYHQFFIEEILSSVCISEDLLQNYSDVYFKLKKSDDDNLQKDF |
| | U112 | KSAKDTIKKQISEYIKDSEKFKNLFNQNLIDAKKGQESDLILWLKQSKD |
| | (FnCas12a) | NGIELFKANSDITDIDEALEIIKSFKGWTTYFKGFHENRKNVYSSNDIP |
| | | TSIIYRIVDDNLPKFLENKAKYESLKDKAPEAINYEQIKKDLAEELTFD |
| | | IDYKTSEVNQRVFSLDEVFEIANFNNYLNQSGITKFNTIIGGKFVNGEN |
| | | TKRKGINEYINLYSQQINDKTLKKYKMSVLFKQILSDTESKSFVIDKLE |
| | | DDSDVVTTMQSFYEQIAAFKTVEEKSIKETLSLLFDDLKAQKLDLSKIY |
| | | FKNDKSLTDLSQQVFDDYSVIGTAVLEYITQQIAPKNLDNPSKKEQELI |
| | | AKKTEKAKYLSLETIKLALEEFNKHRDIDKQCRFEEILANFAAIPMIFD |
| | | EIAQNKDNLAQISIKYQNQGKKDLLQASAEDDVKAIKDLLDQTNNLLHK |
| | | LKIFHISQSEDKANILDKDEHFYLVFEECYFELANIVPLYNKIRNYITQ |
| | | KPYSDEKFKLNFENSTLANGWDKNKEPDNTAILFIKDDKYYLGVMNKKN |
| | | NKIFDDKAIKENKGEGYKKIVYKLLPGANKMLPKVFFSAKSIKFYNPSE |
| | | DILRIRNHSTHTKNGSPQKGYEKFEFNIEDCRKFIDFYKQSISKHPEWK |
| | | DFGFRFSDTQRYNSIDEFYREVENQGYKLTFENISESYIDSVVNQGKLY |
| | | LFQIYNKDFSAYSKGRPNLHTLYWKALFDERNLQDVVYKLNGEAELFYR |
| | | KQSIPKKITHPAKEAIANKNKDNPKKESVFEYDLIKDKRFTEDKFFFHC |
| | | PITINFKSSGANKFNDEINLLLKEKANDVHILSIDRGERHLAYYTLVDG |
| | | KGNIIKQDTFNIIGNDRMKTNYHDKLAAIEKDRDSARKDWKKINNIKEM |
| | | KEGYLSQVVHEIAKLVIEYNAIVVFEDLNFGFKRGRFKVEKQVYQKLEK |
| | | MLIEKLNYLVFKDNEFDKTGGVLRAYQLTAPFETFKKMGKQTGIIYYVP |
| | | AGFTSKICPVTGFVNQLYPKYESVSKSQEFFSKFDKICYNLDKGYFEFS |
| | | FDYKNFGDKAAKGKWTIASFGSRLINFRNSDKNHNWDTREVYPTKELEK |
| | | LLKDYSIEYGHGECIKAAICGESDKKFFAKLTSVLNTILQMRNSKTGTE |
| | | LDYLISPVADVNGNFFDSRQAPKNMPQDADANGAYHIGLKGLMLLGRIK |
| | | NNQEGKKLNLVIKNEEYFEFVQNRNN |
| SEQ ID NO: 24 | Porphyromonas | MKTQHFFEDFTSLYSLSKTIRFELKPIGKTLENIKKNGLIRRDEQRLDD |
| | macacae | YEKLKKVIDEYHEDFIANILSSFSFSEEILQSYIQNLSESEARAKIEKT |
| | (PmCas12a) | MRDTLAKAFSEDERYKSIFKKELVKKDIPVWCPAYKSLCKKFDNFTTSL |
| | | VPFHENRKNLYTSNEITASIPYRIVHVNLPKFIQNIEALCELQKKMGAD |
| | | LYLEMMENLRNVWPSFVKTPDDLCNLKTYNHLMVQSSISEYNRFVGGYS |
| | | TEDGTKHQGINEWINIYRQRNKEMRLPGLVFLHKQILAKVDSSSFISDT |
| | | LENDDQVFCVLRQFRKLFWNTVSSKEDDAASLKDLFCGLSGYDPEAIYV |
| | | SDAHLATISKNIFDRWNYISDAIRRKTEVLMPRKKESVERYAEKISKQI |
| | | KKRQSYSLAELDDLLAHYSEESLPAGFSLLSYFTSLGGQKYLVSDGEVI |
| | | LYEEGSNIWDEVLIAFRDLQVILDKDFTEKKLGKDEEAVSVIKKALDSA |
| | | LRLRKFFDLLSGTGAEIRRDSSFYALYTDRMDKLKGLLKMYDKVRNYLT |
| | | KKPYSIEKFKLHFDNPSLLSGWDKNKELNNLSVIFRQNGYYYLGIMTPK |
| | | GKNLFKTLPKLGAEEMFYEKMEYKQIAEPMLMLPKVFFPKKTKPAFAPD |
| | | QSVVDIYNKKTFKTGQKGFNKKDLYRLIDFYKEALTVHEWKLFNFSFSP |
| | | TEQYRNIGEFFDEVREQAYKVSMVNVPASYIDEAVENGKLYLFQIYNKD |
| | | FSPYSKGIPNLHTLYWKALFSEQNQSRVYKLCGGGELFYRKASLHMQDT |
| | | TVHPKGISIHKKNLNKKGETSLFNYDLVKDKRFTEDKFFFHVPISINYK |
| | | NKKITNVNQMVRDYIAQNDDLQIIGIDRGERNLLYISRIDTRGNLLEQF |
| | | SLNVIESDKGDLRTDYQKILGDREQERLRRRQEWKSIESIKDLKDGYMS |
| | | QVVHKICNMVVEHKAIVVLENLNLSFMKGRKKVEKSVYEKFERMLVDKL |
| | | NYLVVDKKNLSNEPGGLYAAYQLTNPLFSFEELHRYPQSGILFFVDPWN |
| | | TSLTDPSTGFVNLLGRINYTNVGDARKFFDRFNAIRYDGKGNILFDLDL |
| | | SRFDVRVETQRKLWTLTTFGSRIAKSKKSGKWMVERIENLSLCFLELFE |
| | | QFNIGYRVEKDLKKAILSQDRKEFYVRLIYLFNLMMQIRNSDGEEDYIL |
| | | SPALNEKNLQFDSRLIEAKDLPVDADANGAYNVARKGLMVVQRIKRGDH |
| | | ESIHRIGRAQWLRYVQEGIVE |
| SEQ ID NO: 25 | Moraxella bovoculi | MLFQDFTHLYPLSKTVRFELKPIDRTLEHIHAKNFLSQDETMADMHQKV |
| | 237 | KVILDDYHRDFIADMMGEVKLTKLAEFYDVYLKFRKNPKDDELQKQLKD |
| | (MbCas12a) | LQAVLRKEIVKPIGNGGKYKAGYDRLFGAKLFKDGKELGDLAKFVIAQE |
| | | GESSPKLAHLAHFEKFSTYFTGFHDNRKNMYSDEDKHTAIAYRLIHENL |
| | | PRFIDNLQILTTIKQKHSALYDQIINELTASGLDVSLASHLDGYHKLLT |
| | | QEGITAYNTLLGGISGEAGSPKIQGINELINSHHNQHCHKSERIAKLRP |
| | | LHKQILSDGMSVSFLPSKFADDSEMCQAVNEFYRHYADVFAKVQSLFDG |
| | | FDDHQKDGIYVEHKNLNELSKQAFGDFALLGRVLDGYYVDVVNPEFNER |
| | | FAKAKTDNAKAKLTKEKDKFIKGVHSLASLEQAIEHYTARHDDESVQAG |
| | | KLGQYFKHGLAGVDNPIQKIHNNHSTIKGFLERERPAGERALPKIKSGK |
| | | NPEMTQLRQLKELLDNALNVAHFAKLLTTKTTLDNQDGNFYGEFGVLYD |
| | | ELAKIPTLYNKVRDYLSQKPFSTEKYKLNFGNPTLLNGWDLNKEKDNFG |
| | | VILQKDGCYYLALLDKAHKKVFDNAPNTGKSIYQKMIYKYLEVRKQFPK |
| | | VFFSKEAIAINYHPSKELVEIKDKGRQRSDDERLKLYRFILECLKIHPK |
| | | YDKKFEGAIGDIQLFKKDKKGREVPISEKDLFDKINGIFSSKPKLEMED |
| | | FFIGEFKRYNPSQDLVDQYNIYKKIDSNDNRKKENFYNNHPKFKKDLVR |
| | | YYYESMCKHEEWEESFEFSKKLQDIGCYVDVNELFTEIETRRLNYKISF |
| | | CNINADYIDELVEQGQLYLFQIYNKDFSPKAHGKPNLHTLYFKALFSED |
| | | NLADPIYKLNGEAQIFYRKASLDMNETTIHRAGEVLENKNPDNPKKRQF |
| | | VYDIIKDKRYTQDKFMLHVPITMNFGVQGMTIKEFNKKVNQSIQQYDEV |
| | | NVIGIDRGERHLLYLTVINSKGEILEQCSLNDITTASANGTQMTTPYHK |
| | | ILDKREIERLNARVGWGEIETIKELKSGYLSHVVHQISQLMLKYNAIVV |
| | | LEDLNFGFKRGRFKVEKQIYQNFENALIKKLNHLVLKDKADDEIGSYKN |
| | | ALQLTNNFTDLKSIGKQTGFLFYVPAWNTSKIDPETGFVDLLKPRYENI |
| | | AQSQAFFGKFDKICYNADKDYFEFHIDYAKFTDKAKNSRQIWTICSHGD |
| | | KRYVYDKTANQNKGAAKGINVNDELKSLFARFIHINEKQPNLVMDICQN |
| | | NDKEFHKSLMYLLKTLLALRYSNASSDEDFILSPVANDEGVFFNSALAD |
| | | DTQPQNADANGAYHIALKGLWLLNELKNSDDLNKVKLAIDNQTWLNFAQ |
| | | NR |
| SEQ ID NO: 26 | Moraxella bovoculi | MGIHGVPAALFQDFTHLYPLSKTVRFELKPIGRTLEHIHAKNFLSQDET |
| | AAX08_00205 | MADMYQKVKVILDDYHRDFIADMMGEVKLTKLAEFYDVYLKFRKNPKDD |
| | (Mb2Cas12a) | GLQKQLKDLQAVLRKESVKPIGSGGKYKTGYDRLFGAKLFKDGKELGDL |
| | | AKFVIAQEGESSPKLAHLAHFEKFSTYFTGFHDNRKNMYSDEDKHTAIA |
| | | YRLIHENLPRFIDNLQILTTIKQKHSALYDQIINELTASGLDVSLASHL |
| | | DGYHKLLTQEGITAYNRIIGEVNGYTNKHNQICHKSERIAKLRPLHKQI |
| | | LSDGMGVSFLPSKFADDSEMCQAVNEFYRHYTDVFAKVQSLFDGFDDHQ |
| | | KDGIYVEHKNLNELSKQAFGDFALLGRVLDGYYVDVVNPEFNERFAKAK |
| | | TDNAKAKLTKEKDKFIKGVHSLASLEQAIEHHTARHDDESVQAGKLGQY |
| | | FKHGLAGVDNPIQKIHNNHSTIKGFLERERPAGERALPKIKSGKNPEMT |
| | | QLRQLKELLDNALNVAHFAKLLTTKTTLDNQDGNFYGEFGVLYDELAKI |
| | | PTLYNKVRDYLSQKPFSTEKYKLNFGNPTLLNGWDLNKEKDNFGVILQK |
| | | DGCYYLALLDKAHKKVFDNAPNTGKNVYQKMVYKLLPGPNKMLPKVFFA |
| | | KSNLDYYNPSAELLDKYAKGTHKKGDNFNLKDCHALIDFFKAGINKHPE |
| | | WQHFGFKFSPTSSYRDLSDFYREVEPQGYQVKFVDINADYIDELVEQGK |
| | | LYLFQIYNKDFSPKAHGKPNLHTLYFKALFSEDNLADPIYKLNGEAQIF |
| | | YRKASLDMNETTIHRAGEVLENKNPDNPKKRQFVYDIIKDKRYTQDKFM |
| | | LHVPITMNFGVQGMTIKEFNKKVNQSIQQYDEVNVIGIDRGERHLLYLT |
| | | VINSKGEILEQRSLNDITTASANGTQVTTPYHKILDKREIERLNARVGW |
| | | GEIETIKELKSGYLSHVVHQINQLMLKYNAIVVLEDLNFGFKRGRFKVE |
| | | KQIYQNFENALIKKLNHLVLKDKADDEIGSYKNALQLTNNFTDLKSIGK |
| | | QTGFLFYVPAWNTSKIDPETGFVDLLKPRYENIAQSQAFFGKFDKICYN |
| | | TDKGYFEFHIDYAKFTDKAKNSRQKWAICSHGDKRYVYDKTANQNKGAA |
| | | KGINVNDELKSLFARYHINDKQPNLVMDICQNNDKEFHKSLMCLLKTLL |
| | | ALRYSNASSDEDFILSPVANDEGVFFNSALADDTQPQNADANGAYHIAL |
| | | KGLWLLNELKNSDDLNKVKLAIDNQTWLNFAQNR |
| SEQ ID NO: 27 | Moraxella bovoculi | MGIHGVPAALFQDFTHLYPLSKTVRFELKPIGKTLEHIHAKNFLNQDET |
| | AAX11_00205 | MADMYQKVKAILDDYHRDFIADMMGEVKLTKLAEFYDVYLKFRKNPKDD |
| | (Mb3Cas12a) | GLQKQLKDLQAVLRKEIVKPIGNGGKYKAGYDRLFGAKLFKDGKELGDL |
| | | AKFVIAQEGESSPKLAHLAHFEKFSTYFTGFHDNRKNMYSDEDKHTAIA |
| | | YRLIHENLPRFIDNLQILATIKQKHSALYDQIINELTASGLDVSLASHL |
| | | DGYHKLLTQEGITAYNTLLGGISGEAGSRKIQGINELINSHHNQHCHKS |
| | | ERIAKLRPLHKQILSDGMGVSFLPSKFADDSEVCQAVNEFYRHYADVFA |
| | | KVQSLFDGFDDYQKDGIYVEYKNLNELSKQAFGDFALLGRVLDGYYVDV |
| | | VNPEFNERFAKAKTDNAKAKLTKEKDKFIKGVHSLASLEQAIEHYTARH |
| | | DDESVQAGKLGQYFKHGLAGVDNPIQKIHNNHSTIKGFLERERPAGERA |
| | | LPKIKSDKSPEIRQLKELLDNALNVAHFAKLLTTKTTLHNQDGNFYGEF |
| | | GALYDELAKIATLYNKVRDYLSQKPFSTEKYKLNFGNPTLLNGWDLNKE |
| | | KDNFGVILQKDGCYYLALLDKAHKKVFDNAPNTGKSVYQKMIYKLLPGP |
| | | NKMLPKVFFAKSNLDYYNPSAELLDKYAQGTHKKGDNFNLKDCHALIDF |
| | | FKAGINKHPEWQHFGFKFSPTSSYQDLSDFYREVEPQGYQVKFVDINAD |
| | | YINELVEQGQLYLFQIYNKDFSPKAHGKPNLHTLYFKALFSEDNLVNPI |
| | | YKLNGEAEIFYRKASLDMNETTIHRAGEVLENKNPDNPKKRQFVYDIIK |
| | | DKRYTQDKFMLHVPITMNFGVQGMTIKEFNKKVNQSIQQYDEVNVIGID |
| | | RGERHLLYLTVINSKGEILEQRSLNDITTASANGTQMTTPYHKILDKRE |
| | | IERLNARVGWGEIETIKELKSGYLSHVVHQISQLMLKYNAIVVLEDLNF |
| | | GFKRGRFKVEKQIYQNFENALIKKLNHLVLKDKADDEIGSYKNALQLTN |
| | | NFTDLKSIGKQTGFLFYVPAWNTSKIDPETGFVDLLKPRYENIAQSQAF |
| | | FGKFDKICYNADRGYFEFHIDYAKFNDKAKNSRQIWKICSHGDKRYVYD |
| | | KTANQNKGATIGVNVNDELKSLFTRYHINDKQPNLVMDICQNNDKEFHK |
| | | SLMYLLKTLLALRYSNASSDEDFILSPVANDEGVFFNSALADDTQPQNA |
| | | DANGAYHIALKGLWLLNELKNSDDLNKVKLAIDNQTWLNFAQNR |
| SEQ ID NO: 28 | Thiomicrospira sp. | MGIHGVPAATKTFDSEFFNLYSLQKTVRFELKPVGETASFVEDFKNEGL |
| | XS5 | KRVVSEDERRAVDYQKVKEIIDDYHRDFIEESLNYFPEQVSKDALEQAF |
| | (TsCas12a) | HLYQKLKAAKVEEREKALKEWEALQKKLREKVVKCFSDSNKARFSRIDK |
| | | KELIKEDLINWLVAQNREDDIPTVETFNNFTTYFTGFHENRKNIYSKDD |
| | | HATAISFRLIHENLPKFFDNVISFNKLKEGFPELKFDKVKEDLEVDYDL |
| | | KHAFEIEYFVNFVTQAGIDQYNYLLGGKTLEDGTKKQGMNEQINLFKQQ |
| | | QTRDKARQIPKLIPLFKQILSERTESQSFIPKQFESDQELFDSLQKLHN |
| | | NCQDKFTVLQQAILGLAEADLKKVFIKTSDLNALSNTIFGNYSVFSDAL |
| | | NLYKESLKTKKAQEAFEKLPAHSIHDLIQYLEQFNSSLDAEKQQSTDTV |
| | | LNYFIKTDELYSRFIKSTSEAFTQVQPLFELEALSSKRRPPESEDEGAK |
| | | GQEGFEQIKRIKAYLDTLMEAVHFAKPLYLVKGRKMIEGLDKDQSFYEA |
| | | FEMAYQELESLIIPIYNKARSYLSRKPFKADKFKINFDNNTLLSGWDAN |
| | | KETANASILFKKDGLYYLGIMPKGKTFLFDYFVSSEDSEKLKQRRQKTA |
| | | EEALAQDGESYFEKIRYKLLPGASKMLPKVFFSNKNIGFYNPSDDILRI |
| | | RNTASHTKNGTPQKGHSKVEFNLNDCHKMIDFFKSSIQKHPEWGSFGFT |
| | | FSDTSDFEDMSAFYREVENQGYVISFDKIKETYIQSQVEQGNLYLFQIY |
| | | NKDFSPYSKGKPNLHTLYWKALFEEANLNNVVAKLNGEAEIFFRRHSIK |
| | | ASDKVVHPANQAIDNKNPHTEKTQSTFEYDLVKDKRYTQDKFFFHVPIS |
| | | LNFKAQGVSKFNDKVNGFLKGNPDVNIIGIDRGERHLLYFTVVNQKGEI |
| | | LVQESLNTLMSDKGHVNDYQQKLDKKEQERDAARKSWTTVENIKELKEG |
| | | YLSHVVHKLAHLIIKYNAIVCLEDLNFGFKRGRFKVEKQVYQKFEKALI |
| | | DKLNYLVFKEKELGEVGHYLTAYQLTAPFESFKKLGKQSGILFYVPADY |
| | | TSKIDPTTGFVNFLDLRYQSVEKAKQLLSDFNAIRFNSVQNYFEFEIDY |
| | | KKLTPKRKVGTQSKWVICTYGDVRYQNRRNQKGHWETEEVNVTEKLKAL |
| | | FASDSKTTTVIDYANDDNLIDVILEQDKASFFKELLWLLKLTMTLRHSK |
| | | IKSEDDFILSPVKNEQGEFYDSRKAGEVWPKDADANGAYHIALKGLWNL |
| | | QQINQWEKGKTLNLAIKNQDWFSFIQEKPYQE |
| SEQ ID NO: 29 | Butyrivibrio sp. | MGIHGVPAAYYQNLTKKYPVSKTIRNELIPIGKTLENIRKNNILESDVK |
| | NC3005 | RKQDYEHVKGIMDEYHKQLINEALDNYMLPSLNQAAEIYLKKHVDVEDR |
| | (BsCas12a) | EEFKKTQDLLRREVTGRLKEHENYTKIGKKDILDLLEKLPSISEEDYNA |
| | | LESFRNFYTYFTSYNKVRENLYSDEEKSSTVAYRLINENLPKFLDNIKS |
| | | YAFVKAAGVLADCIEEEEQDALFMVETFNMTLTQEGIDMYNYQIGKVNS |
| | | AINLYNQKNHKVEEFKKIPKMKVLYKQILSDREEVFIGEFKDDETLLSS |
| | | IGAYGNVLMTYLKSEKINIFFDALRESEGKNVYVKNDLSKTTMSNIVFG |
| | | SWSAFDELLNQEYDLANENKKKDDKYFEKRQKELKKNKSYTLEQMSNLS |
| | | KEDISPIENYIERISEDIEKICIYNGEFEKIVVNEHDSSRKLSKNIKAV |
| | | KVIKDYLDSIKELEHDIKLINGSGQELEKNLVVYVGQEEALEQLRPVDS |
| | | LYNLTRNYLTKKPFSTEKVKLNFNKSTLLNGWDKNKETDNLGILFFKDG |
| | | KYYLGIMNTTANKAFVNPPAAKTENVFKKVDYKLLPGSNKMLPKVFFAK |
| | | SNIGYYNPSTELYSNYKKGTHKKGPSFSIDDCHNLIDFFKESIKKHEDW |
| | | SKFGFEFSDTADYRDISEFYREVEKQGYKLTFTDIDESYINDLIEKNEL |
| | | YLFQIYNKDFSEYSKGKLNLHTLYFMMLFDQRNLDNVVYKLNGEAEVFY |
| | | RPASIAENELVIHKAGEGIKNKNPNRAKVKETSTFSYDIVKDKRYSKYK |
| | | FTLHIPITMNFGVDEVRRFNDVINNALRTDDNVNVIGIDRGERNLLYVV |
| | | VINSEGKILEQISLNSIINKEYDIETNYHALLDEREDDRNKARKDWNTI |
| | | ENIKELKTGYLSQVVNVVAKLVLKYNAIICLEDLNFGFKRGRQKVEKQV |
| | | YQKFEKMLIEKLNYLVIDKSREQVSPEKMGGALNALQLTSKFKSFAELG |
| | | KQSGITYYVPAYLTSKIDPTTGFVNLFYIKYENIEKAKQFFDGFDFIRF |
| | | NKKDDMFEFSFDYKSFTQKACGIRSKWIVYTNGERIIKYPNPEKNNLFD |
| | | EKVINVTDEIKGLFKQYRIPYENGEDIKEIIISKAEADFYKRLFRLLHQ |
| | | TLQMRNSTSDGTRDYIISPVKNDRGEFFCSEFSEGTMPKDADANGAYNI |
| | | ARKGLWVLEQIRQKDEGEKVNLSMTNAEWLKYAQLHLL |
| SEQ ID NO: 30 | AacCas12b | MAVKSIKVKLRLDDMPEIRAGLWKLHKEVNAGVRYYTEWLSLLRQENLY |
| | | RRSPNGDGEQECDKTAEECKAELLERLRARQVENGHRGPAGSDDELLQL |
| | | ARQLYELLVPQAIGAKGDAQQIARKFLSPLADKDAVGGLGIAKAGNKPR |
| | | WVRMREAGEPGWEEEKEKAETRKSADRTADVLRALADFGLKPLMRVYTD |
| | | SEMSSVEWKPLRKGQAVRTWDRDMFQQAIERMMSWESWNQRVGQEYAKL |
| | | VEQKNRFEQKNFVGQEHLVHLVNQLQQDMKEASPGLESKEQTAHYVTGR |
| | | ALRGSDKVFEKWGKLAPDAPFDLYDAEIKNVQRRNTRRFGSHDLFAKLA |
| | | EPEYQALWREDASFLTRYAVYNSILRKLNHAKMFATFTLPDATAHPIWT |
| | | RFDKLGGNLHQYTFLFNEFGERRHAIRFHKLLKVENGVAREVDDVTVPI |
| | | SMSEQLDNLLPRDPNEPIALYFRDYGAEQHFTGEFGGAKIQCRRDQLAH |
| | | MHRRRGARDVYLNVSVRVQSQSEARGERRPPYAAVFRLVGDNHRAFVHF |
| | | DKLSDYLAEHPDDGKLGSEGLLSGLRVMSVDLGLRTSASISVFRVARKD |
| | | ELKPNSKGRVPFFFPIKGNDNLVAVHERSQLLKLPGETESKDLRAIREE |
| | | RQRTLRQLRTQLAYLRLLVRCGSEDVGRRERSWAKLIEQPVDAANHMTP |
| | | DWREAFENELQKLKSLHGICSDKEWMDAVYESVRRVWRHMGKQVRDWRK |
| | | DVRSGERPKIRGYAKDVVGGNSIEQIEYLERQYKFLKSWSFFGKVSGQV |
| | | IRAEKGSRFAITLREHIDHAKEDRLKKLADRIIMEALGYVYALDERGKG |
| | | KWVAKYPPCQLILLEELSEYQFNNDRPPSENNQLMQWSHRGVFQELINQ |
| | | AQVHDLLVGTMYAAFSSRFDARTGAPGIRCRRVPARCTQEHNPEPFPWW |
| | | LNKFVVEHTLDACPLRADDLIPTGEGEIFVSPFSAEEGDFHQIHADLNA |
| | | AQNLQQRLWSDFDISQIRLRCDWGEVDGELVLIPRLTGKRTADSYSNKV |
| | | FYTNTGVTYYERERGKKRRKVFAQEKLSEEEAELLVEADEAREKSVVLM |
| | | RDPSGIINRGNWTRQKEFWSMVNQRIEGYLVKQIRSRVPLQDSACENTG |
| | | DI |
| SEQ ID NO: 147 | Cas12 | MKKIDNFVGCYPVSKTLRFKAIPIGKTQENIEKKRLVEEDEVRAKDYKA |
| | Variant | VKKLIDRYHREFIEGVLDNVKLDGLEEYYMLFNKSDREESDNKKIEIME |
| | | ERFRRVISKSFKNNEEYKKIFSKKIIEEILPNYIKDEEEKELVKGFKGF |
| | | YTAFVGYAQNRENMYSDEKKSTAISYRIVNENMPRFITNIKVFEKAKSI |
| | | LDVDKINEINEYILNNDYYVDDFFNIDFFNYVLNQKGIDIYNAIIGGIV |
| | | TGDGRKIQGLNECINLYNQENKKIRLPQFKPLYKQILSESESMSFYIDE |
| | | IESDDMLIDMLKESLQIDSTINNAIDDLKVLFNNIFDYDLSGIFINNGL |
| | | PITTISNDVYGQWSTISDGWNERYDVLSNAKDKESEKYFEKRRKEYKKV |
| | | KSFSISDLQELGGKDLSICKKINEIISEMIDDYKSKIEEIQYLFDIKEL |
| | | EKPLVTDLNKIELIKNSLDGLKRIERYVIPFLGTGKEQNRDEVFYGYFI |
| | | KCIDAIKEIDGVYNKTRNYLTKKPYSKDKFKLYFENPQLMGGWDRNKES |
| | | DYRSTLLRKNGKYYVAIIDKSSSNCMMNIEEDENDNYEKINYKLLPGPN |
| | | KMLPKVFFSKKNREYFAPSKEIERIYSTGTFKKDTNFVKKDCENLITFY |
| | | KDSLDRHEDWSKSFDFSFKESSAYRDISEFYRDVEKQGYRVSFDLLSSN |
| | | AVNTLVEEGKLYLFQLYNKDFSEKSHGIPNLHTMYFRSLFDDNNKGNIR |
| | | LNGGAEMFMRRASLNKQDVTVHKANQPIKNKNLLNPKKTTTLPYDVYKD |
| | | KRFTEDQYEVHIPITMNKVPNNPYKINHMVREQLVKDDNPYVIGIDRGE |
| | | RNLIYVVVVDGQGHIVEQLSLNEIINENNGISIRTDYHTLLDAKERERD |
| | | ESRKQWKQIENIKELKEGYISQVVHKICELVEKYDAVIALEDLNSGFKN |
| | | SRVKVEKQVYQKFEKMLITKLNYMVDKKKDYNKPGGVLNGYQLTTQFES |
| | | FSKMGTQNGIMFYIPAWLTSKMDPTTGFVDLLKPKYKNKADAQKFFSQF |
| | | DSIRYDNQEDAFVFKVNYTKFPRTDADYNKEWEIYTNGERIRVFRNPKK |
| | | NNEYDYETVNVSERMKELFDSYDLLYDKGELKETICEMEESKFFEELIK |
| | | LFRLTLQMRNSISGRTDVDYLISPVKNSNGYFYNSNDYKKEGAKYPKDA |
| | | DANGAYNIARKVLWAIEQFKMADEDKLDKTKISIKNQEWLEYAQTHCE |
| SEQ ID NO: 148 | Cas12 | MATLVSFTKQYQVQKTLRFELIPQGKTQANIDAKGFINDDLKRDENYMK |
| | Variant | VKGVIDELHKNFIEQTLVNVDYDWRSLATAIKNYRKDRSDTNKKNLEKT |
| | | QEAARKEIIAWFEGKRGNSAFKNNQKSFYGKLFKKELFSEILRSDDLEY |
| | | DEETQDAIACFDKFTTYFVGFHENRKNMYSTEAKSTSVAYRVVNENFSK |
| | | FLSNCEAFSVLEAVCPNVLVEAEQELHLHKAFSDLKLSDVFKVEAYNKY |
| | | LSQTGIDYYNQIIGGISSAEGVRKIRGVNEVVNNAIQQNDELKVALRNK |
| | | QFTMVQLFKQILSDRSTLSFVSEQFTSDQEVITVVKQFNDDIVNNKVLA |
| | | VVKTLFENFNSYDLEKIYINSKELASVSNALLKDWSKIRNAVLENKIIE |
| | | LGANPPKTKISAVEKEVKNKDFSIAELASYNDKYLDKEGNDKEICSIAN |
| | | VVLEAVGALEIMLAESLPADLKTLENKNKVKGILDAYENLLHLLNYFKV |
| | | SAVNDVDLAFYGAFEKVYVDISGVMPLYNKVRNYATKKPYSVEKFKLNF |
| | | AMPTLADGWDKNKERDNGSIILLKDGQYYLGVMNPQNKPVIDNAVCNDA |
| | | KGYQKMVYKMFPEISKMVTKCSTQLNAVKAHFEDNTNDFVLDDTDKFIS |
| | | DLTITKEIYDLNNVLYDGKKKFQIDYLRNTGDFAGYHKALETWIDFVKE |
| | | FLSKYRSTAIYDLTTLLPTNYYEKLDVFYSDVNNLCYKIDYENISVEQV |
| | | NEWVEEGNLYLFKIYNKDFATGSTGKPNLHTMYWNAVFAEENLHDVVVK |
| | | LNGGAELFYRPKSNMPKVEHRVGEKLVNRKNVNGEPIADSVHKEIYAYA |
| | | NGKISKSELSENAQEELPLAIIKDVKHNITKDKRYLSDKYFFHVPITLN |
| | | YKANGNPSAFNTKVQAFLKNNPDVNIIGIDRGERNLLYVVVIDQQGNII |
| | | DKKQVSYNKVNGYDYYEKLNQREKERIEARQSWGAVGKIKELKEGYLSL |
| | | VVREIADMMVKYNAIVVMENLNAGFKRVRGGIAEKAVYQKFEKMLIDKL |
| | | NYLVFKDVEAKEAGGVLNAYQLTDKFDSFEKMGNQSGFLFYVPAAYTSK |
| | | IDPVTGFANVFSTKHITNTEAKKEFICSFNSLRYDEAKDKFVLECDLNK |
| | | FKIVANSHIKNWKFIIGGKRIVYNSKNKTYMEKYPCEDLKATLNASGID |
| | | FSSSEIINLLKNVPANREYGKLFDETYWAIMNTLQMRNSNALTGEDYII |
| | | SAVADDNEKVFDSRTCGAELPKDADANGAYHIALKGLYLLQRIDISEEG |
| | | EKVDLSIKNEEWFKFVQQKEYAR |
| SEQ ID NO: 149 | Cas12 | MKEQFINRYPLSKTLRFSLIPVGETENNFNKNLLLKKDKQRAENYEKVK |
| | Variant | CYIDRFHKEYIESVLSKARIEKVNEYANLYWKSNKDDSDIKAMESLEND |
| | | MRKQISKQLTSTEIYKKRLFGKELICEDLPSFLTDKDERETVECFRSFT |
| | | TYFKGFNTNRENMYSSDGKSTAIAYRCINDNLPRFLDNVKSFQKVFDNL |
| | | SDETITKLNTDLYNIFGRNIEDIFSVDYFEFVLTQSGIEIYNSMIGGYT |
| | | CSDKTKIQGLNECINLYNQQVAKNEKSKKLPLMKPLYKQILSEKDSVSF |
| | | IPEKFNSDNEVLHAIDDYYTGHIGDFDLLTELLQSLNTYNANGIFVKSG |
| | | VAITDISNGAFNSWNVLRSAWNEKYEALHPVTSKTKIDKYIEKQDKIYK |
| | | AIKSFSLFELQSLGNENGNEITDWYISSINESNSKIKEAYLQAQKLLNS |
| | | DYEKSYNKRLYKNEKATELVKNLLDAIKEFQKLIKPLNGTGKEENKDEL |
| | | FYGKFTSYYDSIADIDRLYDKVRNYITQKPYSKDKIKLNFDNPQLLGGW |
| | | DKNKESDYRTVLLHKDGLYYLAVMDKSHSKAFVDAPEITSDDKDYYEKM |
| | | EYKLLPGPNKMLPKVFFASKNIDTFQPSDRILDIRKRESFKKGATFNKA |
| | | ECHEFIDYFKDSIKKHDDWSQFGFKFSPTESYNDISEFYREISDQGYSV |
| | | RFNKISKNYIDGLVNNGYIYLFQIYNKDFSKYSKGTPNLHTLYFKMLFD |
| | | ERNLSNVVYKLNGEAEMFYREASIGDKEKITHYANQPIKNKNPDNEKKE |
| | | SVFEYDIVKDKRFTKRQFSLHLPITINFKAHGQEFLNYDVRKAVKYKDD |
| | | NYVIGIDRGERNLIYISVINSNGEIVEQMSLNEIISDNGHKVDYQKLLD |
| | | TKEKERDKARKNWTSVENIKELKEGYISQVVHKICELVIKYDAVIAMED |
| | | LNFGFKRGRFPVEKQVYQKFENMLISKLNLLIDKKAEPTEDGGLLRAYQ |
| | | LTNKFDGVNKAKQNGIIFYVPAWDTSKIDPATGFVNLLKPKCNTSVPEA |
| | | KKLFETIDDIKYNANTDMFEFYIDYSKFPRCNSDFKKSWTVCTNSSRIL |
| | | TFRNKEKNNKWDNKQIVLTDEFKSLFNEFGIDYKGNLKDSILSISNADF |
| | | YRRLIKLLSLTLQMRNSITGSTLPEDDYLISPVANKSGEFYDSRNYKGT |
| | | NAALPCDADANGAYNIARKALWAINVLKDTPDDMLNKAKLSITNAEWLE |
| | | YTQK |
| SEQ ID NO: 150 | Cas12 | MNNPRGAFGGFTNLYSLSKTLRFELKPYLEIPEGEKGKLFGDDKEYYKN |
| | Variant | CKTYTEYYLKKANKEYYDNEKVKNTDLQLVNFLHDERIEDAYQVLKPVF |
| | | DTLHEEFITDSLESAEAKKIDFGNYYGLYEKQKSEQNKDEKKKIDKPLE |
| | | TERGKLRKAFTPIYEAEGKNLKNKAGKEKKDKDILKESGFKVLIEAGIL |
| | | KYIKNNIDEFADKKLKNNEGKEITKKDIETALGAENIEGIFDGFFTYFS |
| | | GFNQNRENYYSTEEKATAVASRIVDENLSKFCDNILLYRKNENDYLKIF |
| | | NFLKNKGKDLKLKNSKFGKENEPEFIPAYDMKNDEKSFSVADFVNCLSQ |
| | | GEIEKYNAKIANANYLINLYNQNKDGNSSKLSMFKILYKQIGCGEKKDF |
| | | IKTIKDNAELKQILEKACEAGKKYFIRGKSEDGGVSNIFDFTDYIQSHE |
| | | NYKGVYWSDKAINTISGKYFANWDTLKNKLGDAKVFNKNTGEDKADVKY |
| | | KVPQAVMLSELFAVLDDNAGEDWREKGIFFKASLFEGDQNKSEIIKNAN |
| | | RPSQALLKMICDDMESLAKNFIDSGDKILKISDRDYQKDENKQKIKNWL |
| | | DNALWINQILKYFKVKANKIKGDSIDARIDSGLDMLVFSSDNPAEDYDM |
| | | IRNYLTQKPQDEINKLKLNFENSSLAGGWDENKEKDNSCIILKDEQDKQ |
| | | YLAVMKYENTKVFEQKNSQLYIADNAAWKKMIYKLVPGASKTLPKVFFS |
| | | KKWTANRPTPSDIVEIYQKGSFKKENVDFNDKKEKDESRKEKNREKIIA |
| | | ELQKTCWMDIRYNIDGKIESAKYVNKEKLAKLIDFYKENLKKYPSEEES |
| | | WDRLFAFGFSDTKSYKSIDQFYIEVDKQGYKLEFVTINKARLDEYVRDG |
| | | KIYLFEIRSRDNNLVNGEEKTSAKNLQTIYWNAAFGGDDNKPKLNGEAE |
| | | IFYRPAIAENKLNKKKDKNGKEIIDGYRFSKEKFIFHCPITLNFCLKET |
| | | KINDKLNAALAKPENGQGVYFLGIDRGEKHLAYYSLVNQKGEILEQGTL |
| | | NLPFLDKNGKSRSIKVEKKSFEKDSNGGIIKDKDGNDKIKIEFVECWNY |
| | | NDLLDARAGDRDYARKNWTTIGTIKELKDGYISQVVRKIVDLSIYKNTE |
| | | TKEFREMPAFIVLEDLNIGFKRGROKIEKOVYOKLELALAKKLNFLVDK |
| | | KADIGEIGSVTKAIQLTPPVNNFGDMENRKQFGNMLYIRADYTSQTDPA |
| | | TGWRKSIYLKSGSESNVKEQIEKSFFDIRYESGDYCFEYRDRHGKMWQL |
| | | YSSKNGVSLDRFHGERNNSKNVWESEKQPLNEMLDILFDEKRFDKSKSL |
| | | YEQMFKGGVALTRLPKEINKKDKPAWESLRFVIILIQQIRNTGKNGDDR |
| | | NGDFIQSPVRDEKTGEHFDSRIYLDKEQKGEKADLPTSGDANGAYNIAR |
| | | KGIVVAEHIKRGFDKLYISDEEWDTWLAGDEIWDKWLKENRESLTKTRK |
| SEQ ID NO: 151 | Cas12 | MNGNRIIVYREFVGVTPVAKTLRNELRPIGHTQEHIIHNGLIQEDELRQ |
| | Variant | EKSTELKNIMDDYYREYIDKSLSGVTDLDFTLLFELMNLVQSSPSKDNK |
| | | KALEKEQSKMREQICTHMQSDSNYKNIFNAKFLKEILPDFIKNYNQYDA |
| | | KDKAGKLETLALFNGFSTYFTDFFEKRKNVFTKEAVSTSIAYRIVHENS |
| | | LTFLANMTSYKKISEKALDEIEVIEKNNQDKMGDWELNQIFNPDFYNMV |
| | | LIQSGIDFYNEICGVVNAHMNLYCQQTKNNYNLFKMRKLHKQILAYTST |
| | | SFEVPKMFEDDMSVYNAVNAFIDETEKGNIIGKLKDIVNKYDELDEKRI |
| | | YISKDFYETLSCFMSGNWNLITGCVENFYDENIHAKGKSKEEKVKKAVK |
| | | EDKYKSINDVNDLVEKYIDEKERNEFKNSNAKQYIREISNIITDTETAH |
| | | LEYDEHISLIESEEKADEMKKRLDMYMNMYHWAKAFIVDEVLDRDEMFY |
| | | SDIDDIYNILENIVPLYNRVRNYVTQKPYNSKKIKLNFQSPTLANGWSQ |
| | | SKEFDNNAIILIRDNKYYLAIFNAKNKPDKKIIQGNSDKKNDNDYKKMV |
| | | YNLLPGANKMLPKVFLSKKGIETFKPSDYIISGYNAHKHIKTSENFDIS |
| | | FCRDLIDYFKNSIEKHAEWRKYEFKFSATDSYNDISEFYREVEMQGYRI |
| | | DWTYISEADINKLDEEGKIYLFQIYNKDFAENSTGKENLHTMYFKNIFS |
| | | EENLKDIIIKLNGQAELFYRRASVKNPVKHKKDSVLVNKTYKNQLDNGD |
| | | VVRIPIPDDIYNEIYKMYNGYIKENDLSEAAKEYLDKVEVRTAQKDIVK |
| | | DYRYTVDKYFIHTPITINYKVTARNNVNDMAVKYIAQNDDIHVIGIDRG |
| | | ERNLIYISVIDSHGNIVKQKSYNILNNYDYKKKLVEKEKTREYARKNWK |
| | | SIGNIKELKEGYISGVVHEIAMLMVEYNAIIAMEDLNYGFKRGRFKVER |
| | | QVYQKFESMLINKLNYFASKGKSVDEPGGLLKGYQLTYVPDNIKNLGKQ |
| | | CGVIFYVPAAFTSKIDPSTGFISAFNFKSISTNASRKQFFMQFDEIRYC |
| | | AEKDMFSFGFDYNNFDTYNITMSKTQWTVYTNGERLQSEFNNARRTGKT |
| | | KSINLTETIKLLLEDNEINYADGHDVRIDMEKMDEDKNSEFFAQLLSLY |
| | | KLTVQMRNSYTEAEEQEKGISYDKIISPVINDEGEFFDSDNYKESDDKE |
| | | CKMPKDADANGAYCIALKGLYEVLKIKSEWTEDGFDRNCLKLPHAEWLD |
| | | FIQNKRYE |
| SEQ ID NO: 152 | Cas12 | MKKIDSFVNYYPLSKTLRFSLIPVGKTEDNFNAKLLLEEDEKRAIEYEK |
| | Variant | VKRYIDRYHKHFIETVLANFHLDDVNEYAELYYKAGKDDKDLKYMEKLE |
| | | GKMRKSISAAFTKDKKYKEIFGQEIIKNILPEFLENEDEKESVKMFQGF |
| | | FTYFTGFNDNRKNMYTHEAQTTAISYRCINENLPKFLDNVQSFAKIKES |
| | | ISSDIMNKLDEVCMDLYGVYAQDMFCTDYFSFVLSQSGIDRYNNIIGGY |
| | | VDDKGVKIQGINEYINLYNQQVDEKNKRLPLMKKLYKQILIEKESISFI |
| | | PEKFESDNIVINAISDYYHNNVENLFDDFNKLFNEFSEYDDNGIFVTSG |
| | | LAVTDISNAVFGSWNIISDSWNEEYKDSHPMKKTTNAEKYYEDMKKEYK |
| | | KNLSFTIAELQRLGEAGCNDECKGDIKEYYKTTVAEKIENIKNAYEISK |
| | | DLLASDYEKSNDKKLCKNDSAISLLKNLLDSIKDLEKTIKPLLGTGKEE |
| | | NKDDVFYGKFTNLYEMISEIDRLYDKVRNYVTQKPYSKDKIKLNFENPQ |
| | | HLGGWDKNKERDYRSVLLKKEDKYYLAIMDKSNNKAFIDFPDDGECYEK |
| | | IEYKLLPGPNKMLPKVFFASSNIEYFAPSKKILEIRSRESFKKGDMFNL |
| | | KDCHEFIDFFKESIKKHEDWSQFGFEFSPTEKYNDISEFYNEVKIQGYS |
| | | LKYKNVSKKYIDELIECGQLYLFQIYNKDFSVYAKGNPNLHTMYFKMLF |
| | | DERNLANVVYQLNGGAEMFYRKASIKDSEKIVHHANQPIKNKNADNVKK |
| | | ESVFEYDIIKDKRFTKRQFSIHIPITLNFKAKGQNFINNDVRMALKKAD |
| | | ENYVIGIDRGERNLLYICVINSKGEIVEQKSLNEIIGDNGYRVDYHKLL |
| | | DKKEAERDEARKSWGTIENIKELKEGYLSQIVHEISKLVIKYDAVIAIE |
| | | DLNSGFKKGRFKVEKQVYQKFENMLCTKLNYLVDKNADANECGGLLKAY |
| | | QLTNKEDGANRGRQNGIIFSVPAWLTSKIDPVTGFADLLRPKYKSVSES |
| | | VEFISKIDNIRYNSKEDYFEFDIDYSKFPNSTASYKKKWTVCTYGERII |
| | | NVRNKEKNNMWDNKTIVLTDEFKKLFADFGVDVSKNIKESVLAIDSKDF |
| | | YYRFINLLANTLQLRNSEVGNVDVDYLISPVKGVDGSFYDSRLVKEKTL |
| | | PENADANGAYNIARKALWAIDVLKQTKDEELKNANLSIKNAEWLEYVQK |
| SEQ ID NO: 153 | Cas12 | MRTMVTFEDFTKQYQVSKTLRFELIPQGKTLENMKRDGIISVDRQRNED |
| | Variant | YQKAKGILDKLYKYILDFTMETVVIDWEALATATEEFRKSKDKKTYEKV |
| | | QSKIRTALLEHVKKQKVGTEDLFKGMFSSKIITGEVLAAFPEIRLSDEE |
| | | NLILEKFKDFTTYFTGFFENRKNVFTDEALSTSFTYRLVNDNFIKFFDN |
| | | CIVFKNVVNISPHMAKSLETCASDLGIFPGVSLEEVFSISFYNRLLTQT |
| | | GIDQFNQLLGGISGKEGEHKQQGLNEIINLAMQQNLEVKEVLKNKAHRF |
| | | TPLFKQILSDRSTMSFIPDAFADDDEVLSAVDAYRKYLSEKNIGDRAFQ |
| | | LISDMEAYSPELMRIGGKYVSVLSQLLFYSWSEIRDGVKAYKESLITGK |
| | | KTKKELENIDKEIKYGVTLQEIKEALPKKDIYEEVKKYAMSVVKDYHAG |
| | | LAEPLPEKIETDDERASIKHIMDSMLGLYRFLEYFSHDSIEDTDPVFGE |
| | | CLDTILDDMNETVPLYNKVRNFSTRKVYSTEKFKLNFNNSSLANGWDKN |
| | | KEQANGAILLRKEGEYFLGIFNSKNKPKLVSDGGAGIGYEKMIYKQFPD |
| | | FKKMLPKCTISLKDTKAHFQKSDEDFTLQTDKFEKSIVITKQIYDLGTQ |
| | | TVNGKKKFQVDYPRLTGDMEGYRAALKEWIDFGKEFIQAYTSTAIYDTS |
| | | LFRDSSDYPDLPSFYKDVDNICYKLTFEWIPDAVIDDCIDDGSLYLFKL |
| | | HNKDFSSGSIGKPNLHTLYWKALFEEENLSDVVVKLNGQAELFYRPKSL |
| | | TRPVVHEEGEVIINKTTSTGLPVPDDVYVELSKFVRNGKKGNLTDKAKN |
| | | WLDKVTVRKMPHAITKDRRFTVDKFFFHVPITLNYKADSSPYRFNDFVR |
| | | QYIKDCSDVKIIGIDRGERNLIYAVVIDGKGNIIEQRSFNTVGTYNYQE |
| | | KLEQKEKERQTARQDWATVTKIKDLKKGYLSAVVHELSKMIVKYKAIVA |
| | | LENLNVGFKRMRGGIAERSVYQQFEKALIDKLNYLVFKDEEQSGYGGVL |
| | | NAYQLTDKFESFSKMGQQTGFLFYVPAAYTSKIDPLTGFINPFSWKHVK |
| | | NREDRRNFLNLFSKLYYDVNTHDFVLAYHHSNKDSKYTIKGNWEIADWD |
| | | ILIQENKEVFGKTGTPYCVGKRIVYMDDSTTGHNRMCAYYPHTELKKLL |
| | | SEYGIEYTSGQDLLKIIQEFDDDKLVKGLFYIIKAALQMRNSNSETGED |
| | | YISSPIEGRPGICFDSRAEADTLPYDADANGAFHIAMKGLLLTERIRND |
| | | DKLAISNEEWLNYIQEMRG |
| SEQ ID NO: 154 | Cas12 | MNKDIRKNFTDFVGISEIQKTLRFILIPIGKTAQNIDKYNMFEDDEIRH |
| | Variant | EYYPILKEACDDFYRNHIDQQFENLELDWSKLDEALASEDRDLINETRA |
| | | TYRQVLFNRLKNSVDIKGDSKKNKTLSLESSDKNLGKKKTKNTFQYNFN |
| | | DLFKAKLIKAILPLYIEYIYEGEKLENAKKALKMYNRFTSRLSNFWQAR |
| | | ANIFTDDEISTGSPYRLVNDNFTIFRINNSIYTKNKPFIEEDILEFEKK |
| | | LKSKKIIKDFESVDDYFTVNAFNKLCTQNGIDKYNSILGGFTTKEREKV |
| | | KGLNELFNLAQQSINKGKKGEYRKNIRLGKLTKLKKQILAISDSTSFLI |
| | | EQIEDDQDLYNKIKDFFELLLKEEIENENIFTQYANLQKLIEQADLSKI |
| | | YINAKHLNKISHQVTGKWDSLNKGIALLLENININEESKEKSEVISNGQ |
| | | TKDISSEAYKRYLQIQSEEKDIERLRTQIYFSLEDLEKALDLVLIDENM |
| | | DRSDKSILSYVQSPDLNVNFERDLTDLYSRIMKLEENNEKLLANHSAID |
| | | LIKEFLDLIMLRYSRWQILFCDSNYELDQTFYPIYDAVMEILSNIIRLY |
| | | NLARNYLSRKPDRMKKKKINFNNPTLADGWSESKIPDNSSMLFIKDGMY |
| | | YLGIIKNRAAYSELLEAESLQSSEKKKSENSSYERMNYHFLPDAFRSIP |
| | | KSSIAMKAVKEHFEINQKTADLLLDTDKFSKPLRITKEIFDMQYVDLHK |
| | | NKKKYQVDYLRDTGDKKGYRKALNTWLNFCKDFISKYKGRNLFDYSKIK |
| | | DADHYETVNEFYNDVDKYSYHIFFTSVAETTVEKFISEGKLYLFQLYNK |
| | | DFSPHSTGKPNLHTIYWRALFSEENLTSKNIKLNGQAEIFFRPKQIETP |
| | | FTHKKGSILVNRFDVNGNPIPINVYQEIKGFKNNVIKWDDLNKTTQEGL |
| | | ENDQYLYFESEFEIIKDRRYTEDQLFFHVPISFNWDIGSNPKINDLATQ |
| | | YIVNSNDIHIIGIDRGENHLIYYSVIDLQGAIVEQGSLNTITEYTENKF |
| | | LNNKTNNLRKIPYKDILQQREDERADARIKWHAIDKIKDLKDGYLGQIV |
| | | HFLAKLIIKYNAIVILEDLNYGFKRGRFKVERQVYQKFEMALMKKLNVL |
| | | VFKDYDIDEIGGPLKPWQLTRPIDSYERMGRQNGILFYVPAAYTSAVDP |
| | | VTGFANLFYLNNVKNSEKFHFFSKFESIKYHSDQDMFSFAFDYNNFGTT |
| | | TRINDLSKSKWQVFTNHERSVWNNKEKNYVTQNLTDLIKKLLRTYNIEF |
| | | KNNQNVLDSILKIENNTDKENFARELFRLFRLTIQLRNTTVNENNTEIT |
| | | ENELDYIISPVKDKNGNFFDSRDELKNLPDNGDANGAYNIARKGLLYIE |
| | | QLQESIKTGKLPTLSISTLDWFNYIMK |
| SEQ ID NO: 155 | Cas12 | MTPIFCNFVVYQIMLFNNNININVKTMNKKHLSDFTNLFPVSKTLRFRL |
| | Variant | EPQGKTMENIVKAQTIETDEERSHDYEKTKEYIDDYHRQFIDDTLDKFA |
| | | FKVESTGNNDSLQDYLDAYLSANDNRTKQTEEIQTNLRKAIVSAFKMQP |
| | | QFNLLFKKEMVKHLLPQFVDTDDKKRIVAKFNDFTTYFTGFFTNRENMY |
| | | SDEAKSTSIAYRIVNQNLIKFVENMLTFKSHILPILPQEQLATLYDDFK |
| | | EYLNVASIAEMFELDHFSIVLTQRQIEVYNSVIGGRKDENNKQIKPGLN |
| | | QYINQHNQAVKDKSARLPLLKPLFNQILSEKAGVSFLPKQFKSASEVVK |
| | | SLNEAYAELSPVLAAIQDVVTNITDYDCNGIFIKNDLGLTDIAQRFYGN |
| | | YDAVKRGLRNQYELETPMHNGQKAEKYEEQVAKHLKSIESVSLAQINQV |
| | | VTDGGDICDYFKAFGATDDGDIQRENLLASINNAHTAISPVLNKENAND |
| | | NELRKNTMLIKDLLDAIKRLQWFAKPLLGAGDETNKDQVFYGKFEPLYN |
| | | QLDETISPLYDKVRSYLTKKPYSLDKFKINFEKSNLLGGWDPGADRKYQ |
| | | YNAVILRKDNDFYLGIMRDEATSKRKCIQVLDCNDEGLDENFEKVEYKQ |
| | | IKPSQNMPRCAFAKKECEENADIMELKRKKNAKSYNTNKDDKNALIRHY |
| | | QRYLDRTYPEFGFVYKDADEYDTVKAFTDSMDSQDYKLSFLQVSETGLN |
| | | KLVDEGDLYLFKITNKDFSSYAKGRPNLHTIYWRMLFDPKNLANVVYKL |
| | | EGKAEVFFRRKSLASTTTHKAKQAIKNKSRYNEAVKPQSTFDYDIIKDR |
| | | RFTADKFEFHVPIKMNFKAAGWNSTRLTNEVREFIKSQGVRHIIGIDRG |
| | | ERHLLYLTMIDMDGNIVKQCSLNAPAQDNARASEVDYHQLLDSKEADRL |
| | | AARRNWGTIENIKELKQGYLSQVVHLLATMMVDNDAILVLENLNAGFMR |
| | | GRQKVEKSVYQKFEKMLIDKLNYIVDKGQSPDKPTGALHAVQLTGLYSD |
| | | FNKSNMKRANVRQCGFVFYIPAWNTSKIDPVTGFVNLFDTHLSSMGEIK |
| | | AFFSKFDSIRYNQDKGWFEFKFDYSRFTTRAEGCRTQWTVCTYGERIWT |
| | | HRSKNQNNQFVNDTVNVTQQMLQLLQDCGIDPNGNLKEAIANIDSKKSL |
| | | ETLLHLFKLTVQMRNSVTGSEVDYMISPVADERGHFFDSRESDEHLPAN |
| | | ADANGAFNIARKGLMVVRQIMATDDVSKIKFAVSNKDWLRFAQHIDD |
| SEQ ID NO: 156 | Cas12 | MNKGGYVIMEKMTEKNRWENQFRITKTIKEEIIPTGYTKVNLQRVNMLK |
| | Variant | REMERNEDLKKMKEICDEYYRNMIDVSLRLEQVRTLGWESLIHKYRMLN |
| | | KDEKEIKALEKEQEDLRKKISKGFGEKKAWTGEQFIKKILPQYLMDHYT |
| | | GEELEEKLRIVKKFKGCTMFLSTFFKNRENIFTDKPIHTAVGHRITSEN |
| | | AMLFAANINTYEKMESNVTLEIERLQREFWRRGINISEIFTDAYYVNVL |
| | | TQKQIEAYNKICGDINQHMNEYCQKQKLKFSEFRMRELKKQILAVVGEH |
| | | FEIPEKIESTKEVYRELNEYYESLKELHGQFEELKSVQLKYSQIYVQKK |
| | | GYDRISRYIGGQWDLIQECMKKDCASGMKGTKKNHDAKIEEEVAKVKYQ |
| | | SIEHIQKLVCTYEEDRGHKVTDYVDEFIVSVCDLLGADHIITRDGERIE |
| | | LPLQYEPGTDLLKNDTINQRRLSDIKTILDWHMDMLEWLKTFLVNDLVI |
| | | KDEEFYMAIEELNERMQCVISVYNRIRNYVTQKGYEPEKIRICFDKGTI |
| | | LTGWTTGDNYQYSGFLLMRNDKYYLGIINTNEKSVRKILDGNEECKDEN |
| | | DYIRVGYHLINDASKQLPRIFVMPKAGKKSEILMKDEQCDYIWDGYCHN |
| | | KHNESKEFMRELIDYYKRSIMNYDKWEGYCFKFSSTESYDNMQDFYKEV |
| | | REQSYNISFSYINENVLEQLDKDGKIYLFQVYNKDFAAGSTGTPNLHTM |
| | | YLQNLFSSQNLELKRLRLGGNAELFYRPGTEKDVTHRKGSILVDRTYVR |
| | | EEKDGIEVRDTVPEKEYLEIYRYLNGKQKGDLSESAKQWLDKVHYREAP |
| | | CDIIKDKRYAQEKYFLHFSVEINPNAEGQTALNDNVRRWLSEEEDIHVI |
| | | GIDRGERNLIYVSLMDGKGRIKDQKSYNIVNSGNKEPVDYLAKLKVREK |
| | | ERDEARRNWKAIGKIKDIKTGYLSYVVHEIVEMAVREKAIIVMEDLNYG |
| | | FKRGRFKVERQVYQKFEEMLINKLNYVVDKQLSVDEPGGLLRGYQLAFI |
| | | PKDKKSSMRQNGIVFYVPAGYTSKIDPTTGFVNIFKFPQFGKGDDDGNG |
| | | KDYDKIRAFFGKFDEIRYECDEKVTADNTREVKERYRFDFDYSKFETHL |
| | | VHMKKTKWTVYAEGERIKRKKVGNYWTSEVISDIALRMSNTLNIAGIEY |
| | | KDGHNLVNEICALRGKQAGIILNELLEIVRLTVQLRNSTTEGDVDERDE |
| | | IISPVLNEKYGCFYHSTEYKQQNGDVLPKDADANGAYCIGLKGIYEIRQ |
| | | IKNKWKEDMTKGEGKALNEGMRISHDQWFEFIQNMNKGE |
| SEQ ID NO: 157 | Cas12 | MNELVKNRCKQTKTICQKLIPIGKTRETIEKYNLMEIDRKIAANKELMN |
| | Variant | KLFSLIAGKHINDTLSKCTDLDFEPLLTSLSSLNNAKENDRDNLREYYD |
| | | SVFEEKKTLAEEISSRLTAVKFAGKDFFTKNIPDFLETYEGDDKNEMSE |
| | | LVSLVIENTVTAGYVKKLEKIDRSMEYRLVSGTVVKRVLTDNADIYEKN |
| | | IEKAKDFDYGVLNIDEASQFTTLVAKDYANYLTADGIAIYNVGIGKINL |
| | | ALNEYCQKNKEYSYNKLALLPLQKMLYGEKLSLFEKLEDFTSDEELINS |
| | | YNKFAKTVNESGLAEIIKKAVPSYDEIVIKPNKISNYSNSITGHWSLVN |
| | | RIMKDYLENNGIKNADKYMEKGLTLSEIGDALENKNIKHSDFISNLIND |
| | | LGHTYTEIKENKESLKKDESVNALIIKKELDMLLSILQNLKVFDIDNEM |
| | | FDTGFGIEVSKAIEILGYGVPLYNKIRNYITKKPDPKKKFMTKFGSATI |
| | | GTGITTSVEGSKKATFLKDGDAVFLLLYNTAGCKANNVSVSNLADLINS |
| | | SLEIENSGKCYQKMIYQTPGDIKKQIPRVFVYKSEDDDLIKDFKAGLHK |
| | | TDLSFLNGRLIPYLKEAFATHETYKNYTFSYRNSYESYDEFCEHMSEQA |
| | | YILEWKWIDKKLIDDLVEDGSLLMFRVWNRFMKKKEGKISKHAKIVNEL |
| | | FSDENASNAAIKLLSVFDIFYRDKQIDNPIVHKAGTTLYNKRTKDGEVI |
| | | VDYTTMVKNKEKRPNVYTTTKKYDIIKDRRYTEEQFEIHLHVNIGKEEN |
| | | KEKLETSKVINEKKNTLVVTRSNEHLLYVVIFDENDNILLKKSLNTVKG |
| | | MNFKSKLEVVEIQKKENMQSWKTVGSNQALMEGYLSFAIKEIADLVKEY |
| | | DAILVLEQNSVGKNILNERVYTRFKEMLITNLSLDVDYENKDFYSYTEL |
| | | GGKVASWRDCVTNGICIQVPSAYKYKDPTTSFSTISMYAKTTAEKSKKL |
| | | KQIKSFKYNRERGLFELVIAKGVGLENNIVCDSFGSRSIIENDISKEVS |
| | | CTLKIEKYLIDAGIEYNDEKEVLKDLDTAAKTDAVHKAVTLLLKCFNES |
| | | PDGRYYISPCGEHFTLCDAPEVLSAINYYIRSRYIREQIVEGVKKMEYK |
| | | KTILLAK |
| SEQ ID NO: 158 | Cas12 | MNYKTGLEDFIGKESLSKTLRNALIPTESTKIHMEEMGVIRDDELRAEK |
| | Variant | QQELKEIMDDYYRTFIEEKLGQIQGIQWNSLFQKMEETMEDISVRKDLD |
| | | KIQNEKRKEICCYFTSDKRFKDLFNAKLITDILPNFIKDNKEYTEEEKA |
| | | EKEQTRVLFQRFATAFTNYFNQRRNNFSEDNISTAISFRIVNENSEIHL |
| | | QNMRAFQRIEQQYPEEVCGMEEEYKDMLQEWQMKHIYSVDFYDRELTQP |
| | | GIEYYNGICGKINEHMNQFCQKNRINKNDFRMKKLHKQILCKKSSYYEI |
| | | PFRFESDQEVYDALNEFIKTMKKKEIIRRCVHLGQECDDYDLGKIYISS |
| | | NKYEQISNALYGSWDTIRKCIKEEYMDALPGKGEKKEEKAEAAAKKEEY |
| | | RSIADIDKIISLYGSEMDRTISAKKCITEICDMAGQISIDPLVCNSDIK |
| | | LLQNKEKTTEIKTILDSFLHVYQWGQTFIVSDIIEKDSYFYSELEDVLE |
| | | DFEGITTLYNHVRSYVTQKPYSTVKFKLHFGSPTLANGWSQSKEYDNNA |
| | | ILLMRDQKFYLGIFNVRNKPDKQIIKGHEKEEKGDYKKMIYNLLPGPSK |
| | | MLPKVFITSRSGQETYKPSKHILDGYNEKRHIKSSPKFDLGYCWDLIDY |
| | | YKECIHKHPDWKNYDFHFSDTKDYEDISGFYREVEMQGYQIKWTYISAD |
| | | EIQKLDEKGQIFLFQIYNKDFSVHSTGKDNLHTMYLKNLFSEENLKDIV |
| | | LKLNGEAELFFRKASIKTPIVHKKGSVLVNRSYTQTVGNKEIRVSIPEE |
| | | YYTEIYNYLNHIGKGKLSSEAQRYLDEGKIKSFTATKDIVKNYRYCCDH |
| | | YFLHLPITINFKAKSDVAVNERTLAYIAKKEDIHIIGIDRGERNLLYIS |
| | | VVDVHGNIREQRSFNIVNGYDYQQKLKDREKSRDAARKNWEEIEKIKEL |
| | | KEGYLSMVIHYIAQLVVKYNAVVAMEDLNYGFKTGRFKVERQVYQKFET |
| | | MLIEKLHYLVFKDREVCEEGGVLRGYQLTYIPESLKKVGKQCGFIFYVP |
| | | AGYTSKIDPTTGFVNLFSFKNLTNRESRQDFVGKFDEIRYDRDKKMFEF |
| | | SFDYNNYIKKGTILASTKWKVYTNGTRLKKIVVNGKYTSQSMEVELTDA |
| | | MEKMLQRAGIEYHDGKDLKGQIVEKGIEAEIIDIFRLTVQMRNSRSESE |
| | | DREYDRLISPVLNDKGEFFDTATADKTLPQDADANGAYCIALKGLYEVK |
| | | QIKENWKENEQFPRNKLVQDNKTWFDFMQKKRYL |
| SEQ ID NO: 159 | Cas12 | MEDKQFLERYKEFIGLNSLSKTLRNSLIPVGSTLKHIQEYGILEEDSLR |
| | Variant | AQKREELKGIMDDYYRNYIEMHLRDVHDIDWNELFEALTEVKKNQTDDA |
| | | KKRLEKIQEKKRKEIYQYLSDDAVFSEMFKEKMISGILPDFIRCNEGYS |
| | | EEEKEEKLKTVALFHRFTSSFNDFFLNRKNVFTKEAIVTAIGYRVVHEN |
| | | AEIFLENMVAFQNIQKSAESQISIIERKNEHYFMEWKLSHIFTADYYMM |
| | | LMTQKAIEHYNEMCGVVNQQMREYCQKEKKNWNLYRMKRLHKQILSNAS |
| | | TSFKIPEKYENDAEVYESVNSFLQNVMEKTVMERIAVLKNSTDNFDLSK |
| | | IYITAPYYEKISNYLCGSWNTITDCLTHYYEQQIAGKGARKDQKVKAAV |
| | | KADKWKSLSEIEQLLKEYARAEEVKRKPEEYIAEIENIVSLKEAHLLEY |
| | | HPEVNLIENEKYATEIKDVLDNYMELFHWMKWFYIEEAVEKEVNFYGEL |
| | | DDLYEEIKDIVPLYNKVRNYVTQKPYSDTKIKLNFGTPTLANGWSKSKE |
| | | YDYNAILLQKDGKYYMGIFNPIQKPEKEIIEGHSQPLEGNEYKKMVYYY |
| | | LPSANKMLPKVLLSKKGMEIYQPSEYIINGYKERRHIKSEEKFDLQFCH |
| | | DLIDYFKSGIERNSDWKVFGFDFSDTDTYQDISGFYREVEDQGYKIDWT |
| | | YIKEADIDRLNEEGKLYLFQIYNKDFSEKSTGRENLHTMYLKNLFSEEN |
| | | VREQVLKLNGEAEIFFRKSSVKKPIIHKKGTMLVNRTYMEEVNGNSVRR |
| | | NIPEKEYQEIYNYKNHRLKGELSTEAKKYLEKAVCHETKKDIVKDYRYS |
| | | VDKFFIHLPITINYRASGKETLNSVAQRYIAHQNDMHVIGIDRGERNLI |
| | | YVSVINMQGEIKEQKSFNIINEFNYKEKLKEREQSRGAARRNWKEIGQI |
| | | KDLKEGYLSGVIHEIAKMMIKYHAIIAMEDLNYGFKRGRFKVERQVYQK |
| | | FENMLIQKLNYLVFKDRPADEDGGVLRGYQLAYIPDSVKKMGRQCGMIF |
| | | YVPAAFTSKIDPTTGFVDIFKHKVYTTEQAKREFILSFDEICYDVERQL |
| | | FRFTFDYANFVTQNVTLARNNWTIYTNGTRAQKEFGNGRMRDKEDYNPK |
| | | DKMVELLESEGIEFKSGKNLLPALKKVSNAKVFEELQKIVRFTVQLRNS |
| | | KSEENDVDYDHVISPVLNEEGNFFDSSKYKNKEEKKESLLPVDADANGA |
| | | YCIALKGLYIMQAIQKNWSEEKALSPDVLRLNNNDWFDYIQNKRYR |
| SEQ ID NO: 160 | Cas12 | MEKSLNDFIGLYSVSKTLRFELKPVSETLENIKKFHFLEEDKKKANDYK |
| | Variant | DVKKIIDNYHKYFIDDVLKNASFNWKKLEEAIREYNKNKSDDSALVAEQ |
| | | KKLGDAILKLFTSDKRYKALTAATPKELFESILPDWFGEQCNQDLNKAA |
| | | LKTFQKFTSYFTGFQENRKNVYSAEAIPTAVPYRIVNDNFPKFLQNVLI |
| | | FKTIQEKCPQIIDEVEKELSSYLGKEKLAGIFTLESFNKYLGQGGKENQ |
| | | RGIDFYNQIIGGVVEKEGGINLRGVNQFLNLYWQQHPDFTKEDRRIKMV |
| | | PLYKQILSDRSSLSFKIESIENDEELKNALLECADKLELKNDEKKSIFE |
| | | EVCDLFSSVKNLDLSGIYINRKDINSVSRILTGDWSWLQSRMNVYAEEK |
| | | FTTKAEKARWQKSLDDEGENKSKGFYSLTDLNEVLEYSSENVAETDIRI |
| | | TDYFEHRCRYYVDKETEMFVQGSELVALSLQEMCDDILKKRKAMNTVLE |
| | | NLSSENKLREKTDDVAVIKEYLDAVQELLHRIKPLKVNGVGDSTFYSVY |
| | | DSIYSALSEVISVYNKTRNYITKKAASPEKYKLNFDNPTLADGWDLNKE |
| | | QANTSVILRKDGMFYLGIMNPKNKPKFAEKYDCGNESCYEKMIYKQFDA |
| | | TKQIPKCSTQKKEVQKYFLSGATEPYILNDKKSFKSELIITKDIWFMNN |
| | | HVWDGEKFVPKRDNETRPKKFQIGYFKQTGDFDGYKNALSNWISFCKNF |
| | | LQSYLSATVYDYNFKNSEEYEGLDEFYNYLNATCYKLNFINIPETEINK |
| | | MVSEGKLYLFQIYNKDFASGSTGMPNMHTLYWKNLFSDENLKNVCLKLN |
| | | GEAELFYRPAGIKEPVIHKEGSYLVNRTTEDGESIPEKIYFEIYKNANG |
| | | KLEKLSDEAQNYISNHEVVIKKAGHEIIKDRHYTEPKFLFHVPLTINFK |
| | | ASGNSYSINENVRKFLKNNPDVNIIGLDRGERHLIYLSLINQKGEIIKQ |
| | | FTFNEVERNKNGRTIKVNYHEKLDQREKERDAARKSWQAIGKIAELKEG |
| | | YLSAVIHQLTKLMVEYNAVVVMEDLNFGFKRGRFHVEKQVYQKFEHILI |
| | | DKSNYLVFKDRGLNEPGGVLNGYQIAGQFESFQKLGKQSGMLFYVPAGY |
| | | TSKIDPKTGFVSMMNFKDLTNVHKKRDFFSKFDNIHYDEANGSFVFTFD |
| | | YKKFDGKAKEEMKLTKWSVYSRDKRIVYFAKTKSYEDVLPTEKLQKIFE |
| | | SNGIDYKSGNNIQDSVMAIGADLKEGAKPSKEISDFWDGLLSNFKLILQ |
| | | MRNSNARTGEDYIISPVMADDGTFFDSREEFKKGEDAKLPLDADANGAY |
| | | HIALKGLSLINKINLSKDEELKKFDMKISNADWFKFAQEKNYAK |
| SEQ ID NO: 161 | Cas12 | MEEKKMSKIEKFIGKYKISKTLRFRAVPVGKTQDNIEKKGILEKDKKRS |
| | Variant | EDYEKVKAYLDSLHRDFIENTLKKVKLNELNEYACLFFSGTKDDGDKKK |
| | | MEKLEEKMRKTISNEFCNDEMYKKIFSEKILSENNEEDVSDIVSSYKGF |
| | | FTSLNGYVNNRKNLYVSDAKPTSIAYRCINENLPKFLRNVECYKKVVQV |
| | | IPKEQIEYMSNNLNLSPYRIEDCFNIDFFEFCLSQGGIDLYNTFIGGYS |
| | | KKDGTKVQGINEIVNLYNQKNKKDKEKYKLPQFTPLFKQILSDRDTKSF |
| | | SIEKLENIYEVVELVKKSYSDEMFDDIETVFSNLNYYDASGIYVKNGPA |
| | | ITHISMNLTKDWATIRNNWNYEYDEKHSTKKNKNIEKYEDTRNTMYKKI |
| | | DSFTLEYISRLVGKDIDELVKYFENEVANFVMDIKKTYSKLTPLFDRCQ |
| | | KENFDISEDEVNDIKGYLDNVKLLESFMKSFTINGKENNIDYVFYGKFT |
| | | DDYDKLHEFDHIYNKVRNYITTSRKPYKLDKYKLYFDNPQLLGGWDINK |
| | | EKDYRTVMLTKDGKYYFAIIDKGEHPFDNIPKDYFDNNGYYKKIIYRQI |
| | | PNAAKYLSSKQIVPQNPPEEVKRILDKKKADSKSLTEEEKNIFIDYIKS |
| | | DFLKNYKLLFDKNNNPYFNFAFRESSTYESLNEFFEDVERQAYSVRYEN |
| | | LPADYIDNLVNEGKIYLFEIYSKDFSEYSKGTNNLHTMYFKALFDNDNL |
| | | KNTVFKLSGNAELFIRPASIKKDELVIHPKNQLLQNKNPLNPKKQSIFD |
| | | YDLVKDKRFFENQYMLHISIEINKNERDAKKIKNINEMVRKELKDSDDN |
| | | YIIGIDRGERNLLYVCVINSAGKIVEQMSLNEIINEYNGIKHTVDYQGL |
| | | LDKCEKERNAQRQSWKSIENIKELKDGYISQVVHKLCQLVEKYDAIIAM |
| | | ENLNGGFKRGRTKFEKQVYQKFENKLINKMEYMADKKRKTTENGGILRG |
| | | YQLTNGCINNSYQNGFIFYVPAWLTSKIDPTTGFVDLLKPKYTNVEEAH |
| | | LWINKFNSITYDKKLDMFAFNINYSQFPRADIDYRKIWTFYTNGYRIET |
| | | FRNSEKNNEFDWKEVHLTSVIKKLLEEYQINYISGKNIIDDLIQIKDKP |
| | | FWNSFIKYIRLTLQMRNSITGRTDVDYIISPVINNEGTFYDSRKDLDEI |
| | | TLPQDADANGAYNIARKALWIIEKLKESPDEELNKVKLAITQREWLEYA |
| | | QINI |
| SEQ ID NO: 162 | Cas12 | MIIHNCYIGGSFMKKIDSFTNCYSLSKTLRFKLIPIGATQSNFDLNKML |
| | Variant | DEDKKRAENYSKAKSIIDKYHRFFIDKVLSSVTENKAFDSFLEDVRAYA |
| | | ELYYRSNKDDSDKASMKTLESKMRKFIALALQSDEGFKDLFGQNLIKKT |
| | | LPEFLESDTDKEIIAEFDGFSTYFTGFFNNRKNMYSADDQPTAISYRCI |
| | | NDNLPKFLDNVRTFKNSDVASILNDNLKILNEDFDGIYGTSAEDVFNVD |
| | | YFPFVLSQKGIEAYNSILGGYTNSDGSKIKGLNEYINLYNQKNENIHRI |
| | | PKMKQLFKQILSERESVSFIPEKFDSDDDVLSSINDYYLERDGGKVLSI |
| | | EKTVEKIEKLFSAVTDYSTDGIFVKNAAELTAVCSGAFGYWGTVQNAWN |
| | | NEYDALNGYKETEKYIDKRKKAYKSIESFSLADIQKYADVSESSETNAE |
| | | VTEWLRNEIKEKCNLAVQGYESSKDLISKPYTESKKLFNNDNAVELIKN |
| | | ALDSVKELENVLRLLLGTGKEESKDENFYGEFLPCYERICEVDSLYDKV |
| | | RNYMTQKLYKTDKIKLNFQNPQFLGGWDRNKEADYSAVLLRRNSLYYIA |
| | | IMPSGYKRVFEKIPAPKADETVYEKVIYKLLPGPNKMLPKVFFSKKGIE |
| | | TFNPPKEILEKYELGTHKTGDGFNLDDCHALIDYFKSALDVHSDWSNFG |
| | | FRFSDTSTYKNIADFYNEVKNQGYKITFCDVPQSYINELVDEGKLYLFQ |
| | | LYNKDFSEHSKGTPNLHTLYFKMLFDERNLENVVFKLNGEAEMFYREAS |
| | | ISKDDMIVHPKNQPIKNKNEQNSRKQSTFEYDIVKDRRYTVDQFMLHIP |
| | | ITLNFTANGGTNINNEVRKALKDCDKNYVIGIDRGERNLLYICVVDSEG |
| | | RIIEQYSLNEIINEYNGNTYSTDYHALLDKKEKERLESRKAWKTVENIK |
| | | ELKEGYISQVVHKICELVEKYDAVIVMEDLNLGFKQGRSGKFEKSVYQK |
| | | FEKMLIDKLNYFADKKKSPEEIGSVLNAYQLTNAFESFEKMGKQNGFIF |
| | | YVPAYLTSKIDPTTGFADLLHPSSKQSKESMRDFVGRFDSITFNKTENY |
| | | FEFELDYNKFPRCNTDYRKKWTVCTYGSRIKTFRNPEKNSEWDNKTVEL |
| | | TPAFMALFEKYSIDVNGDIKAQIMSVDKKDFFVELIGLLRLTLQMRNSE |
| | | TGKVDRDYLISPVKNSEGVFYNSDDYKGIENASLPKDADANGAYNIARK |
| | | GLWIIEQIKACENDAELNKIRLAISNAEWLEYAQKK |
| SEQ ID NO: 163 | Cas12 | MKEQFVNQYPISKTLRFSLIPIGKTEENFNKNLLLKEDEKKAEEYQKVK |
| | Variant | GYIDRYHKFFIETALCNINFEGFEEYSLLYYKCSKDDNDLKTMEDIEIK |
| | | LRKQISKTMTSHKLYKDLFGENMIKTILPNFLDSDEEKNSLEMFRGFYT |
| | | YFSGFNTNRKNMYTEEAKSTSIAYRCINDNLPKFLDNSKSFEKIKCALN |
| | | KEELKAKNEEFYEIFQIYATDIFNIDFFNFVLTQPGIDKYNGIIGGYTC |
| | | SDGTKVQGLNEIINLYNQQIAKDDKSKRLPLLKMLYKQILSDRETVSFI |
| | | PEKFSSDNEVLESINNYFSKNVSNAIKSLKELFQGFEAYNMNGIFISSG |
| | | VAITDLSNAVFGDWNAISTAWEKAYFETNPPKKNKSQEKYEEELKANYK |
| | | KIKSFSLDEIQRLGSIAKSPDSIGSVAEYYKITVTEKIDNITELYDGSK |
| | | ELLNCNYSESYDKKLIKNDTVIEKVKTLLDAVKSLEKLIKPLVGTGKED |
| | | KDELFYGTFLPLYTSLSAVDRLYDKVRNYATQKPYSKDKIKLNFNCSSF |
| | | LSGWATDYSSNGGLIFEKDGLYYLGIVNKKFTTEEIDYLQQNADENPAQ |
| | | RIVYDFQKPDNKNTPRLFIRSKGTNYSPSVKEYNLPVEEIVELYDKRYF |
| | | TTEYRNKNPELYKASLVKLIDYFKLGFTRHESYRHYDFKWKKSEEYNDI |
| | | SEFYKDVEISCYSLKQEKINYNTLLNFVAENRIYLFQIYNKDFSKYSKG |
| | | TPNLHTRYFKALFDENNLSDVVFKLNGGSEMFFRKASIKDNEKVVHPAN |
| | | QPIDNKNPDNSKKQSTFDYELIKDKRFTKHQFSIHIPITMNFKARGRDF |
| | | INNDIRKAIKSEYKPYVIGIDRGERNLIYISVINNNGEIVEQMSLNDII |
| | | SDNGYKVDYQRLLDRKEKERDNARKSWGTIENIKELKEGYISQVIHKIC |
| | | ELVIKYDAVIAMEDLNFGFKRGRFNVEKQVYQKFENMLISKLNYLCDKK |
| | | SEANSEGGLLKAYQLTNKFDGVNKGKQNGIIFYVPAWLTSKIDPVTGFV |
| | | DLLHPKYISVEETHSLFEKLDDIRYNFEKDMFEFDIDYSKLPKCNADFK |
| | | QKWTVCTNADRIMTFRNSEKNNEWDNKRILLSDEFKRLFEEFGIDYCHN |
| | | LKNKILSISNKDFCYRFIKLFALTMQMRNSITGSTNPEDDYLISPVRDE |
| | | NGVFYDSRNFIGSKAGLPIDADANGAYNIARKGLWAINAIKSTADDMLD |
| | | KVDLSISNAKWLEYVQK |
| SEQ ID NO: 164 | Cas12 | MADLSQFTHKYQVPKTLRFELIPQGKTLENLSAYGMVADDKQRSENYKK |
| | Variant | LKPVIDRIYKYFIEESLKNTNLDWNPLYEAIREYRKEKTTATITNLKEQ |
| | | QDICRRAIASRFEGKVPDKGDKSVKDFNKKQSKLFKELFGKELFTDSVL |
| | | EQLPGVSLSDEDKALLKSFDKFTTYFVGFYDNRKNVFSSDDISTGIPHR |
| | | LVQENFPKFIDNCDDYKRLVLVAPELKEKLEKAAEATKIFEDVSLDEIF |
| | | SIKFYNRLLQQNQIDQFNQLLGGIAGAPGTPKIQGLNETLNLSMQQDKT |
| | | LEQKLKSVPHRFSPLYKQILSDRSSLSFIPESFSCDAEVLLAVQEYLDN |
| | | LKTEHVIEDLKEVFNRLTTLDLKHIYVNSTKVTAFSQALFGDWNLCREQ |
| | | LRVYKMSNGNEKITKKALGELESWLKNSDIAFTELQEALADEALPAKVN |
| | | LKVQEAISGLNEQMAKSLPKELKIPEEKEELKALLDAIQEVYHTLEWFI |
| | | VSDDVETDTDFYVPLKETLQIIQPIIPLYNKVRNFATQKPYSVEKFKLN |
| | | FANPTLADGWDENKEQQNCAVLFQKGNNYYLGILNPKNKPDFDNVDTEK |
| | | QGNCYQKMVYKQFPDFSKMMPKCTTQLKEVKQHFEGKDSDYILNNKNFI |
| | | KPLTITREVYDLNNVLYDGKKKFQIDYLRKTKDEDGYYTALHTWIDFAK |
| | | KFVASYKSTSIYDTSTILPPEKYEKLNEFYGALDNLFYQIKFENIPEEI |
| | | IDTYVEDGKLFLFQIYNKDFAAGATGAPNLHTIYWKAVFDPENVKDVVV |
| | | KLNGQAELFYRPKSNMDVIRHKVGEKLVNRTLKDGSILTDELHKELYLY |
| | | ANGSLKKGLSEDAKIILDKNLAVIYDVI-IHEIVKDRRFTTDKFFFHVP |
| | | LTLNYKCDKNPVKFNAEVQEYLKENPDTYVIGIDRGERNLIYAVVIDPK |
| | | GRIVEQKSFNVINGFDYHGKLDQREKERVKARQAWTAVGKIKELKQGYL |
| | | SLVVHEISKMMVRYQAVVVLENLNVGFKRVRSGIAEKAVYQQFEKMLIN |
| | | KLNYLMFKDAGGTEPGSVLNAYQLTDRFESFAKMGLQTGFLFYIPAAFT |
| | | SKIDPATGFVDPFRWGAIKTLADKREFLSGFESLKFDSTTGNFILHFDV |
| | | SKNKNFQKKLEGFVPDWDIIIEANKMKTGKGATYIAGKRIEFVRDNNSQ |
| | | GHYEDYLPCNALAETLRQCDIPYEEGKDILPLILEKNDSKLLHSVFKVV |
| | | RLTLQMRNSNAETGEDYISSPVEDVSGSCFDSRMENEKLPKDADANGAY |
| | | HIALKGMLALERLRKDEKMAISNNDWLNYIQEKRA |
| SEQ ID NO: 165 | Cas12 | MTNFDNFTKKYVNSKTIRLEAIPVGKTLKNIEKMGFIAADRQRDEDYQK |
| | Variant | AKSVIDHIYKAFMDDCLKDLFLDWDPLYEAVVACWRERSPEGRQALQIM |
| | | QADYRKKIADRFRNHELYGSLFTKKIFDGSVAQRLPDLEQSAEEKSLLS |
| | | NFNKFTSYFRDFFDKRKRLFSDDEKHSAIAYRLINENFLKFVANCEAFR |
| | | RMTERVPELREKLQNTGSLQVYNGLALDEVFSADFYNQLIVQKQIDLYN |
| | | QLIGGIAGEPGTPNIQGLNATINLALQGDSSLHEKLAGIPHRFNPLYKQ |
| | | ILSDVSTLSFVPSAFQSDGEMLAAVRGFKVQLESGRVLQNVRRLFNGLE |
| | | TEADLSRVYVNNSKLAAFSSMFFGRWNLCSDALFAWKKGKQKKITNKKL |
| | | TEIKKWLKNSDIAIAEIQEAFGEDFPRGKINEKIQAQADALHSQLALPI |
| | | PENLKALCAKDGLKSMLDTVLGLYRMLQWFIVGDDNEKDSDFYFGLGKI |
| | | LGSLDPVLVLYNRVRNYITKKPYSLTKFRLNFDNSQLLNGWDENNLDTN |
| | | CASIFIKDGKYYLGISNKNNRPQFDTVATSGKSGYQRMVYKQFANWGRD |
| | | LPHSTTQMKKVKKHFSASDADYVLDGDKFIRPLIITKEIFDLNNVKFNG |
| | | KKKLQVDYLRNTGDREGYTHALHTWINFAKDFCACYKSTSIYDISCLRP |
| | | TDQYDNLMDFYADLGNLSHRIVWQTIPEEAIDNYVEQGQLFLFQLYNKD |
| | | FAPGADGKPNLHTLYWKAVFNPENLEDVVVKLNGKAELFYRPRSNMDVV |
| | | RHKVGEKLVNRKLKNGLTLPSRLHEEIYRYVNGTLNKDLSADARSVLPL |
| | | AVVRDVQHEIIKDRRFTADKFFFHASLTFNFKSSDKPVGFNEDVREYLR |
| | | EHPDTYVVGVDRGERNLIYIVVIDPQGNIVEQRSFNMINGIDYWSLLDQ |
| | | KEKERVEAKQAWETVGKIKDLKCGYLSFLIHEITKIIIKYHAVVILENL |
| | | SLGFKRVRTGIAEKAVYQQFERMLVTKLGYVVFKDRAGKAPGGVLNAYQ |
| | | LTDNTRTAENTGIQNGFLFYVPAAFTSRVDPATGFFDFYDWGKIKTATD |
| | | KKNFIAGFNSVRYERSTGDFIVHVGAKNLAVRRVAEDVRTEWDIVIEAN |
| | | VRKMGIDGNSYISGKRIRYRSGEQGHGQYENHLPCQELIRALQQYGIQY |
| | | ETGKDILPAILQQDDAKLTDTVFDVFRLALQMRNTSAETGEDYFNSVVR |
| | | DRSGRCFDTRRAEAAMPKEADANDAYHIALKGLFVLEKLRKGESIGIKN |
| | | TEWLRYVQQRHS |
| SEQ ID NO: 166 | Cas12 | MENYGGFTGLYPLQKTLKFELRPQGRTMEHLVSSNFFEEDRDRAEKYKI |
| | Variant | VKKVIDNYHKDFINECLSKRSFDWTPLMKTSEKYYASKEKNGKKKQDLD |
| | | QKIIPTIENLSEKDRKELELEQKRMRKEIVSVFKEDKRFKYLFSEKLFS |
| | | ILLKDEDYSKEKLTEKEILALKSFNKFSGYFIGLHKNRANFYSEGDEST |
| | | AIAYRIVNENFPKFLSNLKKYREVCEKYPEIIQDAEQSLAGLNIKMDDI |
| | | FPMENFNKVMTQDGIDLYNLAIGGKAQALGEKQKGLNEFLNEVNQSYKK |
| | | GNDRIRMTPLFKQILSERTSYSYILDAFDDNSQLITSINGFFTEVEKDK |
| | | EGNTFDRAVGLIASYMKYDLSRVYIRKADLNKVSMEIFGSWERLGGLLR |
| | | IFKSELYGDVNAEKTSKKVDKWLNSGEFSLSDVINAIAGSKSAETFDEY |
| | | ILKMRVARGEIDNALEKIKCINGNFSEDENSKMIIKAILDSVQRLFHLF |
| | | SSFQVRADFSQDGDFYAEYNEIYEKLFAIVPLYNRVRNYLTKNNLSMKK |
| | | IKLNFKNPALANGWDLNKEYDNTAVIFLREGKYYLGIMNPSKKKNIKFE |
| | | EGSGTGPFYKKMAYKLLPDPNKMLPKVFFAKKNINYYNPSDEIVKGYKA |
| | | GKYKKGENFDIDFCHKLIDFFKESIQKNEDWRAFNYLFSATESYKDISD |
| | | FYSEVEDQGYRMYFLNVPVANIDEYVEKGDLFLFQIYNKDFASGAKGNK |
| | | DMHTIYWNAAFSDENLRNVVVKLNGEAELFYRDKSIIEPICHKKGEMLV |
| | | NRTCFDKTPVPDKIHKELFDYHNGRAKTLSIEAKGYLDRVGVFQASYEI |
| | | IKDRRYSENKMYFHVPLKLNFKADGKKNLNKMVIEKFLSDKDVHIIGID |
| | | RGERNLLYYSVIDRRGNIIDQDSLNIIDGFDYQKKLGQREIERREARQS |
| | | WNSIGKIKDLKEGYLSKAVHKVSKMVLEYNAIVVLEDLNFGFKRGRFKV |
| | | EKQVYQKFEKMLIDKLNYLVFKEVLDSRDAGGVLNAYQLTTQLESFNKL |
| | | GKQSGILFYVPAAYTSKIDPTTGFVSLFNTSRIESDSEKKDFLSGFDSI |
| | | VYSAKDGGIFAFKFDYRNRNFQREKTDHKNIWTVYTNGDRIKYKGRMKG |
| | | YEITSPTKRIKDVLSSSGIRYDDGQELRDSIIQSGNKVLINEVYNSFID |
| | | TLQMRNSDGEQDYIISPVKNRNGEFFRTDPDRRELPVDADANGAYHIAL |
| | | RGELLMQKIAEDFDPKSDKFTMPKMEHKDWFEFMQTRGD |
| SEQ ID NO: 167 | Cas12 | MLHAFTNQYQLSKTLRFGATLKEDEKKCKSHEELKGFVDISYENMKSSA |
| | Variant | TIAESLNENELVKKCERCYSEIVKFHNAWEKIYYRTDQIAVYKDFYRQL |
| | | SRKARFDAGKQNSQLITLASLCGMYQGAKLSRYITNYWKDNITRQKSFL |
| | | KDFSQQLHQYTRALEKSDKAHTKPNLINFNKTFMVLANLVNEIVIPLSN |
| | | GAISFPNISKLEDGEESHLIEFALNDYSQLSELIGELKDAIATNGGYTP |
| | | FAKVTLNHYTAEQKPHVFKNDIDAKIRELKLIGLVETLKGKSSEQIEEY |
| | | FSNLDKFSTYNDRNQSVIVRTQCFKYKPIPFLVKHQLAKYISEPNGWDE |
| | | DAVAKVLDAVGAIRSPAHDYANNQEGFDLNHYPIKVAFDYAWEQLANSL |
| | | YTTVTFPQEMCEKYLNSIYGCEVSKEPVFKFYADLLYIRKNLAVLEHKN |
| | | NLPSNQEEFICKINNTFENIVLPYKISQFETYKKDILAWINDGHDHKKY |
| | | TDAKQQLGFIRGGLKGRIKAEEVSQKDKYGKIKSYYENPYTKLTNEFKQ |
| | | ISSTYGKTFAELRDKFKEKNEITKITHFGIIIEDKNRDRYLLASELKHE |
| | | QINHVSTILNKLDKSSEFITYQVKSLTSKTLIKLIKNHTTKKGAISPYA |
| | | DFHTSKTGFNKNEIEKNWDNYKREQVLVEYVKDCLTDSTMAKNQNWAEF |
| | | GWNFEKCNSYEDIEHEIDQKSYLLQSDTISKQSIASLVEGGCLLLPIIN |
| | | QDITSKERKDKNQFSKDWNHIFEGSKEFRLHPEFAVSYRTPIEGYPVQK |
| | | RYGRLQFVCAFNAHIVPQNGEFINLKKQIENFNDEDVQKRNVTEFNKKV |
| | | NHALSDKEYVVIGIDRGLKQLATLCVLDKRGKILGDFEIYKKEFVRAEK |
| | | RSESHWEHTQAETRHILDLSNLRVETTIEGKKVLVDQSLTLVKKNRDTP |
| | | DEEATEENKQKIKLKQLSYIRKLQHKMQTNEQDVLDLINNEPSDEEFKK |
| | | RIEGLISSFGEGQKYADLPINTMREMISDLQGVIARGNNQTEKNKIIEL |
| | | DAADNLKQGIVANMIGIVNYIFAKYSYKAYISLEDLSRAYGGAKSGYDG |
| | | RYLPSTSQDEDVDFKEQQNQMLAGLGTYQFFEMQLLKKLQKIQSDNTVL |
| | | RFVPAFRSADNYRNILRLEETKYKSKPFGVVHFIDPKFTSKKCPVCSKT |
| | | NVYRDKDDILVCKECGFRSDSQLKERENNIHYIHNGDDNGAYHIALKSV |
| | | ENLIQMK |
| SEQ ID NO: 168 | Cas12 | MKNGINLFKTKTTKTKGVDMEKYQITKTIRFKLLPDNAHEIVEKVKSLK |
| | Variant | TSNVDELMDEVKNVHLKGLELLFALKKYFYFDGNQCKSFKSTLEIKARW |
| | | LRLYTPDQYYLKKSSKNSYQLKSLSYFKDVFNDWLFNWEESVSELAIIY |
| | | EKYKICQHQRDSRADIALLIKKLSMKEYFPFISDLIDCVNDKNSNKTFL |
| | | MKLSEELSVLLEKCNSRALPYQSNGIVVGKASLNYYTVSKSEKMLQNEY |
| | | EDVCQSLDKNYDITEMKVILYKEKLDNLNFKDVTIANAYNLLKENKALQ |
| | | KRLFSEYVSQGKVLSLIKTELPLFSNINDNDFEKYKEWSNEIKKLADKK |
| | | NTFCKKTQQDKIKDIQNKISELKKKRGALFQYKFTSFQKHCDNYKKVAV |
| | | QYGKLKARKKAIEKDEIEANLLRYWSVILEQEDKHSLVLIPKNNAKDAK |
| | | QYIETINTKGGKYIIHEILDSLTLRALNKLCFNAVDIEKGQMVRENTFY |
| | | QGIKEEFERNKINCDNQGVLKIQGLYSFKTEGGQINEKEAVEFFKEVLK |
| | | SNYAREVLNLPYDLESNIFQKEYTNLDQFRQDLEKCCYALHSKIGKDDL |
| | | DEFTRRFEAQVFDITSIDLKSKKEKTKTTGEMKKHTQLWLEFWKGAIEQ |
| | | NFATRVNPELSIFWRAPKSSREKKYGKGSDLYDPNKNNRYLYEQYTLAL |
| | | TITENAGSHFKDIAFKDTSKIKEAIKEFNMSLSQSKYCFGIDRGNAELV |
| | | SLCLIKNEKDFPFEKFPVYRLRDLTYQGDFKDKHDQMRYGVAIKNISYF |
| | | IDQEDLFEKNNLSAIDMTTAKLIKNKIVLNGDVLTYLKLKEETAKHKLT |
| | | QFFQGSSINKNSRVYFDEDENVFKITTNRNHNPEEIIYFYRGEYGAIKN |
| | | KNDLEDILNEYLCKMETGESEIVLLNRVNHLRDAISANIVGILSYLIDL |
| | | FPETIVALENLAKGTIDRHVSQSYENITRRFEWALYRKLLNKQLAPPEL |
| | | KENILLREGDDKIDQFGIIHFVEEKNTSKDCPNCRKTTQQTNDNKFKEK |
| | | KFVCKSCGFDTSKDRKGMDSLNSPDTVAAYNVARKKFES |
| SEQ ID NO: 169 | Cas12 | MAKETKEFKTFDDFTNLYEVQKTLRFELEAVPETEIVLENRGIWYKRDK |
| | Variant | KRADEKPIVKFYMDILHREFTDEALEKIKESGVLNLSGYFKLFEELRRL |
| | | QNHGANTKEEKKLKLEEIRAKKREISNELSQIRRVFSVRGFDVVDSDWK |
| | | KKYTIEGKKIKNDKSKTYLILSENILNFLENRFTSKEVERLRSIDKKHV |
| | | EDYGNVVNSGGENIFATFKGFFGYFDSLIKNRENFYETDGKAGRVATRS |
| | | VDENLNFFAENLHIFSTDLPKALKDDLSDTQKAIFERSYYKNCLLQKDI |
| | | KSYNLIIGDINKEINKHRQQRDTKIKFLNTLFKQILSIEEKEQYKHIEI |
| | | NNDEDLIRAIRDFISLNESKISEGTKIFNQFIQRCLQKEDLGQIYLPKD |
| | | SVNTIAHRIFKPWDEIMALFDRKYFVSLEEIKDLTESSVWKERVLEESK |
| | | TKSLIFKDTHIHTIISGQEIFSNFILILEKEYKNQFSGFISETRRGKAA |
| | | FVGYDESLKNLRATIKWFEGKNLKLSETEKVEWIKAIKDYADAALRIFQ |
| | | MTKYLWLPVVGDEEDKDYLRIKAEIDQLTKDNDFYNKINAFIDGYKPEP |
| | | FIYRSSFQEYLTRRPFSTDKFKINFENSRLLDGWDKDMIDDRMGILLQR |
| | | DGDYFLGILNKEDRHCLDNLVDVKSEDKNSYALMQFKQLTGLYRQLPRM |
| | | AFPKKKQPVLEANAEIKKIKEDFDFLQKQKKEREVNVNVVFDNKKLNLL |
| | | INHYAEFLKENYKDEKCYDFSLLNKEKVYESLSDFYADVDKITYSLSFI |
| | | QVSIDQLIKTGKILLFRLKNKDLLKGSLGQNKNLHTYYFHALFERENLS |
| | | QGRIRLGAQAEIFFRPASIEKEKDKNRSNALKKSPKTRYVKEILKNKRY |
| | | SEDKVFLHLPIQLNADAYDLPSINQNVFEFIKNRQEKVKIIGIDRGEKN |
| | | LAYYSVISQNSNGKIKIEEPPRDLNLGYLEPLDELENKRQDERKAWQSI |
| | | SEIKSKRDGYISYAVSKIVELMLKYQAIIVLEDLSGKFKRSRMKFEKAP |
| | | YQQLELALIKKLNYLVKKNSKSGKPGHYLSAYQLTEPVGSYKEMGKQTG |
| | | IIFYTQAGYTSRTCPTCGWRKRVQGLYYKDRTSAQRRFDPKTGVKIFYD |
| | | SVNDRFVFQYHPVYEQKELKEWDKEIYSDVTRIRWNNEEKKNNEYRKGD |
| | | ITLKIKRLFRDRGIDLSRNINEQLVNVGDASFWEELINLLRLITEIRNI |
| | | DNENNRDFIECPHCHFQSENGFHGVAWNGDANGAYNIARKGLLITKAVC |
| | | DPEKNVGDITWSDLKVDMKDWDAATDEWAKKNPEK |
| SEQ ID NO: 170 | Cas12 | MENEKIFSDLTNRYQVVKTLPFELKPVPRTRVLLGLDNPNKGEIFSKDR |
| | Variant | ERAENFTIIKKYIDRLHSLFINESLKKADIDFSNFYKQYGKNINTKNNK |
| | | NIDDDNDINDDEKEDSENDNLKKYRQEIANLFNKSKYKSWVNVGKDGDK |
| | | ISGMLFEKGLIDLLRTHFSDNLNEDIEIPELFSNKKIKDTRKLKEIINS |
| | | FGKDGKDGQNFTTYFSVSFHNNRKNYYKSDGKMGRVSTRIVDENLERFC |
| | | KNIYLYKEIIGKNEIKEIFSGNWDIYLQKKPNFSNDKTYKKLDEFKNDK |
| | | YDWEMIFRDVNSYNKYFLQSDIEFYNYIRGKLNQDINEYNGKKRDSKEK |
| | | INSQFENLRNQVHGEKKNYDDDFEIDEDNIIQFINEIFVRHNQNKMRFS |
| | | EKLFSDFIDLLMVDNGDKLDKVYFSQKAVENAIARYYFVEETTNEGREP |
| | | LLISLLLQNAGKDRKKLSNKPIKLGDIKFVLDQANNKPAEDIFKNRYVL |
| | | SESNNDGIINANDKNHWANLLRLIKKDFYFHKDNLIKSQDKLALETKYN |
| | | KGSDEGERQIETIKNFAESAKAILRMTKYFDLRKNGVIQNVIGGKDPIH |
| | | EEVDKYFDGDVLSGEESCRISKYYDALRNFITKKAWSADKIILNFDCSE |
| | | FLGGWDRSQEQKKRGIILRHRDGDEERYYLAVLGKNGKQYFENRTLFKG |
| | | CESSDWQKIEYNVIQKPHMSLPKNLITPFFKKDKITNERFIDRSKKGAK |
| | | ALIEIDINPSDEFLNNYNLGKHTKENLDKSFLCDYFKYLMDAIAKYYKG |
| | | EFNFNFPDVSNFDNTQPFYSFIEKNAYSIKYFGISSKEIEKLIADCYYK |
| | | EDVYLFQIYCKDFEIDPKIGKAKYGNEFRTKAEIRKSKGEEAGNENLNT |
| | | KYFKLLFDEKNLKNQNGIVYKLNGGAKMFYRPSSIKKDEKIDGKWRYKE |
| | | DKYSLNITITCNFSSKKDDLSIDKDINKKIAEVNANSDFRIISIDRGEK |
| | | NLAYCCVMDENANILDIKSLNRITRYDKNGKAIKEKNMFHEVKDGKLCY |
| | | GEPVYDFYKDYQNLLDEREIKRLVNRRSWNVIEDIKNLKKGYVALLINY |
| | | ICKAVVIAINEGKYPIIVLESLDKGMLHNRVKIEKQIYRGVEEGLVRKL |
| | | NYFVDKKTDNVLNAWQLLAKFETVGSSLDRKKQLGIIFYVDPGYTSITC |
| | | PCCGFRQRKYIKAERAEENFKEIKIKFDGKRYSFAYDYRCIDDNGKEKS |
| | | KEDIIYSNVKRLLRSGRNGRAVQIEDVTDELTNLFKKHNINIEQDINEQ |
| | | LAGKDNKFWKQLLWWFNAIEQIRNTQSLRRKFNTEENKLEILENNDCDF |
| | | ILCPHCYFDSNKDKFQNKIWNGDANGAFNIGRKGIIDIFEIKKHQRMLS |
| | | DFMEQWGIDKLPKANGGNQAVIEIVKNDKKYNLCILNNKKIPYYCLRIG |
| | | KEKIDSIADDRKCNQLPDLMVNWKKWDMWLDKWGK |
| SEQ ID NO: 171 | Cas12 | MPEVKNVFQDFTNLYELSKTLRFELKPVPETEKILELNAAKTKKFPKDL |
| | Variant | YRAENFEIIKKYTDELHRTYIRETLNNVNIDYLKFLEIFRINGKKKNEM |
| | | TDENEESDENNEKDDIQKIKKELRSKIGNLFNKWNNDKDNKFKDWVKID |
| | | VGKKEKEVSGDLFGKELITILKNYFKNKLDSKVNVPMLFFNEQEIKNGE |
| | | AKKQRKLEAVFENFDKFTTYFTDSFYNNRKNYYKTEGRVGQVATRIIDE |
| | | NLPRFCSNLIAFNEVVSLYSTLLNNFDLGWKEYLNEKKINQTWVEKFEL |
| | | SNYDWKALFNDVNYYNQCLLQEGIDKYNYIIKKLNKDINEYTQNKYKSV |
| | | EKGNNNNPDINFFQKLHKQIHGERDFKLIEIDIDENNIFTKILPEFILH |
| | | SDMKLMTKIDEEVGVEEIVGAERIIKIFIKQELKDLEKIYLSRRAIETI |
| | | SAKWFHSWETLKDLILGYLNKDLLESKKRKKVPDFVDFNIIKIVLENNK |
| | | DDYKDLFKRKYFEADKNEFVDWIDSSGGTKKLEFGGENWINFLNVFEYE |
| | | FGTLLTEYKKNKNALLYLIDKKIDYDKNNEVGQTAAIKNFADSALGIFR |
| | | MVSYFALRKKGVMVEPKNGKDEIFYAFVDRYLDGDDNDREEQNKIVQYY |
| | | NTLRNFVTQKAWSIDKVRLCFDCGEFLKGWDKDKIHERLGIILRNNNKF |
| | | YLGILNKNHKQIFIKIKSHDNNNFYYVIYDYKQLNNVYRQIPRLAFPSR |
| | | SVKKGDAYMLRAIQERKKKFFLEDEEFIELQEIKNEYDKIGNDLSKEKL |
| | | TKLIEYYKKVVISNYSSLYNVSNLNNKKFNSINEFNQYVENLMYSLIPT |
| | | RISPDFIKEKISKGELYLFQIYNKDFELDESIGKEKFGEDFAPVIMDGK |
| | | NNLHTEYFKLLFNDSNLKNPNGVVFKLSGGAKMFYRPATENLPIKKDRD |
| | | GNIIKNKKGENVIVGQRYKEDKYFLHLPIILNFVNKGKNYSINDMVNKA |
| | | ITNASDDQDKFRIIGLDRGEKHLVYYSVINERQEIIEIGSLNNISRKDN |
| | | KGEIIEEKNWYHDKFGNIEKEPTKEYHKDYHNLLDQREIERLKSRQSWE |
| | | KIENIKELKEGYISAVINKICNLVIKAIKENKIPIVALENLNSGMKRGR |
| | | IKIDKQIYQKLELKLAKKLNFLVDKKEKNYLSAWQFTPKIETFSGDIEK |
| | | KNQVGIIFYVDPAFTSATCPNCGFRKRIKMDPQNAKKKIKDMEITYENG |
| | | IYKFDYPIENGENDVVYSDVERLKWDNEKKKVIKTKNVSDDFGKLFEDI |
| | | KDKNNLKKELLSIGEENKEFWKEFSRCFNLLLRIRNSKLIKRKLNDDTG |
| | | KVEIIADDDLADRDRDFIYCPQCHFHSEGGDVFGEFVKKKYLGKDNFEF |
| | | NGDANGAYNIARKTIIAVNKIKDYQLGLNHFIEKYRISELPNNGKDKKN |
| | | IFYNNNSYILSFFEVQDEKFRKVKVYGLKKDGDRQIIQKKEMWYRRYPD |
| | | IFVNNKEWDKFVQNKS |
| SEQ ID NO: 172 | Cas12 | MLFFMSTDITNKPREKGVFDNFTNLYEFSKTLTFGLIPLKWDDNKKMIV |
| | Variant | EDEDFSVLRKYGVIEEDKRIAESIKIAKFYLNILHRELIGKVLGSLKFE |
| | | KKNLENYDRLLGEIEKNNKNENISEDKKKEIRKNFKKELSIAQDILLKK |
| | | VGEVFESNGSGILSSKNCLDELTKRFTRQEVDKLRRENKDIGVEYPDVA |
| | | YREKDGKEETKSFFAMDVGYLDDFHKNRKQLYSVKGKKNSLGRRILDNF |
| | | EIFCKNKKLYEKYKNLDIDFSEIERNFNLTLEKVFDFDNYNERLTQEGL |
| | | DEYAKILGGESNKQERTANIHGLNQIINLYIQKKQSEQKAEQKETGKKK |
| | | IKFNKKDYPTFTCLQKQILSQVFRKEIIIESDRDLIRELKFFVEESKEK |
| | | VDKARGIIEFLLNHEENDIDLAMVYLPKSKINSFVYKVFKEPQDFLSVF |
| | | QDGASNLDFVSFDKIKTHLENNKLTYKIFFKTLIKENHDFESFLILLQQ |
| | | EIDLLIDGGETVTLGGKKESITSLDEKKNRLKEKLGWFEGKVRENEKMK |
| | | DEEEGEFCSTVLAYSQAVLNITKRAEIFWLNEKQDAKVGEDNKDMIFYK |
| | | KFDEFADDGFAPFFYFDKFGNYLKRRSRNTTKEIKLHFGNDDLLEGWDM |
| | | NKEPEYWSFILRDRNQYYLGIGKKDGEIFHKKLGNSVEAVKEAYELENE |
| | | ADFYEKIDYKQLNIDRFEGIAFPKKTKTEEAFRQVCKKRADEFLGGDTY |
| | | EFKILLAIKKEYDDFKARRQKEKDWDSKFSKEKMSKLIEYYITCLGKRD |
| | | DWKRFNLNFRQPKEYEDRSDFVRHIQRQAYWIDPRKVSKDYVDKKVAEG |
| | | EMFLFKVHNKDFYDFERKSEDKKNHTANLFTQYLLELFSCENIKNIKSK |
| | | DLIESIFELDGKAEIRFRPKTDDVKLKIYQKKGKDVTYADKRDGNKEKE |
| | | VIQHRRFAKDALTLHLKIRLNFGKHVNLFDFNKLVNTELFAKVPVKILG |
| | | MDRGENNLIYYCFLDEHGEIENGKCGSLNRVGEQIITLEDDKKVKEPVD |
| | | YFQLLVDREGQRDWEQKNWQKMTRIKDLKKAYLGNVVSWISKEMLSGIK |
| | | EGVVTIGVLEDLNSNFKRTRFFRERQVYQGFEKALVNKLGYLVDKKYDN |
| | | YRNVYQFAPIVDSVEEMEKNKQIGTLVYVPASYTSKICPHPKCGWRERL |
| | | YMKNSASKEKIVGLLKSDGIKISYDQKNDRFYFEYQWEQEHKSDGKKKK |
| | | YSGVDKVFSNVSRMRWDVEQKKSIDFVDGTDGSITNKLKSLLKGKGIEL |
| | | DNINQQIVNQQKELGVEFFQSIIFYFNLIMQIRNYDKEKSGSEADYIQC |
| | | PSCLFDSRKPEMNGKLSAITNGDANGAYNIARKGFMQLCRIRENPQEPM |
| | | KLITNREWDEAVREWDIYSAAQKIPVLSEEN |
| SEQ ID NO: 173 | Cas12 | MTIKKHKPFTNFECLTPVQKTLRFRLIPVGRTTEFVKCRNIIEADRKRS |
| | Variant | EMYPLLKELADRFYREFMTDQLSNLLFDWSPLVEALLLARNNTDPRENQ |
| | | RIASLVRDEQKKYRTLLLKRLSGQVDRNGTPLPKNTASVNKKYYDDLFK |
| | | ARFVTETLPAYLEHLKNKPDGRISDELFDAYKDALDSYQKFTSRLTNFW |
| | | QARKNIFTDEDIATGFAYRIVHEIVPDYLFNRRVYEQHKLDFPEPLDLL |
| | | ETELKKKNLIANDESLDALFTIPAINRLLTQKGVDLHNAVIGGFFTDDH |
| | | TKVQGFNELANLKNQTLKNVSDNSEIKPVGKMTRLKKHILSISESTSFL |
| | | FEQIESDDDLLARIIEFNNTLSEPDIDGLSIADINDQLYNIMTGVDPST |
| | | ILVHARNLNKLSHEASLSWNRLRDGLYQMATESPYREDERFKRYIDASE |
| | | EERDLSKLKNDIYFSLQELQFALDQSIDLEEEATPTEDIFLPFEFPGMD |
| | | LKSELTVLFRSIEQLISSETKLIGNPDAIATIKKYLDAIMARYSIWNLL |
| | | SCEAVELQDDLFYPEYDRVMGSLSNIILLYNLARNYLSRKPSSKEKFRL |
| | | NFDKPTLADGWSESKVPDNFSVLLRKDDLFYLGILKDRKAYRVLSYENC |
| | | DETAKNIKGYYERMIYHFSPDAYRMIPKCSTARKDVKKHFGEQGETTGY |
| | | TLYPGASNFVKPFTIPYEIYRLQTELVNDKKRYQADYLKQTEDEEGYRQ |
| | | AVTAWIDFCKSYLESYEGTSTFDYSHLLKSEDYEDVNQFYADVDRASYS |
| | | IYFEKVSVDLIHTMVDRGDLYLFQLYNKDFSPHSTGKPNLHTMYWRALF |
| | | SNDNLQNNTIKLNGQAELFYRPKQVEQPTVHLQGSYLLNRFDKHGDVIP |
| | | AGLYCEIYNHINERHPEGYTLSEEATQGLLDGRFVYREAPFELVKDKRY |
| | | TEDQLFLHVPLEFNWTASANVPFENLANEYIKKDSDLHIIGIDRGERNL |
| | | LYYSVINLQGDIVKQGSLNTLIQQTTLKGETVERQIPYQSMLKQREDER |
| | | AEARQNWQSIDRIKDLKEGYLSHVIYKLSRLIIKYHAIVVMENLNVGFK |
| | | RGRFKVERQVYQKFEVALINKLNALSFKEYEPNELGGVMRPWQLARRVV |
| | | SPEDTRSQNGIVFYVPASYTSIVDPVTGFANLFYLNRIRNKDLNSFYGH |
| | | FQEIRYDHEFDRFIFRFNYADFGVFCRIKNVPSRTWNLVSGERKAFNPK |
| | | RRMIEKRDTTDEIKKALEAHGIAYQNEQNLLPLLLENENLLARIHRSFR |
| | | LVLQLRNSDSDRDDIVSPALDKENNTFDSGQQPYESSLPINADANGAYN |
| | | IARKGLLLVDKVKNDKRAVLSNREWFEYLMAEE |
| SEQ ID NO: 174 | Cas12 | MENKDYSLSRFTKQYQNSKTVRFALTPIGRTEEYIIQNQYIEAARRKNQ |
| | Variant | AYKIVKPIIDEKFRSMIDDVLTHCEKQDWVTLDKLILQYQNNKCRENMD |
| | | ALAEQQEEIRKNISEEFTKSDEYKNFFGKEDSKKLFKIFLPEYLNQINA |
| | | SESDKEAVNEFQKFKTYFSNFLIVRADIFKADNKHNTIPYRIVNENFMI |
| | | FAGNKRTFSNIIRLIPNALEEIAKDGMKKEEWSFYNIQNVDSWFEPDSF |
| | | QMCMSQKGIQKYNFIIGLVNSYINLYTQQNPQATEVKRSRLKLRMLHKQ |
| | | ILSDRVNPSWLPEQFKEGEEGEKQIYEAILALENDLIKNCFDKKYDLWI |
| | | QSIDIQNPRIYIAASEMARVSSALHMGWNGLNDVRKTILLKSDKKQAKV |
| | | EKILKQDVSLKDLSDTLNRYADIYKEEQIPSLYQYIEYGSELLQDCAIT |
| | | RKEYHDLLNGNSNTLSLNQNEKLIEGLKAYLDSYQAIVHFLNVFIVGDE |
| | | LDKDTDFYAELDGLVESLSEIVPLYNKVRNYITRKVYSLDKMRIMFERS |
| | | DFLGGWGQSFDTKEALLFQKDNLYYIGIIEKKYTNMDVEYLHEGIKEGN |
| | | RAIRFIYNFQKADNKNIPRTFIRSKGTNYAPAVRKYNLPIESIIDIYDV |
| | | GKFKTNYKKINEKEYYESLEKLIDYFKDGILKNENYKKFHFNWKPSNEY |
| | | ENINEFYNDTNNACFLLEKEEINYDHLKEQANQGKIYLFQISSKDFNEG |
| | | SKGTPNLQTMYWRELFSNQNCKDGVIKLCGGASIYMRDASIKQPVVHRK |
| | | NAWLINKWYKVNGQNVVIPDNTYVKFTKIAQERMNEDELTPQERQLWNS |
| | | GLIQKKKATHDIMKDRRFTKKQYMLHAPLTINYKQQDSPRYFNEKVRSF |
| | | LKDNPDINIIGIDRGEKNLIYITIIDQKGNILKGMQKSFNQIEEKGKEG |
| | | RTIDYYSKLESVEARHDAARKNWKQIGTIRELKEGYLSQVVHEITQLMI |
| | | QYNAVIVMENLNMGFKKGRMKVEKSVYQKFEKMLIDKMNYLAFKRDMQG |
| | | NAIDPYEVGGVMNGYQLTDRFTSFADMGSQNGFIFYVPAAYTSVIDPVT |
| | | GFVNVFQKTEFKTNDFLHRFDSISWNDKEQSFVFTFDYQNFKCNGTCYQ |
| | | NKWSLYADVDRIETIIKNNQVDRIEPCNPNQKLIDFFDKKGIIYRDGHN |
| | | IVDDLEKYDSKTISEIIHNFKLILQLRNSMRNPDTGEHDYIASPVMEIN |
| | | EERFDSRKRNPELPQDADANGAYHIALKGLMFLQKINEYADSDGNMDNR |
| | | KLKITNEEWFKYMQTRKEHTYF |
| SEQ ID NO: 175 | Cas12 | MSNKTSSITTTNKLSYTGFHNNGKQSKTLMFELKPIGRTTEHLDRKGYL |
| | Variant | ADDIDRAESYKTFKEIADNFHKNLIEESLATFTFSDTLKDYFDLWLSPV |
| | | RTNEDTPKLRKMEAKLRKELSSALKQHPSFAATSSGKRLIDEALYPNAS |
| | | DKERQCLDRFKGRSSYLDSYTEVRSFIYTDLCKHNTIAYRVVNENLKIY |
| | | LENILAYEKLMQTAVNGKLETVKEMFHDLYPTFSMDISIFFTSYGFDYC |
| | | LSQNAITRYNILLGGWSDDNGIFIHKGLNNYINEYNQTVPRNKRLPKLN |
| | | KLQKMILSEENSMSFIIDKFENDVDLANAIRYWLKNCQFDALNLLIWTL |
| | | DVHYNLDEIHFKNDNQGKNISDLSQALFKNHHVIRDAWDYDYDIVNAKA |
| | | KSRQKPERYAEKRDKAFKKINSFSLSYLANILSQYDNQYANFVAQFKTR |
| | | ISVHIQNVQQMIADKTLDMRLDPLMLLKSISSDTKLVEDIKRVLDSLKD |
| | | MQRMLTPLLGEGTEPNRDAMFYSDFEPLMNYVDTLTPLYNKVRNYITKK |
| | | PYSTKKTSLYFGASNFGSGFDVTKLPVSHTIIMRDKGCYYLAVIDNNKL |
| | | IDKLYDHNDNDGYEYMVYKQIPSPIKYFSLKNILPQDPPDDIRQLLEDR |
| | | KNGAKWSHDDETRFIDYIVNEFLPTYPPIHDKNGNPYFSWKFKNPDEYE |
| | | SLNEFFDDVSKQAYQTSFRFVSRDFVDDAVENGDIFFFQIYNQDFSPAS |
| | | HGKPSPHTLWFRALFSDVNLETKDIRLKGNATAYFRPASIFYTDEKWRK |
| | | GHHYEQLKNKFKFPIIKDKRYALDKFFFHITLEINCNATVEKYFNNRVN |
| | | EEIRKADRYNILAINRGERNLLYAVVMDQDGTILEQKSFNIIKSELPNK |
| | | TVKETDYWKKLHAREKERDTARKSWKSIECIKDLKKGYLSYVVKTITDM |
| | | MFEYNAVLVMENLDIEMKRSRQKIEKNVYAQFQNAIIQKLSMYVNKDID |
| | | LHIARTAPGGTLNPYQLTYIPASRTKTPKQNGFVFFLNPWNITEIDPTT |
| | | GFVDLFQTCFRTKNEYKDFFAKFKDIRYNEAQGWFEFDTDYTYFRDKEK |
| | | AGKRTRWNICSYGTRLRRFRNPDKNYAEDAMTVYPTQMLKDLFDEYNIP |
| | | YAPASAKSTSISIKDDIIQIDKLDFYKKLLYILKLIVQLRNTSPSSTEQ |
| | | EDDYIISPVINEDTNWFYDSRDYNEESLLPCNTDANGAYSLALKCNMVI |
| | | DRIKNTIPGEPVDMYISNADWLDARQ |
| SEQ ID NO: 176 | Cas12 | MNSKTSIFDFSNIFGRDITLRFKLTPVTINSKGEVKDANGADPYRPYLS |
| | Variant | ADEELQEQYELLKTAIDAYHQMYIDKKLKHILCLPLTEKGKDGVEHDTA |
| | | KSKFVKSCLAYIKDYGEKDKKRQTADLRTFISRVFADDNISSLPPYKVK |
| | | SDFITKTLRQWLEQPDTKVEKKEAILDLIEKNGSKLYANCQGLLEARQR |
| | | LYEKDGKSTSVPYRCIDRNLPRFSKDYHLFEKILGDCSDVFDFEQLDKD |
| | | FSEELKGIARLSGIRVESVREVFQPLLYLAYLNQEGIQYLNTIIGTKKE |
| | | KGTSALGLNEYINQYNQKQGIKKKKDGIPMLNKLNNQILFGDEVFIETL |
| | | AEHKEAIPVIKKVVSSLGKLGAFDGECHENKLYQFLLSLSSYAGNIYVN |
| | | TKVVAQISSSLWGDYSILYDAVKHDKNGRLIQKSVTLGELNEKIERLKL |
| | | EDNRDAFEYFRRSQVKDVVHGSSNVGVFEQLKNCYNDFVEKKILKCSFF |
| | | SEDQVLVIQRLFDSILSLQRIFKVFCPSLYEVDSDGLFVAKFSDYWNVL |
| | | RGFDKDYDLLRNLFKRKPYSTDKIRVHFGLSNLMDGFVDSWTDKKDKGT |
| | | QYNGYILRQAHSFVDENTSKELQEFQRYNYYLVISGNVRLFREKGNALV |
| | | CEKKKEKLVASDEFSGFERFDYYQSSINNFNREFKRLTGRDRKSFTDEI |
| | | LQNEGKKELKSTYIENLIKVAKSMKRLTALQNLVSDEKVRKYSENLDYE |
| | | TLSAEIGQILATGRERKYVPVSTNEMKNLLKSSKNNKGEEVRTFMFRIS |
| | | NKDLSYAETMQKGERKSHGAENMHTMYFRALLDTLQNTFDIGTGTVYFR |
| | | KASDKRKMKYDEKNPTHRKGDELAFKNPYNKGKKKSVFGYDLIKDRRYT |
| | | KDSYLFHLSITQNYQKKGNAEDLNAMVRDYIRTQEDLRVIGIDRGERNL |
| | | LYATMIDGEGHILAQKSFNVIGYQGTTASGESFQVETDYHQLLNEKAEK |
| | | MRSLQREWKEMDKIQDMKDGYLSVVVHELAKMVVENNAIIVMEDLNMGF |
| | | MESRQSQLANVYQKFEEKLRNKLQFYVDKRKRNDEPSGLYHALQLAGTE |
| | | TKDNQNGFIFYIPAWNTSKIDSVTGFVNLFNLKYTNIKDAKAFFSTFEK |
| | | IEKNVETGHYDFTFSYSSMARKKMAKRMDGTRDSWTISTHGSRIVREQK |
| | | GNYWEYREIESLTSEFDALFEKYSIDTRCRLKEAIDKCGEAEFFKELIR |
| | | LMKWTLQLRNYDDRGNDYIVSPVCYRGNEYYCSLDYDNEEGMCISKIPC |
| | | QMPKDADANGAFNIARKGLMLCERLKKGEKIGVIKGTEWLQYVQNMSER |
| | | YVGMV |
| SEQ ID NO: 177 | Cas12 | MINTMEQPKKSIWDEFTNLYSLQKTLRFELKPQGKTKELVRTLFINPEE |
| | Variant | HHHKLISDDLELSKNYKKVKKLIDCMHRNIINNVLSKHQFTGEELKKLD |
| | | KNSNAEDNDTETDNADKKDPFAKIRERLTKALNEESKIMFDNKLLNPKK |
| | | GKNKGECELKKWMDKAEDKYFELGNNEKIDKEAVKADMERLEGFFTYFG |
| | | GFNKNRENVYSSKKIATAIPFRIIHDNFPIFKKNIENYKKITEKHPELA |
| | | KLLNEKGANEIFQLEHFNKCLTQDGIDVYNNEKLGIIAKEQGKEQDKGI |
| | | NQLINEYAQKKNKEIKENAKGGEKPKKIKIAVFDKLKKQILSISKTKSF |
| | | QFEVFEDTSDIINGINKRYTFLTEAKEGMSIVDEIKKIIGSVGDEKYSL |
| | | DEIYLKEKFISTLSKKLFNYSRYIEVALEKWYDDRYDDKINKSGTDKRK |
| | | FISAKQFSITSIQDAINYYLEKYEKDEELSKKYTGKNIIVDYFKNPTIT |
| | | IEHKQKEEVISEEKDLFKELEVRRNVIQHILNGDYKKDLKEEKQQDGDS |
| | | EKVKAFLDALLEFNYILNPFIIKDKNLRKEQEKDEEFYNEIKKLQESIF |
| | | EAEILDLYNQTRNYITKKPYKLDKFKLTFGSGYFLSGWSNDMEEREGSI |
| | | LIKYNEDRSKNYYLIIMAKPLTDDDKKQLFSDNGTHSKICIYEFQKMDM |
| | | KNFPRMFINSKGSNPAPAIEKYNLPIKTIWADYQKYKNLNQKGKDKFLE |
| | | ENPDFRHNLIGYFKICAEKHESLAPFKHQFSSIWKPTKEYENLAQFYKD |
| | | TLEACYNLKFENVNFDNISQLVSSGKLHLFKIHNKDFNPGSTGKKNLHT |
| | | LYWEMLFDEKNLQDVIFKLSGGAELFYREASILKNKIIHKIGEKVLKKF |
| | | FKLPDGKLEPVPAESIKNLSAYFRKELPEHELTEIDRKYIDNYSIIGKK |
| | | DDKLGIMKDERFTVDKIQFHCPITINFKSKNKNFINDDVLEYLHKRDDV |
| | | HIIGLDRGERHLIYLTMINKDGKIVDNMQFSLNELQRRYKINGNEEIQK |
| | | INYQKLLDTREVSRTEARRNWQTIENIKNLKEGYLSLIVHQLAKLMIEK |
| | | NAIVVMENLNYGFKDSRARVEKQIYQKFESILIKKLQYLVMDKNNLYDS |
| | | GGVLSAYQLTNQEVPAYKYISKQNGFLFYVPPDYTSKIDPETGFINLLD |
| | | TRYYSRKNAVALLNKFDKIYYDRDNKYFRFDFDYNSTDSNGNKNFDKLR |
| | | VDISELTRTKWSVCSHPAKRSITVQINNKWVRQPINDVTDKLIKLFEDK |
| | | QIGYESGKCLKDEILKVEDAKFFEDLLRYLSVLLALRHTYTENGVEYDL |
| | | IISSVEKAPGSNEFFVSGKDNNLPANADANGAYNIARKGLWLLRKLDEI |
| | | DNQELAIKKFNELKHAKEIKKNGEESKEDKGDRKRKKKWVSQWCPNKEW |
| | | LAFAQSMQDVSEK |
| SEQ ID NO: 178 | Cas12 | MNNGTNNFQNFIGISSLQKTLRNALIPTETTQQFIVKNGIIKEDELRGE |
| | Variant | NRQILKDIMDDYYRGFISETLSSIDDIDWTSLFEKMEIQLKNGDNKDTL |
| | | IKEQTEYRKAIHKKFANDDRFKNMFSAKLISDILPEFVIHNNNYSASEK |
| | | EEKTQVIKLFSRFATSFKDYFKNRANCFSADDISSSSCHRIVNDNAEIF |
| | | FSNALVYRRIVKSLSNDDINKISGDMKDSLKEMSLEEIYSYEKYGEFIT |
| | | QEGISFYNDICGKVNSFMNLYCQKNKENKNLYKLQKLHKQILCIADTSY |
| | | EVPYKFESDEEVYQSVNGFLDNISSKHIVERLRKIGDNYNGYNLDKIYI |
| | | VSKFYESVSQKTYRDWETINTALEIHYNNILPGNGKSKADKVKKAVKND |
| | | LQKSITEINELVSNYKLCSDDNIKAETYIHEISHILNNFEAQELKYNPE |
| | | IHLVESELKASELKNVLDVIMNAFHWCSVFMTEELVDKDNNFYAELEEI |
| | | YDEIYPVISLYNLVRNYVTQKPYSTKKIKLNFGIPTLADGWSKSKEYSN |
| | | NAIILMRDNLYYLGIFNAKNKPDKKIIEGNTSENKGDYKKMIYNLLPGP |
| | | NKMIPKVFLSSKTGVETYKPSAYILEGYKQNKHIKSSKDFDITFCHDLI |
| | | DYFKNCIAIHPEWKNFGFDFSDTSTYEDISGFYREVELQGYKIDWTYIS |
| | | EKDIDLLQEKGQLYLFQIYNKDFSKKSTGNDNLHTMYLKNLFSEENLKD |
| | | IVLKLNGEAEIFFRKSSIKNPIIHKKGSILVNRTYEAEEKDQFGNIQIV |
| | | RKNIPENIYQELYKYFNDKSDKELSDEAAKLKNVVGHHEAATNIVKDYR |
| | | YTYDKYFLHMPITINFKANKTGFINDRILQYIAKEKDLHVIGIDRGERN |
| | | LIYVSVIDTCGNIVEQKSFNIVNGYDYQIKLKQQEGARQIARKEWKEIG |
| | | KIKEIKEGYLSLVIHEISKMVIKYNAIIAMEDLSYGFKKGRFKVERQVY |
| | | QKFETMLINKLNYLVFKDISITENGGLLKGYQLTYIPDKLKNVGHQCGC |
| | | IFYVPAAYTSKIDPTTGFVNIFKFKDLTVDAKREFIKKFDSIRYDSEKN |
| | | LFCFTFDYNNFITQNTVMSKSSWSVYTYGVRIKRRFVNGRFSNESDTID |
| | | ITKDMEKTLEMTDINWRDGHDLRQDIIDYEIVQHIFEIFRLTVQMRNSL |
| | | SELEDRDYDRLISPVLNENNIFYDSAKAGDALPKDADANGAYCIALKGL |
| | | YEIKQITENWKEDGKFSRDKLKISNKDWFDFIQNKRYL |
| SEQ ID NO: 179 | Cas12 | MSNLNTFISPEFTGKIKMTKSLKVSMIPIGETEHWIAKHKVFEKDRELF |
| | Variant | DKNLKARPILDEFIKYTVSRALPNLLFDFEAYYLVKKDRTKARAFEKEL |
| | | AKTVTDLILKEMDELKSASLIDSADFVKTTLKKFAGTHDIPGLSRIEAI |
| | | ESLEAASKLTALNGKFNTSRIAIINTLIPKRIIENFDIYLSNMEKIRNV |
| | | YESGEFGFLFERYPDTLLFMEPANYRTVCSPEAIEDYNRFISGYGDSTE |
| | | SWIKGFNQELSEASNSSKSSNGGVRRYSLIKPLHKQHLFETKKFFTFAS |
| | | ISSDDDVRELINSVKGSTEDACLNALAFFSSSDPKTLFVKGSYLHTLSA |
| | | FLYGSANSYILPERIKEGEKARLTAEYDSVAKKTKAVTTRYNVAMNNIS |
| | | KKINEKIFSLADIDAYCCDISKRRSVREILLGIMQEMYAAVYGENGKWS |
| | | NIEAEAVLDSKTKIWKAKNGAVAKAVNDYLTAILEIRKFIRPFALRMEE |
| | | LEELGLDTSSALDAGEITNTLFEAVRAQKLVHAYLTRNDADIALSTQVY |
| | | FGGTQKAAASWWNYETGDIQNRQIALAKKDGMYYFIGTFDERGSYSIEP |
| | | ASPGEDYYEMLDVKKGQDANKQIKKVLFSNKAIREHFADSSNDYVITTK |
| | | VNSPITVRREIFDKYQAGEFKLTSQKIRKGDLVGEKEMTYYREYMDLLF |
| | | QMAKGYTEYSRFNMDTLLPIEEYDTENDLLDDVNTNTIDYRWVRISAAC |
| | | IDDGVRNGDIFVFRAQTSSMYGKRENKKGYTGLFLELVSDENLLVTRGM |
| | | SLNSAMSIYYRAKVHDAITVHKKGDVLVNKFTNARERIPENSYKAICAF |
| | | YNSGKSIEELTIEDRDWLAKATTRICSGEIIKDRRYTKNQYSISISYNI |
| | | NRSVNNRKRVDLATIVDDTASAGRIISVTRGTKDLVYYTVIDDGGSVIE |
| | | ARSLNVINGINYAKMLAQISEERHDSNANFDIPKRVETIKEAYCAFAVH |
| | | EIISAALKHNALIVVELISDAIKDKYSLLDNQVFLKFENVLKNCLMSVK |
| | | VKGARGMEPGSISNPLQLCNADDKSFRNGILYQIPSSYINICPVTGYAD |
| | | IIDYYNIVSAGDIRNFFVRFENIVYNKEKARFEFSFDLKNIPIKLEKCP |
| | | DRTKWTVLGRGEITTYDPLTKSNHYVFDAAQMLAETVSKEGLDPCANIV |
| | | EHIDELSAATLKKMFNTFRNIAKGIVSECDEVPVSYYKSPVIDEADIKN |
| | | KSLDNKSISEIKCYNLDEKARYYLALAKSSSDGENKNRYVSSTAIEWLN |
| | | YIQEKRTHE |

In some cases, a suitable Cas14 programmable nuclease comprises an amino acid sequence having at least 80%, at least 85%, at least 90%, at least 95%, at least 98%, at least 99%, or 100%, amino acid sequence identity to any one of SEQ ID NO: 31-SEQ ID NO: 122.

TABLE 2
Cas14 Sequences

| SEQ | |
|---|---|
| ID | |
| NO | Sequence |
| SEQ | MEVQKTVMKTLSLRILRPLYSQEIEKEIKEEKERRKQAGGTGELDGGFYKKLEKKHSE |
| ID | MFSFDRLNLLLNQLQREIAKVYNHAISELYIATIAQGNKSNKHYISSIVYNRAYGYFYN |
| NO: | AYIALGICSKVEANFRSNELLTQQSALPTAKSDNFPIVLHKQKGAEGEDGGFRISTEGS |
| 31 | DLIFEIPIPFYEYNGENRKEPYKWVKKGGQKPVLKLILSTFRRQRNKGWAKDEGTDAEI |
| | RKVTEGKYQVSQIEINRGKKLGEHQKWFANFSIEQPIYERKPNRSIVGGLDVGIRSPLV |
| | CAINNSFSRYSVDSNDVFKFSKQVFAFRRRLLSKNSLKRKGHGAAHKLEPITEMTEKN |
| | DKFRKKIIERWAKEVTNFFVKNQVGIVQIEDLSTMKDREDHFFNQYLRGFWPYYQMQ |
| | TLIENKLKEYGIEVKRVQAKYTSQLCSNPNCRYWNNYFNFEYRKVNKFPKFKCEKCN |
| | LEISADYNAARNLSTPDIEKFVAKATKGINLPEK |
| SEQ | MEEAKTVSKTLSLRILRPLYSAEIEKEIKEEKERRKQGGKSGELDSGFYKKLEKKHTQM |
| ID | FGWDKLNLMLSQLQRQIARVFNQSISELYIETVIQGKKSNKHYTSKIVYNRAYSVFYN |
| NO: | AYLALGITSKVEANFRSTELLMQKSSLPTAKSDNFPILLHKQKGVEGEEGGFKISADGN |
| 32 | DLIFEIPIPFYEYDSANKKEPFKWIKKGGQKPTIKLILSTFRRQRNKGWAKDEGTDAEIR |
| | KVIEGKYQVSHIEINRGKKLGDHQKWFVNFTIEQPIYERKLDKNIIGGIDVGIKSPLVCA |
| | VNNSFARYSVDSNDVLKFSKQAFAFRRRLLSKNSLKRSGHGSKNKLDPITRMTEKNDR |
| | FRKKIIERWAKEVTNFFIKNQVGTVQIEDLSTMKDRQDNFFNQYLRGFWPYYQMQNLI |
| | ENKLKEYGIETKRIKARYTSQLCSNPSCRHWNSYFSFDHRKTNNFPKFKCEKCALEISA |
| | DYNAARNISTPDIEKFVAKATKGINLPDKNENVILE |
| SEQ | MAKNTITKTLKLRIVRPYNSAEVEKIVADEKNNREKIALEKNKDKVKEACSKHLKVA |
| ID | AYCTTQVERNACLFCKARKLDDKFYQKLRGQFPDAVFWQEISEIFRQLQKQAAEIYNQ |
| NO: | SLIELYYEIFIKGKGIANASSVEHYLSDVCYTRAAELFKNAAIASGLRSKIKSNFRLKEL |
| 33 | KNMKSGLPTTKSDNFPIPLVKQKGGQYTGFEISNHNSDFIIKIPFGRWQVKKEIDKYRP |
| | WEKFDFEQVQKSPKPISLLLSTQRRKRNKGWSKDEGTEAEIKKVMNGDYQTSYIEVKR |
| | GSKIGEKSAWMLNLSIDVPKIDKGVDPSIIGGIDVGVKSPLVCAINNAFSRYSISDNDLF |
| | HFNKKMFARRRILLKKNRHKRAGHGAKNKLKPITILTEKSERFRKKLIERWACEIADFF |
| | IKNKVGTVQMENLESMKRKEDSYFNIRLRGFWPYAEMQNKIEFKLKQYGIEIRKVAPN |
| | NTSKTCSKCGHLNNYFNFEYRKKNKFPHFKCEKCNFKENADYNAALNISNPKLKSTKE |
| | EP |
| SEQ | MERQKVPQIRKIVRVVPLRILRPKYSDVIENALKKFKEKGDDTNTNDFWRAIRDRDTE |
| ID | FFRKELNFSEDEINQLERDTLFRVGLDNRVLFSYFDFLQEKLMKDYNKIISKLFINRQSK |
| NO: | SSFENDLTDEEVEELIEKDVTPFYGAYIGKGIKSVIKSNLGGKFIKSVKIDRETKKVTKL |
| 34 | TAINIGLMGLPVAKSDTFPIKIIKTNPDYITFQKSTKENLQKIEDYETGIEYGDLLVQITIP |
| | WFKNENKDFSLIKTKEAIEYYKLNGVGKKDLLNINLVLTTYHIRKKKSWQIDGSSQSL |
| | VREMANGELEEKWKSFFDTFIKKYGDEGKSALVKRRVNKKSRAKGEKGRELNLDERI |
| | KRLYDSIKAKSFPSEINLIPENYKWKLHFSIEIPPMVNDIDSNLYGGIDFGEQNIATLCVK |
| | NIEKDDYDFLTIYGNDLLKHAQASYARRRIMRVQDEYKARGHGKSRKTKAQEDYSER |
| | MQKLRQKITERLVKQISDFFLWRNKFHMAVCSLRYEDLNTLYKGESVKAKRMRQFIN |
| | KQQLFNGIERKLKDYNSEIYVNSRYPHYTSRLCSKCGKLNLYFDFLKFRTKNIIIRKNPD |
| | GSEIKYMPFFICEFCGWKQAGDKNASANIADKDYQDKLNKEKEFCNIRKPKSKKEDIG |
| | EENEEERDYSRRFNRNSFIYNSLKKDNKLNQEKLFDEWKNQLKRKIDGRNKFEPKEYK |
| | DRFSYLFAYYQEIIKNESES |
| SEQ | MVPTELITKTLQLRVIRPLYFEEIEKELAELKEQKEKEFEETNSLLLESKKIDAKSLKKL |
| ID | KRKARSSAAVEFWKIAKEKYPDILTKPEMEFIFSEMQKMMARFYNKSMTNIFIEMNND |
| NO: | EKVNPLSLISKASTEANQVIKCSSISSGLNRKIAGSINKTKFKQVRDGLISLPTARTETFPI |
| 35 | SFYKSTANKDEIPISKINLPSEEEADLTITLPFPFFEIKKEKKGQKAYSYFNIIEKSGRSNN |
| | KIDLLLSTHRRQRRKGWKEEGGTSAEIRRLMEGEFDKEWEIYLGEAEKSEKAKNDLIK |
| | NMTRGKLSKDIKEQLEDIQVKYFSDNNVESWNDLSKEQKQELSKLRKKKVEELKDW |
| | KHVKEILKTRAKIGWVELKRGKRQRDRNKWFVNITITRPPFINKELDDTKFGGIDLGV |
| | KVPFVCAVHGSPARLIIKENEILQFNKMVSARNRQITKDSEQRKGRGKKNKFIKKEIFN |
| | ERNELFRKKIIERWANQIVKFFEDQKCATVQIENLESFDRTSYK |
| SEQ | MKSDTKDKKIIIHQTKTLSLRIVKPQSIPMEEFTDLVRYHQMIIFPVYNNGAIDLYKKLF |
| ID | KAKIQKGNEARAIKYFMNKIVYAPIANTVKNSYIALGYSTKMQSSFSGKRLWDLRFGE |
| NO: | ATPPTIKADFPLPFYNQSGFKVSSENGEFIIGIPFGQYTKKTVSDIEKKTSFAWDKFTLED |
| 36 | TTKKTLIELLLSTKTRKMNEGWKNNEGTEAEIKRVMDGTYQVTSLEILQRDDSWFVNF |
| | NIAYDSLKKQPDRDKIAGIHMGITRPLTAVIYNNKYRALSIYPNTVMHLTQKQLARIKE |
| | QRTNSKYATGGHGRNAKVTGTDTLSEAYRQRRKKIIEDWIASIVKFAINNEIGTIYLEDI |
| | SNTNSFFAAREQKLIYLEDISNTNSFLSTYKYPISAISDTLQHKLEEKAIQVIRKKAYYV |
| | NQICSLCGHYNKGFTYQFRRKNKFPKMKCQGCLEATSTEFNAAANVANPDYEKLLIK |
| | HGLLQLKK |
| SEQ | MSTITRQVRLSPTPEQSRLLMAHCQQYISTVNVLVAAFDSEVLTGKVSTKDFRAALPS |
| ID | AVKNQALRDAQSVFKRSVELGCLPVLKKPHCQWNNQNWRVEGDQLILPICKDGKTQ |
| NO: | QERFRCAAVALEGKAGILRIKKKRGKWIADLTVTQEDAPESSGSAIMGVDLGIKVPAV |
| 37 | AHIGGKGTRFFGNGRSQRSMRRRFYARRKTLQKAKKLRAVRKSKGKEARWMKTINH |
| | QLSRQIVNHAHALGVGTIKIEALQGIRKGTTRKSRGAAARKNNRMTNTWSFSQLTLFIT |
| | YKAQRQGITVEQVDPAYTSQDCPACRARNGAQDRTYVCSECGWRGHRDTVGAINISR |
| | RAGLSGHRRGATGA |
| SEQ | MIAQKTIKIKLNPTKEQIIKLNSIIEEYIKVSNFTAKKIAEIQESFTDSGLTQGTCSECGKE |
| ID | KTYRKYHLLKKDNKLFCITCYKRKYSQFTLQKVEFQNKTGLRNVAKLPKTYYTNAIR |
| NO: | FASDTFSGFDEIIKKKQNRLNSIQNRLNFWKELLYNPSNRNEIKIKVVKYAPKTDTREH |
| 38 | PHYYSEAEIKGRIKRLEKQLKKFKMPKYPEFTSETISLQRELYSWKNPDELKISSITDKN |
| | ESMNYYGKEYLKRYIDLINSQTPQILLEKENNSFYLCFPITKNIEMPKIDDTFEPVGIDW |
| | GITRNIAVVSILDSKTKKPKFVKFYSAGYILGKRKHYKSLRKHFGQKKRQDKINKLGT |
| | KEDRFIDSNIHKLAFLIVKEIRNHSNKPIILMENITDNREEAEKSMRQNILLHSVKSRLQN |
| | YIAYKALWNNIPTNLVKPEHTSQICNRCGHQDRENRPKGSKLFKCVKCNYMSNADFN |
| | ASINIARKFYIGEYEPFYKDNEKMKSGVNSISM |
| SEQ | LKLSEQENITTGVKFKLKLDKETSEGLNDYFDEYGKAINFAIKVIQKELAEDRFAGKVR |
| ID | LDENKKPLLNEDGKKIWDFPNEFCSCGKQVNRYVNGKSLCQECYKNKFTEYGIRKRM |
| NO: | YSAKGRKAEQDINIKNSTNKISKTHFNYAIREAFILDKSIKKQRKERFRRLREMKKKLQ |
| 39 | EFIEIRDGNKILCPKIEKQRVERYIHPSWINKEKKLEDFRGYSMSNVLGKIKILDRNIKRE |
| | EKSLKEKGQINFKARRLMLDKSVKFLNDNKISFTISKNLPKEYELDLPEKEKRLNWLKE |
| | KIKIIKNQKPKYAYLLRKDDNFYLQYTLETEFNLKEDYSGIVGIDRGVSHIAVYTFVHN |
| | NGKNERPLFLNSSEILRLKNLQKERDRFLRRKHNKKRKKSNMRNIEKKIQLILHNYSKQ |
| | IVDFAKNKNAFIVFEKLEKPKKNRSKMSKKSQYKLSQFTFKKLSDLVDYKAKREGIKV |
| | LYISPEYTSKECSHCGEKVNTQRPFNGNSSLFKCNKCGVELNADYNASINIAKKGLNIL |
| | NSTN |
| SEQ | MEESIITGVKFKLRIDKETTKKLNEYFDEYGKAINFAVKIIQKELADDRFAGKAKLDQN |
| ID | KNPILDENGKKIYEFPDEFCSCGKQVNKYVNNKPFCQECYKIRFTENGIRKRMYSAKG |
| NO: | RKAEHKINILNSTNKISKTHFNYAIREAFILDKSIKKQRKKRNERLRESKKRLQQFIDMR |
| 40 | DGKREICPTIKGQKVDRFIHPSWITKDKKLEDFRGYTLSIINSKIKILDRNIKREEKSLKE |
| | KGQIIFKAKRLMLDKSIRFVGDRKVLFTISKTLPKEYELDLPSKEKRLNWLKEKIEIIKN |
| | QKPKYAYLLRKNIESEKKPNYEYYLQYTLEIKPELKDFYDGAIGIDRGINHIAVCTFISN |
| | DGKVTPPKFFSSGEILRLKNLQKERDRFLLRKHNKNRKKGNMRVIENKINLILHRYSKQ |
| | IVDMAKKLNASIVFEELGRIGKSRTKMKKSQRYKLSLFIFKKLSDLVDYKSRREGIRVT |
| | YVPPEYTSKECSHCGEKVNTQRPFNGNYSLFKCNKCGIQLNSDYNASINIAKKGLKIPN |
| | ST |
| SEQ | LWTIVIGDFIEMPKQDLVTTGIKFKLDVDKETRKKLDDYFDEYGKAINFAVKIIQKNLK |
| ID | EDRFAGKIALGEDKKPLLDKDGKKIYNYPNESCSCGNQVRRYVNAKPFCVDCYKLKF |
| NO: | TENGIRKRMYSARGRKADSDINIKNSTNKISKTHFNYAIREGFILDKSLKKQRSKRIKKL |
| 41 | LELKRKLQEFIDIRQGQMVLCPKIKNQRVDKFIHPSWLKRDKKLEEFRGYSLSVVEGKI |
| | KIFNRNILREEDSLRQRGHVNFKANRIMLDKSVRFLDGGKVNFNLNKGLPKEYLLDLP |
| | KKENKLSWLNEKISLIKLQKPKYAYLLRREGSFFIQYTIENVPKTFSDYLGAIGIDRGISH |
| | IAVCTFVSKNGVNKAPVFFSSGEILKLKSLQKQRDLFLRGKHNKIRKKSNMRNIDNKIN |
| | LILHKYSRNIVNLAKSEKAFIVFEKLEKIKKSRFKMSKSLQYKLSQFTFKKLSDLVEYK |
| | AKIEGIKVDYVPPEYTSKECSHCGEKVDTQRPFNGNSSLFKCNKCRVQLNADYNASINI |
| | AKKSLNISN |
| SEQ | MSKTTISVKLKIIDLSSEKKEFLDNYFNEYAKATTFCQLRIRRLLRNTHWLGKKEKSSK |
| ID | KWIFESGICDLCGENKELVNEDRNSGEPAKICKRCYNGRYGNQMIRKLFVSTKKREVQ |
| NO: | ENMDIRRVAKLNNTHYHRIPEEAFDMIKAADTAEKRRKKNVEYDKKRQMEFIEMFND |
| 42 | EKKRAARPKKPNERETRYVHISKLESPSKGYTLNGIKRKIDGMGKKIERAEKGLSRKKI |
| | FGYQGNRIKLDSNWVRFDLAESEITIPSLFKEMKLRITGPTNVHSKSGQIYFAEWFERIN |
| | KQPNNYCYLIRKTSSNGKYEYYLQYTYEAEVEANKEYAGCLGVDIGCSKLAAAVYYD |
| | SKNKKAQKPIEIFTNPIKKIKMRREKLIKLLSRVKVRHRRRKLMQLSKTEPIIDYTCHKT |
| | ARKIVEMANTAKAFISMENLETGIKQKQQARETKKQKFYRNMFLFRKLSKLIEYKALL |
| | KGIKIVYVKPDYTSQTCSSCGADKEKTERPSQAIFRCLNPTCRYYQRDINADFNAAVNI |
| | AKKALNNTEVVTTLL |
| SEQ | MARAKNQPYQKLTTTTGIKFKLDLSEEEGKRFDEYFSEYAKAVNFCAKVIYQLRKNL |
| ID | KFAGKKELAAKEWKFEISNCDFCNKQKEIYYKNIANGQKVCKGCHRTNFSDNAIRKK |
| NO: | MIPVKGRKVESKFNIHNTTKKISGTHRHWAFEDAADIIESMDKQRKEKQKRLRREKRK |
| 43 | LSYFFELFGDPAKRYELPKVGKQRVPRYLHKIIDKDSLTKKRGYSLSYIKNKIKISERNI |
| | ERDEKSLRKASPIAFGARKIKMSKLDPKRAFDLENNVFKIPGKVIKGQYKFFGTNVANE |
| | HGKKFYKDRISKILAGKPKYFYLLRKKVAESDGNPIFEYYVQWSIDTETPAITSYDNIL |
| | GIDAGITNLATTVLIPKNLSAEHCSHCGNNHVKPIFTKFFSGKELKAIKIKSRKQKYFLR |
| | GKHNKLVKIKRIRPIEQKVDGYCHVVSKQIVEMAKERNSCIALEKLEKPKKSKFRQRR |
| | REKYAVSMFVFKKLATFIKYKAAREGIEIIPVEPEGTSYTCSHCKNAQNNQRPYFKPNS |
| | KKSWTSMFKCGKCGIELNSDYNAAFNIAQKALNMTSA |
| SEQ | MDEKHFFCSYCNKELKISKNLINKISKGSIREDEAVSKAISIHNKKEHSLILGIKFKLFIEN |
| ID | KLDKKKLNEYFDNYSKAVTFAARIFDKIRSPYKFIGLKDKNTKKWTFPKAKCVFCLEE |
| NO: | KEVAYANEKDNSKICTECYLKEFGENGIRKKIYSTRGRKVEPKYNIFNSTKELSSTHYN |
| 44 | YAIRDAFQLLDALKKQRQKKLKSIFNQKLRLKEFEDIFSDPQKRIELSLKPHQREKRYIH |
| | LSKSGQESINRGYTLRFVRGKIKSLTRNIEREEKSLRKKTPIHFKGNRLMIFPAGIKFDFA |
| | SNKVKISISKNLPNEFNFSGTNVKNEHGKSFFKSRIELIKTQKPKYAYVLRKIKREYSKL |
| | RNYEIEKIRLENPNADLCDFYLQYTIETESRNNEEINGIIGIDRGITNLACLVLLKKGDKK |
| | PSGVKFYKGNKILGMKIAYRKHLYLLKGKRNKLRKQRQIRAIEPKINLILHQISKDIVKI |
| | AKEKNFAIALEQLEKPKKARFAQRKKEKYKLALFTFKNLSTLIEYKSKREGIPVIYVPPE |
| | KTSQMCSHCAINGDEHVDTQRPYKKPNAQKPSYSLFKCNKCGIELNADYNAAFNIAQ |
| | KGLKTLMLNHSH |
| SEQ | MLQTLLVKLDPSKEQYKMLYETMERFNEACNQIAETVFAIHSANKIEVQKTVYYPIRE |
| ID | KFGLSAQLTILAIRKVCEAYKRDKSIKPEFRLDGALVYDQRVLSWKGLDKVSLVTLQG |
| NO: | RQIIPIKFGDYQKARMDRIRGQADLILVKGVFYLCVVVEVSEESPYDPKGVLGVDLGIK |
| 45 | NLAVDSDGEVHSGEQTTNTRERLDSLKARLQSKGTKSAKRHLKKLSGRMAKFSKDVN |
| | HCISKKLVAKAKGTLMSIALEDLQGIRDRVTVRKAQRRNLHTWNFGLLRMFVDYKAK |
| | IAGVPLVFVDPRNTSRTCPSCGHVAKANRPTRDEFRCVSCGFAGAADHIAAMNIAFRA |
| | EVSQPIVTRFFVQSQAPSFRVG |
| SEQ | MDEEPDSAEPNLAPISVKLKLVKLDGEKLAALNDYFNEYAKAVNFCELKMQKIRKNL |
| ID | VNIRGTYLKEKKAWINQTGECCICKKIDELRCEDKNPDINGKICKKCYNGRYGNQMIR |
| NO: | KLFVSTNKRAVPKSLDIRKVARLHNTHYHRIPPEAADIIKAIETAERKRRNRILFDERRY |
| 46 | NELKDALENEEKRVARPKKPKEREVRYVPISKKDTPSKGYTMNALVRKVSGMAKKIE |
| | RAKRNLNKRKKIEYLGRRILLDKNWVRFDFDKSEISIPTMKEFFGEMRFEITGPSNVMS |
| | PNGREYFTKWFDRIKAQPDNYCYLLRKESEDETDFYLQYTWRPDAHPKKDYTGCLGI |
| | DIGGSKLASAVYFDADKNRAKQPIQIFSNPIGKWKTKRQKVIKVLSKAAVRHKTKKLE |
| | SLRNIEPRIDVHCHRIARKIVGMALAANAFISMENLEGGIREKQKAKETKKQKFSRNMF |
| | VFRKLSKLIEYKALMEGVKVVYIVPDYTSQLCSSCGTNNTKRPKQAIFMCQNTECRYF |
| | GKNINADFNAAINIAKKALNRKDIVRELS |
| SEQ | MEKNNSEQTSITTGIKFKLKLDKETKEKLNNYFDEYGKAINFAVRIIQMQLNDDRLAG |
| ID | KYKRDEKGKPILGEDGKKILEIPNDFCSCGNQVNHYVNGVSFCQECYKKRFSENGIRK |
| NO: | RMYSAKGRKAEQDINIKNSTNKISKTHFNYAIREAFNLDKSIKKQREKRFKKLKDMKR |
| 47 | KLQEFLEIRDGKRVICPKIEKQKVERYIHPSWINKEKKLEEFRGYSLSIVNSKIKSFDRNI |
| | QREEKSLKEKGQINFKAQRLMLDKSVKFLKDNKVSFTISKELPKTFELDLPKKEKKLN |
| | WLNEKLEIIKNQKPKYAYLLRKENNIFLQYTLDSIPEIHSEYSGAVGIDRGVSHIAVYTF |
| | LDKDGKNERPFFLSSSGILRLKNLQKERDKFLRKKHNKIRKKGNMRNIEQKINLILHEY |
| | SKQIVNFAKDKNAFIVFELLEKPKKSRERMSKKIQYKLSQFTFKKLSDLVDYKAKREGI |
| | KVIYVEPAYTSKDCSHCGERVNTQRPFNGNFSLFKCNKCGIVLNSDYNASLNIARKGL |
| | NISAN |
| SEQ | MAEEKFFFCEKCNKDIKIPKNYINKQGAEEKARAKHEHRVHALILGIKFKIYPKKEDIS |
| ID | KLNDYFDEYAKAVTFTAKIVDKLKAPFLFAGKRDKDTSKKKWVFPVDKCSFCKEKTE |
| NO: | INYRTKQGKNICNSCYLTEFGEQGLLEKIYATKGRKVSSSFNLFNSTKKLTGTHNNYV |
| 48 | VKESLQLLDALKKQRSKRLKKLSNTRRKLKQFEEMFEKEDKRFQLPLKEKQRELRFIH |
| | VSQKDRATEFKGYTMNKIKSKIKVLRRNIEREQRSLNRKSPVFFRGTRIRLSPSVQFDD |
| | KDNKIKLTLSKELPKEYSFSGLNVANEHGRKFFAEKLKLIKENKSKYAYLLRRQVNKN |
| | NKKPIYDYYLQYTVEFLPNIITNYNGILGIDRGINTLACIVLLENKKEKPSFVKFFSGKGI |
| | LNLKNKRRKQLYFLKGVHNKYRKQQKIRPIEPRIDQILHDISKQIIDLAKEKRVAISLEQ |
| | LEKPQKPKFRQSRKAKYKLSQFNFKTLSNYIDYKAKKEGIRVIYIAPEMTSQNCSRCA |
| | MKNDLHVNTQRPYKNTSSLFKCNKCGVELNADYNAAFNIAQKGLKILNS |
| SEQ | MISLKLKLLPDEEQKKLLDEMFWKWASICTRVGFGRADKEDLKPPKDAEGVWFSLTQ |
| ID | LNQANTDINDLREAMKHQKHRLEYEKNRLEAQRDDTQDALKNPDRREISTKRKDLFR |
| NO: | PKASVEKGFLKLKYHQERYWVRRLKEINKLIERKTKTLIKIEKGRIKFKATRITLHQGSF |
| 49 | KIRFGDKPAFLIKALSGKNQIDAPFVVVPEQPICGSVVNSKKYLDEITTNFLAYSVNAM |
| | LFGLSRSEEMLLKAKRPEKIKKKEEKLAKKQSAFENKKKELQKLLGRELTQQEEAIIEE |
| | TRNQFFQDFEVKITKQYSELLSKIANELKQKNDFLKVNKYPILLRKPLKKAKSKKINNL |
| | SPSEWKYYLQFGVKPLLKQKSRRKSRNVLGIDRGLKHLLAVTVLEPDKKTFVWNKLY |
| | PNPITGWKWRRRKLLRSLKRLKRRIKSQKHETIHENQTRKKLKSLQGRIDDLLHNISRK |
| | IVETAKEYDAVIVVEDLQSMRQHGRSKGNRLKTLNYALSLFDYANVMQLIKYKAGIE |
| | GIQIYDVKPAGTSQNCAYCLLAQRDSHEYKRSQENSKIGVCLNPNCQNHKKQIDADLN |
| | AARVIASCYALKINDSQPFGTRKRFKKRTTN |
| SEQ | METLSLKLKLNPSKEQLLVLDKMFWKWASICTRLGLKKAEMSDLEPPKDAEGVWFSK |
| ID | TQLNQANTDVNDLRKAMQHQGKRIEYELDKVENRRNEIQEMLEKPDRRDISPNRKDL |
| NO: | FRPKAAVEKGYLKLKYHKLGYWSKELKTANKLIERKRKTLAKIDAGKMKFKPTRISL |
| 50 | HTNSFRIKFGEEPKIALSTTSKHEKIELPLITSLQRPLKTSCAKKSKTYLDAAILNFLAYS |
| | TNAALFGLSRSEEMLLKAKKPEKIEKRDRKLATKRESFDKKLKTLEKLLERKLSEKEK |
| | SVFKRKQTEFFDKFCITLDETYVEALHRIAEELVSKNKYLEIKKYPVLLRKPESRLRSKK |
| | LKNLKPEDWTYYIQFGFQPLLDTPKPIKTKTVLGIDRGVRHLLAVSIFDPRTKTFTFNRL |
| | YSNPIVDWKWRRRKLLRSIKRLKRRLKSEKHVHLHENQFKAKLRSLEGRIEDHFHNLS |
| | KEIVDLAKENNSVIVVENLGGMRQHGRGRGKWLKALNYALSHFDYAKVMQLIKYKA |
| | ELAGVFVYDVAPAGTSINCAYCLLNDKDASNYTRGKVINGKKNTKIGECKTCKKEFD |
| | ADLNAARVIALCYEKRLNDPQPFGTRKQFKPKKP |
| SEQ | MKALKLQLIPTRKQYKILDEMFWKWASLANRVSQKGESKETLAPKKDIQKIQFNATQ |
| ID | LNQIEKDIKDLRGAMKEQQKQKERLLLQIQERRSTISEMLNDDNNKERDPHRPLNFRP |
| NO: | KGWRKFHTSKHWVGELSKILRQEDRVKKTIERIVAGKISFKPKRIGIWSSNYKINFFKR |
| 51 | KISINPLNSKGFELTLMTEPTQDLIGKNGGKSVLNNKRYLDDSIKSLLMFALHSRFFGL |
| | NNTDTYLLGGKINPSLVKYYKKNQDMGEFGREIVEKFERKLKQEINEQQKKIIMSQIKE |
| | QYSNRDSAFNKDYLGLINEFSEVFNQRKSERAEYLLDSFEDKIKQIKQEIGESLNISDWD |
| | FLIDEAKKAYGYEEGFTEYVYSKRYLEILNKIVKAVLITDIYFDLRKYPILLRKPLDKIK |
| | KISNLKPDEWSYYIQFGYDSINPVQLMSTDKFLGIDRGLTHLLAYSVFDKEKKEFIINQL |
| | EPNPIMGWKWKLRKVKRSLQHLERRIRAQKMVKLPENQMKKKLKSIEPKIEVHYHNI |
| | SRKIVNLAKDYNASIVVESLEGGGLKQHGRKKNARNRSLNYALSLFDYGKIASLIKYK |
| | ADLEGVPMYEVLPAYTSQQCAKCVLEKGSFVDPEIIGYVEDIGIKGSLLDSLFEGTELSS |
| | IQVLKKIKNKIELSARDNHNKEINLILKYNFKGLVIVRGQDKEEIAEHPIKEINGKFAILD |
| | FVYKRGKEKVGKKGNQKVRYTGNKKVGYCSKHGQVDADLNASRVIALCKYLDINDP |
| | ILFGEQRKSFK |
| SEQ | MVTRAIKLKLDPTKNQYKLLNEMFWKWASLANRFSQKGASKETLAPKDGTQKIQFN |
| ID | ATQLNQIKKDVDDLRGAMEKQGKQKERLLIQIQERLLTISEILRDDSKKEKDPHRPQNF |
| NO: | RPFGWRRFHTSAYWSSEASKLTRQVDRVRRTIERIKAGKINFKPKRIGLWSSTYKINFL |
| 52 | KKKINISPLKSKSFELDLITEPQQKIIGKEGGKSVANSKKYLDDSIKSLLIFAIKSRLFGLN |
| | NKDKPLFENIITPNLVRYHKKGQEQENFKKEVIKKFENKLKKEISQKQKEIIFSQIERQY |
| | ENRDATFSEDYLRAISEFSEIFNQRKKERAKELLNSFNEKIRQLKKEVNGNISEEDLKIL |
| | EVEAEKAYNYENGFIEWEYSEQFLGVLEKIARAVLISDNYFDLKKYPILIRKPTNKSKKI |
| | TNLKPEEWDYYIQFGYGLINSPMKIETKNFMGIDRGLTHLLAYSIFDRDSEKFTINQLEL |
| | NPIKGWKWKLRKVKRSLQHLERRMRAQKGVKLPENQMKKRLKSIEPKIESYYHNLSR |
| | KIVNLAKANNASIVVESLEGGGLKQHGRKKNSRHRALNYALSLFDYGKIASLIKYKSD |
| | LEGVPMYEVLPAYTSQQCAKCVLKKGSFVEPEIIGYIEEIGFKENLLTLLFEDTGLSSVQ |
| | VLKKSKNKMTLSARDKEGKMVDLVLKYNFKGLVISQEKKKEEIVEFPIKEIDGKFAVL |
| | DSAYKRGKERISKKGNQKLVYTGNKKVGYCSVHGQVDADLNASRVIALCKYLGINEP |
| | IVFGEQRKSFK |
| SEQ | LDLITEPIQPHKSSSLRSKEFLEYQISDFLNFSLHSLFFGLASNEGPLVDFKIYDKIVIPKPE |
| ID | ERFPKKESEEGKKLDSFDKRVEEYYSDKLEKKIERKLNTEEKNVIDREKTRIWGEVNK |
| NO: | LEEIRSIIDEINEIKKQKHISEKSKLLGEKWKKVNNIQETLLSQEYVSLISNLSDELTNKK |
| 53 | KELLAKKYSKFDDKIKKIKEDYGLEFDENTIKKEGEKAFLNPDKFSKYQFSSSYLKLIG |
| | EIARSLITYKGFLDLNKYPIIFRKPINKVKKIHNLEPDEWKYYIQFGYEQINNPKLETENI |
| | LGIDRGLTHILAYSVFEPRSSKFILNKLEPNPIEGWKWKLRKLRRSIQNLERRWRAQDN |
| | VKLPENQMKKNLRSIEDKVENLYHNLSRKIVDLAKEKNACIVFEKLEGQGMKQHGRK |
| | KSDRLRGLNYKLSLFDYGKIAKLIKYKAEIEGIPIYRIDSAYTSQNCAKCVLESRRFAQP |
| | EEISCLDDFKEGDNLDKRILEGTGLVEAKIYKKLLKEKKEDFEIEEDIAMFDTKKVIKEN |
| | KEKTVILDYVYTRRKEIIGTNHKKNIKGIAKYTGNTKIGYCMKHGQVDADLNASRTIA |
| | LCKNFDINNPEIWK |
| SEQ | MSDESLVSSEDKLAIKIKIVPNAEQAKMLDEMFKKWSSICNRISRGKEDIETLRPDEGK |
| ID | ELQFNSTQLNSATMDVSDLKKAMARQGERLEAEVSKLRGRYETIDASLRDPSRRHTNP |
| NO: | QKPSSFYPSDWDISGRLTPRFHTARHYSTELRKLKAKEDKMLKTINKIKNGKIVFKPKR |
| 54 | ITLWPSSVNMAFKGSRLLLKPFANGFEMELPIVISPQKTADGKSQKASAEYMRNALLG |
| | LAGYSINQLLFGMNRSQKMLANAKKPEKVEKFLEQMKNKDANFDKKIKALEGKWLL |
| | DRKLKESEKSSIAVVRTKFFKSGKVELNEDYLKLLKHMANEILERDGFVNLNKYPILSR |
| | KPMKRYKQKNIDNLKPNMWKYYIQFGYEPIFERKASGKPKNIMGIDRGLTHLLAVAV |
| | FSPDQQKFLFNHLESNPIMHWKWKLRKIRRSIQHMERRIRAEKNKHIHEAQLKKRLGSI |
| | EEKTEQHYHIVSSKIINWAIEYEAAIVLESLSHMKQRGGKKSVRTRALNYALSLFDYEK |
| | VARLITYKARIRGIPVYDVLPGMTSKTCATCLLNGSQGAYVRGLETTKAAGKATKRK |
| | NMKIGKCMVCNSSENSMIDADLNAARVIAICKYKNLNDPQPAGSRKVFKRF |
| SEQ | MLALKLKIMPTEKQAEILDAMFWKWASICSRIAKMKKKVSVKENKKELSKKIPSNSDI |
| ID | WFSKTQLCQAEVDVGDHKKALKNFEKRQESLLDELKYKVKAINEVINDESKREIDPN |
| NO: | NPSKFRIKDSTKKGNLNSPKFFTLKKWQKILQENEKRIKKKESTIEKLKRGNIFFNPTKIS |
| 55 | LHEEEYSINFGSSKLLLNCFYKYNKKSGINSDQLENKFNEFQNGLNIICSPLQPIRGSSKR |
| | SFEFIRNSIINFLMYSLYAKLFGIPRSVKALMKSNKDENKLKLEEKLKKKKSSFNKTVK |
| | EFEKMIGRKLSDNESKILNDESKKFFEIIKSNNKYIPSEEYLKLLKDISEEIYNSNIDFKPY |
| | KYSILIRKPLSKFKSKKLYNLKPTDYKYYLQLSYEPFSKQLIATKTILGIDRGLKHLLAV |
| | SVFDPSQNKFVYNKLIKNPVFKWKKRYHDLKRSIRNRERRIRALTGVHIHENQLIKKLK |
| | SMKNKINVLYHNVSKNIVDLAKKYESTIVLERLENLKQHGRSKGKRYKKLNYVLSNF |
| | DYKKIESLISYKAKKEGVPVSNINPKYTSKTCAKCLLEVNQLSELKNEYNRDSKNSKIG |
| | ICNIHGQIDADLNAARVIALCYSKNLNEPHFK |
| SEQ | VINLFGYKFALYPNKTQEELLNKHLGECGWLYNKAIEQNEYYKADSNIEEAQKKFELL |
| ID | PDKNSDEAKVLRGNISKDNYVYRTLVKKKKSEINVQIRKAVVLRPAETIRNLAKVKKK |
| NO: | GLSVGRLKFIPIREWDVLPFKQSDQIRLEENYLILEPYGRLKFKMHRPLLGKPKTFCIKR |
| 56 | TATDRWTISFSTEYDDSNMRKNDGGQVGIDVGLKTHLRLSNENPDEDPRYPNPKIWK |
| | RYDRRLTILQRRISKSKKLGKNRTRLRLRLSRLWEKIRNSRADLIQNETYEILSENKLIAI |
| | EDLNVKGMQEKKDKKGRKGRIRAQEKGLHRSISDAAFSEFRRVLEYKAKRFGSEVKP |
| | VSAIDSSKECHNCGNKKGMPLESRIYECPKCGLKIDRDLNSAKVILARATGVRPGSNA |
| | RADTKISATAGASVQTEGTVSEDFRQQMETSDQKPMQGEGSKEPPMNPEHKSSGRGS |
| | KHVNIGCKNKVGLYNEDENSRSTEKQIMDENRSTTEDMVEIGALHSPVLTT |
| SEQ | MIASIDYEAVSQALIVFEFKAKGKDSQYQAIDEAIRSYRFIRNSCLRYWMDNKKVGKY |
| ID | DLNKYCKVLAKQYPFANKLNSQARQSAAECSWSAISRFYDNCKRKVSGKKGFPKFKK |
| NO: | HARSVEYKTSGWKLSENRKAITFTDKNGIGKLKLKGTYDLHFSQLEDMKRVRLVRRA |
| 57 | DGYYVQFCISVDVKVETEPTGKAIGLDVGIKYFLADSSGNTIENPQFYRKAEKKLNRA |
| | NRRKSKKYIRGVKPQSKNYHKARCRYARKHLRVSRQRKEYCKRVAYCVIHSNDVVA |
| | YEDLNVKGMVKNRHLAKSISDVAWSTFRHWLEYFAIKYGKLTIPVAPHNTSQNCSNC |
| | DKKVPKSLSTRTHICHHCGYSEDRDVNAAKNILKKALSTVGQTGSLKLGEIEPLLVLEQ |
| | SCTRKFDL |
| SEQ | LAEENTLHLTLAMSLPLNDLPENRTRSELWRRQWLPQKKLSLLLGVNQSVRKAAADC |
| ID | LRWFEPYQELLWWEPTDPDGKKLLDKEGRPIKRTAGHMRVLRKLEEIAPFRGYQLGS |
| NO: | AVKNGLRHKVADLLLSYAKRKLDPQFTDKTSYPSIGDQFPIVWTGAFVCYEQSITGQL |
| 58 | YLYLPLFPRGSHQEDITNNYDPDRGPALQVFGEKEIARLSRSTSGLLLPLQFDKWGEAT |
| | FIRGENNPPTWKATHRRSDKKWLSEVLLREKDFQPKRVELLVRNGRIFVNVACEIPTK |
| | PLLEVENFMGVSFGLEHLVTVVVINRDGNVVHQRQEPARRYEKTYFARLERLRRRGG |
| | PFSQELETFHYRQVAQIVEEALRFKSVPAVEQVGNIPKGRYNPRLNLRLSYWPFGKLA |
| | DLTSYKAVKEGLPKPYSVYSATAKMLCSTCGAANKEGDQPISLKGPTVYCGNCGTRH |
| | NTGFNTALNLARRAQELFVKGVVAR |
| SEQ | MSQSLLKWHDMAGRDKDASRSLQKSAVEGVLLHLTASHRVALEMLEKSVSQTVAVT |
| ID | MEAAQQRLVIVLEDDPTKATSRKRVISADLQFTREEFGSLPNWAQKLASTCPEIATKY |
| NO: | ADKHINSIRIAWGVAKESTNGDAVEQKLQWQIRLLDVTMFLQQLVLQLADKALLEQIP |
| 59 | SSIRGGIGQEVAQQVTSHIQLLDSGTVLKAELPTISDRNSELARKQWEDAIQTVCTYAL |
| | PFSRERARILDPGKYAAEDPRGDRLINIDPMWARVLKGPTVKSLPLLFVSGSSIRIVKLT |
| | LPRKHAAGHKHTFTATYLVLPVSREWINSLPGTVQEKVQWWKKPDVLATQELLVGK |
| | GALKKSANTLVIPISAGKKRFFNHILPALQRGFPLQWQRIVGRSYRRPATHRKWFAQLT |
| | IGYTNPSSLPEMALGIHFGMKDILWWALADKQGNILKDGSIPGNSILDFSLQEKGKIER |
| | QQKAGKNVAGKKYGKSLLNATYRVVNGVLEFSKGISAEHASQPIGLGLETIRFVDKAS |
| | GSSPVNARHSNWNYGQLSGIFANKAGPAGFSVTEITLKKAQRDLSDAEQARVLAIEAT |
| | KRFASRIKRLATKRKDDTLFV |
| SEQ | VEPVEKERFYYRTYTFRLDGQPRTQNLTTQSGWGLLTKAVLDNTKHYWEIVHHARIA |
| ID | NQPIVFENPVIDEQGNPKLNKLGQPRFWKRPISDIVNQLRALFENQNPYQLGSSLIQGT |
| NO: | YWDVAENLASWYALNKEYLAGTATWGEPSFPEPHPLTEINQWMPLTFSSGKVVRLLK |
| 60 | NASGRYFIGLPILGENNPCYRMRTIEKLIPCDGKGRVTSGSLILFPLVGIYAQQHRRMTD |
| | ICESIRTEKGKLAWAQVSIDYVREVDKRRRMRRTRKSQGWIQGPWQEVFILRLVLAHK |
| | APKLYKPRCFAGISLGPKTLASCVILDQDERVVEKQQWSGSELLSLIHQGEERLRSLRE |
| | QSKPTWNAAYRKQLKSLINTQVFTIVTFLRERGAAVRLESIARVRKSTPAPPVNFLLSH |
| | WAYRQITERLKDLAIRNGMPLTHSNGSYGVRFTCSQCGATNQGIKDPTKYKVDIESET |
| | FLCSICSHREIAAVNTATNLAKQLLDE |
| SEQ | MNDTETSETLTSHRTVCAHLHVVGETGSLPRLVEAALAELITLNGRATQALLSLAKNG |
| ID | LVLRRDKEENLIAAELTLPCRKNKYADVAAKAGEPILATRINNKGKLVTKKWYGEGN |
| NO: | SYHIVRFTPETGMFTVRVFDRYAFDEELLHLHSEVVFGSDLPKGIKAKTDSLPANFLQA |
| 61 | VFTSFLELPFQGFPDIVVKPAMKQAAEQLLSYVQLEAGENQQAEYPDTNERDPELRLV |
| | EWQKSLHELSVRTEPFEFVRARDIDYYAETDRRGNRFVNITPEWTKFAESPFARRLPLK |
| | IPPEFCILLRRKTEGHAKIPNRIYLGLQIFDGVTPDSTLGVLATAEDGKLFWWHDHLDE |
| | FSNLEGKPEPKLKNKPQLLMVSLEYDREQRFEESVGGDRKICLVTLKETRNFRRGWNG |
| | RILGIHFQHNPVITWALMDHDAEVLEKGFIEGNAFLGKALDKQALNEYLQKGGKWVG |
| | DRSFGNKLKGITHTLASLIVRLAREKDAWIALEEISWVQKQSADSVANHEIVEQPHHSL |
| | TR |
| SEQ | MNDTETSETLTSHRTVCAHLHVVGETGSLPRLVEAALAELITLNGRATQALLSLAKNG |
| ID | LVLRRDKEENLIAAELTLPCRKNKYADVAAKAGEPILATRINNKGKLVTKKWYGEGN |
| NO: | SYHIVRFTPETGMFTVRVFDRYAFDEELLHLHSEVVFGSDLPKGIKAKTDSLPANFLQA |
| 62 | VFTSFLELPFQGFPDIVVKPAMKQAAEQLLSYVQLEAGENQQAEYPDTNERDPELRLV |
| | EWQKSLHELSVRTEPFEFVRARDIDYYAETDRRGNRFVNITPEWTKFAESPFARRLPLK |
| | IPPEFCILLRRKTEGHAKIPNRIYLGLQIFDGVTPDSTLGVLATAEDGKLFWWHDHLDE |
| | FSNLEGKPEPKLKNKPQLLMVSLEYDREQRFEESVGGDRKICLVTLKETRNFRRGRHG |
| | HTRTDRLPAGNTLWRADFATSAEVAAPKWNGRILGIHFQHNPVITWALMDHDAEVLE |
| | KGFIEGNAFLGKALDKQALNEYLQKGGKWVGDRSFGNKLKGITHTLASLIVRLAREK |
| | DAWIALEEISWVQKQSADSVANRRFSMWNYSRLATLIEWLGTDIATRDCGTAAPLAH |
| | KVSDYLTHFTCPECGACRKAGQKKEIADTVRAGDILTCRKCGFSGPIPDNFIAEFVAKK |
| | ALERMLKKKPV |
| SEQ | MAKRNFGEKSEALYRAVRFEVRPSKEELSILLAVSEVLRMLFNSALAERQQVFTEFIAS |
| ID | LYAELKSASVPEEISEIRKKLREAYKEHSISLFDQINALTARRVEDEAFASVTRNWQEET |
| NO: | LDALDGAYKSFLSLRRKGDYDAHSPRSRDSGFFQKIPGRSGFKIGEGRIALSCGAGRKL |
| 63 | SFPIPDYQQGRLAETTKLKKFELYRDQPNLAKSGRFWISVVYELPKPEATTCQSEQVAF |
| | VALGASSIGVVSQRGEEVIALWRSDKHWVPKIEAVEERMKRRVKGSRGWLRLLNSGK |
| | RRMHMISSRQHVQDEREIVDYLVRNHGSHFVVTELVVRSKEGKLADSSKPERGGSLG |
| | LNWAAQNTGSLSRLVRQLEEKVKEHGGSVRKHKLTLTEAPPARGAENKLWMARKLR |
| | ESFLKEV |
| SEQ | LAKNDEKELLYQSVKFEIYPDESKIRVLTRVSNILVLVWNSALGERRARFELYIAPLYE |
| ID | ELKKFPRKSAESNALRQKIREGYKEHIPTFFDQLKKLLTPMRKEDPALLGSVPRAYQEE |
| NO: | TLNTLNGSFVSFMTLRRNNDMDAKPPKGRAEDRFHEISGRSGFKIDGSEFVLSTKEQKL |
| 64 | RFPIPNYQLEKLKEAKQIKKFTLYQSRDRRFWISIAYEIELPDQRPFNPEEVIYIAFGASSI |
| | GVISPEGEKVIDFWRPDKHWKPKIKEVENRMRSCKKGSRAWKKRAAARRKMYAMTQ |
| | RQQKLNHREIVASLLRLGFHFVVTEYTVRSKPGKLADGSNPKRGGAPQGFNWSAQNT |
| | GSFGEFILWLKQKVKEQGGTVQTFRLVLGQSERPEKRGRDNKIEMVRLLREKYLESQT |
| | IVV |
| SEQ | MAKGKKKEGKPLYRAVRFEIFPTSDQITLFLRVSKNLQQVWNEAWQERQSCYEQFFG |
| ID | SIYERIGQAKKRAQEAGFSEVWENEAKKGLNKKLRQQEISMQLVSEKESLLQELSIAFQ |
| NO: | EHGVTLYDQINGLTARRIIGEFALIPRNWQEETLDSLDGSFKSFLALRKNGDPDAKPPR |
| 65 | QRVSENSFYKIPGRSGFKVSNGQIYLSFGKIGQTLTSVIPEFQLKRLETAIKLKKFELCRD |
| | ERDMAKPGRFWISVAYEIPKPEKVPVVSKQITYLAIGASRLGVVSPKGEFCLNLPRSDY |
| | HWKPQINALQERLEGVVKGSRKWKKRMAACTRMFAKLGHQQKQHGQYEVVKKLLR |
| | HGVHFVVTELKVRSKPGALADASKSDRKGSPTGPNWSAQNTGNIARLIQKLTDKASE |
| | HGGTVIKRNPPLLSLEERQLPDAQRKIFIAKKLREEFLADQK |
| SEQ | MAKREKKDDVVLRGTKMRIYPTDRQVTLMDMWRRRCISLWNLLLNLETAAYGAKN |
| ID | TRSKLGWRSIWARVVEENHAKALIVYQHGKCKKDGSFVLKRDGTVKHPPRERFPGDR |
| NO: | KILLGLFDALRHTLDKGAKCKCNVNQPYALTRAWLDETGHGARTADIIAWLKDFKGE |
| 66 | CDCTAISTAAKYCPAPPTAELLTKIKRAAPADDLPVDQAILLDLFGALRGGLKQKECD |
| | HTHARTVAYFEKHELAGRAEDILAWLIAHGGTCDCKIVEEAANHCPGPRLFIWEHELA |
| | MIMARLKAEPRTEWIGDLPSHAAQTVVKDLVKALQTMLKERAKAAAGDESARKTGF |
| | PKFKKQAYAAGSVYFPNTTMFFDVAAGRVQLPNGCGSMRCEIPRQLVAELLERNLKP |
| | GLVIGAQLGLLGGRIWRQGDRWYLSCQWERPQPTLLPKTGRTAGVKIAASIVFTTYDN |
| | RGQTKEYPMPPADKKLTAVHLVAGKQNSRALEAQKEKEKKLKARKERLRLGKLEKG |
| | HDPNALKPLKRPRVRRSKLFYKSAARLAACEAIERDRRDGFLHRVTNEIVHKFDAVSV |
| | QKMSVAPMMRRQKQKEKQIESKKNEAKKEDNGAAKKPRNLKPVRKLLRHVAMARG |
| | RQFLEYKYNDLRGPGSVLIADRLEPEVQECSRCGTKNPQMKDGRRLLRCIGVLPDGTD |
| | CDAVLPRNRNAARNAEKRLRKHREAHNA |
| SEQ | MNEVLPIPAVGEDAADTIMRGSKMRIYPSVRQAATMDLWRRRCIQLWNLLLELEQAA |
| ID | YSGENRRTQIGWRSIWATVVEDSHAEAVRVAREGKKRKDGTFRKAPSGKEIPPLDPA |
| NO: | MLAKIQRQMNGAVDVDPKTGEVTPAQPRLFMWEHELQKIMARLKQAPRTHWIDDLP |
| 67 | SHAAQSVVKDLIKALQAMLRERKKRASGIGGRDTGFPKFKKNRYAAGSVYFANTQLR |
| | FEAKRGKAGDPDAVRGEFARVKLPNGVGWMECRMPRHINAAHAYAQATLMGGRIW |
| | RQGENWYLSCQWKMPKPAPLPRAGRTAAIKIAAAIPITTVDNRGQTREYAMPPIDRERI |
| | AAHAAAGRAQSRALEARKRRAKKREAYAKKRHAKKLERGIAAKPPGRARIKLSPGFY |
| | AAAAKLAKLEAEDANAREAWLHEITTQIVRNFDVIAVPRMEVAKLMKKPEPPEEKEE |
| | QVKAPWQGKRRSLKAARVMMRRTAMALIQTTLKYKAVDLRGPQAYEEIAPLDVTAA |
| | ACSGCGVLKPEWKMARAKGREIMRCQEPLPGGKTCNTVLTYTRNSARVIGRELAVRL |
| | AERQKA |
| SEQ | MTTQKTYNFCFYDQRFFELSKEAGEVYSRSLEEFWKIYDETGVWLSKFDLQKHMRNK |
| ID | LERKLLHSDSFLGAMQQVHANLASWKQAKKVVPDACPPRKPKFLQAILFKKSQIKYK |
| NO: | NGFLRLTLGTEKEFLYLKWDINIPLPIYGSVTYSKTRGWKINLCLETEVEQKNLSENKY |
| 68 | LSIDLGVKRVATIFDGENTITLSGKKFMGLMHYRNKLNGKTQSRLSHKKKGSNNYKKI |
| | QRAKRKTTDRLLNIQKEMLHKYSSFIVNYAIRNDIGNIIIGDNSSTHDSPNMRGKTNQKI |
| | SQNPEQKLKNYIKYKFESISGRVDIVPEPYTSRKCPHCKNIKKSSPKGRTYKCKKCGFIF |
| | DRDGVGAINIYNENVSFGQIISPGRIRSLTEPIGMKFHNEIYFKSYVAA |
| SEQ | MSVRSFQARVECDKQTMEHLWRTHKVFNERLPEIIKILFKMKRGECGQNDKQKSLYK |
| ID | SISQSILEANAQNADYLLNSVSIKGWKPGTAKKYRNASFTWADDAAKLSSQGIHVYDK |
| NO: | KQVLGDLPGMMSQMVCRQSVEAISGHIELTKKWEKEHNEWLKEKEKWESEDEHKKY |
| 69 | LDLREKFEQFEQSIGGKITKRRGRWHLYLKWLSDNPDFAAWRGNKAVINPLSEKAQIR |
| | INKAKPNKKNSVERDEFFKANPEMKALDNLHGYYERNFVRRRKTKKNPDGFDHKPTF |
| | TLPHPTIHPRWFVFNKPKTNPEGYRKLILPKKAGDLGSLEMRLLTGEKNKGNYPDDWI |
| | SVKFKADPRLSLIRPVKGRRVVRKGKEQGQTKETDSYEFFDKHLKKWRPAKLSGVKLI |
| | FPDKTPKAAYLYFTCDIPDEPLTETAKKIQWLETGDVTKKGKKRKKKVLPHGLVSCAV |
| | DLSMRRGTTGFATLCRYENGKIHILRSRNLWVGYKEGKGCHPYRWTEGPDLGHIAKH |
| | KREIRILRSKRGKPVKGEESHIDLQKHIDYMGEDRFKKAARTIVNFALNTENAASKNGF |
| | YPRADVLLLENLEGLIPDAEKERGINRALAGWNRRHLVERVIEMAKDAGFKRRVFEIP |
| | PYGTSQVCSKCGALGRRYSIIRENNRREIRFGYVEKLFACPNCGYCANADHNASVNLN |
| | RRFLIEDSFKSYYDWKRLSEKKQKEEIETIESKLMDKLCAMHKISRGSISK |
| SEQ | MHLWRTHCVFNQRLPALLKRLFAMRRGEVGGNEAQRQVYQRVAQFVLARDAKDSV |
| ID | DLLNAVSLRKRSANSAFKKKATISCNGQAREVTGEEVFAEAVALASKGVFAYDKDDM |
| NO: | RAGLPDSLFQPLTRDAVACMRSHEELVATWKKEYREWRDRKSEWEAEPEHALYLNL |
| 70 | RPKFEEGEAARGGRFRKRAERDHAYLDWLEANPQLAAWRRKAPPAVVPIDEAGKRRI |
| | ARAKAWKQASVRAEEFWKRNPELHALHKIHVQYLREFVRPRRTRRNKRREGFKQRPT |
| | FTMPDPVRHPRWCLFNAPQTSPQGYRLLRLPQSRRTVGSVELRLLTGPSDGAGFPDAW |
| | VNVRFKADPRLAQLRPVKVPRTVTRGKNKGAKVEADGFRYYDDQLLIERDAQVSGV |
| | KLLFRDIRMAPFADKPIEDRLLSATPYLVFAVEIKDEARTERAKAIRFDETSELTKSGKK |
| | RKTLPAGLVSVAVDLDTRGVGFLTRAVIGVPEIQQTHHGVRLLQSRYVAVGQVEARA |
| | SGEAEWSPGPDLAHIARHKREIRRLRQLRGKPVKGERSHVRLQAHIDRMGEDRFKKA |
| | ARKIVNEALRGSNPAAGDPYTRADVLLYESLETLLPDAERERGINRALLRWNRAKLIE |
| | HLKRMCDDAGIRHFPVSPFGTSQVCSKCGALGRRYSLARENGRAVIRFGWVERLFACP |
| | NPECPGRRPDRPDRPFTCNSDHNASVNLHRVFALGDQAVAAFRALAPRDSPARTLAV |
| | KRVEDTLRPQLMRVHKLADAGVDSPF |
| SEQ | MATLVYRYGVRAHGSARQQDAVVSDPAMLEQLRLGHELRNALVGVQHRYEDGKRA |
| ID | VWSGFASVAAADHRVTTGETAVAELEKQARAEHSADRTAATRQGTAESLKAARAAV |
| NO: | KQARADRKAAMAAVAEQAKPKIQALGDDRDAEIKDLYRRFCQDGVLLPRCGRCAGD |
| 71 | LRSDGDCTDCGAAHEPRKLYWATYNAIREDHQTAVKLVEAKRKAGQPARLRFRRWT |
| | GDGTLTVQLQRMHGPACRCVTCAEKLTRRARKTDPQAPAVAADPAYPPTDPPRDPAL |
| | LASGQGKWRNVLQLGTWIPPGEWSAMSRAERRRVGRSHIGWQLGGGRQLTLPVQLH |
| | RQMPADADVAMAQLTRVRVGGRHRMSVALTAKLPDPPQVQGLPPVALHLGWRQRP |
| | DGSLRVATWACPQPLDLPPAVADVVVSHGGRWGEVIMPARWLADAEVPPRLLGRRD |
| | KAMEPVLEALADWLEAHTEACTARMTPALVRRWRSQGRLAGLTNRWRGQPPTGSAE |
| | ILTYLEAWRIQDKLLWERESHLRRRLAARRDDAWRRVASWLARHAGVLVVDDADIA |
| | ELRRRDDPADTDPTMPASAAQAARARAALAAPGRLRHLATITATRDGLGVHTVASAG |
| | LTRLHRKCGHQAQPDPRYAASAVVTCPGCGNGYDQDYNAAMLMLDRQQQP |
| SEQ | MSRVELHRAYKFRLYPTPAQVAELAEWERQLRRLYNLAHSQRLAAMQRHVRPKSPG |
| ID | VLKSECLSCGAVAVAEIGTDGKAKKTVKHAVGCSVLECRSCGGSPDAEGRTAHTAAC |
| NO: | SFVDYYRQGREMTQLLEEDDQLARVVCSARQETLRDLEKAWQRWHKMPGFGKPHF |
| 72 | KKRIDSCRIYFSTPKSWAVDLGYLSFTGVASSVGRIKIRQDRVWPGDAKFSSCHVVRD |
| | VDEWYAVFPLTFTKEIEKPKGGAVGINRGAVHAIADSTGRVVDSPKFYARSLGVIRHR |
| | ARLLDRKVPFGRAVKPSPTKYHGLPKADIDAAAARVNASPGRLVYEARARGSIAAAE |
| | AHLAALVLPAPRQTSQLPSEGRNRERARRFLALAHQRVRRQREWFLHNESAHYAQSY |
| | TKIAIEDWSTKEMTSSEPRDAEEMKRVTRARNRSILDVGWYELGRQIAYKSEATGAEF |
| | AKVDPGLRETETHVPEAIVRERDVDVSGMLRGEAGISGTCSRCGGLLRASASGHADAE |
| | CEVCLHVEVGDVNAAVNVLKRAMFPGAAPPSKEKAKVTIGIKGRKKKRAA |
| SEQ | MSRVELHRAYKFRLYPTPVQVAELSEWERQLRRLYNLGHEQRLLTLTRHLRPKSPGV |
| ID | LKGECLSCDSTQVQEVGADGRPKTTVRHAEQCPTLACRSCGALRDAEGRTAHTVACA |
| NO: | FVDYYRQGREMTELLAADDQLARVVCSARQEVLRDLDKAWQRWRKMPGFGKPRFK |
| 73 | RRTDSCRIYFSTPKAWKLEGGHLSFTGAATTVGAIKMRQDRNWPASVQFSSCHVVRD |
| | VDEWYAVFPLTFVAEVARPKGGAVGINRGAVHAIADSTGRVVDSPRYYARALGVIRH |
| | RARLFDRKVPSGHAVKPSPTKYRGLSAIEVDRVARATGFTPGRVVTEALNRGGVAYA |
| | ECALAAIAVLGHGPERPLTSDGRNREKARKFLALAHQRVRRQREWFLHNESAHYART |
| | YSKIAIEDWSTKEMTASEPQGEETRRVTRSRNRSILDVGWYELGRQLAYKTEATGAEF |
| | AQVDPGLKETETNVPKAIADARDVDVSGMLRGEAGISGTCSKCGGLLRAPASGHADA |
| | ECEICLNVEVGDVNAAVNVLKRAMFPGDAPPASGEKPKVSIGIKGRQKKKKAA |
| SEQ | MEAIATGMSPERRVELGILPGSVELKRAYKFRLYPMKVQQAELSEWERQLRRLYNLA |
| ID | HEQRLAALLRYRDWDFQKGACPSCRVAVPGVHTAACDHVDYFRQAREMTQLLEVD |
| NO: | AQLSRVICCARQEVLRDLDKAWQRWRKKLGGRPRFKRRTDSCRIYLSTPKHWEIAGR |
| 74 | YLRLSGLASSVGEIRIEQDRAFPEGALLSSCSIVRDVDEWYACLPLTFTQPIERAPHRSV |
| | GLNRGVVHALADSDGRVVDSPKFFERALATVQKRSRDLARKVSGSRNAHKARIKLAK |
| | AHQRVRRQRAAFLHQESAYYSKGFDLVALEDMSVRKMTATAGEAPEMGRGAQRDL |
| | NRGILDVGWYELARQIDYKRLAHGGELLRVDPGQTTPLACVTEEQPARGISSACAVCG |
| | IPLARPASGNARMRCTACGSSQVGDVNAAENVLTRALSSAPSGPKSPKASIKIKGRQK |
| | RLGTPANRAGEASGGDPPVRGPVEGGTLAYVVEPVSESQSDT |
| SEQ | MTVRTYKYRAYPTPEQAEALTSWLRFASQLYNAALEHRKNAWGRHDAHGRGFRFW |
| ID | DGDAAPRKKSDPPGRWVYRGGGGAHISKNDQGKLLTEFRREHAELLPPGMPALVQHE |
| NO: | VLARLERSMAAFFQRATKGQKAGYPRWRSEHRYDSLTFGLTSPSKERFDPETGESLGR |
| 75 | GKTVGAGTYHNGDLRLTGLGELRILEHRRIPMGAIPKSVIVRRSGKRWFVSIAMEMPS |
| | VEPAASGRPAVGLDMGVVTWGTAFTADTSAAAALVADLRRMATDPSDCRRLEELER |
| | EAAQLSEVLAHCRARGLDPARPRRCPKELTKLYRRSLHRLGELDRACARIRRRLQAAH |
| | DIAEPVPDEAGSAVLIEGSNAGMRHARRVARTQRRVARRTRAGHAHSNRRKKAVQA |
| | YARAKERERSARGDHRHKVSRALVRQFEEISVEALDIKQLTVAPEHNPDPQPDLPAHV |
| | QRRRNRGELDAAWGAFFAALDYKAADAGGRVARKPAPHTTQECARCGTLVPKPISLR |
| | VHRCPACGYTAPRTVNSARNVLQRPLEEPGRAGPSGANGRGVPHAVA |
| SEQ | MNCRYRYRIYPTPGQRQSLARLFGCVRVVWNDALFLCRQSEKLPKNSELQKLCITQA |
| ID | KKTEARGWLGQVSAIPLQQSVADLGVAFKNFFQSRSGKRKGKKVNPPRVKRRNNRQ |
| NO: | GARFTRGGFKVKTSKVYLARIGDIKIKWSRPLPSEPSSVTVIKDCAGQYFLSFVVEVKP |
| 76 | EIKPPKNPSIGIDLGLKTFASCSNGEKIDSPDYSRLYRKLKRCQRRLAKRQRGSKRRER |
| | MRVKVAKLNAQIRDKRKDFLHKLSTKVVNENQVIALEDLNVGGMLKNRKLSRAISQA |
| | GWYEFRSLCEGKAEKHNRDFRVISRWEPTSQVCSECGYRWGKIDLSVRSIVCINCGVE |
| | HDRDDNASVNIEQAGLKVGVGHTHDSKRTGSACKTSNGAVCVEPSTHREYVQLTLFD |
| | W |
| SEQ | MKSRWTFRCYPTPEQEQHLARTFGCVRFVWNWALRARTDAFRAGERIGYPATDKAL |
| ID | TLLKQQPETVWLNEVSSVCLQQALRDLQVAFSNFFDKRAAHPSFKRKEARQSANYTE |
| NO: | RGFSFDHERRILKLAKIGAIKVKWSRKAIPHPSSIRLIRTASGKYFVSLVVETQPAPMPE |
| 77 | TGESVGVDFGVARLATLSNGERISNPKHGAKWQRRLAFYQKRLARATKGSKRRMRIK |
| | RHVARIHEKIGNSRSDTLHKLSTDLVTRFDLICVEDLNLRGMVKNHSLARSLHDASIGS |
| | AIRMIEEKAERYGKNVVKIDRWFPSSKTCSDCGHIVEQLPLNVREWTCPECGTTHDRD |
| | ANAAANILAVGQTVSAHGGTVRRSRAKASERKSQRSANRQGVNRA |
| SEQ | KEPLNIGKTAKAVFKEIDPTSLNRAANYDASIELNCKECKFKPFKNVKRYEFNFYNNW |
| ID | YRCNPNSCLQSTYKAQVRKVEIGYEKLKNEILTQMQYYPWFGRLYQNFFHDERDKMT |
| NO: | SLDEIQVIGVQNKVFFNTVEKAWREIIKKRFKDNKETMETIPELKHAAGHGKRKLSNK |
| 78 | SLLRRRFAFVQKSFKFVDNSDVSYRSFSNNIACVLPSRIGVDLGGVISRNPKREYIPQEIS |
| | FNAFWKQHEGLKKGRNIEIQSVQYKGETVKRIEADTGEDKAWGKNRQRRFTSLILKL |
| | VPKQGGKKVWKYPEKRNEGNYEYFPIPIEFILDSGETSIRFGGDEGEAGKQKHLVIPFN |
| | DSKATPLASQQTLLENSRFNAEVKSCIGLAIYANYFYGYARNYVISSIYHKNSKNGQAI |
| | TAIYLESIAHNYVKAIERQLQNLLLNLRDFSFMESHKKELKKYFGGDLEGTGGAQKRR |
| | EKEEKIEKEIEQSYLPRLIRLSLTKMVTKQVEM |
| SEQ | ELIVNENKDPLNIGKTAKAVFKEIDPTSINRAANYDASIELACKECKFKPFNNTKRHDFS |
| ID | FYSNWHRCSPNSCLQSTYRAKIRKTEIGYEKLKNEILNQMQYYPWFGRLYQNFFNDQR |
| NO: | DKMTSLDEIQVTGVQNKIFFNTVEKAWREIIKKRFRDNKETMRTIPDLKNKSGHGSRK |
| 79 | LSNKSLLRRRFAFAQKSFKLVDNSDVSYRAFSNNVACVLPSKIGVDIGGIINKDLKREYI |
| | PQEITFNVFWKQHDGLKKGRNIEIHSVQYKGEIVKRIEADTGEDKAWGKNRQRRFTSL |
| | ILKITPKQGGKKIWKFPEKKNASDYEYFPIPIEFILDNGDASIKFGGEEGEVGKQKHLLIP |
| | FNDSKATPLSSKQMLLETSRFNAEVKSTIGLALYANYFVSYARNYVIKSTYHKNSKKG |
| | QIVTEIYLESISQNFVRAIQRQLQSLMLNLKDWGFMQTHKKELKKYFGSDLEGSKGGQ |
| | KRREKEEKIEKEIEASYLPRLIRLSLTKSVTKAEEM |
| SEQ | PEEKTSKLKPNSINLAANYDANEKFNCKECKFHPFKNKKRYEFNFYNNLHGCKSCTKS |
| ID | TNNPAVKRIEIGYQKLKFEIKNQMEAYPWFGRLRINFYSDEKRKMSELNEMQVTGVK |
| NO: | NKIFFDAIECAWREILKKRFRESKETLITIPKLKNKAGHGARKHRNKKLLIRRRAFMKK |
| 80 | NFHFLDNDSISYRSFANNIACVLPSKVGVDIGGIISPDVGKDIKPVDISLNLMWASKEGI |
| | KSGRKVEIYSTQYDGNMVKKIEAETGEDKSWGKNRKRRQTSLLLSIPKPSKQVQEFDF |
| | KEWPRYKDIEKKVQWRGFPIKIIFDSNHNSIEFGTYQGGKQKVLPIPFNDSKTTPLGSK |
| | MNKLEKLRFNSKIKSRLGSAIAANKFLEAARTYCVDSLYHEVSSANAIGKGKIFIEYYL |
| | EILSQNYIEAAQKQLQRFIESIEQWFVADPFQGRLKQYFKDDLKRAKCFLCANREVQTT |
| | CYAAVKLHKSCAEKVKDKNKELAIKERNNKEDAVIKEVEASNYPRVIRLKLTKTITNK |
| | AM |
| SEQ | SESENKIIEQYYAFLYSFRDKYEKPEFKNRGDIKRKLQNKWEDFLKEQNLKNDKKLSN |
| ID | YIFSNRNFRRSYDREEENEEGIDEKKSKPKRINCFEKEKNLKDQYDKDAINASANKDG |
| NO: | AQKWGCFECIFFPMYKIESGDPNKRIIINKTRFKLFDFYLNLKGCKSCLRSTYHPYRSNV |
| 81 | YIESNYDKLKREIGNFLQQKNIFQRMRKAKVSEGKYLTNLDEYRLSCVAMHFKNRWL |
| | FFDSIQKVLRETIKQRLKQMRESYDEQAKTKRSKGHGRAKYEDQVRMIRRRAYSAQA |
| | HKLLDNGYITLFDYDDKEINKVCLTAINQEGFDIGGYLNSDIDNVMPPIEISFHLKWKY |
| | NEPILNIESPFSKAKISDYLRKIREDLNLERGKEGKARSKKNVRRKVLASKGEDGYKKI |
| | FTDFFSKWKEELEGNAMERVLSQSSGDIQWSKKKRIHYTTLVLNINLLDKKGVGNLK |
| | YYEIAEKTKILSFDKNENKFWPITIQVLLDGYEIGTEYDEIKQLNEKTSKQFTIYDPNTKI |
| | IKIPFTDSKAVPLGMLGINIATLKTVKKTERDIKVSKIFKGGLNSKIVSKIGKGIYAGYFP |
| | TVDKEILEEVEEDTLDNEFSSKSQRNIFLKSIIKNYDKMLKEQLFDFYSFLVRNDLGVRF |
| | LTDRELQNIEDESFNLEKRFFETDRDRIARWFDNTNTDDGKEKFKKLANEIVDSYKPRL |
| | IRLPVVRVIKRIQPVKQREM |
| SEQ | KYSTRDFSELNEIQVTACKQDEFFKVIQNAWREIIKKRFLENRENFIEKKIFKNKKGRG |
| ID | KRQESDKTIQRNRASVMKNFQLIENEKIILRAPSGHVACVFPVKVGLDIGGFKTDDLEK |
| NO: | NIFPPRTITINVFWKNRDRQRKGRKLEVWGIKARTKLIEKVHKWDKLEEVKKKRLKSL |
| 82 | EQKQEKSLDNWSEVNNDSFYKVQIDELQEKIDKSLKGRTMNKILDNKAKESKEAEGL |
| | YIEWEKDFEGEMLRRIEASTGGEEKWGKRRQRRHTSLLLDIKNNSRGSKEIINFYSYAK |
| | QGKKEKKIEFFPFPLTITLDAEEESPLNIKSIPIEDKNATSKYFSIPFTETRATPLSILGDRV |
| | QKFKTKNISGAIKRNLGSSISSCKIVQNAETSAKSILSLPNVKEDNNMEIFINTMSKNYF |
| | RAMMKQMESFIFEMEPKTLIDPYKEKAIKWFEVAASSRAKRKLKKLSKADIKKSELLL |
| | SNTEEFEKEKQEKLEALEKEIEEFYLPRIVRLQLTKTILETPVM |
| SEQ | KKLQLLGHKILLKEYDPNAVNAAANFETSTAELCGQCKMKPFKNKRRFQYTFGKNYH |
| ID | GCLSCIQNVYYAKKRIVQIAKEELKHQLTDSIASIPYKYTSLFSNTNSIDELYILKQERA |
| NO: | AFFSNTNSIDELYITGIENNIAFKVISAIWDEIIKKRRQRYAESLTDTGTVKANRGHGGT |
| 83 | AYKSNTRQEKIRALQKQTLHMVTNPYISLARYKNNYIVATLPRTIGMHIGAIKDRDPQ |
| | KKLSDYAINFNVFWSDDRQLIELSTVQYTGDMVRKIEAETGENNKWGENMKRTKTSL |
| | LLEILTKKTTDELTFKDWAFSTKKEIDSVTKKTYQGFPIGIIFEGNESSVKFGSQNYFPLP |
| | FDAKITPPTAEGFRLDWLRKGSFSSQMKTSYGLAIYSNKVTNAIPAYVIKNMFYKIARA |
| | ENGKQIKAKFLKKYLDIAGNNYVPFIIMQHYRVLDTFEEMPISQPKVIRLSLTKTQHIIIK |
| | KDKTDSKM |
| SEQ | NTSNLINLGKKAINISANYDANLEVGCKNCKFLSSNGNFPRQTNVKEGCHSCEKSTYEP |
| ID | SIYLVKIGERKAKYDVLDSLKKFTFQSLKYQSKKSMKSRNKKPKELKEFVIFANKNKA |
| NO: | FDVIQKSYNHLILQIKKEINRMNSKKRKKNHKRRLFRDREKQLNKLRLIESSNLFLPRE |
| 84 | NKGNNHVFTYVAIHSVGRDIGVIGSYDEKLNFETELTYQLYFNDDKRLLYAYKPKQN |
| | KIIKIKEKLWNLRKEKEPLDLEYEKPLNKSITFSIKNDNLFKVSKDLMLRRAKFNIQGKE |
| | KLSKEERKINRDLIKIKGLVNSMSYGRFDELKKEKNIWSPHIYREVRQKEIKPCLIKNGD |
| | RIEIFEQLKKKMERLRRFREKRQKKISKDLIFAERIAYNFHTKSIKNTSNKINIDQEAKRG |
| | KASYMRKRIGYETFKNKYCEQCLSKGNVYRNVQKGCSCFENPFDWIKKGDENLLPKK |
| | NEDLRVKGAFRDEALEKQIVKIAFNIAKGYEDFYDNLGESTEKDLKLKFKVGTTINEQ |
| | ESLKL |
| SEQ | TSNPIKLGKKAINISANYDSNLQIGCKNCKFLSYNGNFPRQTNVKEGCHSCEKSTYEPP |
| ID | VYTVRIGERRSKYDVLDSLKKFIFLSLKYRQSKKMKTRSKGIRGLEEFVISANLKKAM |
| NO: | DVIQKSYRHLILNIKNEIVRMNGKKRNKNHKRLLFRDREKQLNKLRLIEGSSFFKPPTV |
| 85 | KGDNSIFTCVAIHNIGRDIGIAGDYFDKLEPKIELTYQLYYEYNPKKESEINKRLLYAYK |
| | PKQNKIIEIKEKLWNLRKEKSPLDLEYEKPLTKSITFLVKRDGVFRISKDLMLRKAKFII |
| | QGKEKLSKEERKINRDLIKIKSNIISLTYGRFDELKKDKTIWSPHIFRDVKQGKITPCIER |
| | KGDRMDIFQQLRKKSERLRENRKKRQKKISKDLIFAERIAYNFHTKSIKNTSNLINIKHE |
| | AKRGKASYMRKRIGNETFRIKYCEQCFPKNNVYKNVQKGCSCFEDPFEYIKKGNEDLI |
| | PNKNQDLKAKGAFRDDALEKQIIKVAFNIAKGYEDFYENLKKTTEKDIRLKFKVGTIIS |
| | EEM |
| SEQ | NNSINLSKKAINISANYDANLQVRCKNCKFLSSNGNFPRQTDVKEGCHSCEKSTYEPPV |
| ID | YDVKIGEIKAKYEVLDSLKKFTFQSLKYQLSKSMKFRSKKIKELKEFVIFAKESKALNV |
| NO: | INRSYKHLILNIKNDINRMNSKKRIKNHKGRLFLDRQKQLSKLKLIEGSSFFVPAKNVG |
| 86 | NKSVFTCVAIHSIGRDIGIAGLYDSFTKPVNEITYQIFFSGERRLLYAYKPKQLKILSIKE |
| | NLWSLKNEKKPLDLLYEKPLGKNLNFNVKGGDLFRVSKDLMIRNAKFNVHGRQRLSD |
| | EERLINRNFIKIKGEVVSLSYGRFEELKKDRKLWSPHIFKDVRQNKIKPCLVMQGQRIDI |
| | FEQLKRKLELLKKIRKSRQKKLSKDLIFGERIAYNFHTKSIKNTSNKINIDSDAKRGRAS |
| | YMRKRIGNETFKLKYCDVCFPKANVYRRVQNGCSCSENPYNYIKKGDKDLLPKKDEG |
| | LAIKGAFRDEKLNKQIIKVAFNIAKGYEDFYDDLKKRTEKDVDLKFKIGTTVLDQKPM |
| | EIFDGIVITWL |
| SEQ | LLTTVVETNNLAKKAINVAANFDANIDRQYYRCTPNLCRFIAQSPRETKEKDAGCSSC |
| ID | TQSTYDPKVYVIKIGKLLAKYEILKSLKRFLFMNRYFKQKKTERAQQKQKIGTELNEM |
| NO: | SIFAKATNAMEVIKRATKHCTYDIIPETKSLQMLKRRRHRVKVRSLLKILKERRMKIKK |
| 87 | IPNTFIEIPKQAKKNKSDYYVAAALKSCGIDVGLCGAYEKNAEVEAEYTYQLYYEYKG |
| | NSSTKRILYCYNNPQKNIREFWEAFYIQGSKSHVNTPGTIRLKMEKFLSPITIESEALDFR |
| | VWNSDLKIRNGQYGFIKKRSLGKEAREIKKGMGDIKRKIGNLTYGKSPSELKSIHVYRT |
| | ERENPKKPRAARKKEDNFMEIFEMQRKKDYEVNKKRRKEATDAAKIMDFAEEPIRHY |
| | HTNNLKAVRRIDMNEQVERKKTSVFLKRIMQNGYRGNYCRKCIKAPEGSNRDENVLE |
| | KNEGCLDCIGSEFIWKKSSKEKKGLWHTNRLLRRIRLQCFTTAKAYENFYNDLFEKKE |
| | SSLDIIKLKVSITTKSM |
| SEQ | ASTMNLAKQAINFAANYDSNLEIGCKGCKFMSTWSKKSNPKFYPRQNNQANKCHSCT |
| ID | YSTGEPEVPIIEIGERAAKYKIFTALKKFVFMSVAYKERRRQRFKSKKPKELKELAICSN |
| NO: | REKAMEVIQKSVVHCYGDVKQEIPRIRKIKVLKNHKGRLFYKQKRSKIKIAKLEKGSFF |
| 88 | KTFIPKVHNNGCHSCHEASLNKPILVTTALNTIGADIGLINDYSTIAPTETDISWQVYYE |
| | FIPNGDSEAVKKRLLYFYKPKGALIKSIRDKYFKKGHENAVNTGFFKYQGKIVKGPIKF |
| | VNNELDFARKPDLKSMKIKRAGFAIPSAKRLSKEDREINRESIKIKNKIYSLSYGRKKTL |
| | SDKDIIKHLYRPVRQKGVKPLEYRKAPDGFLEFFYSLKRKERRLRKQKEKRQKDMSEII |
| | DAADEFAWHRHTGSIKKTTNHINFKSEVKRGKVPIMKKRIANDSFNTRHCGKCVKQG |
| | NAINKYYIEKQKNCFDCNSIEFKWEKAALEKKGAFKLNKRLQYIVKACFNVAKAYESF |
| | YEDFRKGEEESLDLKFKIGTTTTLKQYPQNKARAM |
| SEQ | HSHNLMLTKLGKQAINFAANYDANLEIGCKNCKFLSYSPKQANPKKYPRQTDVHEDG |
| ID | NIACHSCMQSTKEPPVYIVPIGERKSKYEILTSLNKFTFLALKYKEKKRQAFRAKKPKE |
| NO: | LQELAIAFNKEKAIKVIDKSIQHLILNIKPEIARIQRQKRLKNRKGKLLYLHKRYAIKMG |
| 89 | LIKNGKYFKVGSPKKDGKKLLVLCALNTIGRDIGIIGNIEENNRSETEITYQLYFDCLDA |
| | NPNELRIKEIEYNRLKSYERKIKRLVYAYKPKQTKILEIRSKFFSKGHENKVNTGSFNFE |
| | NPLNKSISIKVKNSAFDFKIGAPFIMLRNGKFHIPTKKRLSKEEREINRTLSKIKGRVFRL |
| | TYGRNISEQGSKSLHIYRKERQHPKLSLEIRKQPDSFIDEFEKLRLKQNFISKLKKQRQK |
| | KLADLLQFADRIAYNYHTSSLEKTSNFINYKPEVKRGRTSYIKKRIGNEGFEKLYCETCI |
| | KSNDKENAYAVEKEELCFVCKAKPFTWKKTNKDKLGIFKYPSRIKDFIRAAFTVAKSY |
| | NDFYENLKKKDLKNEIFLKFKIGLILSHEKKNHISIAKSVAEDERISGKSIKNILNKSIKLE |
| | KNCYSCFFHKEDM |
| SEQ | SLERVIDKRNLAKKAINIAANFDANINKGFYRCETNQCMFIAQKPRKTNNTGCSSCLQS |
| ID | TYDPVIYVVKVGEMLAKYEILKSLKRFVFMNRSFKQKKTEKAKQKERIGGELNEMSIF |
| NO: | ANAALAMGVIKRAIRHCHVDIRPEINRLSELKKTKHRVAAKSLVKIVKQRKTKWKGIP |
| 90 | NSFIQIPQKARNKDADFYVASALKSGGIDIGLCGTYDKKPHADPRWTYQLYFDTEDES |
| | EKRLLYCYNDPQAKIRDFWKTFYERGNPSMVNSPGTIEFRMEGFFEKMTPISIESKDFD |
| | FRVWNKDLLIRRGLYEIKKRKNLNRKAREIKKAMGSVKRVLANMTYGKSPTDKKSIP |
| | VYRVEREKPKKPRAVRKEENELADKLENYRREDFLIRNRRKREATEIAKIIDAAEPPIR |
| | HYHTNHLRAVKRIDLSKPVARKNTSVFLKRIMQNGYRGNYCKKCIKGNIDPNKDECR |
| | LEDIKKCICCEGTQNIWAKKEKLYTGRINVLNKRIKQMKLECFNVAKAYENFYDNLA |
| | ALKEGDLKVLKLKVSIPALNPEASDPEEDM |
| SEQ | NASINLGKRAINLSANYDSNLVIGCKNCKFLSFNGNFPRQTNVREGCHSCDKSTYAPE |
| ID | VYIVKIGERKAKYDVLDSLKKFTFQSLKYQIKKSMRERSKKPKELLEFVIFANKDKAFN |
| NO: | VIQKSYEHLILNIKQEINRMNGKKRIKNHKKRLFKDREKQLNKLRLIGSSSLFFPRENKG |
| 91 | DKDLFTYVAIHSVGRDIGVAGSYESHIEPISDLTYQLFINNEKRLLYAYKPKQNKIIELK |
| | ENLWNLKKEKKPLDLEFTKPLEKSITFSVKNDKLFKVSKDLMLRQAKFNIQGKEKLSK |
| | EERQINRDFSKIKSNVISLSYGRFEELKKEKNIWSPHIYREVKQKEIKPCIVRKGDRIELF |
| | EQLKRKMDKLKKFRKERQKKISKDLNFAERIAYNFHTKSIKNTSNKINIDQEAKRGKA |
| | SYMRKRIGNESFRKKYCEQCFSVGNVYHNVQNGCSCFDNPIELIKKGDEGLIPKGKED |
| | RKYKGALRDDNLQMQIIRVAFNIAKGYEDFYNNLKEKTEKDLKLKFKIGTTISTQESN |
| | NKEM |
| SEQ | SNLIKLGKQAINFAANYDANLEVGCKNCKFLSSTNKYPRQTNVHLDNKMACRSCNQS |
| ID | TMEPAIYIVRIGEKKAKYDIYNSLTKFNFQSLKYKAKRSQRFKPKQPKELQELSIAVRK |
| NO: | EKALDIIQKSIDHLIQDIRPEIPRIKQQKRYKNHVGKLFYLQKRRKNKLNLIGKGSFFKV |
| 92 | FSPKEKKNELLVICALTNIGRDIGLIGNYNTIINPLFEVTYQLYYDYIPKKNNKNVQRRL |
| | LYAYKSKNEKILKLKEAFFKRGHENAVNLGSFSYEKPLEKSLTLKIKNDKDDFQVSPSL |
| | RIRTGRFFVPSKRNLSRQEREINRRLVKIKSKIKNMTYGKFETARDKQSVHIFRLERQKE |
| | KLPLQFRKDEKEFMEEFQKLKRRTNSLKKLRKSRQKKLADLLQLSEKVVYNNHTGTL |
| | KKTSNFLNFSSSVKRGKTAYIKELLGQEGFETLYCSNCINKGQKTRYNIETKEKCFSCK |
| | DVPFVWKKKSTDKDRKGAFLFPAKLKDVIKATFTVAKAYEDFYDNLKSIDEKKPYIKF |
| | KIGLILAHVRHEHKARAKEEAGQKNIYNKPIKIDKNCKECFFFKEEAM |
| SEQ | NTTRKKFRKRTGFPQSDNIKLAYCSAIVRAANLDADIQKKHNQCNPNLCVGIKSNEQS |
| ID | RKYEHSDRQALLCYACNQSTGAPKVDYIQIGEIGAKYKILQMVNAYDFLSLAYNLTKL |
| NO: | RNGKSRGHQRMSQLDEVVIVADYEKATEVIKRSINHLLDDIRGQLSKLKKRTQNEHIT |
| 93 | EHKQSKIRRKLRKLSRLLKRRRWKWGTIPNPYLKNWVFTKKDPELVTVALLHKLGRD |
| | IGLVNRSKRRSKQKLLPKVGFQLYYKWESPSLNNIKKSKAKKLPKRLLIPYKNVKLFD |
| | NKQKLENAIKSLLESYQKTIKVEFDQFFQNRTEEIIAEEQQTLERGLLKQLEKKKNEFAS |
| | QKKALKEEKKKIKEPRKAKLLMEESRSLGFLMANVSYALFNTTIEDLYKKSNVVSGCI |
| | PQEPVVVFPADIQNKGSLAKILFAPKDGFRIKFSGQHLTIRTAKFKIRGKEIKILTKTKRE |
| | ILKNIEKLRRVWYREQHYKLKLFGKEVSAKPRFLDKRKTSIERRDPNKLADQTDDRQA |
| | ELRNKEYELRHKQHKMAERLDNIDTNAQNLQTLSFWVGEADKPPKLDEKDARGFGV |
| | RTCISAWKWFMEDLLKKQEEDPLLKLKLSIM |
| SEQ | PKKPKFQKRTGFPQPDNLRKEYCLAIVRAANLDADFEKKCTKCEGIKTNKKGNIVKGR |
| ID | TYNSADKDNLLCYACNISTGAPAVDYVFVGALEAKYKILQMVKAYDFHSLAYNLAK |
| NO: | LWKGRGRGHQRMGGLNEVVIVSNNEKALDVIEKSLNHFHDEIRGELSRLKAKFQNEH |
| 94 | LHVHKESKLRRKLRKISRLLKRRRWKWDVIPNSYLRNFTFTKTRPDFISVALLHRVGR |
| | DIGLVTKTKIPKPTDLLPQFGFQIYYTWDEPKLNKLKKSRLRSEPKRLLVPYKKIELYK |
| | NKSVLEEAIRHLAEVYTEDLTICFKDFFETQKRKFVSKEKESLKRELLKELTKLKKDFS |
| | ERKTALKRDRKEIKEPKKAKLLMEESRSLGFLAANTSYALFNLIAADLYTKSKKACST |
| | KLPRQLSTILPLEIKEHKSTTSLAIKPEEGFKIRFSNTHLSIRTPKFKMKGADIKALTKRK |
| | REILKNATKLEKSWYGLKHYKLKLYGKEVAAKPRFLDKRNPSIDRRDPKELMEQIENR |
| | RNEVKDLEYEIRKGQHQMAKRLDNVDTNAQNLQTKSFWVGEADKPPELDSMEAKKL |
| | GLRTCISAWKWFMKDLVLLQEKSPNLKLKLSLTEM |
| SEQ | KFSKRQEGFLIPDNIDLYKCLAIVRSANLDADVQGHKSCYGVKKNGTYRVKQNGKKG |
| ID | VKEKGRKYVFDLIAFKGNIEKIPHEAIEEKDQGRVIVLGKFNYKLILNIEKNHNDRASLE |
| NO: | IKNKIKKLVQISSLETGEFLSDLLSGKIGIDEVYGIIEPDVFSGKELVCKACQQSTYAPLV |
| 95 | EYMPVGELDAKYKILSAIKGYDFLSLAYNLSRNRANKKRGHQKLGGGELSEVVISAN |
| | YDKALNVIKRSINHYHVEIKPEISKLKKKMQNEPLKVMKQARIRRELHQLSRKVKRLK |
| | WKWGMIPNPELQNIIFEKKEKDFVSYALLHTLGRDIGLFKDTSMLQVPNISDYGFQIYY |
| | SWEDPKLNSIKKIKDLPKRLLIPYKRLDFYIDTILVAKVIKNLIELYRKSYVYETFGEEY |
| | GYAKKAEDILFDWDSINLSEGIEQKIQKIKDEFSDLLYEARESKRQNFVESFENILGLYD |
| | KNFASDRNSYQEKIQSMIIKKQQENIEQKLKREFKEVIERGFEGMDQNKKYYKVLSPNI |
| | KGGLLYTDTNNLGFFRSHLAFMLLSKISDDLYRKNNLVSKGGNKGILDQTPETMLTLE |
| | FGKSNLPNISIKRKFFNIKYNSSWIGIRKPKFSIKGAVIREITKKVRDEQRLIKSLEGVWH |
| | KSTHFKRWGKPRFNLPRHPDREKNNDDNLMESITSRREQIQLLLREKQKQQEKMAGR |
| | LDKIDKEIQNLQTANFQIKQIDKKPALTEKSEGKQSVRNALSAWKWFMEDLIKYQKRT |
| | PILQLKLAKM |
| SEQ | KFSKRQEGFVIPENIGLYKCLAIVRSANLDADVQGHVSCYGVKKNGTYVLKQNGKKSI |
| ID | REKGRKYASDLVAFKGDIEKIPFEVIEEKKKEQSIVLGKFNYKLVLDVMKGEKDRASL |
| NO: | TMKNKSKKLVQVSSLGTDEFLLTLLNEKFGIEEIYGIIEPEVFSGKKLVCKACQQSTYAP |
| 96 | LVEYMPVGELDSKYKILSAIKGYDFLSLAYNLARHRSNKKRGHQKLGGGELSEVVISA |
| | NNAKALNVIKRSLNHYYSEIKPEISKLRKKMQNEPLKVGKQARMRRELHQLSRKVKR |
| | LKWKWGKIPNLELQNITFKESDRDFISYALLHTLGRDIGMFNKTEIKMPSNILGYGFQI |
| | YYDWEEPKLNTIKKSKNTPKRILIPYKKLDFYNDSILVARAIKELVGLFQESYEWEIFGN |
| | EYNYAKEAEVELIKLDEESINGNVEKKLQRIKENFSNLLEKAREKKRQNFIESFESIARL |
| | YDESFTADRNEYQREIQSFIIEKQKQSIEKKLKNEFKKIVEKKFNEQEQGKKHYRVLNP |
| | TIINEFLPKDKNNLGFLRSKIAFILLSKISDDLYKKSNAVSKGGEKGIIKQQPETILDLEFS |
| | KSKLPSINIKKKLFNIKYTSSWLGIRKPKFNIKGAKIREITRRVRDVQRTLKSAESSWYA |
| | STHFRRWGFPRFNQPRHPDKEKKSDDRLIESITLLREQIQILLREKQKGQKEMAGRLDD |
| | VDKKIQNLQTANFQIKQTGDKPALTEKSAGKQSFRNALSAWKWFMENLLKYQNKTP |
| | DLKLKIARTVM |
| SEQ | KWIEPNNIDFNKCLAITRSANLDADVQGHKMCYGIKTNGTYKAIGKINKKHNTGIIEKR |
| ID | RTYVYDLIVTKEKNEKIVKKTDFMAIDEEIEFDEKKEKLLKKYIKAEVLGTGELIRKDL |
| NO: | NDGEKFDDLCSIEEPQAFRRSELVCKACNQSTYASDIRYIPIGEIEAKYKILKAIKGYDFL |
| 97 | SLKYNLGRLRDSKKRGHQKMGQGELKEFVICANKEKALDVIKRSLNHYLNEVKDEIS |
| | RLNKKMQNEPLKVNDQARWRRELNQISRRLKRLKWKWGEIPNPELKNLIFKSSRPEFV |
| | SYALIHTLGRDIGLINETELKPNNIQEYGFQIYYKWEDPELNHIKKVKNIPKRFIIPYKNL |
| | DLFGKYTILSRAIEGILKLYSSSFQYKSFKDPNLFAKEGEKKITNEDFELGYDEKIKKIKD |
| | DFKSYKKALLEKKKNTLEDSLNSILSVYEQSLLTEQINNVKKWKEGLLKSKESIHKQK |
| | KIENIEDIISRIEELKNVEGWIRTKERDIVNKEETNLKREIKKELKDSYYEEVRKDFSDLK |
| | KGEESEKKPFREEPKPIVIKDYIKFDVLPGENSALGFFLSHLSFNLFDSIQYELFEKSRLSS |
| | SKHPQIPETILDL |
| SEQ | FRKFVKRSGAPQPDNLNKYKCIAIVRAANLDADIMSNESSNCVMCKGIKMNKRKTAK |
| ID | GAAKTTELGRVYAGQSGNLLCTACTKSTMGPLVDYVPIGRIRAKYTILRAVKEYDFLS |
| NO: | LAYNLARTRVSKKGGRQKMHSLSELVIAAEYEIAWNIIKSSVIHYHQETKEEISGLRKK |
| 98 | LQAEHIHKNKEARIRREMHQISRRIKRLKWKWHMIPNSELHNFLFKQQDPSFVAVALL |
| | HTLGRDIGMINKPKGSAKREFIPEYGFQIYYKWMNPKLNDINKQKYRKMPKRSLIPYK |
| | NLNVFGDRELIENAMHKLLKLYDENLEVKGSKFFKTRVVAISSKESEKLKRDLLWKGE |
| | LAKIKKDFNADKNKMQELFKEVKEPKKANALMKQSRNMGFLLQNISYGALGLLANR |
| | MYEASAKQSKGDATKQPSIVIPLEMEFGNAFPKLLLRSGKFAMNVSSPWLTIRKPKFVI |
| | KGNKIKNITKLMKDEKAKLKRLETSYHRATHFRPTLRGSIDWDSPYFSSPKQPNTHRRS |
| | PDRLSADITEYRGRLKSVEAELREGQRAMAKKLDSVDMTASNLQTSNFQLEKGEDPR |
| | LTEIDEKGRSIRNCISSWKKFMEDLMKAQEANPVIKIKIALKDESSVLSEDSM |
| SEQ | KFHPENLNKSYCLAIVRAANLDADIQGHINCIGIKSNKSDRNYENKLESLQNVELLCKA |
| ID | CTKSTYKPNINSVPVGEKKAKYSILSEIKKYDFNSLVYNLKKYRKGKSRGHQKLNELR |
| NO: | ELVITSEYKKALDVINKSVNHYLVNIKNKMSKLKKILQNEHIHVGTLARIRRERNRISR |
| 99 | KLDHYRKKWKFVPNKILKNYVFKNQSPDFVSVALLHKLGRDIGLITKTAILQKSFPEYS |
| | LQLYYKYDTPKLNYLKKSKFKSLPKRILISYKYPKFDINSNYIEESIDKLLKLYEESPIYK |
| | NNSKIIEFFKKSEDNLIKSENDSLKRGIMKEFEKVTKNFSSKKKKLKEELKLKNEDKNS |
| | KMLAKVSRPIGFLKAYLSYMLFNIISNRIFEFSRKSSGRIPQLPSCIINLGNQFENFKNEL |
| | QDSNIGSKKNYKYFCNLLLKSSGFNISYEEEHLSIKTPNFFINGRKLKEITSEKKKIRKEN |
| | EQLIKQWKKLTFFKPSNLNGKKTSDKIRFKSPNNPDIERKSEDNIVENIAKVKYKLEDL |
| | LSEQRKEFNKLAKKHDGVDVEAQCLQTKSFWIDSNSPIKKSLEKKNEKVSVKKKMKA |
| | IRSCISAWKWFMADLIEAQKETPMIKLKLALM |
| SEQ | TTLVPSHLAGIEVMDETTSRNEDMIQKETSRSNEDENYLGVKNKCGINVHKSGRGSSK |
| ID | HEPNMPPEKSGEGQMPKQDSTEMQQRFDESVTGETQVSAGATASIKTDARANSGPRV |
| NO: | GTARALIVKASNLDRDIKLGCKPCEYIRSELPMGKKNGCNHCEKSSDIASVPKVESGFR |
| 100 | KAKYELVRRFESFAADSISRHLGKEQARTRGKRGKKDKKEQMGKVNLDEIAILKNESL |
| | IEYTENQILDARSNRIKEWLRSLRLRLRTRNKGLKKSKSIRRQLITLRRDYRKWIKPNPY |
| | RPDEDPNENSLRLHTKLGVDIGVQGGDNKRMNSDDYETSFSITWRDTATRKICFTKPK |
| | GLLPRHMKFKLRGYPELILYNEELRIQDSQKFPLVDWERIPIFKLRGVSLGKKKVKALN |
| | RITEAPRLVVAKRIQVNIESKKKKVLTRYVYNDKSINGRLVKAEDSNKDPLLEFKKQA |
| | EEINSDAKYYENQEIAKNYLWGCEGLHKNLLEEQTKNPYLAFKYGFLNIV |
| SEQ | LDFKRTCSQELVLLPEIEGLKLSGTQGVTSLAKKLINKAANVDRDESYGCHHCIHTRTS |
| ID | LSKPVKKDCNSCNQSTNHPAVPITLKGYKIAFYELWHRFTSWAVDSISKALHRNKVM |
| NO: | GKVNLDEYAVVDNSHIVCYAVRKCYEKRQRSVRLHKRAYRCRAKHYNKSQPKVGRI |
| 101 | YKKSKRRNARNLKKEAKRYFQPNEITNGSSDALFYKIGVDLGIAKGTPETEVKVDVSI |
| | CFQVYYGDARRVLRVRKMDELQSFHLDYTGKLKLKGIGNKDTFTIAKRNESLKWGST |
| | KYEVSRAHKKFKPFGKKGSVKRKCNDYFRSIASWSCEAASQRAQSNLKNAFPYQKAL |
| | VKCYKNLDYKGVKKNDMWYRLCSNRIFRYSRIAEDIAQYQSDKGKAKFEFVILAQSV |
| | AEYDISAIM |
| SEQ | VFLTDDKRKTALRKIRSAFRKTAEIALVRAQEADSLDRQAKKLTIETVSFGAPGAKNA |
| ID | FIGSLQGYNWNSHRANVPSSGSAKDVFRITELGLGIPQSAHEASIGKSFELVGNVVRYT |
| NO: | ANLLSKGYKKGAVNKGAKQQREIKGKEQLSFDLISNGPISGDKLINGQKDALAWWLI |
| 102 | DKMGFHIGLAMEPLSSPNTYGITLQAFWKRHTAPRRYSRGVIRQWQLPFGRQLAPLIH |
| | NFFRKKGASIPIVLTNASKKLAGKGVLLEQTALVDPKKWWQVKEQVTGPLSNIWERS |
| | VPLVLYTATFTHKHGAAHKRPLTLKVIRISSGSVFLLPLSKVTPGKLVRAWMPDINILR |
| | DGRPDEAAYKGPDLIRARERSFPLAYTCVTQIADEWQKRALESNRDSITPLEAKLVTGS |
| | DLLQIHSTVQQAVEQGIGGRISSPIQELLAKDALQLVLQQLFMTVDLLRIQWQLKQEV |
| | ADGNTSEKAVGWAIRISNIHKDAYKTAIEPCTSALKQAWNPLSGFEERTFQLDASIVRK |
| | RSTAKTPDDELVIVLRQQAAEMTVAVTQSVSKELMELAVRHSATLHLLVGEVASKQL |
| | SRSADKDRGAMDHWKLLSQSM |
| SEQ | EDLLQKALNTATNVAAIERHSCISCLFTESEIDVKYKTPDKIGQNTAGCQSCTFRVGYS |
| ID | GNSHTLPMGNRIALDKLRETIQRYAWHSLLFNVPPAPTSKRVRAISELRVAAGRERLFT |
| NO: | VITFVQTNILSKLQKRYAANWTPKSQERLSRLREEGQHILSLLESGSWQQKEVVREDQ |
| 103 | DLIVCSALTKPGLSIGAFCRPKYLKPAKHALVLRLIFVEQWPGQIWGQSKRTRRMRRR |
| | KDVERVYDISVQAWALKGKETRISECIDTMRRHQQAYIGVLPFLILSGSTVRGKGDCPI |
| | LKEITRMRYCPNNEGLIPLGIFYRGSANKLLRVVKGSSFTLPMWQNIETLPHPEPFSPEG |
| | WTATGALYEKNLAYWSALNEAVDWYTGQILSSGLQYPNQNEFLARLQNVIDSIPRKW |
| | FRPQGLKNLKPNGQEDIVPNEFVIPQNAIRAHHVIEWYHKTNDLVAKTLLGWGSQTTL |
| | NQTRPQGDLRFTYTRYYFREKEVPEV |
| SEQ | VPKKKLMRELAKKAVFEAIFNDPIPGSFGCKRCTLIDGARVTDAIEKKQGAKRCAGCE |
| ID | PCTFHTLYDSVKHALPAATGCDRTAIDTGLWEILTALRSYNWMSFRRNAVSDASQKQ |
| NO: | VWSIEELAIWADKERALRVILSALTHTIGKLKNGFSRDGVWKGGKQLYENLAQKDLA |
| 104 | KGLFANGEIFGKELVEADHDMLAWTIVPNHQFHIGLIRGNWKPAAVEASTAFDARWL |
| | TNGAPLRDTRTHGHRGRRFNRTEKLTVLCIKRDGGVSEEFRQERDYELSVMLLQPKN |
| | KLKPEPKGELNSFEDLHDHWWFLKGDEATALVGLTSDPTVGDFIQLGLYIRNPIKAHG |
| | ETKRRLLICFEPPIKLPLRRAFPSEAFKTWEPTINVFRNGRRDTEAYYDIDRARVFEFPET |
| | RVSLEHLSKQWEVLRLEPDRENTDPYEAQQNEGAELQVYSLLQEAAQKMAPKVVIDP |
| | FGQFPLELFSTFVAQLFNAPLSDTKAKIGKPLDSGFVVESHLHLLEEDFAYRDFVRVTF |
| | MGTEPTFRVIHYSNGEGYWKKTVLKGKNNIRTALIPEGAKAAVDAYKNKRCPLTLEA |
| | AILNEEKDRRLVLGNKALSLLAQTARGNLTILEALAAEVLRPLSGTEGVVHLHACVTR |
| | HSTLTESTETDNM |
| SEQ | VEKLFSERLKRAMWLKNEAGRAPPAETLTLKHKRVSGGHEKVKEELQRVLRSLSGTN |
| ID | QAAWNLGLSGGREPKSSDALKGEKSRVVLETVVFHSGHNRVLYDVIEREDQVHQRSS |
| NO: | IMHMRRKGSNLLRLWGRSGKVRRKMREEVAEIKPVWHKDSRWLAIVEEGRQSVVGIS |
| 105 | SAGLAVFAVQESQCTTAEPKPLEYVVSIWFRGSKALNPQDRYLEFKKLKTTEALRGQQ |
| | YDPIPFSLKRGAGCSLAIRGEGIKFGSRGPIKQFFGSDRSRPSHADYDGKRRLSLFSKYA |
| | GDLADLTEEQWNRTVSAFAEDEVRRATLANIQDFLSISHEKYAERLKKRIESIEEPVSA |
| | SKLEAYLSAIFETFVQQREALASNFLMRLVESVALLISLEEKSPRVEFRVARYLAESKE |
| | GFNRKAM |
| SEQ | VVITQSELYKERLLRVMEIKNDRGRKEPRESQGLVLRFTQVTGGQEKVKQKLWLIFEG |
| ID | FSGTNQASWNFGQPAGGRKPNSGDALKGPKSRVTYETVVFHFGLRLLSAVIERHNLK |
| NO: | QQRQTMAYMKRRAAARKKWARSGKKCSRMRNEVEKIKPKWHKDPRWFDIVKEGEP |
| 106 | SIVGISSAGFAIYIVEEPNFPRQDPLEIEYAISIWFRRDRSQYLTFKKIQKAEKLKELQYNP |
| | IPFRLKQEKTSLVFESGDIKFGSRGSIEHFRDEARGKPPKADMDNNRRLTMFSVFSGNL |
| | TNLTEEQYARPVSGLLAPDEKRMPTLLKKLQDFFTPIHEKYGERIKQRLANSEASKRPF |
| | KKLEEYLPAIYLEFRARREGLASNWVLVLINSVRTLVRIKSEDPYIEFKVSQYLLEKED |
| | NKAL |
| SEQ | KQDALFEERLKKAIFIKRQADPLQREELSLLPPNRKIVTGGHESAKDTLKQILRAINGTN |
| ID | QASWNPGTPSGKRDSKSADALAGPKSRVKLETVVFHVGHRLLKKVVEYQGHQKQQH |
| NO: | GLKAFMRTCAAMRKKWKRSGKVVGELREQLANIQPKWHYDSRPLNLCFEGKPSVVG |
| 107 | LRSAGIALYTIQKSVVPVKEPKPIEYAVSIWFRGPKAMDREDRCLEFKKLKIATELRKL |
| | QFEPIVSTLTQGIKGFSLYIQGNSVKFGSRGPIKYFSNESVRQRPPKADPDGNKRLALFS |
| | KFSGDLSDLTEEQWNRPILAFEGIIRRATLGNIQDYLTVGHEQFAISLEQLLSEKESVLQ |
| | MSIEQQRLKKNLGKKAENEWVESFGAEQARKKAQGIREYISGFFQEYCSQREQWAEN |
| | WVQQLNKSVRLFLTIQDSTPFIEFRVARYLPKGEKKKGKAM |
| SEQ | ANHAERHKRLRKEANRAANRNRPLVADCDTGDPLVGICRLLRRGDKMQPNKTGCRS |
| ID | CEQVEPELRDAILVSGPGRLDNYKYELFQRGRAMAVHRLLKRVPKLNRPKKAAGNDE |
| NO: | KKAENKKSEIQKEKQKQRRMMPAVSMKQVSVADFKHVIENTVRHLFGDRRDREIAEC |
| 108 | AALRAASKYFLKSRRVRPRKLPKLANPDHGKELKGLRLREKRAKLKKEKEKQAELAR |
| | SNQKGAVLHVATLKKDAPPMPYEKTQGRNDYTTFVISAAIKVGATRGTKPLLTPQPRE |
| | WQCSLYWRDGQRWIRGGLLGLQAGIVLGPKLNRELLEAVLQRPIECRMSGCGNPLQV |
| | RGAAVDFFMTTNPFYVSGAAYAQKKFKPFGTKRASEDGAAAKAREKLMTQLAKVLD |
| | KVVTQAAHSPLDGIWETRPEAKLRAMIMALEHEWIFLRPGPCHNAAEEVIKCDCTGG |
| | HAILWALIDEARGALEHKEFYAVTRAHTHDCEKQKLGGRLAGFLDLLIAQDVPLDDA |
| | PAARKIKTLLEATPPAPCYKAATSIATCDCEGKFDKLWAIIDATRAGHGTEDLWARTL |
| | AYPQNVNCKCKAGKDLTHRLADFLGLLIKRDGPFRERPPHKVTGDRKLVFSGDKKCK |
| | GHQYVILAKAHNEEVVRAWISRWGLKSRTNKAGYAATELNLLLNWLSICRRRWMDM |
| | LTVQRDTPYIRMKTGRLVVDDKKERKAM |
| SEQ | AKQREALRVALERGIVRASNRTYTLVTNCTKGGPLPEQCRMIERGKARAMKWEPKLV |
| ID | GCGSCAAATVDLPAIEEYAQPGRLDVAKYKLTTQILAMATRRMMVRAAKLSRRKGQ |
| NO: | WPAKVQEEKEEPPEPKKMLKAVEMRPVAIVDFNRVIQTTIEHLWAERANADEAELKA |
| 109 | LKAAAAYFGPSLKIRARGPPKAAIGRELKKAHRKKAYAERKKARRKRAELARSQARG |
| | AAAHAAIRERDIPPMAYERTQGRNDVTTIPIAAAIKIAATRGARPLPAPKPMKWQCSLY |
| | WNEGQRWIRGGMLTAQAYAHAANIHRPMRCEMWGVGNPLKVRAFEGRVADPDGA |
| | KGRKAEFRLQTNAFYVSGAAYRNKKFKPFGTDRGGIGSARKKRERLMAQLAKILDKV |
| | VSQAAHSPLDDIWHTRPAQKLRAMIKQLEHEWMFLRPQAPTVEGTKPDVDVAGNMQ |
| | RQIKALMAPDLPPIEKGSPAKRFTGDKRKKGERAVRVAEAHSDEVVTAWISRWGIQTR |
| | RNEGSYAAQELELLLNWLQICRRRWLDMTAAQRVSPYIRMKSGRMITDAADEGVAPI |
| | PLVENM |
| SEQ | KSISGRSIKHMACLKDMLKSEITEIEEKQKKESLRKWDYYSKFSDEILFRRNLNVSANH |
| ID | DANACYGCNPCAFLKEVYGFRIERRNNERIISYRRGLAGCKSCVQSTGYPPIEFVRRKF |
| NO: | GADKAMEIVREVLHRRNWGALARNIGREKEADPILGELNELLLVDARPYFGNKSAAN |
| 110 | ETNLAFNVITRAAKKFRDEGMYDIHKQLDIHSEEGKVPKGRKSRLIRIERKHKAIHGLD |
| | PGETWRYPHCGKGEKYGVWLNRSRLIHIKGNEYRCLTAFGTTGRRMSLDVACSVLGH |
| | PLVKKKRKKGKKTVDGTELWQIKKATETLPEDPIDCTFYLYAAKPTKDPFILKVGSLK |
| | APRWKKLHKDFFEYSDTEKTQGQEKGKRVVRRGKVPRILSLRPDAKFKVSIWDDPYN |
| | GKNKEGTLLRMELSGLDGAKKPLILKRYGEPNTKPKNFVFWRPHITPHPLTFTPKHDF |
| | GDPNKKTKRRRVFNREYYGHLNDLAKMEPNAKFFEDREVSNKKNPKAKNIRIQAKES |
| | LPNIVAKNGRWAAFDPNDSLWKLYLHWRGRRKTIKGGISQEFQEFKERLDLYKKHED |
| | ESEWKEKEKLWENHEKEWKKTLEIHGSIAEVSQRCVMQSMMGPLDGLVQKKDYVHI |
| | GQSSLKAADDAWTFSANRYKKATGPKWGKISVSNLLYDANQANAELISQSISKYLSK |
| | QKDNQGCEGRKMKFLIKIIEPLRENFVKHTRWLHEMTQKDCEVRAQFSRVSM |
| SEQ | FPSDVGADALKHVRMLQPRLTDEVRKVALTRAPSDRPALARFAAVAQDGLAFVRHL |
| ID | NVSANHDSNCTFPRDPRDPRRGPCEPNPCAFLREVWGFRIVARGNERALSYRRGLAGC |
| NO: | KSCVQSTGFPSVPFHRIGADDCMRKLHEILKARNWRLLARNIGREREADPLLTELSEYL |
| 111 | LVDARTYPDGAAPNSGRLAENVIKRAAKKFRDEGMRDIHAQLRVHSREGKVPKGRLQ |
| | RLRRIERKHRAIHALDPGPSWEAEGSARAEVQGVAVYRSQLLRVGEIHTQQIEPVGIVA |
| | RTLFGVGRTDLDVAVSVLGAPLTKRKKGSKTLESTEDFRIAKARETRAEDKIEVAFVL |
| | YPTASLLRDEIPKDAFPAMRIDRFLLKVGSVQADREILLQDDYYRFGDAEVKAGKNKG |
| | RTVTRPVKVPRLQALRPDAKFRVNVWADPFGAGDSPGTLLRLEVSGVTRRSQPLRLLR |
| | YGQPSTQPANFLCWRPHRVPDPMTFTPRQKFGERRKNRRTRRPRVFERLYQVHIKHLA |
| | HLEPNRKWFEEARVSAQKWAKARAIRRKGAEDIPVVAPPAKRRWAALQPNAELWDL |
| | YAHDREARKRFRGGRAAEGEEFKPRLNLYLAHEPEAEWESKRDRWERYEKKWTAVL |
| | EEHSRMCAVADRTLPQFLSDPLGARMDDKDYAFVGKSALAVAEAFVEEGTVERAQG |
| | NCSITAKKKFASNASRKRLSVANLLDVSDKADRALVFQAVRQYVQRQAENGGVEGR |
| | RMAFLRKLLAPLRQNFVCHTRWLHM |
| SEQ | AARKKKRGKIGITVKAKEKSPPAAGPFMARKLVNVAANVDGVEVHLCVECEADAHG |
| ID | SASARLLGGCRSCTGSIGAEGRLMGSVDVDRERVIAEPVHTETERLGPDVKAFEAGTA |
| NO: | ESKYAIQRGLEYWGVDLISRNRARTVRKMEEADRPESSTMEKTSWDEIAIKTYSQAYH |
| 112 | ASENHLFWERQRRVRQHALALFRRARERNRGESPLQSTQRPAPLVLAALHAEAAAISG |
| | RARAEYVLRGPSANVRAAAADIDAKPLGHYKTPSPKVARGFPVKRDLLRARHRIVGL |
| | SRAYFKPSDVVRGTSDAIAHVAGRNIGVAGGKPKEIEKTFTLPFVAYWEDVDRVVHCS |
| | SFKADGPWVRDQRIKIRGVSSAVGTFSLYGLDVAWSKPTSFYIRCSDIRKKFHPKGFGP |
| | MKHWRQWAKELDRLTEQRASCVVRALQDDEELLQTMERGQRYYDVFSCAATHATR |
| | GEADPSGGCSRCELVSCGVAHKVTKKAKGDTGIEAVAVAGCSLCESKLVGPSKPRVH |
| | RQMAALRQSHALNYLRRLQREWEALEAVQAPTPYLRFKYARHLEVRSM |
| SEQ | AAKKKKQRGKIGISVKPKEGSAPPADGPFMARKLVNVAANVDGVEVNLCIECEADAH |
| ID | GSAPARLLGGCKSCTGSIGAEGRLMGSVDVDRADAIAKPVNTETEKLGPDVQAFEAG |
| NO: | TAETKYALQRGLEYWGVDLISRNRSRTVRRTEEGQPESATMEKTSWDEIAIKSYTRAY |
| 113 | HASENHLFWERQRRVRQHALALFKRAKERNRGDSTLPREPGHGLVAIAALACEAYAV |
| | GGRNLAETVVRGPTFGTARAVRDVEIASLGRYKTPSPKVAHGSPVKRDFLRARHRIVG |
| | LARAYYRPSDVVRGTSDAIAHVAGRNIGVAGGKPRAVEAVFTLPFVAYWEDVDRVV |
| | HCSSFQVSAPWNRDQRMKIAGVTTAAGTFSLHGGELKWAKPTSFYIRCSDTRRKFRPK |
| | GFGPMKRWRQWAKDLDRLVEQRASCVVRALQDDAALLETMERGQRYYDVFACAVT |
| | HATRGEADRLAGCSRCALTPCQEAHRVTTKPRGDAGVEQVQTSDCSLCEGKLVGPSK |
| | PRLHRTLTLLRQEHGLNYLRRLQREWESLEAVQVPTPYLRFKYARHLEVRSM |
| SEQ | TDSQSESVPEVVYALTGGEVPGRVPPDGGSAEGARNAPTGLRKQRGKIKISAKPSKPGS |
| ID | PASSLARTLVNEAANVDGVQSSGCATCRMRANGSAPRALPIGCVACASSIGRAPQEET |
| NO: | VCALPTTQGPDVRLLEGGHALRKYDIQRALEYWGVDLIGRNLDRQAGRGMEPAEGA |
| 114 | TATMKRVSMDELAVLDFGKSYYASEQHLFAARQRRVRQHAKALKIRAKHANRSGSV |
| | KRALDRSRKQVTALAREFFKPSDVVRGDSDALAHVVGRNLGVSRHPAREIPQTFTLPL |
| | CAYWEDVDRVISCSSLLAGEPFARDQEIRIEGVSSALGSLRLYRGAIEWHKPTSLYIRCS |
| | DTRRKFRPRGGLKKRWRQWAKDLDRLVEQRACCIVRSLQADVELLQTMERAQRFYD |
| | VHDCAATHVGPVAVRCSPCAGKQFDWDRYRLLAALRQEHALNYLRRLQREWESLEA |
| | QQVKMPYLRFKYARKLEVSGPLIGLEVRREPSMGTAIAEM |
| SEQ | AGTAGRRHGSLGARRSINIAGVTDRHGRWGCESCVYTRDQAGNRARCAPCDQSTYAP |
| ID | DVQEVTIGQRQAKYTIFLTLQSFSWTNTMRNNKRAAAGRSKRTTGKRIGQLAEIKITG |
| NO: | VGLAHAHNVIQRSLQHNITKMWRAEKGKSKRVARLKKAKQLTKRRAYFRRRMSRQS |
| 115 | RGNGFFRTGKGGIHAVAPVKIGLDVGMIASGSSEPADEQTVTLDAIWKGRKKKIRLIG |
| | AKGELAVAACRFREQQTKGDKCIPLILQDGEVRWNQNNWQCHPKKLVPLCGLEVSR |
| | KFVSQADRLAQNKVASPLAARFDKTSVKGTLVESDFAAVLVNVTSIYQQCHAMLLRS |
| | QEPTPSLRVQRTITSM |
| SEQ | GVRFSPAQSQVFFRTVIPQSVEARFAINMAAIHDAAGAFGCSVCRFEDRTPRNAKAVH |
| ID | GCSPCTRSTNRPDVFVLPVGAIKAKYDVFMRLLGFNWTHLNRRQAKRVTVRDRIGQL |
| NO: | DELAISMLTGKAKAVLKKSICHNVDKSFKAMRGSLKKLHRKASKTGKSQLRAKLSDL |
| 116 | RERTNTTQEGSHVEGDSDVALNKIGLDVGLVGKPDYPSEESVEVVVCLYFVGKVLILD |
| | AQGRIRDMRAKQYDGFKIPIIQRGQLTVLSVKDLGKWSLVRQDYVLAGDLRFEPKISK |
| | DRKYAECVKRIALITLQASLGFKERIPYYVTKQVEIKNASHIAFVTEAIQNCAENFREM |
| | TEYLMKYQEKSPDLKVLLTQLM |
| SEQ | RAVVGKVFLEQARRALNLATNFGTNHRTGCNGCYVTPGKLSIPQDGEKNAAGCTSCL |
| ID | MKATASYVSYPKPLGEKVAKYSTLDALKGFPWYSLRLNLRPNYRGKPINGVQEVAPV |
| NO: | SKFRLAEEVIQAVQRYHFTELEQSFPGGRRRLRELRAFYTKEYRRAPEQRQHVVNGDR |
| 117 | NIVVVTVLHELGFSVGMFNEVELLPKTPIECAVNVFIRGNRVLLEVRKPQFDKERLLVE |
| | SLWKKDSRRHTAKWTPPNNEGRIFTAEGWKDFQLPLLLGSTSRSLRAIEKEGFVQLAP |
| | GRDPDYNNTIDEQHSGRPFLPLYLYLQGTISQEYCVFAGTWVIPFQDGISPYSTKDTFQP |
| | DLKRKAYSLLLDAVKHRLGNKVASGLQYGRFPAIEELKRLVRMHGATRKIPRGEKDL |
| | LKKGDPDTPEWWLLEQYPEFWRLCDAAAKRVSQNVGLLLSLKKQPLWQRRWLESRT |
| | RNEPLDNLPLSMALTLHLTNEEAL |
| SEQ | AAVYSKFYIENHFKMGIPETLSRIRGPSIIQGFSVNENYINIAGVGDRDFIFGCKKCKYTR |
| ID | GKPSSKKINKCHPCKRSTYPEPVIDVRGSISEFKYKIYNKLKQEPNQSIKQNTKGRMNPS |
| NO: | DHTSSNDGIIINGIDNRIAYNVIFSSYKHLMEKQINLLRDTTKRKARQIKKYNNSGKKK |
| 118 | HSLRSQTKGNLKNRYHMLGMFKKGSLTITNEGDFITAVRKVGLDISLYKNESLNKQEV |
| | ETELCLNIKWGRTKSYTVSGYIPLPINIDWKLYLFEKETGLTLRLFGNKYKIQSKKFLIA |
| | QLFKPKRPPCADPVVKKAQKWSALNAHVQQMAGLFSDSHLLKRELKNRMHKQLDFK |
| | SLWVGTEDYIKWFEELSRSYVEGAEKSLEFFRQDYFCFNYTKQTTM |
| SEQ | PQQQRDLMLMAANYDQDYGNGCGPCTVVASAAYRPDPQAQHGCKRHLRTLGASAV |
| ID | THVGLGDRTATITALHRLRGPAALAARARAAQAASAPMTPDTDAPDDRRRLEAIDAD |
| NO: | DVVLVGAHRALWSAVRRWADDRRAALRRRLHSEREWLLKDQIRWAELYTLIEASGT |
| 119 | PPQGRWRNTLGALRGQSRWRRVLAPTMRATCAETHAELWDALAELVPEMAKDRRG |
| | LLRPPVEADALWRAPMIVEGWRGGHSVVVDAVAPPLDLPQPCAWTAVRLSGDPRQR |
| | WGLHLAVPPLGQVQPPDPLKATLAVSMRHRGGVRVRTLQAMAVDADAPMQRHLQV |
| | PLTLQRGGGLQWGIHSRGVRRREARSMASWEGPPIWTGLQLVNRWKGQGSALLAPD |
| | RPPDTPPYAPDAAVAPAQPDTKRARRTLKEACTVCRCAPGHMRQLQVTLTGDGTWR |
| | RFRLRAPQGAKRKAEVLKVATQHDERIANYTAWYLKRPEHAAGCDTCDGDSRLDGA |
| | CRGCRPLLVGDQCFRRYLDKIEADRDDGLAQIKPKAQEAVAAMAAKRDARAQKVAA |
| | RAAKLSEATGQRTAATRDASHEARAQKELEAVATEGTTVRHDAAAVSAFGSWVARK |
| | GDEYRHQVGVLANRLEHGLRLQELMAPDSVVADQQRASGHARVGYRYVLTAM |
| SEQ | AVAHPVGRGNAGSPGARGPEELPRQLVNRASNVTRPATYGCAPCRHVRLSIPKPVLTG |
| ID | CRACEQTTHPAPKRAVRGGADAAKYDLAAFFAGWAADLEGRNRRRQVHAPLDPQPD |
| NO: | PNHEPAVTLQKIDLAEVSIEEFQRVLARSVKHRHDGRASREREKARAYAQVAKKRRN |
| 120 | SHAHGARTRRAVRRQTRAVRRAHRMGANSGEILVASGAEDPVPEAIDHAAQLRRRIR |
| | ACARDLEGLRHLSRRYLKTLEKPCRRPRAPDLGRARCHALVESLQAAERELEELRRCD |
| | SPDTAMRRLDAVLAAAASTDATFATGWTVVGMDLGVAPRGSAAPEVSPMEMAISVF |
| | WRKGSRRVIVSKPIAGMPIRRHELIRLEGLGTLRLDGNHYTGAGVTKGRGLSEGTEPDF |
| | REKSPSTLGFTLSDYRHESRWRPYGAKQGKTARQFFAAMSRELRALVEHQVLAPMGP |
| | PLLEAHERRFETLLKGQDNKSIHAGGGGRYVWRGPPDSKKRPAADGDWFRFGRGHA |
| | DHRGWANKRHELAANYLQSAFRLWSTLAEAQEPTPYARYKYTRVTM |
| SEQ | WDFLTLQVYERHTSPEVCVAGNSTKCASGTRKSDHTHGVGVKLGAQEINVSANDDR |
| ID | DHEVGCNICVISRVSLDIKGWRYGCESCVQSTPEWRSIVRFDRNHKEAKGECLSRFEY |
| NO: | WGAQSTARSLKRNKLMGGVNLDELAIVQNENVVKTSLKHLFDKRKDRIQANLKAVK |
| 121 | VRMRERRKSGRQRKALRRQCRKLKRYLRSYDPSDIKEGNSCSAFTKLGLDIGISPNKPP |
| | KIEPKVEVVFSLFYQGACDKIVTVSSPESPLPRSWKIKIDGIRALYVKSTKVKFGGRTFR |
| | AGQRNNRRKVRPPNVKKGKRKGSRSQFFNKFAVGLDAVSQQLPIASVQGLWGRAET |
| | KKAQTICLKQLESNKPLKESQRCLFLADNWVVRVCGFLRALSQRQGPTPYIRYRYRCN |
| | M |
| SEQ | ARNVGQRNASRQSKRESAKARSRRVTGGHASVTQGVALINAAANADRDHTTGCEPC |
| ID | TWERVNLPLQEVIHGCDSCTKSSPFWRDIKVVNKGYREAKEEIMRIASGISADHLSRAL |
| NO: | SHNKVMGRLNLDEVCILDFRTVLDTSLKHLTDSRSNGIKEHIRAVHRKIRMRRKSGKT |
| 122 | ARALRKQYFALRRQWKAGHKPNSIREGNSLTALRAVGFDVGVSEGTEPMPAPQTEVV |
| | LSVFYKGSATRILRISSPHPIAKRSWKVKIAGIKALKLIRREHDFSFGRETYNASQRAEK |
| | RKFSPHAARKDFFNSFAVQLDRLAQQLCVSSVENLWVTEPQQKLLTLAKDTAPYGIRE |
| | GARFADTRARLAWNWVFRVCGFTRALHQEQEPTPYCRFTWRSKM |

In some cases, a suitable Cas13 programmable nuclease comprises an amino acid sequence having at least 80%, at least 85%, at least 90%, at least 95%, at least 98%, at least 99%, or 100%, amino acid sequence identity to any one of SEQ ID NO: 123-SEQ ID NO: 140.

TABLE 3
Cas13 Sequences

| SEQ | | |
|---|---|---|
| ID | | |
| NO | Description | Sequence |
| SEQ | Listeria | MWISIKTLIHHLGVLFFCDYMYNRREKKIIEVKTMRITKVEVDRKKVLIS |
| ID | seeligeri | RDKNGGKLVYENEMQDNTEQIMHHKKSSFYKSVVNKTICRPEQKQMK |
| NO: | C2c2 amino | KLVHGLLQENSQEKIKVSDVTKLNISNFLNHRFKKSLYYFPENSPDKSEE |
| 123 | acid | YRIEINLSQLLEDSLKKQQGTFICWESFSKDMELYINWAENYISSKTKLI |
| | sequence | KKSIRNNRIQSTESRSGQLMDRYMKDILNKNKPFDIQSVSEKYQLEKLT |
| | | SALKATFKEAKKNDKEINYKLKSTLQNHERQIIEELKENSELNQFNIEIR |
| | | KHLETYFPIKKTNRKVGDIRNLEIGEIQKIVNHRLKNKIVQRILQEGKLA |
| | | SYEIESTVNSNSLQKIKIEEAFALKFINACLFASNNLRNMVYPVCKKDIL |
| | | MIGEFKNSFKEIKHKKFIRQWSQFFSQEITVDDIELASWGLRGAIAPIRNE |
| | | IIHLKKHSWKKFFNNPTFKVKKSKIINGKTKDVTSEFLYKETLFKDYFYS |
| | | ELDSVPELIINKMESSKILDYYSSDQLNQVFTIPNFELSLLTSAVPFAPSFK |
| | | RVYLKGFDYQNQDEAQPDYNLKLNIYNEKAFNSEAFQAQYSLFKMVY |
| | | YQVFLPQFTTNNDLFKSSVDFILTLNKERKGYAKAFQDIRKMNKDEKPS |
| | | EYMSYIQSQLMLYQKKQEEKEKINHFEKFINQVFIKGFNSFIEKNRLTYI |
| | | CHPTKNTVPENDNIEIPFHTDMDDSNIAFWLMCKLLDAKQLSELRNEMI |
| | | KFSCSLQSTEEISTFTKAREVIGLALLNGEKGCNDWKELFDDKEAWKK |
| | | NMSLYVSEELLQSLPYTQEDGQTPVINRSIDLVKKYGTETILEKLFSSSD |
| | | DYKVSAKDIAKLHEYDVTEKIAQQESLHKQWIEKPGLARDSAWTKKY |
| | | QNVINDISNYQWAKTKVELTQVRHLHQLTIDLLSRLAGYMSIADRDFQF |
| | | SSNYILERENSEYRVTSWILLSENKNKNKYNDYELYNLKNASIKVSSKN |
| | | DPQLKVDLKQLRLTLEYLELFDNRLKEKRNNISHFNYLNGQLGNSILEL |
| | | FDDARDVLSYDRKLKNAVSKSLKEILSSHGMEVTFKPLYQTNHHLKID |
| | | KLQPKKIHHLGEKSTVSSNQVSNEYCQLVRTLLTMK |
| SEQ | Leptotrichia | MKVTKVGGISHKKYTSEGRLVKSESEENRTDERLSALLNMRLDMYIKN |
| ID | buccalis | PSSTETKENQKRIGKLKKFFSNKMVYLKDNTLSLKNGKKENIDREYSET |
| NO: | (Lbu) C2c2 | DILESDVRDKKNFAVLKKIYLNENVNSEELEVFRNDIKKKLNKINSLKY |
| 124 | amino acid | SFEKNKANYQKINENNIEKVEGKSKRNIIYDYYRESAKRDAYVSNVKE |
| | sequence | AFDKLYKEEDIAKLVLEIENLTKLEKYKIREFYHEIIGRKNDKENFAKIIY |
| | | EEIQNVNNMKELIEKVPDMSELKKSQVFYKYYLDKEELNDKNIKYAFC |
| | | HFVEIEMSQLLKNYVYKRLSNISNDKIKRIFEYQNLKKLIENKLLNKLDT |
| | | YVRNCGKYNYYLQDGEIATSDFIARNRQNEAFLRNIIGVSSVAYFSLRNI |
| | | LETENENDITGRMRGKTVKNNKGEEKYVSGEVDKIYNENKKNEVKEN |
| | | LKMFYSYDFNMDNKNEIEDFFANIDEAISSIRHGIVHFNLELEGKDIFAF |
| | | KNIAPSEISKKMFQNEINEKKLKLKIFRQLNSANVFRYLEKYKILNYLKR |
| | | TRFEFVNKNIPFVPSFTKLYSRIDDLKNSLGIYWKTPKTNDDNKTKEIID |
| | | AQIYLLKNIYYGEFLNYFMSNNGNFFEISKEIIELNKNDKRNLKTGFYKL |
| | | QKFEDIQEKIPKEYLANIQSLYMINAGNQDEEEKDTYIDFIQKIFLKGFM |
| | | TYLANNGRLSLIYIGSDEETNTSLAEKKQEFDKFLKKYEQNNNIKIPYEI |
| | | NEFLREIKLGNILKYTERLNMFYLILKLLNHKELTNLKGSLEKYQSANK |
| | | EEAFSDQLELINLLNLDNNRVTEDFELEADEIGKFLDFNGNKVKDNKEL |
| | | KKFDTNKIYFDGENIIKHRAFYNIKKYGMLNLLEKIADKAGYKISIEELK |
| | | KYSNKKNEIEKNHKMQENLHRKYARPRKDEKFTDEDYESYKQAIENIE |
| | | EYTHLKNKVEFNELNLLQGLLLRILHRLVGYTSIWERDLRFRLKGEFPE |
| | | NQYIEEIFNFENKKNVKYKGGQIVEKYIKFYKELHQNDEVKINKYSSAN |
| | | IKVLKQEKKDLYIRNYIAHFNYIPHAEISLLEVLENLRKLLSYDRKLKNA |
| | | VMKSVVDILKEYGFVATFKIGADKKIGIQTLESEKIVHLKNLKKKKLMT |
| | | DRNSEELCKLVKIMFEYKMEEKKSEN |
| SEQ | Leptotrichia | MGNLFGHKRWYEVRDKKDFKIKRKVKVKRNYDGNKYILNINENNNKE |
| ID | shahii (Lsh) | KIDNNKFIRKYINYKKNDNILKEFTRKFHAGNILFKLKGKEGIIRIENNDD |
| NO: | C2c2 | FLETEEVVLYIEAYGKSEKLKALGITKKKIIDEAIRQGITKDDKKIEIKRQ |
| 125 | protein | ENEEEIEIDIRDEYTNKTLNDCSIILRIIENDELETKKSIYEIFKNINMSLYK |
| | | IIEKIIENETEKVFENRYYEEHLREKLLKDDKIDVILTNFMEIREKIKSNLE |
| | | ILGFVKFYLNVGGDKKKSKNKKMLVEKILNINVDLTVEDIADFVIKELE |
| | | FWNITKRIEKVKKVNNEFLEKRRNRTYIKSYVLLDKHEKFKIERENKKD |
| | | KIVKFFVENIKNNSIKEKIEKILAEFKIDELIKKLEKELKKGNCDTEIFGIF |
| | | KKHYKVNFDSKKFSKKSDEEKELYKIIYRYLKGRIEKILVNEQKVRLKK |
| | | MEKIEIEKILNESILSEKILKRVKQYTLEHIMYLGKLRHNDIDMTTVNTD |
| | | DFSRLHAKEELDLELITFFASTNMELNKIFSRENINNDENIDFFGGDREK |
| | | NYVLDKKILNSKIKIIRDLDFIDNKNNITNNFIRKFTKIGTNERNRILHAIS |
| | | KERDLQGTQDDYNKVINIIQNLKISDEEVSKALNLDVVFKDKKNIITKIN |
| | | DIKISEENNNDIKYLPSFSKVLPEILNLYRNNPKNEPFDTIETEKIVLNALI |
| | | YVNKELYKKLILEDDLEENESKNIFLQELKKTLGNIDEIDENIIENYYKN |
| | | AQISASKGNNKAIKKYQKKVIECYIGYLRKNYEELFDFSDFKMNIQEIK |
| | | KQIKDINDNKTYERITVKTSDKTIVINDDFEYIISIFALLNSNAVINKIRNR |
| | | FFATSVWLNTSEYQNIIDILDEIMQLNTLRNECITENWNLNLEEFIQKMK |
| | | EIEKDFDDFKIQTKKEIFNNYYEDIKNNILTEFKDDINGCDVLEKKLEKIV |
| | | IFDDETKFEIDKKSNILQDEQRKLSNINKKDLKKKVDQYIKDKDQEIKSK |
| | | ILCRIIFNSDFLKKYKKEIDNLIEDMESENENKFQEIYYPKERKNELYIYK |
| | | KNLFLNIGNPNFDKIYGLISNDIKMADAKFLFNIDGKNIRKNKISEIDAIL |
| | | KNLNDKLNGYSKEYKEKYIKKLKENDDFFAKNIQNKNYKSFEKDYNR |
| | | VSEYKKIRDLVEFNYLNKIESYLIDINWKLAIQMARFERDMHYIVNGLR |
| | | ELGIIKLSGYNTGISRAYPKRNGSDGFYTTTAYYKFFDEESYKKFEKICY |
| | | GFGIDLSENSEINKPENESIRNYISHFYIVRNPFADYSIAEQIDRVSNLLSY |
| | | STRYNNSTYASVFEVFKKDVNLDYDELKKKFKLIGNNDILERLMKPKK |
| | | VSVLELESYNSDYIKNLIIELLTKIENTNDTL |
| SEQ | Rhodobacter | MQIGKVQGRTISEFGDPAGGLKRKISTDGKNRKELPAHLSSDPKALIGQ |
| ID | capsulatus | WISGIDKIYRKPDSRKSDGKAIHSPTPSKMQFDARDDLGEAFWKLVSEA |
| NO: | C2c2 amino | GLAQDSDYDQFKRRLHPYGDKFQPADSGAKLKFEADPPEPQAFHGRW |
| 126 | acid | YGAMSKRGNDAKELAAALYEHLHVDEKRIDGQPKRNPKTDKFAPGLV |
| | sequence | VARALGIESSVLPRGMARLARNWGEEEIQTYFVVDVAASVKEVAKAA |
| | | VSAAQAFDPPRQVSGRSLSPKVGFALAEHLERVTGSKRCSFDPAAGPSV |
| | | LALHDEVKKTYKRLCARGKNAARAFPADKTELLALMRHTHENRVRNQ |
| | | MVRMGRVSEYRGQQAGDLAQSHYWTSAGQTEIKESEIFVRLWVGAFA |
| | | LAGRSMKAWIDPMGKIVNTEKNDRDLTAAVNIRQVISNKEMVAEAMA |
| | | RRGIYFGETPELDRLGAEGNEGFVFALLRYLRGCRNQTFHLGARAGFLK |
| | | EIRKELEKTRWGKAKEAEHVVLTDKTVAAIRAIIDNDAKALGARLLAD |
| | | LSGAFVAHYASKEHFSTLYSEIVKAVKDAPEVSSGLPRLKLLLKRADGV |
| | | RGYVHGLRDTRKHAFATKLPPPPAPRELDDPATKARYIALLRLYDGPFR |
| | | AYASGITGTALAGPAARAKEAATALAQSVNVTKAYSDVMEGRSSRLRP |
| | | PNDGETLREYLSALTGETATEFRVQIGYESDSENARKQAEFIENYRRDM |
| | | LAFMFEDYIRAKGFDWILKIEPGATAMTRAPVLPEPIDTRGQYEHWQA |
| | | ALYLVMHFVPASDVSNLLHQLRKWEALQGKYELVQDGDATDQADAR |
| | | REALDLVKRFRDVLVLFLKTGEARFEGRAAPFDLKPFRALFANPATFDR |
| | | LFMATPTTARPAEDDPEGDGASEPELRVARTLRGLRQIARYNHMAVLS |
| | | DLFAKHKVRDEEVARLAEIEDETQEKSQIVAAQELRTDLHDKVMKCHP |
| | | KTISPEERQSYAAAIKTIEEHRFLVGRVYLGDHLRLHRLMMDVIGRLID |
| | | YAGAYERDTGTFLINASKQLGAGADWAVTIAGAANTDARTQTRKDLA |
| | | HFNVLDRADGTPDLTALVNRAREMMAYDRKRKNAVPRSILDMLARLG |
| | | LTLKWQMKDHLLQDATITQAAIKHLDKVRLTVGGPAAVTEARFSQDY |
| | | LQMVAAVFNGSVQNPKPRRRDDGDAWHKPPKPATAQSQPDQKPPNKA |
| | | PSAGSRLPPPQVGEVYEGVVVKVIDTGSLGFLAVEGVAGNIGLHISRLR |
| | | RIREDAIIVGRRYRFRVEIYVPPKSNTSKLNAADLVRID |
| SEQ | Carnobacterium | MRITKVKIKLDNKLYQVTMQKEEKYGTLKLNEESRKSTAEILRLKKASF |
| ID | gallinarum | NKSFHSKTINSQKENKNATIKKNGDYISQIFEKLVGVDTNKNIRKPKMS |
| NO: | C2c2 amino | LTDLKDLPKKDLALFIKRKFKNDDIVEIKNLDLISLFYNALQKVPGEHFT |
| 127 | acid | DESWADFCQEMMPYREYKNKFIERKIILLANSIEQNKGFSINPETFSKRK |
| | sequence | RVLHQWAIEVQERGDFSILDEKLSKLAEIYNFKKMCKRVQDELNDLEK |
| | | SMKKGKNPEKEKEAYKKQKNFKIKTIWKDYPYKTHIGLIEKIKENEELN |
| | | QFNIEIGKYFEHYFPIKKERCTEDEPYYLNSETIATTVNYQLKNALISYL |
| | | MQIGKYKQFGLENQVLDSKKLQEIGIYEGFQTKFMDACVFATSSLKNII |
| | | EPMRSGDILGKREFKEAIATSSFVNYHHFFPYFPFELKGMKDRESELIPF |
| | | GEQTEAKQMQNIWALRGSVQQIRNEIFHSFDKNQKFNLPQLDKSNFEFD |
| | | ASENSTGKSQSYIETDYKFLFEAEKNQLEQFFIERIKSSGALEYYPLKSLE |
| | | KLFAKKEMKFSLGSQVVAFAPSYKKLVKKGHSYQTATEGTANYLGLS |
| | | YYNRYELKEESFQAQYYLLKLIYQYVFLPNFSQGNSPAFRETVKAILRIN |
| | | KDEARKKMKKNKKFLRKYAFEQVREMEFKETPDQYMSYLQSEMREE |
| | | KVRKAEKNDKGFEKNITMNFEKLLMQIFVKGFDVFLTTFAGKELLLSSE |
| | | EKVIKETEISLSKKINEREKTLKASIQVEHQLVATNSAISYWLFCKLLDSR |
| | | HLNELRNEMIKFKQSRIKFNHTQHAELIQNLLPIVELTILSNDYDEKNDS |
| | | QNVDVSAYFEDKSLYETAPYVQTDDRTRVSFRPILKLEKYHTKSLIEAL |
| | | LKDNPQFRVAATDIQEWMHKREEIGELVEKRKNLHTEWAEGQQTLGA |
| | | EKREEYRDYCKKIDRFNWKANKVTLTYLSQLHYLITDLLGRMVGFSAL |
| | | FERDLVYFSRSFSELGGETYHISDYKNLSGVLRLNAEVKPIKIKNIKVIDN |
| | | EENPYKGNEPEVKPFLDRLHAYLENVIGIKAVHGKIRNQTAHLSVLQLE |
| | | LSMIESMNNLRDLMAYDRKLKNAVTKSMIKILDKHGMILKLKIDENHK |
| | | NFEIESLIPKEIIHLKDKAIKTNQVSEEYCQLVLALLTTNPGNQLN |
| SEQ | Herbinix | MKLTRRRISGNSVDQKITAAFYRDMSQGLLYYDSEDNDCTDKVIESMD |
| ID | hemicellulosilytica | FERSWRGRILKNGEDDKNPFYMFVKGLVGSNDKIVCEPIDVDSDPDNL |
| NO: | C2c2 | DILINKNLTGFGRNLKAPDSNDTLENLIRKIQAGIPEEEVLPELKKIKEMI |
| 128 | amino acid | QKDIVNRKEQLLKSIKNNRIPFSLEGSKLVPSTKKMKWLFKLIDVPNKTF |
| | sequence | NEKMLEKYWEIYDYDKLKANITNRLDKTDKKARSISRAVSEELREYHK |
| | | NLRTNYNRFVSGDRPAAGLDNGGSAKYNPDKEEFLLFLKEVEQYFKKY |
| | | FPVKSKHSNKSKDKSLVDKYKNYCSYKVVKKEVNRSIINQLVAGLIQQ |
| | | GKLLYYFYYNDTWQEDFLNSYGLSYIQVEEAFKKSVMTSLSWGINRLT |
| | | SFFIDDSNTVKFDDITTKKAKEAIESNYFNKLRTCSRMQDHFKEKLAFFY |
| | | PVYVKDKKDRPDDDIENLIVLVKNAIESVSYLRNRTFHFKESSLLELLKE |
| | | LDDKNSGQNKIDYSVAAEFIKRDIENLYDVFREQIRSLGIAEYYKADMIS |
| | | DCFKTCGLEFALYSPKNSLMPAFKNVYKRGANLNKAYIRDKGPKETGD |
| | | QGQNSYKALEEYRELTWYIEVKNNDQSYNAYKNLLQLIYYHAFLPEVR |
| | | ENEALITDFINRTKEWNRKETEERLNTKNNKKHKNFDENDDITVNTYR |
| | | YESIPDYQGESLDDYLKVLQRKQMARAKEVNEKEEGNNNYIQFIRDVV |
| | | VWAFGAYLENKLKNYKNELQPPLSKENIGLNDTLKELFPEEKVKSPFNI |
| | | KCRFSISTFIDNKGKSTDNTSAEAVKTDGKEDEKDKKNIKRKDLLCFYL |
| | | FLRLLDENEICKLQHQFIKYRCSLKERRFPGNRTKLEKETELLAELEELM |
| | | ELVRFTMPSIPEISAKAESGYDTMIKKYFKDFIEKKVFKNPKTSNLYYHS |
| | | DSKTPVTRKYMALLMRSAPLHLYKDIFKGYYLITKKECLEYIKLSNIIKD |
| | | YQNSLNELHEQLERIKLKSEKQNGKDSLYLDKKDFYKVKEYVENLEQV |
| | | ARYKHLQHKINFESLYRIFRIHVDIAARMVGYTQDWERDMHFLFKALV |
| | | YNGVLEERRFEAIFNNNDDNNDGRIVKKIQNNLNNKNRELVSMLCWN |
| | | KKLNKNEFGAIIWKRNPIAHLNHFTQTEQNSKSSLESLINSLRILLAYDR |
| | | KRQNAVTKTINDLLLNDYHIRIKWEGRVDEGQIYFNIKEKEDIENEPIIHL |
| | | KHLHKKDCYIYKNSYMFDKQKEWICNGIKEEVYDKSILKCIGNLFKFD |
| | | YEDKNKSSANPKHT |
| SEQ | Paludibacter | MRVSKVKVKDGGKDKMVLVHRKTTGAQLVYSGQPVSNETSNILPEKK |
| ID | propionicigenes | RQSFDLSTLNKTIIKFDTAKKQKLNVDQYKIVEKIFKYPKQELPKQIKAE |
| NO: | C2c2 | EILPFLNHKFQEPVKYWKNGKEESFNLTLLIVEAVQAQDKRKLQPYYD |
| 129 | amino acid | WKTWYIQTKSDLLKKSIENNRIDLTENLSKRKKALLAWETEFTASGSID |
| | sequence | LTHYHKVYMTDVLCKMLQDVKPLTDDKGKINTNAYHRGLKKALQNH |
| | | QPAIFGTREVPNEANRADNQLSIYHLEVVKYLEHYFPIKTSKRRNTADDI |
| | | AHYLKAQTLKTTIEKQLVNAIRANIIQQGKTNHHELKADTTSNDLIRIKT |
| | | NEAFVLNLTGTCAFAANNIRNMVDNEQTNDILGKGDFIKSLLKDNTNS |
| | | QLYSFFFGEGLSTNKAEKETQLWGIRGAVQQIRNNVNHYKKDALKTVF |
| | | NISNFENPTITDPKQQTNYADTIYKARFINELEKIPEAFAQQLKTGGAVS |
| | | YYTIENLKSLLTTFQFSLCRSTIPFAPGFKKVFNGGINYQNAKQDESFYE |
| | | LMLEQYLRKENFAEESYNARYFMLKLIYNNLFLPGFTTDRKAFADSVG |
| | | FVQMQNKKQAEKVNPRKKEAYAFEAVRPMTAADSIADYMAYVQSEL |
| | | MQEQNKKEEKVAEETRINFEKFVLQVFIKGFDSFLRAKEFDFVQMPQPQ |
| | | LTATASNQQKADKLNQLEASITADCKLTPQYAKADDATHIAFYVFCKL |
| | | LDAAHLSNLRNELIKFRESVNEFKFHHLLEIIEICLLSADVVPTDYRDLYS |
| | | SEADCLARLRPFIEQGADITNWSDLFVQSDKHSPVIHANIELSVKYGTTK |
| | | LLEQIINKDTQFKTTEANFTAWNTAQKSIEQLIKQREDHHEQWVKAKN |
| | | ADDKEKQERKREKSNFAQKFIEKHGDDYLDICDYINTYNWLDNKMHF |
| | | VHLNRLHGLTIELLGRMAGFVALFDRDFQFFDEQQIADEFKLHGFVNLH |
| | | SIDKKLNEVPTKKIKEIYDIRNKIIQINGNKINESVRANLIQFISSKRNYYN |
| | | NAFLHVSNDEIKEKQMYDIRNHIAHFNYLTKDAADFSLIDLINELRELLH |
| | | YDRKLKNAVSKAFIDLFDKHGMILKLKLNADHKLKVESLEPKKIYHLG |
| | | SSAKDKPEYQYCTNQVMMAYCNMCRSLLEMKK |
| SEQ | Leptotrichia | MYMKITKIDGVSHYKKQDKGILKKKWKDLDERKQREKIEARYNKQIES |
| ID | wadei (Lwa) | KIYKEFFRLKNKKRIEKEEDQNIKSLYFFIKELYLNEKNEEWELKNINLEI |
| NO: | C2c2 amino | LDDKERVIKGYKFKEDVYFFKEGYKEYYLRILFNNLIEKVQNENREKVR |
| 130 | acid | KNKEFLDLKEIFKKYKNRKIDLLLKSINNNKINLEYKKENVNEEIYGINP |
| | sequence | TNDREMTFYELLKEIIEKKDEQKSILEEKLDNFDITNFLENIEKIFNEETEI |
| | | NIIKGKVLNELREYIKEKEENNSDNKLKQIYNLELKKYIENNFSYKKQKS |
| | | KSKNGKNDYLYLNFLKKIMFIEEVDEKKEINKEKFKNKINSNFKNLFVQ |
| | | HILDYGKLLYYKENDEYIKNTGQLETKDLEYIKTKETLIRKMAVLVSFA |
| | | ANSYYNLFGRVSGDILGTEVVKSSKTNVIKVGSHIFKEKMLNYFFDFEIF |
| | | DANKIVEILESISYSIYNVRNGVGHFNKLILGKYKKKDINTNKRIEEDLN |
| | | NNEEIKGYFIKKRGEIERKVKEKFLSNNLQYYYSKEKIENYFEVYEFEIL |
| | | KRKIPFAPNFKRIIKKGEDLFNNKNNKKYEYFKNFDKNSAEEKKEFLKT |
| | | RNFLLKELYYNNFYKEFLSKKEEFEKIVLEVKEEKKSRGNINNKKSGVS |
| | | FQSIDDYDTKINISDYIASIHKKEMERVEKYNEEKQKDTAKYIRDFVEEI |
| | | FLTGFINYLEKDKRLHFLKEEFSILCNNNNNVVDFNININEEKIKEFLKEN |
| | | DSKTLNLYLFFNMIDSKRISEFRNELVKYKQFTKKRLDEEKEFLGIKIEL |
| | | YETLIEFVILTREKLDTKKSEEIDAWLVDKLYVKDSNEYKEYEEILKLFV |
| | | DEKILSSKEAPYYATDNKTPILLSNFEKTRKYGTQSFLSEIQSNYKYSKV |
| | | EKENIEDYNKKEEIEQKKKSNIEKLQDLKVELHKKWEQNKITEKEIEKY |
| | | NNTTRKINEYNYLKNKEELQNVYLLHEMLSDLLARNVAFFNKWERDF |
| | | KFIVIAIKQFLRENDKEKVNEFLNPPDNSKGKKVYFSVSKYKNTVENID |
| | | GIHKNFMNLIFLNNKFMNRKIDKMNCAIWVYFRNYIAHFLHLHTKNEKI |
| | | SLISQMNLLIKLFSYDKKVQNHILKSTKTLLEKYNIQINFEISNDKNEVFK |
| | | YKIKNRLYSKKGKMLGKNNKFEILENEFLENVKAMLEYSE |
| SEQ | Bergeyella | MENKTSLGNNIYYNPFKPQDKSYFAGYFNAAMENTDSVFRELGKRLKG |
| ID | zoohelcum | KEYTSENFFDAIFKENISLVEYERYVKLLSDYFPMARLLDKKEVPIKERK |
| NO: | Cas13b | ENFKKNFKGIIKAVRDLRNFYTHKEHGEVEITDEIFGVLDEMLKSTVLT |
| 131 | | VKKKKVKTDKTKEILKKSIEKQLDILCQKKLEYLRDTARKIEEKRRNQR |
| | | ERGEKELVAPFKYSDKRDDLIAAIYNDAFDVYIDKKKDSLKESSKAKY |
| | | NTKSDPQQEEGDLKIPISKNGVVFLLSLFLTKQEIHAFKSKIAGFKATVID |
| | | EATVSEATVSHGKNSICFMATHEIFSHLAYKKLKRKVRTAEINYGEAEN |
| | | AEQLSVYAKETLMMQMLDELSKVPDVVYQNLSEDVQKTFIEDWNEYL |
| | | KENNGDVGTMEEEQVIHPVIRKRYEDKFNYFAIRFLDEFAQFPTLRFQV |
| | | HLGNYLHDSRPKENLISDRRIKEKITVFGRLSELEHKKALFIKNTETNED |
| | | REHYWEIFPNPNYDFPKENISVNDKDFPIAGSILDREKQPVAGKIGIKVK |
| | | LLNQQYVSEVDKAVKAHQLKQRKASKPSIQNIIEEIVPINESNPKEAIVF |
| | | GGQPTAYLSMNDIHSILYEFFDKWEKKKEKLEKKGEKELRKEIGKELEK |
| | | KIVGKIQAQIQQIIDKDTNAKILKPYQDGNSTAIDKEKLIKDLKQEQNILQ |
| | | KLKDEQTVREKEYNDFIAYQDKNREINKVRDRNHKQYLKDNLKRKYP |
| | | EAPARKEVLYYREKGKVAVWLANDIKRFMPTDFKNEWKGEQHSLLQK |
| | | SLAYYEQCKEELKNLLPEKVFQHLPFKLGGYFQQKYLYQFYTCYLDKR |
| | | LEYISGLVQQAENFKSENKVFKKVENECFKFLKKQNYTHKELDARVQSI |
| | | LGYPIFLERGFMDEKPTIIKGKTFKGNEALFADWFRYYKEYQNFQTFYD |
| | | TENYPLVELEKKQADRKRKTKIYQQKKNDVFTLLMAKHIFKSVFKQDS |
| | | IDQFSLEDLYQSREERLGNQERARQTGERNTNYIWNKTVDLKLCDGKIT |
| | | VENVKLKNVGDFIKYEYDQRVQAFLKYEENIEWQAFLIKESKEEENYP |
| | | YVVEREIEQYEKVRREELLKEVHLIEEYILEKVKDKEILKKGDNQNFKY |
| | | YILNGLLKQLKNEDVESYKVFNLNTEPEDVNINQLKQEATDLEQKAFV |
| | | LTYIRNKFAHNQLPKKEFWDYCQEKYGKIEKEKTYAEYFAEVFKKEKE |
| | | ALIK |
| SEQ | Prevotella | MEDDKKTTDSIRYELKDKHFWAAFLNLARHNVYITVNHINKILEEGEIN |
| ID | intermedia | RDGYETTLKNTWNEIKDINKKDRLSKLIIKHFPFLEAATYRLNPTDTTKQ |
| NO: | Cas13b | KEEKQAEAQSLESLRKSFFVFIYKLRDLRNHYSHYKHSKSLERPKFEEG |
| 132 | | LLEKMYNIFNASIRLVKEDYQYNKDINPDEDFKHLDRTEEEFNYYFTKD |
| | | NEGNITESGLLFFVSLFLEKKDAIWMQQKLRGFKDNRENKKKMTNEVF |
| | | CRSRMLLPKLRLQSTQTQDWILLDMLNELIRCPKSLYERLREEDREKFR |
| | | VPIEIADEDYDAEQEPFKNTLVRHQDRFPYFALRYFDYNEIFTNLRFQID |
| | | LGTYHFSIYKKQIGDYKESHHLTHKLYGFERIQEFTKQNRPDEWRKFVK |
| | | TFNSFETSKEPYIPETTPHYHLENQKIGIRFRNDNDKIWPSLKTNSEKNEK |
| | | SKYKLDKSFQAEAFLSVHELLPMMFYYLLLKTENTDNDNEIETKKKEN |
| | | KNDKQEKHKIEEIIENKITEIYALYDTFANGEIKSIDELEEYCKGKDIEIGH |
| | | LPKQMIAILKDEHKVMATEAERKQEEMLVDVQKSLESLDNQINEEIENV |
| | | ERKNSSLKSGKIASWLVNDMMRFQPVQKDNEGKPLNNSKANSTEYQL |
| | | LQRTLAFFGSEHERLAPYFKQTKLIESSNPHPFLKDTEWEKCNNILSFYR |
| | | SYLEAKKNFLESLKPEDWEKNQYFLKLKEPKTKPKTLVQGWKNGFNLP |
| | | RGIFTEPIRKWFMKHRENITVAELKRVGLVAKVIPLFFSEEYKDSVQPFY |
| | | NYHFNVGNINKPDEKNFLNCEERRELLRKKKDEFKKMTDKEKEENPSY |
| | | LEFKSWNKFERELRLVRNQDIVTWLLCMELFNKKKIKELNVEKIYLKNI |
| | | NTNTTKKEKNTEEKNGEEKNIKEKNNILNRIMPMRLPIKVYGRENFSKN |
| | | KKKKIRRNTFFTVYIEEKGTKLLKQGNFKALERDRRLGGLFSFVKTPSK |
| | | AESKSNTISKLRVEYELGEYQKARIEIIKDMLALEKTLIDKYNSLDTDNF |
| | | NKMLTDWLELKGEPDKASFQNDVDLLIAVRNAFSHNQYPMRNRIAFA |
| | | NINPFSLSSANTSEEKGLGIANQLKDKTHKTIEKIIEIEKPIETKE |
| SEQ | Prevotella | MQKQDKLFVDRKKNAIFAFPKYITIMENKEKPEPIYYELTDKHFWAAFL |
| ID | buccae | NLARHNVYTTINHINRRLEIAELKDDGYMMGIKGSWNEQAKKLDKKV |
| NO: | Cas13b | RLRDLIMKHFPFLEAAAYEMTNSKSPNNKEQREKEQSEALSLNNLKNV |
| 133 | | LFIFLEKLQVLRNYYSHYKYSEESPKPIFETSLLKNMYKVFDANVRLVK |
| | | RDYMHHENIDMQRDFTHLNRKKQVGRTKNIIDSPNFHYHFADKEGNM |
| | | TIAGLLFFVSLFLDKKDAIWMQKKLKGFKDGRNLREQMTNEVFCRSRIS |
| | | LPKLKLENVQTKDWMQLDMLNELVRCPKSLYERLREKDRESFKVPFDI |
| | | FSDDYNAEEEPFKNTLVRHQDRFPYFVLRYFDLNEIFEQLRFQIDLGTYH |
| | | FSIYNKRIGDEDEVRHLTHHLYGFARIQDFAPQNQPEEWRKLVKDLDHF |
| | | ETSQEPYISKTAPHYHLENEKIGIKFCSAHNNLFPSLQTDKTCNGRSKFN |
| | | LGTQFTAEAFLSVHELLPMMFYYLLLTKDYSRKESADKVEGIIRKEISNI |
| | | YAIYDAFANNEINSIADLTRRLQNTNILQGHLPKQMISILKGRQKDMGK |
| | | EAERKIGEMIDDTQRRLDLLCKQTNQKIRIGKRNAGLLKSGKIADWLVN |
| | | DMMRFQPVQKDQNNIPINNSKANSTEYRMLQRALALFGSENFRLKAYF |
| | | NQMNLVGNDNPHPFLAETQWEHQTNILSFYRNYLEARKKYLKGLKPQ |
| | | NWKQYQHFLILKVQKTNRNTLVTGWKNSFNLPRGIFTQPIREWFEKHN |
| | | NSKRIYDQILSFDRVGFVAKAIPLYFAEEYKDNVQPFYDYPFNIGNRLKP |
| | | KKRQFLDKKERVELWQKNKELFKNYPSEKKKTDLAYLDFLSWKKFER |
| | | ELRLIKNQDIVTWLMFKELFNMATVEGLKIGEIHLRDIDTNTANEESNNI |
| | | LNRIMPMKLPVKTYETDNKGNILKERPLATFYIEETETKVLKQGNFKAL |
| | | VKDRRLNGLFSFAETTDLNLEEHPISKLSVDLELIKYQTTRISIFEMTLGL |
| | | EKKLIDKYSTLPTDSFRNMLERWLQCKANRPELKNYVNSLIAVRNAFS |
| | | HNQYPMYDATLFAEVKKFTLFPSVDTKKIELNIAPQLLEIVGKAIKEIEK |
| | | SENKN |
| SEQ | Porphyromonas | MNTVPASENKGQSRTVEDDPQYFGLYLNLARENLIEVESHVRIKFGKK |
| ID | gingivalis | KLNEESLKQSLLCDHLLSVDRWTKVYGHSRRYLPFLHYFDPDSQIEKD |
| NO: | Cas13b | HDSKTGVDPDSAQRLIRELYSLLDFLRNDFSHNRLDGTTFEHLEVSPDIS |
| 134 | | SFITGTYSLACGRAQSRFAVFFKPDDFVLAKNRKEQLISVADGKECLTV |
| | | SGFAFFICLFLDREQASGMLSRIRGFKRTDENWARAVHETFCDLCIRHP |
| | | HDRLESSNTKEALLLDMLNELNRCPRILYDMLPEEERAQFLPALDENSM |
| | | NNLSENSLDEESRLLWDGSSDWAEALTKRIRHQDRFPYLMLRFIEEMDL |
| | | LKGIRFRVDLGEIELDSYSKKVGRNGEYDRTITDHALAFGKLSDFQNEE |
| | | EVSRMISGEASYPVRFSLFAPRYAIYDNKIGYCHTSDPVYPKSKTGEKR |
| | | ALSNPQSMGFISVHDLRKLLLMELLCEGSFSRMQSDFLRKANRILDETA |
| | | EGKLQFSALFPEMRHRFIPPQNPKSKDRREKAETTLEKYKQEIKGRKDK |
| | | LNSQLLSAFDMDQRQLPSRLLDEWMNIRPASHSVKLRTYVKQLNEDCR |
| | | LRLRKFRKDGDGKARAIPLVGEMATFLSQDIVRMIISEETKKLITSAYYN |
| | | EMQRSLAQYAGEENRRQFRAIVAELRLLDPSSGHPFLSATMETAHRYTE |
| | | GFYKCYLEKKREWLAKIFYRPEQDENTKRRISVFFVPDGEARKLLPLLIR |
| | | RRMKEQNDLQDWIRNKQAHPIDLPSHLFDSKVMELLKVKDGKKKWNE |
| | | AFKDWWSTKYPDGMQPFYGLRRELNIHGKSVSYIPSDGKKFADCYTHL |
| | | MEKTVRDKKRELRTAGKPVPPDLAADIKRSFHRAVNEREFMLRLVQED |
| | | DRLMLMAINKMMTDREEDILPGLKNIDSILDEENQFSLAVHAKVLEKE |
| | | GEGGDNSLSLVPATIEIKSKRKDWSKYIRYRYDRRVPGLMSHFPEHKAT |
| | | LDEVKTLLGEYDRCRIKIFDWAFALEGAIMSDRDLKPYLHESSSREGKS |
| | | GEHSTLVKMLVEKKGCLTPDESQYLILIRNKAAHNQFPCAAEMPLIYRD |
| | | VSAKVGSIEGSSAKDLPEGSSLVDSLWKKYEMIIRKILPILDPENRFFGKL |
| | | LNNMSQPINDL |
| SEQ | Bacteroides | MESIKNSQKSTGKTLQKDPPYFGLYLNMALLNVRKVENHIRKWLGDV |
| ID | pyogenes | ALLPEKSGFHSLLTTDNLSSAKWTRFYYKSRKFLPFLEMFDSDKKSYEN |
| NO: | Cas13b | RRETAECLDTIDRQKISSLLKEVYGKLQDIRNAFSHYHIDDQSVKHTALI |
| 135 | | ISSEMHRFIENAYSFALQKTRARFTGVFVETDFLQAEEKGDNKKFFAIG |
| | | GNEGIKLKDNALIFLICLFLDREEAFKFLSRATGFKSTKEKGFLAVRETF |
| | | CALCCRQPHERLLSVNPREALLMDMLNELNRCPDILFEMLDEKDQKSF |
| | | LPLLGEEEQAHILENSLNDELCEAIDDPFEMIASLSKRVRYKNRFPYLML |
| | | RYIEEKNLLPFIRFRIDLGCLELASYPKKMGEENNYERSVTDHAMAFGR |
| | | LTDFHNEDAVLQQITKGITDEVRFSLYAPRYAIYNNKIGFVRTSGSDKIS |
| | | FPTLKKKGGEGHCVAYTLQNTKSFGFISIYDLRKILLLSFLDKDKAKNIV |
| | | SGLLEQCEKHWKDLSENLFDAIRTELQKEFPVPLIRYTLPRSKGGKLVSS |
| | | KLADKQEKYESEFERRKEKLTEILSEKDFDLSQIPRRMIDEWLNVLPTSR |
| | | EKKLKGYVETLKLDCRERLRVFEKREKGEHPLPPRIGEMATDLAKDIIR |
| | | MVIDQGVKQRITSAYYSEIQRCLAQYAGDDNRRHLDSIIRELRLKDTKN |
| | | GHPFLGKVLRPGLGHTEKLYQRYFEEKKEWLEATFYPAASPKRVPRFV |
| | | NPPTGKQKELPLIIRNLMKERPEWRDWKQRKNSHPIDLPSQLFENEICRL |
| | | LKDKIGKEPSGKLKWNEMFKLYWDKEFPNGMQRFYRCKRRVEVFDKV |
| | | VEYEYSEEGGNYKKYYEALIDEVVRQKISSSKEKSKLQVEDLTLSVRRV |
| | | FKRAINEKEYQLRLLCEDDRLLFMAVRDLYDWKEAQLDLDKIDNMLG |
| | | EPVSVSQVIQLEGGQPDAVIKAECKLKDVSKLMRYCYDGRVKGLMPYF |
| | | ANHEATQEQVEMELRHYEDHRRRVFNWVFALEKSVLKNEKLRRFYEE |
| | | SQGGCEHRRCIDALRKASLVSEEEYEFLVHIRNKSAHNQFPDLEIGKLPP |
| | | NVTSGFCECIWSKYKAIICRIIPFIDPERRFFGKLLEQK |
| SEQ | Cas13c | MTEKKSIIFKNKSSVEIVKKDIFSQTPDNMIRNYKITLKISEKNPRVVEAE |
| ID | | IEDLMNSTILKDGRRSARREKSMTERKLIEEKVAENYSLLANCPMEEVD |
| NO: | | SIKIYKIKRFLTYRSNMLLYFASINSFLCEGIKGKDNETEEIWHLKDNDV |
| 136 | | RKEKVKENFKNKLIQSTENYNSSLKNQIEEKEKLLRKESKKGAFYRTIIK |
| | | KLQQERIKELSEKSLTEDCEKIIKLYSELRHPLMHYDYQYFENLFENKEN |
| | | SELTKNLNLDIFKSLPLVRKMKLNNKVNYLEDNDTLFVLQKTKKAKTL |
| | | YQIYDALCEQKNGFNKFINDFFVSDGEENTVFKQIINEKFQSEMEFLEKR |
| | | ISESEKKNEKLKKKFDSMKAHFHNINSEDTKEAYFWDIHSSSNYKTKYN |
| | | ERKNLVNEYTELLGSSKEKKLLREEITQINRKLLKLKQEMEEITKKNSLF |
| | | RLEYKMKIAFGFLFCEFDGNISKFKDEFDASNQEKIIQYHKNGEKYLTYF |
| | | LKEEEKEKFNLEKMQKIIQKTEEEDWLLPETKNNLFKFYLLTYLLLPYE |
| | | LKGDFLGFVKKHYYDIKNVDFMDENQNNIQVSQTVEKQEDYFYHKIRL |
| | | FEKNTKKYEIVKYSIVPNEKLKQYFEDLGIDIKYLTGSVESGEKWLGEN |
| | | LGIDIKYLTVEQKSEVSEEKIKKFL |
| SEQ | Cas13c | MEKDKKGEKIDISQEMIEEDLRKILILFSRLRHSMVHYDYEFYQALYSG |
| ID | | KDFVISDKNNLENRMISQLLDLNIFKELSKVKLIKDKAISNYLDKNTTIH |
| NO: | | VLGQDIKAIRLLDIYRDICGSKNGFNKFINTMITISGEEDREYKEKVIEHF |
| 137 | | NKKMENLSTYLEKLEKQDNAKRNNKRVYNLLKQKLIEQQKLKEWFGG |
| | | PYVYDIHSSKRYKELYIERKKLVDRHSKLFEEGLDEKNKKELTKINDEL |
| | | SKLNSEMKEMTKLNSKYRLQYKLQLAFGFILEEFDLNIDTFINNFDKDK |
| | | DLIISNFMKKRDIYLNRVLDRGDNRLKNIIKEYKFRDTEDIFCNDRDNNL |
| | | VKLYILMYILLPVEIRGDFLGFVKKNYYDMKHVDFIDKKDKEDKDTFF |
| | | HDLRLFEKNIRKLEITDYSLSSGFLSKEHKVDIEKKINDFINRNGAMKLP |
| | | EDITIEEFNKSLILPIMKNYQINFKLLNDIEISALFKIAKDRSITFKQAIDEI |
| | | KNEDIKKNSKKNDKNNHKDKNINFTQLMKRALHEKIPYKAGMYQIRN |
| | | NISHIDMEQLYIDPLNSYMNSNKNNITISEQIEKIIDVCVTGGVTGKELNN |
| | | NIINDYYMKKEKLVFNLKLRKQNDIVSIESQEKNKREEFVFKKYGLDYK |
| | | DGEINIIEVIQKVNSLQEELRNIKETSKEKLKNKETLFRDISLINGTIRKNI |
| | | NFKIKEMVLDIVRMDEIRHINIHIYYKGENYTRSNIIKFKYAIDGENKKY |
| | | YLKQHEINDINLELKDKFVTLICNMDKHPNKNKQTINLESNYIQNVKFII |
| | | P |
| SEQ | Cas13c | MENKGNNKKIDFDENYNILVAQIKEYFTKEIENYNNRIDNIIDKKELLKY |
| ID | | SEKKEESEKNKKLEELNKLKSQKLKILTDEEIKADVIKIIKIFSDLRHSLM |
| NO: | | HYEYKYFENLFENKKNEELAELLNLNLFKNLTLLRQMKIENKTNYLEG |
| 138 | | REEFNIIGKNIKAKEVLGHYNLLAEQKNGFNNFINSFFVQDGTENLEFKK |
| | | LIDEHFVNAKKRLERNIKKSKKLEKELEKMEQHYQRLNCAYVWDIHTS |
| | | TTYKKLYNKRKSLIEEYNKQINEIKDKEVITAINVELLRIKKEMEEITKSN |
| | | SLFRLKYKMQIAYAFLEIEFGGNIAKFKDEFDCSKMEEVQKYLKKGVK |
| | | YLKYYKDKEAQKNYEFPFEEIFENKDTHNEEWLENTSENNLFKFYILTY |
| | | LLLPMEFKGDFLGVVKKHYYDIKNVDFTDESEKELSQVQLDKMIGDSF |
| | | FHKIRLFEKNTKRYEIIKYSILTSDEIKRYFRLLELDVPYFEYEKGTDEIGI |
| | | FNKNIILTIFKYYQIIFRLYNDLEIHGLFNISSDLDKILRDLKSYGNKNINF |
| | | REFLYVIKQNNNSSTEEEYRKIWENLEAKYLRLHLLTPEKEEIKTKTKEE |
| | | LEKLNEISNLRNGICHLNYKEIIEEILKTEISEKNKEATLNEKIRKVINFIKE |
| | | NELDKVELGFNFINDFFMKKEQFMFGQIKQVKEGNSDSITTERERKEKN |
| | | NKKLKETYELNCDNLSEFYETSNNLRERANSSSLLEDSAFLKKIGLYKV |
| | | KNNKVNSKVKDEEKRIENIKRKLLKDSSDIMGMYKAEVVKKLKEKLILI |
| | | FKHDEEKRIYVTVYDTSKAVPENISKEILVKRNNSKEEYFFEDNNKKYV |
| | | TEYYTLEITETNELKVIPAKKLEGKEFKTEKNKENKLMLNNHYCFNVKI |
| | | IY |
| SEQ | Cas13c | MEEIKHKKNKSSIIRVIVSNYDMTGIKEIKVLYQKQGGVDTFNLKTIINL |
| ID | | ESGNLEIISCKPKEREKYRYEFNCKTEINTISITKKDKVLKKEIRKYSLEL |
| NO: | | YFKNEKKDTVVAKVTDLLKAPDKIEGERNHLRKLSSSTERKLLSKTLCK |
| 139 | | NYSEISKTPIEEIDSIKIYKIKRFLNYRSNFLIYFALINDFLCAGVKEDDINE |
| | | VWLIQDKEHTAFLENRIEKITDYIFDKLSKDIENKKNQFEKRIKKYKTSL |
| | | EELKTETLEKNKTFYIDSIKTKITNLENKITELSLYNSKESLKEDLIKIISIF |
| | | TNLRHSLMHYDYKSFENLFENIENEELKNLLDLNLFKSIRMSDEFKTKN |
| | | RTNYLDGTESFTIVKKHQNLKKLYTYYNNLCDKKNGFNTFINSFFVTDG |
| | | IENTDFKNLIILHFEKEMEEYKKSIEYYKIKISNEKNKSKKEKLKEKIDLL |
| | | QSELINMREHKNLLKQIYFFDIHNSIKYKELYSERKNLIEQYNLQINGVK |
| | | DVTAINHINTKLLSLKNKMDKITKQNSLYRLKYKLKIAYSFLMIEFDGD |
| | | VSKFKNNFDPTNLEKRVEYLDKKEEYLNYTAPKNKFNFAKLEEELQKI |
| | | QSTSEMGADYLNVSPENNLFKFYILTYIMLPVEFKGDFLGFVKNHYYNI |
| | | KNVDFMDESLLDENEVDSNKLNEKIENLKDSSFFNKIRLFEKNIKKYEIV |
| | | KYSVSTQENMKEYFKQLNLDIPYLDYKSTDEIGIFNKNMILPIFKYYQN |
| | | VFKLCNDIEIHALLALANKKQQNLEYAIYCCSKKNSLNYNELLKTFNRK |
| | | TYQNLSFIRNKIAHLNYKELFSDLFNNELDLNTKVRCLIEFSQNNKFDQI |
| | | DLGMNFINDYYMKKTRFIFNQRRLRDLNVPSKEKIIDGKRKQQNDSNN |
| | | ELLKKYGLSRTNIKDIFNKAWY |
| SEQ | Cas13c | MKVRYRKQAQLDTFIIKTEIVNNDIFIKSIIEKAREKYRYSFLFDGEEKYH |
| ID | | FKNKSSVEIVKNDIFSQTPDNMIRNYKITLKISEKNPRVVEAEIEDLMNST |
| NO: | | ILKDGRRSARREKSMTERKLIEEKVAENYSLLANCPIEEVDSIKIYKIKRF |
| 140 | | LTYRSNMLLYFASINSFLCEGIKGKDNETEEIWHLKDNDVRKEKVKENF |
| | | KNKLIQSTENYNSSLKNQIEEKEKLSSKEFKKGAFYRTIIKKLQQERIKEL |
| | | SEKSLTEDCEKIIKLYSELRHPLMHYDYQYFENLFENKENSELTKNLNL |
| | | DIFKSLPLVRKMKLNNKVNYLEDNDTLFVLQKTKKAKTLYQIYDALCE |
| | | QKNGFNKFINDFFVSDGEENTVFKQIINEKFQSEMEFLEKRISESEKKNE |
| | | KLKKKLDSMKAHFRNINSEDTKEAYFWDIHSSRNYKTKYNERKNLVNE |
| | | YTKLLGSSKEKKLLREEITKINRQLLKLKQEMEEITKKNSLFRLEYKMKI |
| | | AFGFLFCEFDGNISKFKDEFDASNQEKIIQYHKNGEKYLTSFLKEEEKEK |
| | | FNLEKMQKIIQKTEEEDWLLPETKNNLFKFYLLTYLLLPYELKGDFLGF |
| | | VKKHYYDIKNVDFMDENQNNIQVSQTVEKQEDYFYHKIRLFEKNTKK |
| | | YEIVKYSIVPNEKLKQYFEDLGIDIKYLTGSVESGEKWLGENLGIDIKYL |
| | | TVEQKSEVSEEKNKKVSLKNNGMFNKTILLFVFKYYQIAFKLFNDIELY |
| | | SLFFLREKSEKPFEVFLEELKDKMIGKQLNFGQLLYVVYEVLVKNKDLD |
| | | KILSKKIDYRKDKSFSPEIAYLRNFLSHLNYSKFLDNFMKINTNKSDENK |
| | | EVLIPSIKIQKMIQFIEKCNLQNQIDFDFNFVNDFYMRKEKMFFIQLKQIF |
| | | PDINSTEKQKKSEKEEILRKRYHLINKKNEQIKDEHEAQSQLYEKILSLQ |
| | | KIFSCDKNNFYRRLKEEKLLFLEKQGKKKISMKEIKDKIASDISDLLGILK |
| | | KEITRDIKDKLTEKFRYCEEKLLNISFYNHQDKKKEEGIRVFLIRDKNSD |
| | | NFKFESILDDGSNKIFISKNGKEITIQCCDKVLETLMIEKNTLKISSNGKIIS |
| | | LIPHYSYSIDVKY |

Some programmable nucleases can exhibit a high turnover rate. Turnover rate quantifies how many molecules of a detector nucleic acid each programmable nuclease is cleaving per minute. Programmable nucleases with a higher turnover rate are more efficient and transcollateral cleavage in the DETECTR assay methods disclosed herein.

Turnover rate is quantified as the max transcleaving velocity (max slope in a plot of signal versus time in a DETECTR assay) divided by the amount of programmable nuclease complexed with the guide nucleic acid present in the DETECTR assay, wherein the programmable nuclease is at saturation with respect to its active site for transcollateral cleavage of detector nucleic acids.

Turnover rate can be quantified with the following equation:

$$Turnover rate = \frac{\begin{matrix}maximum transcleaving velocity ( \frac{AU}{min} ) \text{/} \\ signal normzalization factor ( \frac{AU}{nM} )\end{matrix}}{\begin{matrix}concentration of programmable nuclease \\ complexed with guide nucleic acid ( nM )\end{matrix}}$$

Signal normalization factor is based on a standard curve and is the amount of signal produced from a known quantity of detector nucleic acid (substrate of transcollateral cleavage). The turnover rate is, thus, expressed as cleaved detector nucleic acid molecules per minute divided by the concentration of the programmable nuclease complexed with guide nucleic acid (can also be referred to as “nucleoprotein” or “ribonucleoprotein”). Therefore, a programmable nuclease with a high turnover rate exhibits superior and highly efficient transcollateral cleavage of detector nucleic acids in the DETECTR assay methods disclosed herein. For example, a programmable nuclease having at least 60% sequence identity to SEQ ID NO: 147 can exhibit high a turnover rate of at least about 0.1 cleaved detector molecules per minute. A programmable nuclease having a sequence of SEQ ID NO: 147 exhibits a turnover rate of at least about 0.1 cleaved detector molecules per minute. For example, a programmable nuclease (e.g., SEQ ID NO: 147) that recognizes a PAM of YYN complexed with a guide nucleic acid comprises a turnover rate of at least about 0.1 cleaved detector molecules per minute. The programmable nuclease may be a Type V programmable nuclease. A programmable nuclease having a sequence of SEQ ID NO: 147 exhibits a turnover rate that is higher than the turnover rate of SEQ ID NO: 21. For example, a programmable nuclease having a sequence of SEQ ID NO: 147 can exhibit a turnover rate that is at least about 2-fold higher than the turnover rate of SEQ ID NO: 21. In some embodiments, a programmable nuclease having a sequence of SEQ ID NO: 147 complexed with a guide nucleic acid can exhibit a turnover rate that is at least about 2-fold higher than the turnover rate of SEQ ID NO: 21 complexed with a guide nucleic acid. Thus, a programmable nuclease of SEQ ID NO: 147 is superior and more efficient at transcollateral cleavage in the DETECTR assay methods disclosed herein than a programmable nuclease of SEQ ID NO: 21.

In some embodiments, programmable nucleases with a high turnover rate have a turnover rate of at least about 0.1 cleaved detector molecules per minute. In some embodiments, programmable nucleases with a high turnover rate have a turnover rate of at least about 0.5 cleaved detector molecules per minute. In some embodiments, programmable nucleases with a high turnover rate have a turnover rate of at least about 1 cleaved detector molecules per minute. In some embodiments, programmable nucleases with a high turnover rate have a turnover rate of at least about 2 cleaved detector molecules per minute. In some embodiments, programmable nucleases with a high turnover rate have a turnover rate of at least about 3 cleaved detector molecules per minute. In some embodiments, programmable nucleases with a high turnover rate have a turnover rate of at least about 4 cleaved detector molecules per minute. In some embodiments, programmable nucleases with a high turnover rate have a turnover rate of at least about 5 cleaved detector molecules per minute. In some embodiments, programmable nucleases with a high turnover rate have a turnover rate of at least about 10 cleaved detector molecules per minute. In some embodiments, programmable nucleases with a high turnover rate have a turnover rate of at least about 15 cleaved detector molecules per minute. In some embodiments, programmable nucleases with a high turnover rate have a turnover rate of at least about 20 cleaved detector molecules per minute. In some embodiments, programmable nucleases with a high turnover rate have a turnover rate of at least about 25 cleaved detector molecules per minute. In some embodiments, programmable nucleases with a high turnover rate have a turnover rate of at least about 30 cleaved detector molecules per minute. In some embodiments, programmable nucleases with a high turnover rate have a turnover rate of at least about 35 cleaved detector molecules per minute. In some embodiments, programmable nucleases with a high turnover rate have a turnover rate of at least about 40 cleaved detector molecules per minute. In some embodiments, programmable nucleases with a high turnover rate have a turnover rate of at least about 45 cleaved detector molecules per minute. In some embodiments, programmable nucleases with a high turnover rate have a turnover rate of at least about 50 cleaved detector molecules per minute. In some embodiments, programmable nucleases with a high turnover rate have a turnover rate of at least about 60 cleaved detector molecules per minute. In some embodiments, programmable nucleases with a high turnover rate have a turnover rate of at least about 70 cleaved detector molecules per minute. In some embodiments, programmable nucleases with a high turnover rate have a turnover rate of at least about 80 cleaved detector molecules per minute. In some embodiments, programmable nucleases with a high turnover rate have a turnover rate of at least about 90 cleaved detector molecules per minute. In some embodiments, programmable nucleases with a high turnover rate have a turnover rate of at least about 100 cleaved detector molecules per minute. In some embodiments, programmable nucleases with a high turnover rate have a turnover rate of at least about 0.1 to 0.5 cleaved detector molecules per minute. In some embodiments, programmable nucleases with a high turnover rate have a turnover rate of at least about 0.5 to 1 cleaved detector molecules per minute. In some embodiments, programmable nucleases with a high turnover rate have a turnover rate of at least about 1 to 1.5 cleaved detector molecules per minute. In some embodiments, programmable nucleases with a high turnover rate have a turnover rate of at least about 1.5 to 2 cleaved detector molecules per minute. In some embodiments, programmable nucleases with a high turnover rate have a turnover rate of at least about 2 to 2.5 cleaved detector molecules per minute. In some embodiments, programmable nucleases with a high turnover rate have a turnover rate of at least about 2.5 to 3 cleaved detector molecules per minute. In some embodiments, programmable nucleases with a high turnover rate have a turnover rate of at least about 3 to 3.5 cleaved detector molecules per minute. In some embodiments, programmable nucleases with a high turnover rate have a turnover rate of at least about 3.5 to 4 cleaved detector molecules per minute. In some embodiments, programmable nucleases with a high turnover rate have a turnover rate of at least about 4 to 4.5 cleaved detector molecules per minute. In some embodiments, programmable nucleases with a high turnover rate have a turnover rate of at least about 4.5 to 5 cleaved detector molecules per minute. In some embodiments, programmable nucleases with a high turnover rate have a turnover rate of at least about 5 to 10 cleaved detector molecules per minute. In some embodiments, programmable nucleases with a high turnover rate have a turnover rate of at least about 10 to 15 cleaved detector molecules per minute. In some embodiments, programmable nucleases with a high turnover rate have a turnover rate of at least about 15 to 20 cleaved detector molecules per minute. In some embodiments, programmable nucleases with a high turnover rate have a turnover rate of at least about 20 to 25 cleaved detector molecules per minute. In some embodiments, programmable nucleases with a high turnover rate have a turnover rate of at least about 25 to 30 cleaved detector molecules per minute. In some embodiments, programmable nucleases with a high turnover rate have a turnover rate of at least about 30 to 35 cleaved detector molecules per minute. In some embodiments, programmable nucleases with a high turnover rate have a turnover rate of at least about 35 to 40 cleaved detector molecules per minute. In some embodiments, programmable nucleases with a high turnover rate have a turnover rate of at least about 40 to 45 cleaved detector molecules per minute. In some embodiments, programmable nucleases with a high turnover rate have a turnover rate of at least about 45 to 50 cleaved detector molecules per minute. In some embodiments, programmable nucleases with a high turnover rate have a turnover rate of at least about 50 to 60 cleaved detector molecules per minute. In some embodiments, programmable nucleases with a high turnover rate have a turnover rate of at least about 60 to 70 cleaved detector molecules per minute. In some embodiments, programmable nucleases with a high turnover rate have a turnover rate of at least about 70 to 80 cleaved detector molecules per minute. In some embodiments, programmable nucleases with a high turnover rate have a turnover rate of at least about 80 to 90 cleaved detector molecules per minute. In some embodiments, programmable nucleases with a high turnover rate have a turnover rate of at least about 90 to 100 cleaved detector molecules per minute.

Any of the programmable nucleases disclosed herein are compatible for use in any method of diagnosis, wherein the programmable nuclease detects the modification according to any of the method disclosed herein (e.g., assaying for a modification state and using the DETECTR assay methods disclosed herein). Any of the programmable nucleases disclosed herein are compatible for use in any method of diagnosis, wherein the programmable nuclease detects the target nucleic acid according to any of the methods disclosed herein (e.g., assaying for a modification state in a target nucleic acid or ssDNA amplification and DETECTR assay-based detection of the target nucleic acid). Any of the programmable nucleases disclosed herein are compatible for use in a method of diagnosis, wherein the programmable nuclease detects the SNP according to any of the methods disclosed herein. Any of the programmable nucleases disclosed herein are compatible for use in any method of assaying for a modification state according to the methods disclosed herein. Any of the programmable nucleases disclosed herein are compatible for use in assaying for a target nucleic acid in a sample according to the methods disclosed herein. In some embodiments, the programmable nuclease is any Cas12 or Cas13 disclosed herein.

The trans cleavage activity of the CRISPR enzyme can be activated when the crRNA is complexed with the segment of the target nucleic acid.

When a guide nucleic acid binds to a segment of the target nucleic acid, the programmable nuclease's trans cleavage activity can be initiated, and detector nucleic acids can be cleaved, resulting in the detection of fluorescence. Detector nucleic acids can comprise a detection moiety, wherein the detector nucleic acid can be cleaved by the activated programmable nuclease, thereby generating a detectable fluorescent signal. Detector nucleic acids can be a single-stranded nucleic acid sequence comprising deoxyribonucleotides. The detector nucleic acid can be a single-stranded nucleic acid sequence comprising ribonucleotides. The detector nucleic acid can be a single-stranded nucleic acid sequence comprising at least one deoxyribonucleotide and at least one ribonucleotide. The detector nucleic acid, in some cases, is a single-stranded nucleic acid comprising at least one ribonucleotide residue at an internal position that functions as a cleavage site. The detector nucleic acid can comprise at least 2, 3, 4, 5, 6, 7, 8, 9, or 10 ribonucleotide residues at an internal position. Sometimes the ribonucleotide residues are continuous. Sometimes, the ribonucleotide residues are interspersed in between non-ribonucleotide residues. The detector nucleic acid can be only ribonucleotide residues. The detector nucleic acid can be only deoxyribonucleotide residues. The detector nucleic acid can comprise nucleotides resistant to cleavage by a programmable nuclease. The detector nucleic acid can comprise synthetic nucleotides. The detector nucleic acid can comprise at least one ribonucleotide residue and at least one non-ribonucleotide residue. The detector nucleic acid can be from 5-20, 5-15, 5-10, 7-20, 7-15, or 7-10 nucleotides in length. The detector nucleic acid can comprise at least one uracil ribonucleotide. The detector nucleic acid can comprise at least two uracil ribonucleotides. Sometimes the detector nucleic acid has only uracil ribonucleotides. The detector nucleic acid can comprise at least one adenine ribonucleotide. The detector nucleic acid can have at least two adenine ribonucleotides. The detector nucleic acid can have only adenine ribonucleotides. The detector nucleic acid can have at least one cytosine ribonucleotide. The detector nucleic acid can have at least two cytosine ribonucleotides. The detector nucleic acid can have at least one guanine ribonucleotide. The detector nucleic acid comprises at least two guanine ribonucleotides. The detector nucleic acid can have only unmodified ribonucleotides, only unmodified deoxyribonucleotides, or a combination thereof. The detector nucleic acid can be from 5 to12 nucleotides in length. The detector nucleic acid can be at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. The detector nucleic acid can be 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a CRISPR enzyme comprising Cas13, a detector nucleic acid can be 5, 8, or 10 nucleotides in length. For cleavage by a CRISPR enzyme comprising a Cas12 protein, a detector nucleic acid can be 10 nucleotides in length.

The detector nucleic acid can comprise a detection moiety capable of generating a first detectable signal. The detector nucleic acid can be an ssDNA fluorescence-quenching (FQ) reporter molecule. The detection moiety can be on one side of the cleavage site. Optionally, a quenching moiety can be on the other side of the cleavage site. Sometimes the quenching moiety is a fluorescence quenching moiety. The quenching moiety can be 5′ to the cleavage site and the detection moiety can be 3′ to the cleavage site. The detection moiety can be 5′ to the cleavage site and the quenching moiety can be 3′ to the cleavage site. Sometimes the quenching moiety is at the 5′ terminus of the detector nucleic acid. Sometimes the detection moiety is at the 3′ terminus of the detector nucleic acid. The detection moiety can be at the 5′ terminus of the detector nucleic acid. The quenching moiety can be at the 3′ terminus of the detector nucleic acid. The detector nucleic acid can be at least one population of detector nucleic acid capable of generating a first detectable signal. The detector nucleic acid can be a population of the detector nucleic acid capable of generating a first detectable signal. Optionally, there are more than one population of detector nucleic acid. There can be 2, 3, 4, 5, 6, 7, 8, 9, 10, 11, 12, 13, 14, 15, 20, 30, 40, 50, greater than 50, or any number spanned by the range of this list of different populations of detector nucleic acids canahle of generating a unique detectable signal.

TABLE 4
Exemplary Single Stranded Detector Nucleic Acid

| 5′ | | |
|---|---|---|
| Detection | | 3′ |
| Moiety* | Sequence (SEQ ID NO:) | Quencher* |
| /56-FAM/ | rUrUrUrUrU (SEQ ID NO: 1) | /3IABkFQ/ |
| /5IRD700/ | rUrUrUrUrU (SEQ ID NO: 1) | /3IRQC1N/ |
| /5TYE665/ | rUrUrUrUrU (SEQ ID NO: 1) | /3IAbRQSp/ |
| /5Alex594N/ | rUrUrUrUrU (SEQ ID NO: 1) | /3IAbRQSp/ |
| /5ATTO633N/ | rUrUrUrUrU (SEQ ID NO: 1) | /3IAbRQSp/ |
| /56-FAM/ | rUrUrUrUrUrUrUrU | /3IABkFQ/ |
| | (SEQ ID NO: 2) | |
| /5IRD700/ | rUrUrUrUrUrUrUrU | /3IRQC1N/ |
| | (SEQ ID NO: 2) | |
| /5TYE665/ | rUrUrUrUrUrUrUrU | /3IAbRQSp/ |
| | (SEQ ID NO: 2) | |
| /5Alex594N/ | rUrUrUrUrUrUrUrU | /3IAbRQSp/ |
| | (SEQ ID NO: 2) | |
| /5ATTO633N/ | rUrUrUrUrUrUrUrU | /3IAbRQSp/ |
| | (SEQ ID NO: 2) | |
| /56-FAM/ | rUrUrUrUrUrUrUrUrUrU | /3IABkFQ/ |
| | (SEQ ID NO: 3) | |
| /5IRD700/ | rUrUrUrUrUrUrUrUrUrU | /3IRQC1N/ |
| | (SEQ ID NO: 3) | |
| /5TYE665/ | rUrUrUrUrUrUrUrUrUrU | /3IAbRQSp/ |
| | (SEQ ID NO: 3) | |
| /5Alex594N/ | rUrUrUrUrUrUrUrUrUrU | /3IAbRQSp/ |
| | (SEQ ID NO: 3) | |
| /5ATTO633N/ | rUrUrUrUrUrUrUrUrUrU | /3IAbRQSp/ |
| | (SEQ ID NO: 3) | |
| /56-FAM/ | TTTTrUrUTTTT (SEQ ID NO: 4) | /3IABkFQ/ |
| /5IRD700/ | TTTTrUrUTTTT (SEQ ID NO: 4) | /3IRQC1N/ |
| /5TYE665/ | TTTTrUrUTTTT (SEQ ID NO: 4) | /3IAbRQSp/ |
| /5Alex594N/ | TTTTrUrUTTTT (SEQ ID NO: 4) | /3IAbRQSp/ |
| /5ATTO633N/ | TTTTrUrUTTTT (SEQ ID NO: 4) | /3IAbRQSp/ |
| /56-FAM/ | TTrUrUTT (SEQ ID NO: 5) | /3IABkFQ/ |
| /5IRD700/ | TTrUrUTT (SEQ ID NO: 5) | /3IRQC1N/ |
| /5TYE665/ | TTrUrUTT (SEQ ID NO: 5) | /3IAbRQSp/ |
| /5Alex594N/ | TTrUrUTT (SEQ ID NO: 5) | /3IAbRQSp/ |
| /5ATTO633N/ | TTrUrUTT (SEQ ID NO: 5) | /3IAbRQSp/ |
| /56-FAM/ | TArArUGC (SEQ ID NO: 6) | /3IABkFQ/ |
| /5IRD700/ | TArArUGC (SEQ ID NO: 6) | /3IRQC1N/ |
| /5TYE665/ | TArArUGC (SEQ ID NO: 6) | /3IAbRQSp/ |
| /5Alex594N/ | TArArUGC (SEQ ID NO: 6) | /3IAbRQSp/ |
| /5ATTO633N/ | TArArUGC (SEQ ID NO: 6) | /3IAbRQSp/ |
| /56-FAM/ | TArUrGGC (SEQ ID NO: 7) | /3IABkFQ/ |
| /5IRD700/ | TArUrGGC (SEQ ID NO: 7) | /3IRQC1N/ |
| /5TYE665/ | TArUrGGC (SEQ ID NO: 7) | /3IAbRQSp/ |
| /5Alex594N/ | TArUrGGC (SEQ ID NO: 7) | /3IAbRQSp/ |
| /5ATTO633N/ | TArUrGGC (SEQ ID NO: 7) | /3IAbRQSp/ |
| /56-FAM/ | rUrUrUrUrU (SEQ ID NO: 8) | /3IABkFQ/ |
| /5IRD700/ | rUrUrUrUrU (SEQ ID NO: 8) | /3IRQC1N/ |
| /5TYE665/ | rUrUrUrUrU (SEQ ID NO: 8) | /3IAbRQSp/ |
| /5Alex594N/ | rUrUrUrUrU (SEQ ID NO: 8) | /3IAbRQSp/ |
| /5ATTO633N/ | rUrUrUrUrU (SEQ ID NO: 8) | /3IAbRQSp/ |
| /56-FAM/ | TTATTATT (SEQ ID NO: 9) | /3IABkFQ/ |
| /56-FAM/ | TTATTATT (SEQ ID NO: 9) | /3IABkFQ/ |
| /5IRD700/ | TTATTATT (SEQ ID NO: 9) | /3IRQC1N/ |
| /5TYE665/ | TTATTATT (SEQ ID NO: 9) | /3IAbRQSp/ |
| /5Alex594N/ | TTATTATT (SEQ ID NO: 9) | /3IAbRQSp/ |
| /5ATTO633N/ | TTATTATT (SEQ ID NO: 9) | /3IAbRQSp/ |
| /56-FAM/ | TTTTTT (SEQ ID NO: 10) | /3IABkFQ/ |
| /56-FAM/ | TTTTTTTT (SEQ ID NO: 11) | /3IABkFQ/ |
| /56-FAM/ | TTTTTTTTTT (SEQ ID NO: 12) | /3IABkFQ/ |
| /56-FAM/ | TTTTTTTTTTTT | /3IABkFQ/ |
| | (SEQ ID NO: 13) | |
| /56-FAM/ | TTTTTTTTTTTTTT | /3IABkFQ/ |
| | (SEQ ID NO: 14) | |
| /56-FAM/ | AAAAAA (SEQ ID NO: 15) | /3IABkFQ/ |
| /56-FAM/ | CCCCCC (SEQ ID NO: 16) | /3IABkFQ/ |
| /56-FAM/ | GGGGGG (SEQ ID NO: 17) | /3IABkFQ/ |

/56-FAM/: 5′ 6-Fluorescein (Integrated DNA Technologies)
/3IABkFQ/: 3′ Iowa Black FQ (Integrated DNA Technologies)
/5IRD700/: 5′ IRDye 700 (Integrated DNA Technologies)
/5TYE665/: 5′ TYE 665 (Integrated DNA Technologies)
/5Alex594N/: 5′ Alexa Fluor 594 (NHS Ester) (Integrated DNA Technologies)
/5ATTO633N/: 5′ ATTO TM 633 (NHS Ester) (Integrated DNA Technologies)
/3IRQC1N/: 3′ IRDye QC-1 Quencher (Li-Cor)
/3IAbRQSp/: 3′ Iowa Black RQ (Integrated DNA Technologies)
rU: uracil ribonucleotide
rG: guanine ribonucleotide
*This Table refers to the detection moiety and quencher moiety as their tradenames and their source is identified. However, alternatives, generics, or non-tradename moieties with similar function from other sources can also be used.

A detection moiety can be an infrared fluorophore. A detection moiety can be a fluorophore that emits fluorescence in the range of from 500 nm and 720 nm. In some cases, the detection moiety emits fluorescence at a wavelength of 700 nm or higher. In other cases, the detection moiety emits fluorescence at about 660 nm or about 670 nm. In some cases, the detection moiety emits fluorescence at in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 680 to 690, 690 to 700, 700 to 710, 710 to 720, or 720 to 730 nm. A detection moiety can be a fluorophore that emits a fluorescence in the same range as 6-Fluorescein, IRDye 700, TYE 665, Alex Fluor, or ATTO TM 633 (NHS Ester). A detection moiety can be fluorescein amidite, 6-Fluorescein, IRDye 700, TYE 665, Alex Fluor 594, or ATTO TM 633 (NHS Ester). A detection moiety can be a fluorophore that emits a fluorescence in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A detection moiety can be fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Any of the detection moieties described herein can be from any commercially available source, can be an alternative with a similar function, a generic, or a non-tradename of the detection moieties listed.

A detection moiety can be chosen for use based on the type of sample to be tested. For example, a detection moiety that is an infrared fluorophore is used with a urine sample. As another example, SEQ ID NO: 1 with a fluorophore that emits a fluorescence around 520 nm is used for testing in non-urine samples, and SEQ ID NO: 8 with a fluorophore that emits a fluorescence around 700 nm is used for testing in urine samples.

A quenching moiety can be chosen based on its ability to quench the detection moiety. A quenching moiety can be a non-fluorescent fluorescence quencher. A quenching moiety can quench a detection moiety that emits fluorescence in the range of from 500 nm and 720 nm. A quenching moiety can quench a detection moiety that emits fluorescence in the range of from 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety emits fluorescence at in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 680 to 690, 690 to 700, 700 to 710, 710 to 720, or 720 to 730 nm. A quenching moiety can quench fluorescein amidite, 6-Fluorescein, IRDye 700, TYE 665, Alex Fluor 594, or ATTO TM 633 (NHS Ester). A quenching moiety can be Iowa Black RQ, Iowa Black FQ or IRDye QC-1 Quencher. A quenching moiety can quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety can be Iowa Black RQ (Integrated DNA Technologies), Iowa Black FQ (Integrated DNA Technologies) or IRDye QC-1 Quencher (LiCor). Any of the quenching moieties described herein can be from any commercially available source, can be an alternative with a similar function, a generic, or a non-tradename of the quenching moieties listed.

The generation of the detectable signal from the release of the detection moiety can indicate that cleavage by the programmable nuclease has occurred and that the sample contains the target nucleic acid. The detection moiety can comprise a fluorescent dye. Sometimes the detection moiety comprises a fluorescence resonance energy transfer (FRET) pair. The detection moiety can comprise an infrared (IR) dye. The detection moiety can comprise an ultraviolet (UV) dye. Alternatively, or in combination, the detection moiety comprises a polypeptide. Sometimes the detection moiety comprises a biotin. Sometimes the detection moiety comprises at least one of avidin or streptavidin. The detection moiety can comprise a polysaccharide, a polymer, or a nanoparticle. The detection moiety can comprise a gold nanoparticle or a latex nanoparticle.

### Modified Nucleic Acids

Methods disclosed herein using programmable nucleases (e.g., CRISPR Cas systems) assay for a modification state of a segment of a target nucleic acid. The modification state of a segment of a target nucleic acid can be modified or an unmodified. For example, a modification state can be the presence (modified) or absence (unmodified) of any modification disclosed herein on a nucleic acid base. The segment of the target nucleic acid can be a region of bases. Assaying for the modification state can be detection of at least one or more than one bases comprising the modification, indicating the segment of the target nucleic acid is modified. Assaying for the modification state can be detection of at least one or more bases comprising the unmodified nucleic acids, indicating the segment of the target nucleic acid is unmodified. The particular methods disclosed herein, using programmable nucleases, can be tailored to sensitively and specifically assay for the modification state (modified or unmodified). Disclosed herein are methods of assaying for or detecting a nucleic acid modification, including modifications to DNA or to RNA. The methods described herein use a programmable nuclease, such as the various CRISPR/Cas systems disclosed herein, to detect modified nucleic acids. For example, a method of detection comprises contacting a programmable nuclease (e.g., any of the CRISPR enzymes disclosed herein) that is sensitive to the modification of a nucleic acid to a sample comprising a modified nucleic acid. A method of detection can comprise contacting a sample comprising a modified nucleic acid to an enzyme composition comprising a CRISPR enzyme, wherein the enzyme composition exhibits cleavage sensitivity to the modification of the nucleic acid. A method of detection can comprise contacting a sample comprising a modified nucleic acid to a reagent that differentially reacts to modified bases and to a CRISPR enzyme. Detection of nucleic acids having modifications can be used to diagnose or identify diseases associated with the modification of target nucleic acid sequences. Detection of nucleic acids having modifications such as methylation or other modifications that interfere with endonuclease activity are applicable to a number of fields, such as clinically, as a diagnostic, in laboratories as a research tool, and in agricultural applications.

The modified nucleic acids can be single stranded nucleic acids. The modified nucleic acids can be double stranded nucleic acids. The modified nucleic acids can be prepared into single stranded nucleic acids before or during the methods of assaying or detection described herein. Nucleic acid modifications can comprise any functional changes to the genomic expression that do not alter the sequence of the nucleic acid. The modified nucleic acids can be DNA. The modified nucleic acids can be RNA.

Nucleic acids can be modified. For example, a modified nucleic acid can comprise a nucleic acid with an epigenetic modification. A modified nucleic acid can comprise nucleic acid that is modified to induce a chromatin state. A modified nucleic acid can be an adenosine-to-inosine (A-to-I) edited nucleic acid. Modified nucleic acids can comprise a modification variable region. The modification variable region can be a region of a target nucleic acid sequence that may comprise a modified nucleotide and be region that binds to a guide nucleic acid. Nucleic acids can be modified by methylation. A nucleic acid modification can be 5-hydroxymethylcytidine or hydroxymethyl deoxycytidine in DNA, 5-formylcytidine, 5-carboxylcytidine, 5-hydroxymethyluridine, 5-methylcytidine, 3 -methylcytidine, N6-methyladenosine, N6, 2′-O-dimethyladenine, N1-methyladenine, N1-methylguanine, 5-methylcytidine in RNA, or 5-hydroxymethylcytidine in RNA. A modified nucleic acid (e.g., a modified ribonucleic acid or a modified deoxyribonucleic acid) may comprise a modified nitrogenous base. A modified nitrogenous base can be an adenine to hypoxanthine edited nitrogenous base. Nucleic acids may be modified by methylation of the nitrogenous base. A modified nitrogenous base may be 5-hydroxymethylcytosine (5hmC), 5-formylcytosine (5fC), 5-carboxylcytosine (5caC), 5-hydroxymethyluracil (5hmU), 5-methylcytosine (5mC), 3 -methylcytosine (3mC), N6-methyladenine (m6A), N6, 2′-0-dimethyladenine (m6Am), N1-methyladenine (m1 A), N1-methylguanine (m1G), 5-methylcytosine (m5C) in RNA, or 5-hydroxymethylcytosine (hm5C). A modified nucleic acid molecule can comprise a modification variable region. The modification variable region can be a region of a target nucleic acid sequence that may comprise a modified nucleotide and where a guide nucleic acid binds to the target nucleic acid molecule. A modified nucleic acid can be DNA. A modified nucleic acid can be RNA. Modified RNA can be tRNA, rRNA, mRNA, tmRNA, snRNA, scRNA, snoRNA, miRNA, non-coding RNA, long non-coding RNA, or viral RNA. A methylated nucleic acid can be DNA. A methylated nucleic acid can comprise a methylation variable region. The methylation variable region can be a region of a target nucleic acid sequence that may comprise a methylated nucleotide and binds to a guide nucleic acid.

A common form of nucleic acid modification is methylation, for example, methylation of a base of a nucleic acid such as a DNA or RNA molecule. Methylation can occur at a cytosine to form 5-methylcytosine (5mC), which is a methylation of the position 5 on the pyrimidine ring. Methylation of DNA can occur at CpG dinucleotides. Methylation of DNA can occur at CpG dinucleotides within CpG islands. DNA methylation can also be non-CpG methylation, such as at a CAC sequence. For example, non-CpG methylation occurs in embryonic stem cells, during neural development, and in hematopoietic stem cells. In some plants and organisms, methylation of DNA can also occur at CHG or CHH sequences, wherein H can be A, T, or C. DNA methylation can stably silence gene expression. DNA methylation can be 5-hydroxymethylcytidine, 5-formylcytidine, 5-carboxylcytidine, 5-hydroxymethyluridine, 3 -methylcytidine, or N6-methyladenosine. DNA methylation may comprise a methylated DNA nitrogenous base. A methylated DNA nitrogenous base may be 5-hydroxymethylcytosine (5hmC), 5-formylcytosine (5fC), 5-carboxylcytosine (5caC), 5-hydroxymethyluracil (5hmU), 3 -methylcytosine (3mC), N6-methyladenine (m6A). This methylation can be found in the methylation variable region of a target nucleic acid. Another modification state that can be assayed for using the methods disclosed herein is acetylation, for example, acetylation of a base of a nucleic acid such as a DNA or RNA molecule.

Some modified nucleic acids are ribonucleic acids. RNA can be modified through a number of covalent changes to the base or backbone of the molecule. Examples include A-to-I editing. RNA can be modified by methylation. Methylated of RNA can be N6-methyladenosine (e.g., comprising an N6-methyladenine (m6A) nitrogenous base). m6A-modifications are generally found near start of the 3′ untranslated region (3′UTR) of mRNAs and at canonical DRACH motifs (D=A, G, or U; R=G or A; A=A; C=C; H=A, C, or U). m6A can mark mRNAs for degradation or to promote translation. Methylated RNA can be 5-methylcytidine. m6A and 5-mC can affect RNA stability and mRNA translation. Methylated RNA can be N6, 2′-O-dimethyladenosine, N1-methyladenosine, N1-methylguanosine, or 5-hydroxymethylcytidine (hm5C). Methylated RNA may comprise a methylated RNA nitrogenous base. A methylated RNA nitrogenous base may be N6-methyladenine (m6A), 5-methylcytosine (5-mC), N6, 2′-O-dimethyladenine (m6Am), N1-methyladenine (m1A), N1-methylguanine (m1G), or 5-hydroxymethylcytosine (hm5C). This methylation can be found in the methylation variable region of the target nucleic acid.

A nucleic acid used in the methods described herein can be from a sample. A sample can be from a subject. The subject can be a single-cell eukaryotic organism; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. The subject can be a nematode, protozoan, helminth, or malarial parasite. The sample can comprise nucleic acids from a cell lysate from a eukaryotic cell, a mammalian cell, a human cell, a prokaryotic cell, or a plant cell. The sample can comprise nucleic acids expressed by a cell. The sample can comprise nucleic acids generated by in vitro methods. Additionally, the nucleic acids can be from a cell lysate.

A sample can be a biological sample. A biological sample from the subject can be blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. A tissue sample can be dissociated or liquified prior to the method of the detection of the present disclosure. A sample can be from an environmental sample, such as from soil, air, or water. The environmental sample can be taken as a swab from a surface of interest or taken directly from the surface of interest. The sample can be diluted with a buffer or a fluid or concentrated prior to use in the detection methods described herein. The sample used for detection as described herein can comprise at least one target nucleic acid that can bind to a guide nucleic acid as described herein. The target nucleic acid can be a portion of a nucleic acid. A portion of a nucleic acid can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A portion of a nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The sequence of the target nucleic acid can be reverse complementary to the sequence of a guide nucleic acid. The target nucleic acid can comprise the site of the nucleic acid modification to be detected.

A sample comprising a segment of a target nucleic acid can be used for cancer testing. The sample can comprise at least one segment of the target nucleic acid that can bind to a guide nucleic acid as described herein. The segment of the target nucleic acid, in some cases, is a portion of a nucleic acid with a modification that affects the expression of a cancer gene. A cancer gene can be any gene whose aberrant expression is associated with cancer, such as overexpression of an oncogene, suppression of tumor suppressor gene, or disregulation of a checkpoint inhibitor gene or gene associated with cellular growth, cellular metabolism, or the cell cycle. A modification that affects the expression of a cancer gene can be a modification within the cancer gene, a modification of RNA associated with the expression of a cancer gene, a modification of a nucleic acid associated with regulation of expression of a cancer gene, such as an RNA or a promoter, enhancer, or repressor of the cancer gene. In some cases, the segment of the target nucleic acid is a portion of a nucleic acid from a genomic locus of a cancer gene or an RNA expressed from a genomic locus of a cancer gene. In some cases, the segment of the target nucleic acid is a portion of a nucleic acid from a nucleic acid associated with regulation of a cancer gene, such as an RNA or a promoter, enhancer, or repressor of the cancer gene. For example, a segment of a target nucleic acid can comprise a modification, such as methylation, that affects colon cancer, bladder cancer, stomach cancer, breast cancer, non-small-cell lung cancer, pancreatic cancer, esophageal cancer, cervical cancer, ovarian cancer, hepatocellular cancer, and acute myeloid leukemia. The modified nucleic acid can be DNA or RNA. For example, a methylated target DNA segment comprises hypermethylated CpG islands in the TFPI2 promoter and indicates gastric/colorectal cancer. A methylated target nucleic acid segment can comprise a methylated DNA encoding APC, p16INK4A, or DAPK11, and can indicate lung cancer. A methylated target nucleic acid segment can comprise a methylated DNA encoding RASSF1A, p16INK4A, or CDH1, and can indicate breast cancer. A methylated target nucleic acid segment can comprise a methylated target nucleic acid encoding GSTP1 and can indicate prostate cancer. A methylated target nucleic acid segment can comprise an RNA with misregulated m6A and can indicate breast cancer, glioblastoma, acute myeloid leukemia, lung adenocarcinoma, or endometrial cancer. A methylated target nucleic acid segment can comprise an RNA with misregulated m6A encoding NANOG. A methylated target nucleic acid segment can comprise an RNA with misregulated m6A encoding FOXM1. A methylated target nucleic acid segment can comprise an RNA with misregulated m6A encoding MYC. A methylated target nucleic acid segment can comprise an RNA with misregulated m6A encoding YAP. A subject with a cancer, such as breast cancer, glioblastoma, acute myeloid leukemia, lung adenocarcinoma, or endometrial cancer, can have 1, more than 1, more than 10, more than 100, more than 200, more than 500, more than 1,000, more than 10,000, more than 100,000, or more than 1,000,000 misregulated m6A RNA transcripts per cell.

A sample comprising a segment of a target nucleic acid can be used for genetic disorder testing. The sample can comprise at least one segment of the target sequence that can bind to a guide nucleic acid as described herein. The segment of the target nucleic acid, in some cases, is a portion of a nucleic acid with a modification that affects the expression of a gene associated with a genetic disorder. A gene associated with a genetic disorder can be a gene whose overexpression is associated with a genetic disorder, from a gene associated with abnormal cellular growth resulting in a genetic disorder, or from a gene associated with abnormal cellular metabolism resulting in a genetic disorder. A modification that affects the expression of a gene associated with a genetic disorder can be a modification within the gene associated with a genetic disorder, a modification of RNA associated with a gene of the genetic disorder, or a modification of a nucleic acid associated with regulation of expression of a gene associated with a genetic disorder, such as an RNA or a promoter, enhancer, or repressor of the gene associated with the genetic disorder. In some cases, the segment of the target nucleic acid is a portion of a nucleic acid from a genomic locus of a gene associated with a genetic disorder or an RNA from a genomic locus of a gene associated with a genetic disorder. The segment of the target nucleic acid, in some cases, is a portion of a nucleic acid from a nucleic acid associated with regulation of a gene associated with a genetic disorder, such as an RNA or a promoter, enhancer, or repressor of the gene associated with the genetic disorder. For example, a segment of the target nucleic acid can comprise a modification, such as methylation, that affects Parkinson's disease, Rett Syndrome, or Immunodeficiency Centromere instability and Facial anomalies (ICF) Syndrome. The modified nucleic acid segment can be DNA or RNA. For example, a methylated target DNA segment comprises hypermethylated CpG islands in SNCA and indicates Parkinson's disease.

A sample comprising a segment of a target nucleic acid can be a laboratory sample or used in research testing. The sample can comprise at least one segment of the target sequence that can bind to a guide nucleic acid as described herein. The segment of the target nucleic acid, in some cases, is a portion of a nucleic acid with a modification of interest. A nucleic acid with a modification of interest can be any nucleic acid comprising a modification, wherein the effect of the modification on the target nucleic acid is being studied. For example, the studied modification affects gene expression. The modification that affects the expression of a gene can be a modification within the gene, a modification of RNA associated with the gene, or a modification of a nucleic acid associated with regulation of expression of the gene, such as an RNA or a promoter, enhancer, or repressor of the gene.

A sample comprising a segment of the target nucleic acid can be an agricultural sample or used in agricultural testing. The sample can comprise at least one segment of the target sequence that can bind to a guide nucleic acid as described herein. The segment of the target nucleic acid, in some cases, is a portion of a nucleic acid with a modification of interest. A nucleic acid with a modification of interest can be any nucleic acid comprising a modification, wherein the effect of the modification on target nucleic is being studied. For example, the studied modification affects gene expression. The modification that affects the expression of a gene can be a modification within the gene, a modification of RNA associated with the gene, or a modification of a nucleic acid associated with regulation of expression of the gene, such as an RNA or a promoter, enhancer, or repressor of the gene.

A number of target nucleic acids are consistent with the methods and compositions disclosed herein. Some methods described herein can detect a segment of a target nucleic acid present in the sample in various concentrations or amounts as a target nucleic acid population. The sample can have at least 2 target nucleic acids. The sample can have at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. The methods as described herein can detect target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

A number of target nucleic acid populations are consistent with the methods disclosed herein. Some methods described herein detect two or more target nucleic acid populations present in the sample in various concentrations or amounts. The sample can have at least 2 target nucleic acid populations. The sample can have at least 3, 4, 5, 6, 7, 8, 9, 10, 20, 30, 40, or 50 target nucleic acid populations. The method can detect target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

### Assaying for a Modification State of a Segment of a Target Nucleic Acid

Disclosed herein are methods of assaying for (e.g., detecting) a nucleic acid modification using a programmable nuclease system such as the CRISPR/Cas system. The methods disclosed herein can be used to determine the modification status of a target nucleic acid, e.g., to determine a modification state (e.g., if a sample comprises the modified target nucleic acid orunmodified target nucleic acids), using a programmable nuclease system such as the CRISPR/Cas system. As discussed above, a modified nucleic acid can be a modified DNA or modified RNA. For example, a modified DNA is a methylated DNA or a modified RNA is a methylated RNA. A method of detection can comprise contacting a programmable nuclease that is sensitive to the modification of a target DNA to a sample comprising the modified DNA. A method of detection can comprise contacting a sample comprising a modified target DNA to an enzyme composition comprising a programmable nuclease, wherein the enzyme composition exhibits cleavage sensitivity to the modification of the target DNA. A method of detection can comprise contacting a sample comprising a modified target DNA to a reagent that differentially reacts to the modified bases of the target DNA and to a programmable nuclease. A method of detection can comprise contacting a CRISPR enzyme that is sensitive to the modification of a nucleic acid to a sample comprising a modified nucleic acid. A method of detection can comprise contacting a sample comprising a modified nucleic acid to an enzyme composition comprising a CRISPR enzyme, wherein the enzyme composition exhibits cleavage sensitivity to the modification of the nucleic acid. A method of detection can comprise contacting a sample comprising a modified nucleic acid to a reagent that differentially reacts to modified bases and to a CRISPR enzyme. Detection of nucleic acids having modifications can be used to diagnose or identify diseases associated with the modification of target nucleic acid sequences. Detection of nucleic acids having modifications such as methylation or other modifications that interfere with endonuclease activity are applicable to a number of fields, such as clinically, as a diagnostic, in laboratories as a research tool, and in agricultural applications.

Methods disclosed herein can include a method of assaying for a modification state of a segment of a target nucleic acid, which can include the steps of contacting a sample comprising the target nucleic acid to: a guide nucleic acid that hybridizes to the segment of the target nucleic acid; a detector nucleic acid; and a programmable nuclease that cleaves the detector nucleic acid upon hybridization of the guide nucleic acid to the segment of the target nucleic acid; and assaying for a signal produced by cleavage of the detector nucleic acid to determine the modification state of the segment of the target nucleic acid. The modification state can be modified or unmodified. Assaying for the modification state can be carried out with several of the methods disclosed herein. For example, a target nucleic acid in a sample can be contacted with a guide nucleic acid, a detector nucleic acid, and a programmable nuclease and the sample can be assayed for a first signal (e.g., background subtracted fluorescence). This first signal can be compared to a second signal from a second sample having the target nucleic acid in the unmodified state (e.g., a control unmodified sample). If the first signal is less than the second signal (e.g., due to the guide nucleic acid being unable to hybridize to a modified target nucleic acid), this can indicate that the modification state of the nucleic acid in the original sample is modified. If the first signal is substantially the same or greater than the second signal, this can indicate that the modification state of the nucleic acid in the original sample is unmodified. Other methods disclosed herein are also compatible with assaying for the modification state. For example, DNA modification states can be assayed with DNA modification reagents (e.g., a modification-specific restriction enzyme that cleaves modified nucleic acids or sodium bisulfite that converts unmethylated cytosine into uracil) and DETECTR reagents (e.g., a guide nucleic acid that hybridizes to a modified DNA sequence). If a signal above background is measured from the DETECTR reaction, this indicates that the sample contains target DNA that is modified. Assaying for an unmodified DNA modification state can also be carried out using sodium bisulfite conversion of unmethylated cytosines into uracils with the inclusion of a guide nucleic acid sequence that hybridizes to unmodified DNA sequences. In these cases, if a signal above background is measured from the DETECTR reaction, this indicates that the sample contains target DNA that is unmodified. The target nucleic acid can be target RNA or target DNA. The modification can be any of the modifications described herein (e.g., methylation of a base).

A programmable nuclease system can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and can generate a detectable signal. The detectable signal can be immobilized on a support medium for detection. The detectable signal can be visualized to assess whether a target nucleic acid comprises a modification. The programmable nuclease can be a CRISPR-Cas (clustered regularly interspaced short palindromic repeats - CRISPR associated) nucleoprotein complex with trans cleavage activity, which can be activated by binding of a guide nucleic acid with a target nucleic acid. The CRISPR-Cas nucleoprotein complex can comprise a Cas protein complexed with a guide nucleic acid, which can also be referred to as CRISPR enzyme. A guide nucleic acid can be a CRISPR RNA (crRNA).

The CRISPR/Cas system used to detect a modified target nucleic acids can comprise CRISPR RNAs (crRNAs), Cas proteins, and detector nucleic acids.

A guide nucleic acid (gRNA) sequence may hybridize to a target sequence of a target nucleic acid. The term “gRNA” may be used interchangeably with the term “crRNA.” A gRNA comprises a repeat region corresponding to a specific programmable nuclease (e.g., a Cas protein). In some embodiments, the repeat region may comprise mutations or truncations with respect to the repeat sequences in pre-crRNA. The repeat sequence interacts with the programmable nuclease (e.g., a Cas protein), allowing for the gRNA and the programmable nuclease to form a complex. This complex may be referred to as a nucleoprotein. A spacer sequence may be positioned 3′ of the repeat region. The spacer sequence may hybridize to a target sequence of the target nucleic acid, wherein the target sequence is a segment of a target nucleic acid. The spacer sequences may be reverse complementary to the target sequence. In some cases, the spacer sequence may be sufficiently reverse complementary to a target sequence to allow for hybridization, however, may not necessarily be 100% reverse complementary. In some embodiments, a programmable nuclease (e.g., a Cas protein) may cleave a precursor RNA (“pre-crRNA”) to produce a gRNA, also referred to as a “mature guide RNA.” A programmable nuclease (e.g., a Cas protein) that cleaves pre-crRNA to produce a mature guide RNA is said to have pre-crRNA processing activity.

A guide nucleic acid can comprise a sequence that is reverse complementary to the sequence of a target nucleic acid. A guide nucleic acid can be a crRNA. The guide nucleic acid can bind specifically to the target nucleic acid. In some cases, the guide nucleic acid is not naturally occurring and made by artificial combination of otherwise separate segments of sequence. The artificial combination can be performed by chemical synthesis, by genetic engineering techniques, or by the artificial manipulation of isolated segments of nucleic acids. The targeting region of a guide nucleic acid can be 20 nucleotides in length. The targeting region of the guide nucleic acid can have a length of at least 10, 11, 12, 13, 14, 15, 16, 17, 18, 19, 20, 21, 22, 23, 24, 25, 26, 27, 28, 29, or 30 nucleotides. The targeting region of the guide nucleic acid can be 10, 11, 12, 13, 14, 15, 16, 17, 18, 19, 20, 21, 22, 23, 24, 25, 26, 27, 28, 29, or 30 nucleotides in length. The targeting region of guide nucleic acid can have a length from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. It is understood that the sequence of a polynucleotide need not be 100% reverse complementary to that of its target nucleic acid to be specifically hybridizable or hybridizable or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid.

For assaying for or detection of the modified nucleic acid, the segment of the target nucleic acid comprising a modification can be contacted with a guide nucleic acid and a programmable nuclease. The binding and activation of the programmable nuclease can be dependent on the modification status of the target nucleic acid. For example, if the segment of the target nucleic acid has a modification in the region that binds to the guide nucleic acid, which prevents the segment of the target nucleic acid from binding to the guide nucleic acid, then trans cleavage by the programmable nuclease is not initiated. Therefore, the detector nucleic acid cannot be cleaved, resulting in the absence of a fluorescent signal indicating the presence of the modified target nucleic acid (e.g., the target nucleic acid comprises the modification). Furthermore, if the segment of the target nucleic acid does not have a modification in the region that binds to the guide nucleic acid, which allows the segment of the target nucleic to bind to the guide nucleic acid, then trans cleavage by the programmable nuclease is initiated. Therefore, the detector nucleic acid can be cleaved, resulting the detection of a fluorescent signal indicating the presence of the unmodified target nucleic acid (e.g., the segment of the target nucleic acid is not modified).

For detection of the modified nucleic acid in some cases, the segment of the target nucleic acid comprising a modification can be contacted with an agent that alters the segment of the target nucleic acid before contacting the guide nucleic acid and the programmable nuclease. Some DNA altering agents, also referred to as a DNA modification reagent, can alter the segment of the target nucleic acid when the target nucleic acid modification is not present and cannot alter the segment of the target nucleic acid when the target nucleic acid modification is present. Some nucleic acid modification reagents (e.g., altering agents) can alter the segment of the target nucleic acid when the target nucleic acid modification is present and cannot alter the segment of the target nucleic acid when the target nucleic acid modification is not present. Some nucleic acid modification reagents (e.g., altering agents) can alter the segment of the target nucleic acid when the target nucleic acid does not contain a modification present and cannot alter the segment of the target nucleic acid when the target nucleic acid modification is present.

When the segment of the target nucleic acid is altered, in some cases, the resulting altered segment of the target nucleic acid cannot bind to the guide nucleic acid of the programmable nuclease. Therefore, the trans cleavage activity of the programmable nuclease cannot be initiated, and detector nucleic acid cannot be cleaved, resulting in the absence of a fluorescent signal indicating the presence of the altered segment of the target nucleic acid, and thus, depending on the nucleic acid modification reagent (e.g., altering agent), indicating whether the segment of the target nucleic acid comprises a modification. For example, when the modification reagent (e.g., altering agent) only alters a segment of the target nucleic acid comprising a modification and the guide nucleic acid cannot bind to the altered nucleic acid, absence of a signal indicates the segment of the target nucleic acid is a modified segment of the target nucleic acid and presence of a signal indicates the segment of the target nucleic acid comprises an unmodified nucleic acid. As another example, when the modification reagent (e.g., altering agent) only alters a segment of the target nucleic acid that does not comprise a modification and the guide nucleic acid cannot bind to the altered segment of the target nucleic acid, absence of a signal indicates the segment of the target nucleic acid is an unmodified segment of the target nucleic acid and presence of a signal indicates the segment of the target nucleic acid is a modified segment of the target nucleic acid.

When the altered segment of the target nucleic acid cannot bind to the guide nucleic acid of the programmable nuclease, the unaltered segment of the target nucleic acid can bind to the guide nucleic acid of the programmable nuclease. Therefore, the trans cleavage activity of the programmable nuclease can be initiated, and detector nucleic acid can be cleaved, resulting in the detection of a fluorescent signal indicating the presence of the unaltered segment of the target nucleic acid.

For example, if the segment of the target nucleic acid modification is methylation, then the t segment of the arget nucleic acid can be altered by a methylation-specific nucleic acid modification reagent (e.g., altering agent), such as by a methylation-specific restriction enzyme or by bisulfite conversion. A methylation-specific restriction enzyme can be any restriction enzyme that differentially cleaves a segment of the target nucleic acid depending on its methylation status. For example, a methylation-specific restriction enzyme can target a specific sequence within the target nucleic acid and can cleave the target nucleic acid only if the specific sequence is unmethylated. Therefore, if the segment of the target nucleic acid comprises an unmethylated restriction enzyme site, then the segment of the target nucleic acid can be altered by cleavage performed by the restriction enzyme. When the segment of the target nucleic acid is cleaved by the methylation-specific restriction enzyme, the cleaved fragments of the segment of the target nucleic acid can no longer bind to the guide nucleic acid of the programmable nuclease, and thus, trans cleavage activity cannot be initiated, and detector nucleic acid cannot be cleaved, resulting in the absence of a fluorescent signal, which indicates the segment of the target nucleic acid is an unmodified (here, unmethylated) segment of the target nucleic acid. However, if the segment of the target nucleic acid is methylated, the methylated segment of the target nucleic acid can bind to the guide nucleic acid of the programmable nuclease after contact with methylation-specific restriction enzyme because the methylation of the segment of the target nucleic acid prevents the methylation-specific restriction enzyme from altering the segment of the target nucleic acid by cleavage. Therefore, the trans cleavage activity of the programmable nuclease can be initiated, and detector nucleic acid can be cleaved, resulting in the detection of a fluorescent signal indicating the presence of the modified (here, methylated) segment of the target nucleic acid.

The methods disclosed herein may be used to assay for (e.g., detect) the presence or absence of acetylation. The methods disclosed herein may be used to detect the presence or absence of methylation. For example, detection of methylation may include detection of hypermethylation of CpG islands, which are stretches of DNA with a higher frequency of CG sequences than other regions, in promoter regions for tumor-suppressor genes is common in several types of cancer, such as colon cancers, bladder cancers, and stomach cancers. A gRNA sequence may be designed to target a nucleic acid region that has variable methylation. In some embodiments, the methylation of the nucleic acid region may vary based on a disease state. For example, a gRNA sequence may be designed to target a nucleic acid region that is hypermethylated in cancer. Detection of methylation may enable reliable early detection of many cancer types and other CpG methylation-related diseases.

Bisulfite conversion can also be used to produce methylation-specific nucleic acid alterations in a segment of a target nucleic acid. The bisulfite reaction can alter the segment of the target nucleic acid sequence depending on the methylation status of the segment of the target nucleic acid by producing methylation-specific nucleic acid alterations in the segment of the target nucleic acid. More specifically, during bisulfite conversion unmethylated cytosines in a segment of the target nucleic acid are converted to uracils, thus altering the segment of the target nucleic acid. However, if the cytosines are methylated in the segment of the target nucleic acid (e.g., 5-methylcytosine, 5-hydroxymethylcytosine), then the methylated cytosines remain methylated cytosines during bisulfite conversion and the segment of the target nucleic acid is thus an unaltered segment of the target nucleic acid with respect to the methylated cytosines. A methylation in a segment of the target nucleic acid, for example, leads to a binding mismatch in the segment of the target nucleic acid/guide nucleic acid complex of the programmable nuclease after bisulfite conversion, which then is unable to initiate trans cleavage activity of programmable nuclease. Since the trans cleavage activity of the programmable nuclease cannot be initiated, and subsequently, the detector nucleic acid cannot be cleaved, a fluorescent signal indicating the presence of the segment of the target nucleic acid cannot be detected. The absence of the fluorescent signal can indicate that the segment of the target nucleic acid is methylated. An unmethylated segment of the target nucleic acid, however, can lead to a binding match in the segment of the target nucleic acid/guide nucleic acid complex of the programmable nuclease after bisulfite conversion, which can then initiate trans cleavage activity. Since the trans cleavage activity of the programmable nuclease can be initiated, and subsequently, the detector nucleic acid can be cleaved, a fluorescent signal indicating the presence of the segment of the target nucleic acid can be detected. The detection of the fluorescent signal can indicate that the segment of the target nucleic acid is unmethylated. Therefore, unmethylated segment of the target nucleic acid can induce trans-cleavage of the detector nucleic acid by the programmable nuclease, enabling high-fidelity discrimination between methylated and unmethylated segments of the target nucleic acids.

In some cases, the guide nucleic acid used in the detection reaction (e.g., DETECTR) can bind to a segment of the target nucleic acid comprising a sequence that is unaltered by the bisulfite conversion. The unaltered segment of the target nucleic acid can then bind to guide nucleic acid in which the guide nucleic acid is complementary to the sequence of the unaltered segment of the target nucleic acid. In some cases, the guide nucleic acid used in the detection reaction (e.g., DETECTR) can bind to a segment of the target nucleic acid comprising a sequence that is altered by the bisulfite conversion. The altered segment of the target nucleic acid can then bind to guide nucleic acid in which the guide nucleic acid is complementary to the sequence of the altered segment of the target nucleic acid. Since the trans cleavage activity of the programmable nuclease can be initiated upon the binding of the segment of the target nucleic acid to the guide nucleic acid, and subsequently, the detector nucleic acid can be cleaved, a signal indicating the presence of the segment of the target nucleic acid can be detected in which the absence or presence of the signal indicates the methylation status of the segment of the target nucleic acid. In some cases, the detection of the signal indicates that the segment of the target nucleic acid is methylated when the guide nucleic acid used in the detection reaction (e.g., DETECTR) reaction binds to the unaltered segment of the target nucleic acid. Alternatively, the absence of detection of the signal indicates that the segment of the target nucleic acid is unmethylated when the guide nucleic acid used in the detection reaction (e.g., DETECTR) binds to the unaltered segment of the target nucleic. An unmethylated segment of the target nucleic acid, however, can lead to an altered segment of the target nucleic acid sequence after bisulfite conversion, which cannot then bind to the guide nucleic acid to initiate trans cleavage activity. Since the trans cleavage activity of the programmable nuclease cannot be initiated, and subsequently, the detector nucleic acid cannot be cleaved, a signal indicating the presence of the segment of the target nucleic acid cannot be detected. The absence of the signal can indicate that the segment of the target nucleic acid is unmethylated. Therefore, methylated segment of the target nucleic acid can induce trans-cleavage of the detector nucleic acid by the binding to guide nucleic acid of the programmable nuclease, enabling high-fidelity discrimination between methylated and unmethylated segment of the target DNA. In some cases, the detection of the signal indicates that the t segment of the arget nucleic acid is unmethylated when the guide nucleic acid used in the detection reaction (e.g., DETECTR) binds to the altered segment of the target nucleic acid. Alternatively, the absence of detection of the signal indicates that the segment of the target nucleic acid is methylated when the guide nucleic acid used in the detection reaction (e.g., DETECTR) binds to the altered segment of the target nucleic acid. Therefore, combining bisulfite conversion with a detection reaction (e.g., DETECTR) can enable high-fidelity discrimination between methylated and unmethylated segment of the target nucleic acids.

Additionally, altered or unaltered target nucleic acid (comprising the segment of the target nucleic acid) can be amplified before binding to the guide nucleic acid of the programmable nuclease. This amplification can be PCR amplification or isothermal amplification. This nucleic acid amplification of the sample can improve at least one of sensitivity, specificity, or accuracy of the detection the target nucleic acid. RNA can be first be reverse transcribed and then amplified as described herein. The reagents for nucleic acid amplification can comprise a recombinase, a oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C. The nucleic acid amplification reaction can be performed at a temperature of at least 20° C., 25° C., 30° C., 35° C., 37° C., 40° C., or 45° C. The nucleic acid amplification reaction method may be selected based on compatibility with a programmable nuclease (e.g., any Cas protein disclosed herein). In some embodiments, a nucleic acid amplification reaction method may be selected that may be performed at a temperature at which the Cas protein exhibits nuclease activity. For example, a Cas protein may exhibit nuclease activity of temperatures of about 20° C., about 25° C., about 30° C., about 35° C., about 37° C., about 40° C., or about 45° C. In some embodiments, a nucleic acid amplification reaction method may be selected that may be performed in a buffer in which the Cas protein exhibits nuclease activity. For example, the buffer may comprise a salt concentration in which the Cas protein exhibits nuclease activity.

DNA modifications can be detected using a programmable nuclease with trans cleavage activity detection platform to achieve high-sensitivity detection of DNA modifications, such as DNA methylation. For example, DNA modifications can be detected using a CRISPR-Cas mediated nucleic acid detection platform. Methylation of CpG sites in the human genome is an epigenetic modification that can stably silence gene expression. Hypermethylation of CpG islands in promoter regions of tumor-suppressor genes is extremely common in several types of cancer, such as colon cancers, bladder cancers, and stomach cancers. Due to the high frequency of CpG methylation in specific promoter sequences, a nucleic acid based diagnostic test that is sensitive to DNA methylation can enable simple, reliable early detection of many cancer types and other CpG methylation-related diseases.

RNA modifications can be detected using a programmable nuclease with trans cleavage activity detection platform to achieve high-sensitivity detection of RNA modifications. For example, RNA modifications can be detected using a CRISPR-Cas mediated nucleic acid detection platform. RNA modifications, such as methylation, impact RNA structure, RNA function, and the ability of proteins to bind RNA. N6-methyladenosine (e.g., a nucleic acid with an N6-methyladenine (m6A) nitrogenous base) is the most common RNA modification in messenger RNAs (mRNAs). m6A-modifications are generally found near start of the 3′ untranslated region (3′UTR) of mRNAs and at canonical DRACH motifs. m6A is an RNA modification that can regulate post-transcriptional gene expression by marking mRNAs for degradation when present. Deregulation of m6A-pathway genes has been implicated in variety of cancers, including breast cancer, non-small-cell lung cancer, and acute myeloid leukemia. Increases or decreases in m6A-levels transcriptome-wide can lead to aberrant gene expression and the potential activation of oncogenes. Due to the disease implications of the methylation state of an RNA, a nucleic acid based diagnostic test that is sensitive to RNA methylation can enable simple, reliable early detection of many cancer types and other m6A-related diseases.

Methods for assaying for a modification state of a segment of target RNA can include: contacting a sample comprising the target RNA to: a guide nucleic acid that hybridizes to the segment of the target RNA; a detector nucleic acid; and a programmable nuclease that cleaves the detector nucleic acid upon hybridization of the guide nucleic acid to the segment of the target RNA; and contacting a second sample comprising an RNA having an unmodified segment comprising the same sequence as the segment of the target RNA to: the guide nucleic acid; the detector nucleic acid; and the programmable nuclease that cleaves the detector nucleic acid upon hybridization of the guide nucleic acid to the segment of the unmodified RNA; assaying for a first signal produced by cleavage of the detector nucleic acid in the sample; assaying for a second signal produced by cleavage of the detector nucleic acid in the second sample; and determining the modification state of the target RNA based on a comparison of the first signal to the second signal. The modification state of the segment is modified when the first signal is less than the second signal and the modification state of the segment is unmodified when the first signal is substantially the same as the second signal.

Assaying for the modification state can comprise several of the methods disclosed herein. For example, a segment of a target nucleic acid in a sample can be contacted with a guide nucleic acid, a detector nucleic acid, and a programmable nuclease and the sample can be assayed for a first signal (e.g., background subtracted fluorescence). This first signal can be compared to a second signal from a second sample having the segment of the target nucleic acid in the unmodified state. If the first signal is less than the second signal (e.g., due to the guide nucleic acid being unable to hybridize to a modified segment of the target nucleic acid), this can indicate that the modification state of the segment of the target nucleic acid in the original sample is modified. If the first signal is substantially the same or greater than the second signal, this can indicate that the modification state of the segment of the target nucleic acid in the original sample is unmodified. Other methods disclosed herein are also compatible with assaying for the modification state. For example, DNA modification states can be assayed with DNA modification reagents (e.g., a modification-specific restriction enzyme that cleaves modified nucleic acids or sodium bisulfite that converts unmethylated cytosine into uracil) and DETECTR reagents (e.g., a guide nucleic acid that hybridizes to a modified DNA sequence). If a signal above background is measured from the DETECTR reaction, this indicates that the sample comprises a segment of thetarget DNA that is modified. Assaying for an unmodified DNA modification state can also be comprise using sodium bisulfite conversion of unmethylated cytosines into uracils with the inclusion of a guide nucleic acid sequence that hybridizes to unmodified DNA sequences. In these cases, if a signal above background is measured from the DETECTR reaction, this indicates that the sample comprises a segment of thetarget DNA that is unmodified.

The signal can be the signal from the DETECTR assay, for example, a fluorescence signal. The fluorescence signal can be the fluorescence after background subtraction. In some embodiments, the segment of the target RNA can be reverse transcribed into DNA, amplified, and in vitro transcribed back into the segment of the target RNA. This can allow for sensitive detection of small amounts of the RNA that may be in the sample.

Any of the modifications disclosed herein (e.g., methylation or acetylation) can be present on a nucleic acid in a region 5′ to 3′ of nucleic acid 1 to nucleic acid 16 of a segment. Any of the modifications disclosed herein (e.g., methylation or acetylation) can be present on a nucleic acid in a region 5′ to 3′ of nucleic acid 1 to nucleic acid 8 of a segment. Any of the modifications disclosed herein (e.g., methylation or acetylation) can be present on a nucleic acid in a region 5′ to 3′ of nucleic acid 1 to nucleic acid 50 of a segment. Any of the modifications disclosed herein (e.g., methylation or acetylation) can be present on a nucleic acid in a region 5′ to 3′ of nucleic acid 1 to nucleic acid 5 of a segment. Any of the modifications disclosed herein (e.g., methylation or acetylation) can be present on a nucleic acid in a region 5′ to 3′ of nucleic acid 5 to nucleic acid 10 of a segment. Any of the modifications disclosed herein (e.g., methylation or acetylation) can be present on a nucleic acid in a region 5′ to 3′ of nucleic acid 10 to nucleic acid 15 of a segment. Any of the modifications disclosed herein (e.g., methylation or acetylation) can be present on a nucleic acid in a region 5′ to 3′ of nucleic acid 15 to nucleic acid 20 of a segment. Any of the modifications disclosed herein (e.g., methylation or acetylation) can be present on a nucleic acid in a region 5′ to 3′ of nucleic acid 20 to nucleic acid 25 of a segment. Any of the modifications disclosed herein (e.g., methylation or acetylation) can be present on a nucleic acid in a region 5′ to 3′ of nucleic acid 25 to nucleic acid 30 of a segment. Any of the modifications disclosed herein (e.g., methylation or acetylation) can be present on a nucleic acid in a region 5′ to 3′ of nucleic acid 30 to nucleic acid 35 of a segment. Any of the modifications disclosed herein (e.g., methylation or acetylation) can be present on a nucleic acid in a region 5′ to 3′ of nucleic acid 35 to nucleic acid 40 of a segment. Any of the modifications disclosed herein (e.g., methylation or acetylation) can be present on a nucleic acid in a region 5′ to 3′ of nucleic acid 40 to nucleic acid 45 of a segment. Any of the modifications disclosed herein (e.g., methylation or acetylation) can be present on a nucleic acid in a region 5′ to 3′ of nucleic acid 45 to nucleic acid 50 of a segment. Any of the modifications disclosed herein (e.g., methylation or acetylation) can be present on a nucleic acid in a region 5′ to 3′ of nucleic acid 5 to nucleic acid 20 of a segment. Any of the modifications disclosed herein (e.g., methylation or acetylation) can be present on a nucleic acid in a region 5′ to 3′ of nucleic acid 4 to nucleic acid 30 of a segment. Any of the modifications disclosed herein (e.g., methylation or acetylation) can be present on a nucleic acid in a region 5′ to 3′ of nucleic acid 2 to nucleic acid 40 of a segment. Any of the modifications disclosed herein (e.g., methylation or acetylation) can be present on a nucleic acid in a region 5′ to 3′ of nucleic acid 10 to nucleic acid 20 of a segment. Any of the modifications disclosed herein (e.g., methylation or acetylation) can be present on a nucleic acid in a region 5′ to 3′ of nucleic acid 8 to nucleic acid 20 of a segment. Any of the modifications disclosed herein (e.g., methylation or acetylation) can be present on a nucleic acid in a region 5′ to 3′ of nucleic acid 1 to nucleic acid 4 of a segment. Any of the modifications disclosed herein (e.g., methylation or acetylation) can be present on a nucleic acid in a region 5′ to 3′ of nucleic acid 1 to nucleic acid 3 of a segment. Any of the modifications disclosed herein (e.g., methylation or acetylation) can be present on a nucleic acid in a region 5′ to 3′ of nucleic acid 1 to nucleic acid 10 of a segment.

### Assaying for a Modification State of DNA

A modified target DNA can be detected using a programmable nuclease, such as a CRISPR/Cas system. In some embodiments, the methods disclosed herein assay for a modification state of a segment of a target DNA by contacting a sample comprising the target DNA to: a DNA modification reagent; a guide nucleic acid that hybridizes to the segment of the target DNA; a detector nucleic acid; and a programmable nuclease that cleaves the detector nucleic acid upon hybridization of the guide nucleic acid to the segment of the target DNA; and assaying for a signal produced by cleavage of the detector nucleic acid to determine the modification state of the segment of the target DNA. In some embodiments, the DNA modification reagent is a modification-specific restriction enzyme or sodium bisulfate.

In some embodiments, methods of assaying for a modification state of a segment of a target DNA can including using the modification-specific restriction enzyme. For example, the method includes contacting a sample comprising the target DNA to: a modification-specific restriction enzyme that cleaves the segment of the target DNA when the segment of the target DNA is unmodified; a guide nucleic acid that hybridizes to the segment of the target DNA; a detector nucleic acid; and a programmable nuclease that cleaves the detector nucleic acid upon hybridization of the guide nucleic acid to the segment of the target DNA; and assaying for a signal produced by cleavage of the detector nucleic acid to determine the modification state of the segment of the target DNA. Further, detection of the signal can indicate that the segment of the target DNA is modified.

In some embodiments, methods of assaying for a modification state of a segment of a target DNA can including using sodium bisulfite. For example, the method can include contacting the sample to: sodium bisulfite; a guide nucleic acid that hybridizes to the segment of the target DNA; a detector nucleic acid; and a programmable nuclease that cleaves the detector nucleic acid upon hybridization of the guide nucleic acid to the segment of the target DNA; and assaying for a signal produced by cleavage of the detector nucleic acid to determine the modification state of the segment of the target DNA. Further, detection of the signal indicates the modification state of the segment of the target DNA is unmodified.

Use of sodium bisulfite with a DETECTR reaction can also be carried out to detect that the modification state of a target DNA is unmodified. For example, the methods disclosed herein include a method of assaying for a modification state of a segment of a target DNA, the method comprising: contacting a sample comprising the target DNA to: sodium bisulfite; a guide nucleic acid that hybridizes to a sodium bisulfite converted segment of the target DNA; a detector nucleic acid; a programmable nuclease that cleaves the detector nucleic acid upon hybridization of the guide nucleic acid to the sodium bisulfite converted segment of the target DNA; and assaying for a signal produced by cleavage of the detector nucleic acid to determine the modification state of the segment of the target DNA. Further, the detection of the signal indicates the modification state of the segment of the target DNA is modified.

A method of detection can comprise contacting a programmable nuclease that is sensitive to the modification of a target DNA to a sample comprising the modified DNA. A method of detection can comprise contacting a sample comprising a modified target DNA to an enzyme composition comprising a programmable nuclease, wherein the enzyme composition exhibits cleavage sensitivity to the modification of the target DNA. A method of detection can comprise contacting a sample comprising a modified target DNA to a reagent that differentially reacts to the modified bases of the target DNA and to a programmable nuclease. A method of detection can comprise contacting a CRISPR enzyme that is sensitive to the modification of a target DNA to a sample comprising the modified DNA. A method of detection can comprise contacting a sample comprising a modified target DNA to an enzyme composition comprising a CRISPR enzyme, wherein the enzyme composition exhibits cleavage sensitivity to the modification of the target DNA. A method of detection can comprise contacting a sample comprising a modified target DNA to a reagent that differentially reacts to the modified bases of the target DNA and to a CRISPR enzyme. Detection of DNA with modifications can be used to diagnose or identify diseases associated with the modification of target nucleic acid sequences. Detection of nucleic acids having modifications such as methylation or other modifications that interfere with endonuclease activity are applicable to a number of fields, such as clinically, as a diagnostic, in laboratories as a research tool, and in agricultural applications.

The CRISPR/Cas system used to detect a modified target DNA can be a DNA Endonuclease Targeted CRISPR TransReporter (DETECTR) system. This system can comprise crRNAs, Cas proteins, and detector nucleic acids.

A crRNA can comprise a sequence that is reverse complementary to the sequence of a target DNA. The crRNA can bind specifically to the target DNA. In some cases, the crRNA is not naturally occurring and made by artificial combination of otherwise separate segments of sequence. The artificial combination can be performed by chemical synthesis, by genetic engineering techniques, or by the artificial manipulation of isolated segments of nucleic acids.

The crRNA and Cas protein can form a CRISPR enzyme. A Cas protein can be any Cas protein with trans cleavage activity upon binding of the crRNA to the target DNA. For example, a Cas protein is a Cas12 nuclease. The Cas12 nuclease can be Cas12a, Cas12b, Cas12c, Cas12d, or Cas12e. Sometimes the Cas protein is a type III CRISPR-Cas system. In some cases, the Cas protein is from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pint), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (E1), Lactobacillus salivarius (Ls), or Thermus thermophilus (Tt). The trans cleavage activity of the CRISPR enzyme can be activated when the crRNA is complexed with the target DNA sequence.

When the crRNAs of the CRISPR enzyme binds to a target DNA, the CRISPR enzyme's trans cleavage activity can be initiated, and detector nucleic acids can be cleaved, resulting in the detection of a detectable signal, such as fluorescence. Detector nucleic acids can comprise a detection moiety, wherein the detector nucleic acid can be cleaved by the activated CRISPR enzyme, thereby generating a detectable signal, such as a fluorescent signal. The generation of the detectable signal from the release of the detection moiety can indicate that cleavage by the CRISPR enzyme has occurred and that the sample contains the target nucleic acid.

As described herein, the CRISPR/Cas system can utilize the trans cleavage abilities of CRISPR enzymes to achieve fast and high-fidelity detection of modified DNA of a target DNA within a sample.

For assaying for or detection of the modified DNA modification state, the target DNA comprising a modification can be contacted with an agent that alters the target DNA before contacting the CRISPR enzyme. Some DNA modification reagent (e.g., altering agents) can alter the target DNA when the target DNA modification is not present and cannot alter the target DNA when the target DNA modification is present. For example, the restriction endonuclease HpaII cuts DNA at its restriction site when a modification is not present, but is not able to cut DNA at its restriction site when a modification, such as C5 methylation, is present at its restriction site. Some DNA modification reagent (e.g., altering agents) can alter the target DNA when the target DNA modification is present and cannot alter the target DNA when the target DNA modification is not present. For example, the restriction endonuclease Dpn I cuts DNA when the restriction site comprises an N6 methylation, but is not able to cut DNA when the restriction site is an unmodified restriction site.

When the target DNA is altered, in some cases, the altered target DNA cannot bind to the crRNA of the CRISPR enzyme. Therefore, the trans cleavage activity of the CRISPR enzyme cannot be initiated, and detector nucleic acid cannot be cleaved, resulting in the absence of a signal (e.g., fluorescent signal) indicating the presence of the target DNA. The absence of signal (e.g., fluorescent signal) can indicate that the target DNA does not comprise a modification and, therefore, has an unmodified modification state. Furthermore, when the altered target DNA cannot bind to the crRNA of the CRISPR enzyme, the unaltered target DNA can bind to the crRNA of the CRISPR enzyme. Therefore, the trans cleavage activity of the CRISPR enzyme can be initiated, and detector nucleic acid can be cleaved, resulting in the detection of a signal (e.g., fluorescent signal) indicating the presence of the target DNA. The detection of the signal (e.g., fluorescent signal) can indicate that the target DNA comprises a modification. For example, if the DNA modification is methylation, then the target DNA can be altered by a methylation-specific DNA modification reagent (e.g., altering agent), such as by a methylation-specific restriction enzyme or by bisulfite conversion. A methylation-specific restriction enzyme can be any restriction enzyme that differentially cleaves a target DNA depending on its methylation status. A methylation-specific restriction enzyme can be any restriction enzyme engineered to differentially cleave a target DNA depending on its methylation status. A methylation-specific enzyme can be any restriction enzyme that can cleave a methylated but not an unmethylated nucleic acid residue in the enzyme's restriction site. For example, methylation-specific enzyme can be Dpnl, Mspl, MspJI, LpnPI, FspEI, or McrBC. Dpnl can target the restriction site 5′-GA↓TC-3′ and can cleave DNA only if the internal adenosine residue in the restriction site is methylated. Mspl can target the restriction site 5′-C↓CGG-3′ and can cleave when the second cytosine is methylated. MspJI can cleave methylated cytosine when it is two nucleotides away from adenine or guanine and will leave a four-base overhang on the 5′ side. LpnPI can target the restriction site 5′-C^{m}CDG(N)₁₀↓-3′ and can be used to identify 5-hmC and 5-mC. McrBC can cleave a target site between two methylated cytosines (e.g., GmC or AmC) and can be used when a nucleic acid molecule is densely methylated. A methylation-specific enzyme can be any restriction enzyme that is not able to cleave a methylated nucleic acid residue. A methylation-specific enzyme can be any restriction enzyme that is not able to cleave a methylated cytosine residue. A methylation-specific enzyme can by any one of Aat II, Acc II, DpnII, Aor13H I, Aor51H I, BspT104 I, BssH II, Cfr10 I, Cla I, Cpo I, Eco52, I, Hae II, Hha I, Mlu I, Nae I, Not I, Nru I, Nsb I, PmaC I, Psp1406 I, Pvu I, Sac II, Sal I, Sma I, SnaB I or Epi HpaII. Aat II can target the restriction site 5′-GACGT↓C-3′ and can cleave DNA only if the internal cytosine residue in the restriction site is unmethylated. Acc II can target the restriction site 5′-CG↓CG-3′ and can cleave DNA only if the internal cytosine residue in the restriction site is unmethylated. Aor13H I can target the restriction site 5′-T↓CCGGA-3′ and can cleave DNA only if the internal cytosine residue in the restriction site is unmethylated. Aor51H I can target the restriction site 5′-AGC↓GCT-3′ and can cleave DNA only if the internal cytosine residue in the restriction site is unmethylated. BspT104 I can target the restriction site 5′-TT↓CGAA-3′ and can cleave DNA only if the internal cytosine residue in the restriction site is unmethylated. BssH II can target the restriction site 5′-G↓CGCGC-3′ and can cleave DNA only if the internal cytosine residue in the restriction site is unmethylated. Cfr10 I can target the restriction site 5′-R↓CCGGY-3′, wherein Y can be C or T, and can cleave DNA only if the internal cytosine residue in the restriction site is unmethylated. Cla I can target the restriction site 5′-AT↓CGAT-3′ and can cleave DNA only if the internal cytosine residue in the restriction site is unmethylated. Cpo I can target the restriction site 5′-CG↓GWCCG-3′, wherein W can be A or T, and can cleave DNA only if the internal cytosine residue in the restriction site is unmethylated. Dpn II can target the restriction site 5′-↓GATC-3′ and can cleave DNA only if the internal adenosine residue in the restriction site is unmethylated. Eco52 I can target the restriction site 5′-C↓GGCCG-3′ and can cleave DNA only if the internal cytosine residue in the restriction site is unmethylated. Hae II can target the restriction site 5′-RGCGC↓Y-3′, wherein Y can be C or T, and can cleave DNA only if the internal cytosine residue in the restriction site is unmethylated. Hap II can target the restriction site 5′-C↓CGG-3′ and can cleave DNA only if the internal cytosine residue in the restriction site is unmethylated. Hha I can target the restriction site 5′-GCG↓C-3′ and can cleave DNA only if the internal cytosine residue in the restriction site is unmethylated. Mlu I can target the restriction site 5′-A↓CGCGT-3′ and can cleave DNA only if the internal cytosine residue in the restriction site is unmethylated. Nae I can target the restriction site 5′-GCC↓GGC-3′ and can cleave DNA only if the internal cytosine residue in the restriction site is unmethylated. Not I can target the restriction site 5′-GC↓GGCCGC-3′ and can cleave DNA only if the internal cytosine residue in the restriction site is unmethylated. Nru I can target the restriction site 5′-TCG↓CGA-3′ and can cleave DNA only if the internal cytosine residue in the restriction site is unmethylated. Nsb I can target the restriction site 5′-TCG↓GCA-3′ and can cleave DNA only if the internal cytosine residue in the restriction site is unmethylated. PmaC I can target the restriction site 5′-CAC↓GTG-3′ and can cleave DNA only if the internal cytosine residue in the restriction site is unmethylated. Psp1406 I can target the restriction site 5′-AA↓CGTT-3′ and can cleave DNA only if the internal cytosine residue in the restriction site is unmethylated. Pvu I can target the restriction site 5′-CGAT↓CG-3′ and can cleave DNA only if the internal cytosine residue in the restriction site is unmethylated. Sac II can target the restriction site 5′-CCGC↓GG-3′ and can cleave DNA only if the internal cytosine residue in the restriction site is unmethylated. Sal I can target the restriction site 5′-GJ↓TCGAC-3′ and can cleave DNA only if the internal cytosine residue in the restriction site is unmethylated. Sma I can target the restriction site 5′-CCC↓GGG-3′ and can cleave DNA only if the internal cytosine residue in the restriction site is unmethylated. SnaB I can target the restriction site 5′-TAC↓GTA-3′ and can cleave DNA only if the internal cytosine residue in the restriction site is unmethylated. Epi HpaII can target the restriction site 5′-C↓CGG-3′ and can cleave DNA only if the internal CpG in the restriction site is unmethylated. Therefore, if the target DNA comprises an unmethylated restriction enzyme site, such as an internal unmethylated CpG site, then the target DNA can be altered by cleavage performed by the restriction enzyme, such as an Epi HpaII restriction enzyme. When the target DNA is cleaved, it can no longer bind to the crRNA of CRISPR enzyme, and thus, trans cleavage activity cannot be initiated, and detector nucleic acid cannot be cleaved, resulting in the absence of a signal (e.g., fluorescent signal) indicating the presence of the target DNA. The absence of signal (e.g., fluorescent signal) can indicate that the target DNA is not methylated. Furthermore, the methylated target DNA can bind to the crRNA of the CRISPR enzyme after contact with methylation-specific restriction enzyme, such as Epi HpaII, because the methylation of the target DNA prevents the methylation-specific restriction enzyme from altering the target DNA by cleavage. Therefore, the trans cleavage activity of the CRISPR enzyme can be initiated, and detector nucleic acid can be cleaved, resulting in the detection of a signal (e.g., fluorescent signal) indicating the presence of the target DNA. The assaying for or detection of the signal (e.g., fluorescent signal) can indicate that the target DNA is methylated.

Bisulfite conversion can also be used to produce methylation-specific DNA alterations in target DNA. The bisulfite reaction can alter the target DNA by converting unmethylated cytosines to uracils, but methylated cytosines remain unaltered. The unaltered target DNA can then bind to crRNA in which the crRNA is complementary to the sequence of the target DNA that has not undergone bisulfite conversion. A cytosine methylation in a CpG site of the target DNA, for example, leads to a C-G pair in the target DNA/crRNA duplex of the CRISPR enzyme after bisulfite conversion, which then initiates ssDNase activity of CRISPR enzyme. Since the trans cleavage activity of the CRISPR enzyme can be initiated, and subsequently, the detector nucleic acid can be cleaved, a signal (e.g., fluorescent signal) indicating the presence of the target DNA can be detected. The detection of the signal (e.g., fluorescent signal) can indicate that the target DNA is methylated. An unmethylated CpG site, however, can lead to a U-G mismatch in the DNA/crRNA duplex of the CRISPR enzyme after bisulfite conversion, which cannot then initiate trans cleavage activity. Since the trans cleavage activity of the CRISPR enzyme cannot be initiated, and subsequently, the detector nucleic acid cannot be cleaved, a signal (e.g., fluorescent signal) indicating the presence of the target DNA cannot be detected. The absence of the signal (e.g., fluorescent signal) can indicate that the target DNA is unmethylated. Therefore, methylated target DNA can induce trans-cleavage of the detector nucleic acid by the CRISPR enzyme, enabling high-fidelity discrimination between methylated and unmethylated target DNA.

When the target DNA is altered, in some cases, the altered target DNA binds to the crRNA of the CRISPR enzyme. Therefore, the trans cleavage activity of the CRISPR enzyme can be initiated, and detector nucleic acid can be cleaved, resulting in the detection of a signal (e.g., fluorescent signal) indicating the presence of the target DNA. The detection of the signal (e.g., fluorescent signal) can indicate that the target DNA does not comprise a modification and, therefore, has a modified modification state. Furthermore, when the altered target DNA cannot bind to the crRNA of the CRISPR enzyme, the unaltered target DNA cannot bind to the crRNA of the CRISPR enzyme. Therefore, the trans cleavage activity of the CRISPR enzyme cannot be initiated, and detector nucleic acid cannot be cleaved, resulting in the absence of a signal (e.g., fluorescent signal) indicating the presence of the target DNA. The absence of signal (e.g., fluorescent signal) can indicate that the target DNA comprises a modification and, therefore, has an unmodified modification state. Bisulfite conversion can also be used to produce methylation-specific DNA alterations in target DNA. For example, a bisulfite reaction can alter the target DNA by converting unmethylated cytosines to uracils, but methylated cytosines remain unaltered. The altered target DNA can then bind to crRNA in which the crRNA is complementary to the sequence of the target DNA that has undergone bisulfite conversion. A cytosine methylation in a CpG site of the target DNA, for example, leads to a C-A mismatch in the target DNA/crRNA duplex of the CRISPR enzyme after bisulfite conversion, which then is unable to initiate trans cleavage activity of CRISPR enzyme. Since the trans cleavage activity of the CRISPR enzyme cannot be initiated, and subsequently, the detector nucleic acid cannot be cleaved, a signal (e.g., fluorescent signal) indicating the presence of the target DNA cannot be detected. The absence of signal (e.g., fluorescent signal) can indicate that the target DNA is methylated. An unmethylated CpG site, however, can lead to a U-A pair in the DNA/crRNA duplex of the CRISPR enzyme after bisulfite conversion, which can then initiate trans cleavage activity. Since the trans cleavage activity of the CRISPR enzyme can be initiated, and subsequently, the detector nucleic acid can be cleaved, a signal (e.g., fluorescent signal) indicating the presence of the target DNA can be detected. The detection of the signal (e.g., fluorescent signal) can indicate that the target DNA is unmethylated. Therefore, unmethylated target DNA can induce trans-cleavage of the detector nucleic acid by the CRISPR enzyme, enabling high-fidelity discrimination between methylated and unmethylated target DNA.

The crRNA can have a sequence comprising at least one uracil in a region from nucleic acid residue 5 to 20 that is reverse complementary to a modification variable region (e.g., can have an unmodified modification state or a modified modification state) in the target DNA. The crRNA, in some cases, has a sequence comprising at least one uracil in a region from nucleic acid residue 5 to 10, 10 to 15, or 15 to 20 that is reverse complementary to a modification variable region in the target DNA. The crRNA can have a sequence comprising at least one uracil in a region from nucleic acid residue 5 to 20 that is reverse complementary to a methylation variable region in the target DNA. The crRNA, in some cases, has a sequence comprising at least one uracil in a region from nucleic acid residue 5 to 10, 10 to 15, or 15 to 20 that is reverse complementary to a methylation variable region in the target DNA.

Additionally, altered or unaltered target DNA can be amplified before binding to the crRNA of the CRISPR enzyme. This amplification can be PCR amplification or isothermal amplification. This nucleic acid amplification of the sample can improve at least one of sensitivity, specificity, or accuracy of the detection the target DNA. The reagents for nucleic acid amplification can comprise a recombinase, a oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C. The nucleic acid amplification reaction can be performed at a temperature of at least 20° C., 25° C., 30° C., 35° C., 37° C., 40° C., or 45° C.

### Assaying for a Modification State of RNA

A modified target RNA can be detected using a programmable nuclease, such as a CRISPR/Cas system. Methods for assaying for a modification state of a segment of target RNA can include: contacting a sample comprising the target RNA to: a guide nucleic acid that hybridizes to the segment of the target RNA; a detector nucleic acid; and a programmable nuclease that cleaves the detector nucleic acid upon hybridization of the guide nucleic acid to the segment of the target RNA; and contacting a second sample comprising an RNA having an unmodified segment comprising the same sequence as the segment of the target RNA to: the guide nucleic acid; the detector nucleic acid; and the programmable nuclease that cleaves the detector nucleic acid upon hybridization of the guide nucleic acid to the segment of the unmodified RNA; assaying for a first signal produced by cleavage of the detector nucleic acid in the sample; assaying for a second signal produced by cleavage of the detector nucleic acid in the second sample; and determining the modification state of the target RNA based on a comparison of the first signal to the second signal. The modification state of the segment is modified when the first signal is less than the second signal and the modification state of the segment is unmodified when the first signal is substantially the same as the second signal.

The signal can be the signal from the DETECTR assay, for example, a fluorescence signal. The fluorescence signal can be the fluorescence after background subtraction. In some embodiments, the target RNA can be reverse transcribed into DNA, amplified, and in vitro transcribed back into the target RNA. This can allow for sensitive detection of small amounts of the RNA that may be in the sample.

A method of assaying for or detection can comprise contacting a programmable nuclease that is sensitive to the modification state of a target RNA to a sample comprising the modified RNA. A method of detection can comprise contacting a sample comprising a modified target RNA to an enzyme composition comprising a programmable nuclease, wherein the enzyme composition exhibits cleavage sensitivity to the modification of the target RNA. A method of detection can comprise contacting a sample comprising a modified target RNA to a reagent that differentially reacts to the modified bases of the target RNA and to a programmable nuclease. A method of detection can comprise contacting a CRISPR enzyme that is sensitive to the modification of a target RNA to a sample comprising the modified RNA. A method of detection can comprise contacting a sample comprising a modified target RNA to an enzyme composition comprising a CRISPR enzyme, wherein the enzyme composition exhibits cleavage sensitivity to the modification of the target RNA. A method of detection can comprise contacting a sample comprising a modified target RNA to a reagent that differentially reacts to the modified bases of the target RNA and to a CRISPR enzyme. Detection of RNA having modifications can be used to diagnose or identify diseases associated with the modification of target nucleic acid sequences. Detection of RNA with modifications can be used clinically, in laboratories as a research tool, and in agricultural applications.

The CRISPR/Cas system used to detect a modified target RNA can comprise crRNAs, Cas proteins, and detector nucleic acids.

A crRNA can comprise a sequence that is reverse complementary to the sequence of a target RNA. The crRNA can bind specifically to the target RNA. In some cases, the crRNA is not naturally occurring and made by artificial combination of otherwise separate segments of sequence. The artificial combination can be performed by chemical synthesis, by genetic engineering techniques, or by the artificial manipulation of isolated segments of nucleic acids.

The crRNA and Cas protein can form a CRISPR enzyme. A Cas protein can be any Cas protein with trans cleavage activity upon binding of the crRNA to the target DNA. For example, a Cas protein is a Cas13 nuclease. The Cas13 nuclease can be Cas13a, Cas13b, Cas13c, Cas13d, or Cas13e. Sometimes the Cas protein is a type VI CRISPR-Cas system. In some cases, the Cas protein is from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pint), Capnocytophaga canimorsus (Cca), Porphyromonas gulae (Pgu), Prevotella sp. (Psp), Porphyromonas gingivalis (Pig), Prevotella intermedia (Pini), Enterococcus italicus (E1), Lactobacillus salivarius (Ls), or Thermus thermophilus (Tt). The trans cleavage activity of the CRISPR enzyme can be activated when the crRNA is complexed with the target RNA sequence.

When the crRNAs of the CRISPR enzyme binds to a target RNA, the CRISPR enzyme's trans cleavage activity can be initiated, and detector nucleic acids can be cleaved, resulting in the detection of fluorescence. Detector nucleic acids can comprise a detection moiety, wherein the detector nucleic acid can be cleaved by the activated CRISPR enzyme, thereby generating a detectable fluorescent signal. The generation of the detectable signal from the release of the detection moiety can indicate that cleavage by the CRISPR enzyme has occurred and that the sample contains the target nucleic acid.

As described herein, the CRISPR/Cas system can utilize the trans cleavage abilities of CRISPR enzymes to achieve fast and high-fidelity detection of modified RNA of a target RNA within a sample.

For assaying for or detection of the modified RNA modification state, the target RNA comprising a modification can be contacted with the CRISPR enzyme. The RNA modification, such as m6A, can disrupt interactions between RNA strands, such as those that form hairpins. Therefore, RNA modifications in the target RNA can disrupt the binding of the target RNA to crRNA. Furthermore, specific regions of the crRNA:target RNA interactions can be more sensitive to RNA modification, such as methylation, than other regions. For example, crRNA:target RNA interactions are more sensitive to disruption when the target RNA sequence comprises at least one modification in a region from nucleic acid residue 1 to 4, 5 to 9, or 10 to 15 in the modification variable region or when the target RNA sequence comprises at least one methylation in a region from nucleic acid residue 1 to 4, 5 to 9, or 10 to 15 in the methylation variable region. When the binding of the target RNA to the crRNA is disrupted by a modification in the target RNA, the trans cleavage activity of the CRISPR enzyme cannot be initiated, and detector nucleic acid cannot be cleaved, resulting in the absence of a signal (e.g., fluorescent signal) indicating the presence of the target RNA. The absence of signal (e.g., fluorescent signal) can indicate that the target RNA comprises a modification. Furthermore, while the target RNA comprising a modification cannot bind to the crRNA of the CRISPR enzyme, the target RNA without a modification can bind to the crRNA of the CRISPR enzyme. Therefore, the trans cleavage activity of the CRISPR enzyme can be initiated, and detector nucleic acid can be cleaved, resulting in the detection of a signal (e.g., fluorescent signal) indicating the presence of the target DNA. The detection of the signal (e.g., fluorescent signal) can indicate that the target RNA does not comprise a modification.

The crRNA can have a sequence comprising at least one uracil in a region from nucleic acid residue 5 to 20 that is reverse complementary to a modification variable region in the target RNA. The crRNA, in some cases, has a sequence comprising at least one uracil in a region from nucleic acid residue 5 to 10, 10 to 15, or 15 to 20 that is reverse complementary to a modification variable region in the target RNA. The crRNA can have a sequence comprising at least one uracil in a region from nucleic acid residue 5 to 20 that is reverse complementary to a methylation variable region in the target RNA. The crRNA, in some cases, has a sequence comprising at least one uracil in a region from nucleic acid residue 5 to 10, 10 to 15, or 15 to 20 that is reverse complementary to a methylation variable region in the target RNA.

Additionally, target RNA can be amplified before binding to the crRNA of the CRISPR enzyme. For example, the target RNA can first be reverse transcribed into DNA. Additionally, and/or alternatively, the target RNA can be amplified by primers that enable reverse transcription and amplification. This amplification can be PCR amplification or isothermal amplification. This nucleic acid amplification of the sample can improve at least one of sensitivity, specificity, or accuracy of the detection the target RNA. The reagents for nucleic acid amplification can comprise a recombinase, a oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C. The nucleic acid amplification reaction can be performed at a temperature of at least 20° C., 25° C., 30° C., 35° C., 37° C., 40° C., or 45° C. Amplified DNA can be reverse transcribed back into RNA for detection using the compositions and methods disclosed herein.

Target RNA can be enriched through affinity purification using magnetic beads containing oligonucleotides complementary to the target RNA.

### Detection of Signal

Assaying for a modification state can comprise detecting a signal. Disclosed herein are methods of detecting a nucleic acid modification state using a programmable nuclease system such as the CRISPR/Cas system as discussed above. A modified nucleic acid can be a modified DNA or modified RNA as discussed above. For example, a modified DNA is a methylated DNA or a modified RNA is a methylated RNA. A method of detection can comprise contacting a programmable nuclease that is sensitive to the modification state of a target nucleic acid to a sample comprising the modified nucleic acid. A method of detection can comprise contacting a sample comprising a modified target nucleic acid to an enzyme composition comprising a programmable nuclease, wherein the enzyme composition exhibits cleavage sensitivity to the modification state of the target nucleic acid. A method of detection can comprise contacting a sample comprising a modified target nucleic acid to a reagent that differentially reacts to the modified bases of the target nucleic acid and to a programmable nuclease. A method of detection can comprise contacting a CRISPR enzyme that is sensitive to the modification state of a nucleic acid to a sample comprising a modified nucleic acid. A method of detection can comprise contacting a sample comprising a modified nucleic acid to an enzyme composition comprising a CRISPR enzyme, wherein the enzyme composition exhibits cleavage sensitivity to the modification of the nucleic acid. A method of detection can comprise contacting a sample comprising a modified nucleic acid to a reagent that differentially reacts to modified bases and to a CRISPR enzyme. Detection of nucleic acids having modifications can be used to diagnose or identify diseases associated with the modification state of target nucleic acid sequences. Detection of nucleic acids having modifications such as methylation or other modifications that interfere with endonuclease activity are applicable to a number of fields, such as clinically, as a diagnostic, in laboratories as a research tool, and in agricultural applications.

A programmable nuclease system can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and can generate a signal that is a detectable signal. The detectable signal can be immobilized on a support medium for detection. The detectable signal can be visualized to assess whether a target nucleic acid comprises a modification.

Often, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule in the detection region, where the first detection signal indicates that the sample contained the target nucleic acid. Sometimes the system is capable of detecting more than one type of target nucleic acid, wherein the system comprises more than one type of guide nucleic acid and more than one type of detector nucleic acid. In some cases, the detectable signal is generated directly by the cleavage event. Alternatively, or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium. In some cases, the second detectable signal is generated in a spatially distinct location than the first generated signal.

In some cases, the threshold of detection, for a subject method of detecting a single stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 aM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, fom 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases, the minimum concentration at which a single stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a single stranded target nucleic acid can be detected in a sample is in a range of from 1 aM to 100 pM. In some cases, the minimum concentration at which a single stranded target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the devices, systems, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some cases, the devices, systems, kits, and methods described herein detect a target single-stranded nucleic acid in a sample where the sample is contacted with the reagents for a predetermined length of time sufficient for the trans cleavage to occur or cleavage reaction to reach completion. In some cases, the devices, systems, kits, and methods described herein detect a target single-stranded nucleic acid in a sample where the sample is contacted with the reagents for no greater than 60 minutes. Sometimes the sample is contacted with the reagents for no greater than 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, or 1 minute. Sometimes the sample is contacted with the reagents for at least 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, or 5 minutes.

Some methods as described herein can be a method of detecting a target nucleic acid in a sample comprising contacting the sample comprising the target nucleic acid with a guide nucleic acid targeting a target sequence, a programmable nuclease capable of being activated when complexed with the guide nucleic acid and the target sequence, a single stranded detector nucleic acid comprising a detection moiety, wherein the detector nucleic acid is capable of being cleaved by the activated nuclease, thereby generating a first detectable signal, cleaving the single stranded detector nucleic acid using the programmable nuclease that cleaves as measured by a change in color, and measuring the first detectable signal on the support medium. The cleaving of the single stranded detector nucleic acid using the programmable nuclease may cleave with an efficiency of 50% as measured by a change in color. In some cases, the cleavage efficiency is at least 40%, 50%, 60%, 70%, 80%, 90%, or 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a guide nucleic acid targeting a target sequence, a programmable nuclease capable of being activated when complexed with the guide nucleic acid and the target sequence, and a single stranded detector nucleic acid comprising a detection moiety, wherein the detector nucleic acid is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample.

In some cases, the devices, systems, kits, and methods described herein detect atarget single-stranded nucleic acid with a programmable nuclease and a single-stranded detector nucleic acid in a sample where the sample is contacted with the reagents for a predetermined length of time sufficient for trans cleavage of the single stranded detector nucleic acid. For example, a programmable nuclease is LbuCas13a that detects a target nucleic acid and a single stranded detector nucleic acid comprises two adjacent uracil nucleotides with a green detectable moiety that is detected upon cleavage. As another example, a programmable nuclease is LbaCas13a that detects a target nucleic acid and a single-stranded detector nucleic acid comprises two adjacent adenine nucleotides with a red detectable moiety that is detected upon cleavage.

The methods described herein can also include the use of buffers, which are compatible with the kits and methods disclosed herein. These buffers are compatible with the programmable nuclease system, samples, and support mediums as described herein for detection of a modification state of a target nucleic acid. For example, a buffer comprises 20 mM HEPES pH 6.8, 50 mM KCl, 5 mM MgCl₂, and 5% glycerol. In some instances the buffer comprises from 0 to 100, 0 to 75, 0 to 50, 0 to 25, 0 to 20, 0 to 10, 0 to 5, 5 to 10,5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM HEPES pH 6.8. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer comprises 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl₂. The buffer can comprise 0 to 25, 0 to 20, 0 to 10, 0 to 5, 5 to 10, 5 to 15, 5 to 20, 5 to 25, 5 to 30% glycerol.

As another example, a buffer comprises 100 mM Imidazole pH 7.5; 250 mM KCl, 25 mM MgCl₂, 50 ug/mL BSA, 0.05% Igepal Ca-630, and 25% Glycerol. In some instances the buffer comprises 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM Imidazole pH 7.5. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KC1. In other instances the buffer comprises 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl2. The buffer, in some instances, comprises 0 to 100, 0 to 75, 0 to 50, 0 to 25, 0 to 20, 0 to 10, 0 to 5, 5 to 50, 5 to 75, 5 to 100, 10 to 20, 10 to 50, 10 to 75, 10 to 100, 25 to 50, 25 to 75 25 to 100, 50 to 75, or 50 to 100 ug/mL BSA. In some instances, the buffer comprises 0 to 1, 0 to 0.5, 0 to 0.25, 0 to 0.01, 0 to 0.05, 0 to 0.025, 0 to 0.01, 0.01 to 0.025, 0.01 to 0.05, 0.01 to 0.1, 0.01 to 0.25, 0.01, to 0.5, 0.01 to 1, 0.025 to 0.05, 0.025 to 0.1, 0.025, to 0.5, 0.025 to 1, 0.05 to 0.1, 0.05 to 0.25, 0.05 to 0.5, 0.05 to 0.75, 0.05 to 1, 0.1 to 0.25, 0.1 to 0.5, or 0.1 to 1% Igepal Ca-630. The buffer can comprise 0 to 25, 0 to 20, 0 to 10, 0 to 5, 5 to 10, 5 to 15, 5 to 20, 5 to 25, 5 to 30% glycerol.

A number of detection or visualization devices and methods are consistent with methods disclosed herein. The results from the detection region from a completed assay can be visualized and analyzed in various ways. In some cases, the positive control spot and the detection spot in the detection region is visible by eye, and the results can be read by the user. In some cases, the positive control spot and the detection spot in the detection region is visualized by an imaging device. Often, the imaging device is a digital camera, such a digital camera on a mobile device. The mobile device may have a software program or a mobile application that can capture an image of the support medium, identify the assay being performed, detect the detection region and the detection spot, provide image properties of the detection spot, analyze the image properties of the detection spot, and provide a result. Alternatively, or in combination, the imaging device can capture fluorescence, ultraviolet (UV), infrared (IR), or visible wavelength signals. The imaging device may have an excitation source to provide the excitation energy and captures the emitted signals. In some cases, the excitation source can be a camera flash and optionally a filter. In some cases, the imaging device is used together with an imaging box that is placed over the support medium to create a dark room to improve imaging. The imaging box can be a cardboard box that the imaging device can fit into before imaging. In some instances, the imaging box has optical lenses, mirrors, filters, or other optical elements to aid in generating a more focused excitation signal or to capture a more focused emission signal. Often, the imaging box and the imaging device are small, handheld, and portable to facilitate the transport and use of the assay in remote or low resource settings.

The assay described herein can be visualized and analyzed by a mobile application (app) or a software program. Using the graphic user interface (GUI) of the app or program, an individual can take an image of the support medium, including the detection region, barcode, reference color scale, and fiduciary markers on the housing, using a camera on a mobile device. The program or app reads the barcode or identifiable label for the test type, locate the fiduciary marker to orient the sample, and read the detectable signals, compare against the reference color grid, and determine the presence or absence of the target nucleic acid, which indicates the presence of the gene, virus, or the agent responsible for the disease, cancer, or genetic disorder. The mobile application can present the results of the test to the individual. The mobile application can store the test results in the mobile application. The mobile application can communicate with a remote device and transfer the data of the test results. The test results can be viewable remotely from the remote device by another individual, including a healthcare professional. A remote user can access the results and use the information to recommend action for treatment, intervention, cleanup of an environment.

### Assaying for a Modification State

Disclosed herein are methods of assaying for (e.g., detecting) a nucleic acid modification state (e.g., unmodified or modified) using a programmable nuclease system such as the CRISPR/Cas system. A modified nucleic acid can be a modified DNA or modified RNA. For example, a modified DNA is a methylated DNA or a modified RNA is a methylated RNA. A method of detection can comprise contacting a programmable nuclease that is sensitive to the modification of a target nucleic acid to a sample comprising the modified nucleic acid. A method of detection can comprise contacting a sample comprising a modified target nucleic acid to an enzyme composition comprising a programmable nuclease, wherein the enzyme composition exhibits cleavage sensitivity to the modification of the target nucleic acid. A method of detection can comprise contacting a sample comprising a modified target nucleic acid to a reagent that differentially reacts to the modified bases of the target nucleic acid and to a programmable nuclease. A method of detection can comprise contacting a CRISPR enzyme that is sensitive to the modification of a nucleic acid to a sample comprising a modified nucleic acid. A method of detection can comprise contacting a sample comprising a modified nucleic acid to an enzyme composition comprising a CRISPR enzyme, wherein the enzyme composition exhibits cleavage sensitivity to the modification of the nucleic acid. A method of detection can comprise contacting a sample comprising a modified nucleic acid to a reagent that differentially reacts to modified bases and to a CRISPR enzyme. Detection of nucleic acids having modifications can be used to assess the modification state of a target nucleic acid.

Methods described herein can be used to identify a nucleic acid modification in a target nucleic acid. The methods can be used to identify a modification of a target nucleic acid that affects the expression of a gene. A modification that affects the expression of gene can be a modification of a target nucleic acid within the gene, a modification of a target nucleic acid comprising RNA associated with the expression of a gene, or a target nucleic acid comprising a modification of a nucleic acid associated with regulation of expression of a gene, such as an RNA or a promoter, enhancer, or repressor of the gene. Often, a status of a nucleic acid modification is used to diagnose or identify diseases associated with the modification of target nucleic acid sequences. Detection of nucleic acids having modifications such as methylation or other modifications that interfere with endonuclease activity are applicable to a number of fields, such as clinically, as a diagnostic, in laboratories as a research tool, and in agricultural applications. The methods can be used to identify modifications in synthetic DNA, DNA generated by PCR-based methods, or in vitro transcribed methods.

### Disease Detection

Disclosed herein are methods of assaying for (e.g., detecting) a nucleic acid modification using a programmable nuclease system such as the CRISPR/Cas system. A modified nucleic acid can be a modified DNA or modified RNA. For example, a modified DNA is a methylated DNA or a modified RNA is a methylated RNA. A method of detection can comprise contacting a programmable nuclease that is sensitive to the modification of a target nucleic acid to a sample comprising the modified nucleic acid. A method of detection can comprise contacting a sample comprising a modified target nucleic acid to an enzyme composition comprising a programmable nuclease, wherein the enzyme composition exhibits cleavage sensitivity to the modification of the target nucleic acid. A method of detection can comprise contacting a sample comprising a modified target nucleic acid to a reagent that differentially reacts to the modified bases of the target nucleic acid and to a programmable nuclease. A method of detection can comprise contacting a CRISPR enzyme that is sensitive to the modification of a nucleic acid to a sample comprising a modified nucleic acid. A method of detection can comprise contacting a sample comprising a modified nucleic acid to an enzyme composition comprising a CRISPR enzyme, wherein the enzyme composition exhibits cleavage sensitivity to the modification of the nucleic acid. A method of detection can comprise contacting a sample comprising a modified nucleic acid to a reagent that differentially reacts to modified bases and to a CRISPR enzyme. Detection of nucleic acids having modifications can be used to diagnose or identify diseases associated with the modification of target nucleic acid sequences. Detection of nucleic acids having modifications such as methylation or other modifications that interfere with endonuclease activity are applicable to a number of fields, such as clinically, as a diagnostic, in laboratories as a research tool, and in agricultural applications.

The methods as described herein can be used to identify or diagnose a cancer or genetic disorder associated with a nucleic acid modification in a target nucleic acid. The methods can be used to identify a modification of a target nucleic acid that affects the expression of a cancer gene. A cancer gene can be any gene whose aberrant expression is associated with cancer, such as overexpression of an oncogene, suppression of tumor suppressor gene, or disregulation of a checkpoint inhibitor gene or gene associated with cellular growth, cellular metabolism, or the cell cycle. A modification that affects the expression of cancer gene can be a modification of a target nucleic acid within the cancer gene, a modification of a target nucleic acid comprising RNA associated with the expression of a cancer gene, or a target nucleic acid comprising a modification of a nucleic acid associated with regulation of expression of a cancer gene, such as an RNA or a promoter, enhancer, or repressor of the cancer gene. For example, a target nucleic acid comprising a modification that affects a cancer gene can contribute to or lead to colon cancer, bladder cancer, stomach cancer, breast cancer, non-small-cell lung cancer, pancreatic cancer, esophageal cancer, cervical cancer, ovarian cancer, hepatocellular cancer, and acute myeloid leukemia. The target nucleic acid can be modified DNA of a cancer gene or RNA expressed from a cancer gene. For example, a methylated DNA comprising hypermethylated CpG islands in the TFPI2 promoter indicates gastric/colorectal cancer. A methylated cancer gene can comprise a methylated DNA encoding APC, p16INK4A, or DAPK11, and can indicate lung cancer. A methylated target nucleic acid can comprise a methylated DNA encoding RASSF1A, p16INK4A, or CDH1, and can indicate breast cancer. A methylated target nucleic acid can comprise a methylated target nucleic acid encoding GSTP1 and can indicate prostate cancer. A methylated target nucleic acid can comprise an RNA with misregulated m6A and can indicate breast cancer, glioblastoma, acute myeloid leukemia, lung adenocarcinoma, or endometrial cancer. A methylated target nucleic acid can comprise an RNA with misregulated m6A encoding NANOG. A methylated target nucleic acid can comprise an RNA with misregulated m6A encoding FOXM1. A methylated target nucleic acid can comprise an RNA with misregulated m6A encoding MYC. A methylated target nucleic acid can comprise an RNA with misregulated m6A encoding YAP. A subject with a cancer, such as breast cancer, glioblastoma, acute myeloid leukemia, lung adenocarcinoma, or endometrial cancer, can have 1, more than 1, more than 10, more than 100, more than 200, more than 500, more than 1,000, more than 10,000, more than 100,000, or more than 1,000,000 misregulated m6A RNA transcripts per cell.

The methods can be used to identify a modification that affects the expression of a gene associated with a genetic disorder. A gene associated with a genetic disorder can be a gene whose overexpression is associated with a genetic disorder, from a gene associated with abnormal cellular growth resulting in a genetic disorder, or from a gene associated with abnormal cellular metabolism resulting in a genetic disorder. A modification that affects the expression of a gene associated with a genetic disorder can be a modification within the gene associated with a genetic disorder, a modification of RNA associated with a gene of the genetic disorder, or a modification of a nucleic acid associated with regulation of expression of a gene associated with a genetic disorder, such as an RNA or a promoter, enhancer, or repressor of the gene associated with the genetic disorder. For example, a target nucleic acid can comprise a modification, such as methylation, that affects Parkinson's disease, Rett Syndrome, or Immunodeficiency Centromere instability and Facial anomalies (ICF) Syndrome. The modified nucleic acid can be DNA or RNA. For example, a methylated target DNA comprises hypermethylated CpG islands inSNCA and indicates Parkinson's disease. The methods can be used to identify modifications in synthetic DNA, DNA generated by PCR-based methods, or in vitro transcribed methods.

Methods described herein can be used to identify a nucleic acid modification in a target nucleic acid from a bacteria, virus, or microbe. The methods can be used to identify a modification of a target nucleic acid that affects the expression of a gene. A modification that affects the expression of gene can be a modification of a target nucleic acid within the gene, a modification of a target nucleic acid comprising RNA associated with the expression of a gene, or a target nucleic acid comprising a modification of a nucleic acid associated with regulation of expression of a gene, such as an RNA or a promoter, enhancer, or repressor of the gene. Sometimes, a status of a target nucleic acid modification is used to determine a pathogenicity of a bacteria, virus, or microbe. Often, a status of a nucleic acid modification is used to diagnose or identify diseases associated with the modification of target nucleic acid sequences in the bacteria, virus, or microbe. The methods can be used to identify modifications in synthetic DNA, DNA generated by PCR-based methods, or in vitro transcribed methods.

### Detection as a Research Tool

Disclosed herein are methods of assaying for (e.g., detecting) a nucleic acid modification using a programmable nuclease system such as the CRISPR/Cas system, which can be used in a laboratory and used as a research tool. A modified nucleic acid can be a modified DNA or modified RNA. For example, a modified DNA is a methylated DNA or a modified RNA is a methylated RNA. A method of detection can comprise contacting a programmable nuclease that is sensitive to the modification of a target nucleic acid to a sample comprising the modified nucleic acid. A method of detection can comprise contacting a sample comprising a modified target nucleic acid to an enzyme composition comprising a programmable nuclease, wherein the enzyme composition exhibits cleavage sensitivity to the modification of the target nucleic acid. A method of detection can comprise contacting a sample comprising a modified target nucleic acid to a reagent that differentially reacts to the modified bases of the target nucleic acid and to a programmable nuclease. A method of detection can comprise contacting a CRISPR enzyme that is sensitive to the modification of a nucleic acid to a sample comprising a modified nucleic acid. A method of detection can comprise contacting a sample comprising a modified nucleic acid to an enzyme composition comprising a CRISPR enzyme, wherein the enzyme composition exhibits cleavage sensitivity to the modification of the nucleic acid. A method of detection can comprise contacting a sample comprising a modified nucleic acid to a reagent that differentially reacts to modified bases and to a CRISPR enzyme. Detection of nucleic acids having modifications can be used to diagnose or identify diseases associated with the modification of target nucleic acid sequences. Detection of nucleic acids having modifications such as methylation or other modifications that interfere with endonuclease activity are applicable to a number of fields, such as clinically, as a diagnostic, in laboratories as a research tool, and in agricultural applications.

The methods as described herein can be used to identify a nucleic acid modification in a target nucleic acid. The methods can be used to identify a modification of a target nucleic acid that affects the expression of a gene. A modification that affects the expression of gene can be a modification of a target nucleic acid within the gene, a modification of a target nucleic acid comprising RNA associated with the expression of a gene, or a target nucleic acid comprising a modification of a nucleic acid associated with regulation of expression of a gene, such as an RNA or a promoter, enhancer, or repressor of the gene. The methods can be used to identify modifications in synthetic DNA, DNA generated by PCR-based methods, or in vitro transcribed methods.

### Detection for Agricultural Applications

Disclosed herein are methods of assaying for (e.g., detecting) a nucleic acid modification using a programmable nuclease system such as the CRISPR/Cas system for use in agricultural applications. A modified nucleic acid can be a modified DNA or modified RNA. For example, a modified DNA is a methylated DNA or a modified RNA is a methylated RNA. A method of detection can comprise contacting a programmable nuclease that is sensitive to the modification of a target nucleic acid to a sample comprising the modified nucleic acid. A method of detection can comprise contacting a sample comprising a modified target nucleic acid to an enzyme composition comprising a programmable nuclease, wherein the enzyme composition exhibits cleavage sensitivity to the modification of the target nucleic acid. A method of detection can comprise contacting a sample comprising a modified target nucleic acid to a reagent that differentially reacts to the modified bases of the target nucleic acid and to a programmable nuclease. A method of detection can comprise contacting a CRISPR enzyme that is sensitive to the modification of a nucleic acid to a sample comprising a modified nucleic acid. A method of detection can comprise contacting a sample comprising a modified nucleic acid to an enzyme composition comprising a CRISPR enzyme, wherein the enzyme composition exhibits cleavage sensitivity to the modification of the nucleic acid. A method of detection can comprise contacting a sample comprising a modified nucleic acid to a reagent that differentially reacts to modified bases and to a CRISPR enzyme. Detection of nucleic acids having modifications can be used to diagnose or identify diseases associated with the modification of target nucleic acid sequences. Detection of nucleic acids having modifications such as methylation or other modifications that interfere with endonuclease activity are applicable to a number of fields, such as clinically, as a diagnostic, in laboratories as a research tool, and in agricultural applications.

The methods as described herein can be used to identify a nucleic acid modification in a target nucleic acid of a plant or of a bacteria, virus, or microbe associated with a plant or soil. The methods can be used to identify a modification of a target nucleic acid that affects the expression of a gene. A modification that affects the expression of gene can be a modification of a target nucleic acid within the gene, a modification of a target nucleic acid comprising RNA associated with the expression of a gene, or a target nucleic acid comprising a modification of a nucleic acid associated with regulation of expression of a gene, such as an RNA or a promoter, enhancer, or repressor of the gene. The methods can be used to identify modifications in synthetic DNA, DNA generated by PCR-based methods, or in vitro transcribed methods.

Amplification of ssDNA

Disclosed herein are methods of amplifying ssDNA for use in any of the methods disclosed herein. Reagents comprising a programmable nuclease capable of being complexed with the guide nucleic acid and the target ssDNA are described herein. In some embodiments, the ssDNA may be amplified prior to or concurrent with detection using a CRISPR/Cas system. A ssDNA may be selectively amplified by amplifying ssDNA in a sample. A ssDNA may be selectively amplified by amplifying ssDNA in a sample comprising both ssDNA and dsDNA. A ssDNA may be selectively produced by amplifying ssDNA in a sample. A ssDNA may be selectively produced by amplifying ssDNA in a sample comprising both ssDNA and dsDNA. Selectively producing an ssDNA can be comprise adding amplification reagents to a sample that target a dsDNA or ssDNA in the sample and selectively amplify a target ssDNA segment. This can be achieved through the amplification strategies described herein including, for example, the use of phosphorothioated (PT′d) primers with an exonuclease that specifically degrades non-PT′d amplicons, the use of asymmetric ratios of forward to reverse primers to drive amplification of that target ssDNA, or the use of strand displacement amplification with a set of primers and a strand displacing polymerase. A ssDNA may be detected using the methods or reagents described herein. In some embodiments, an amplified ssDNA may be modified prior to detection using a CRISPR/Cas system, as disclosed herein.

For any of the ssDNA amplification strategies described below, including ssDNA amplification with PT′d primers and exonuclease treatment, asymmetric amplification, and strand displacement amplification, a significant challenge is incorporating the amplification methods with the methods for DETECTR. Selecting the correct components and ratios of the various components used for amplification and DETECTR (e.g., primers, dNTPs, non-CRISPR enzymes, programmable nucleases, polymerases, guide nucleic acids, detector nucleic acids, etc.) is complex and not straightforward,but is crucial to achieving a DETECTR assay that sensitively and specifically detects a target nucleic acid.

ssDNA Amplification with PT′d Primers and Exonuclease Treatment

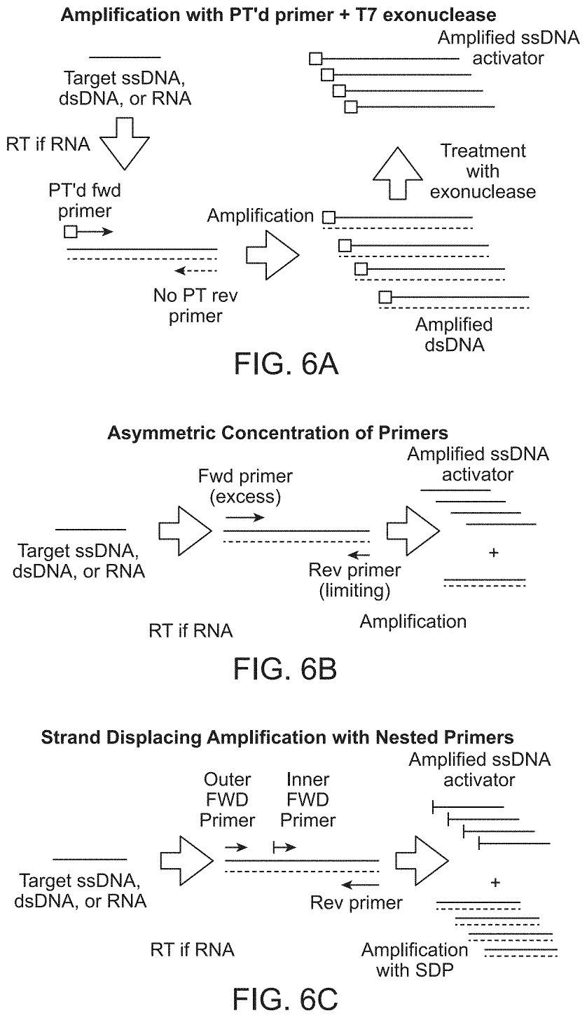

ssDNA amplicons can be generated by amplifying template cDNA, ssDNA, and/or dsDNA with one unmodified primer and one primer whose first four nucleotides on the 5′ end are joined by phosphorothiate bonds, as shown in FIG. 6A. FIG. 6A illustrates a schematic outlining amplification with a phosphorothioated (PT′d) primer followed by treatment with a T7 exonuclease to generate ssDNA amplicons from ssDNA, dsDNA, or RNA. At the top left of the schematic is the target template (e.g., ssDNA, dsDNA, or RNA). If the template is RNA, the RNA is reverse transcribed. One unmodified primer (shown in FIG. 6A) as “No PT rev primer” and one modified, PT′d primer, whose first four nucleotides on the 5′ end are joined by phosphorothiate bonds, are added, which anneal to the target nucleic acid template. Amplification results in amplified target nucleic acid templates (e.g., amplified dsDNA). Amplified target nucleic acid templates are treated with an exonuclease. The exonuclease, while unable to cleave the PT′d strand, will cleave the unmodified strand. The result is the amplified ssDNA activator, which can then be used in the DETECTR system.

The primers used in the present disclosure can be of any length and any number of nucleotides on the 5′ end joined by phosphorothiate bonds to generate the PT′d primer. For example primers consistent with the methods disclosed herein are 18 to 22 nucleotides, 4 to 100 nucleotides, 4 to 8 nucleotides, 8 to 12 nucleotides, 12 to 16 nucleotides, 16 to 20 nucleotides, 20 to 24 nucleotides, 24 to 28 nucleotides, 28 to 32 nucleotides, 32 to 36 nucleotides, 36 to 40 nucleotides, 40 to 44 nucleotides, 44 to 48 nucleotides, 48 to 52 nucleotides, 52 to 56 nucleotides, 56 to 60 nucleotides, 60 to 64 nucleotides, 64 to 68 nucleotides, 68 to 72 nucleotides, 72 to 76 nucleotides, 76 to 80 nucleotides, 80 to 84 nucleotides, 84 to 88 nucleotides, 88 to 92 nucleotides, 92 to 96 nucleotides, 96 to 100 nucleotides, 10 to 100 nucleotides, 20 to 90 nucleotides, 30 to 70 nucleotides, 50 to 70 nucleotides, 15 to 20 nucleotides, 10 to 30 nucleotides, or 15 to 25 nucleotides. Any number of nucleotides on the 5′ end joined by phosphorothiate bonds is also consistent with the methods described herein. For example, the PT′d primer can comprise at least 2 nucleotide joined by phosphorothiate bonds, at least 3 nucleotide joined by phosphorothiate bonds, at least 4 nucleotide joined by phosphorothiate bonds, at least 5 nucleotide joined by phosphorothiate bonds, at least 6 nucleotide joined by phosphorothiate bonds, at least 7 nucleotide joined by phosphorothiate bonds, at least 8 nucleotide joined by phosphorothiate bonds, at least 9 nucleotide joined by phosphorothiate bonds, at least 10 nucleotide joined by phosphorothiate bonds, at least 11 nucleotide joined by phosphorothiate bonds, at least 12 nucleotide joined by phosphorothiate bonds, at least 13 nucleotide joined by phosphorothiate bonds, at least 14 nucleotide joined by phosphorothiate bonds, at least 15 nucleotide joined by phosphorothiate bonds, at least 20 nucleotide joined by phosphorothiate bonds, at least 25 nucleotide joined by phosphorothiate bonds, at least 30 nucleotide joined by phosphorothiate bonds, at least 35 nucleotide joined by phosphorothiate bonds, at least 40 nucleotide joined by phosphorothiate bonds, at least 45 nucleotide joined by phosphorothiate bonds, at least 50 nucleotide joined by phosphorothiate bonds, at least 55 nucleotide joined by phosphorothiate bonds, at least 60 nucleotide joined by phosphorothiate bonds, at least 65 nucleotide joined by phosphorothiate bonds, at least 70 nucleotide joined by phosphorothiate bonds, at least 75 nucleotide joined by phosphorothiate bonds, at least 80 nucleotide joined by phosphorothiate bonds, at least 85 nucleotide joined by phosphorothiate bonds, at least 90 nucleotide joined by phosphorothiate bonds, at least 95 nucleotide joined by phosphorothiate bonds, or at least 100 nucleotide joined by phosphorothiate bonds.

The exonuclease can be any exonuclease that does not cleave PT′d strands, but will otherwise degrade nucleic acids. For example, the exonuclease can comprise a T7 exonuclease, which is a product of T7 Gene6 and can also be referred to as a “T7 Gene 6 exonuclease” or a “T7 (Gene6) Exonuclease”.

This modified primer pair (one PT′d, the other unmodified), can be implemented in many forms of primer-mediated nucleic acid amplification, including thermal amplification techniques such as polymerase chain reaction (PCR) and isothermal techniques such as helicase-dependent amplification (HDA) or circular helicase dependent amplification (cHDA), transcription mediated amplification (TMA), strand displacement amplification (SDA), recombinase polymerase amplification (RPA), loop mediated amplification (LAMP), the exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). Following amplification with this primer pair, the amplified dsDNA will have one strand with an unmodified 5′ end while the other strand has a 5′ end with the PT modification. The amplified dsDNA is then treated by a 5′-3′ exonuclease that is unable to cleave PT′d bonds, such as the T7 Gene 6 exonuclease. Following digestion by this exonuclease, the strand with the unmodified 5′ end will be degraded leaving only the PT′d strand. This PT′d strand is then used for detection by the DETECTR platform.

Provided herein are methods for the generation and detection of ssDNA amplicons in two steps: 1) the initial amplification of the PT-modified dsDNA fragment via PCR or isothermal techniques, and 2) degradation of the unmodified strand by T7 exonuclease and detection of the PT′d strand in the same reaction. The minimal amount of T7 exonuclease necessary for reliable degradation of the unmodified DNA strand in the DETECTR reaction mix is disclosed herein. As shown in FIG. 8B, the addition of 5 U of NEB T7 exonuclease to the DETECTR reaction (total volume of 20 ᵢl.L) is enough to achieve viable detection of the ssDNA activator by Cas14a1. FIG. 8B illustrates the minimum amount of NEB T7 exonuclease added to a 20 μL DETECTR reaction required to achieve a viable fluorescent signal with Cas14a1 DETECTR. As shown in FIG. 8A, 2.5 U of NEB T7 exonuclease allows for PAM-independent detection of ssDNA by LbCas12a. FIG. 8A illustrates the minimum amount of NEB T7 exonuclease added to a 20 μL DETECTR reaction required to achieve a viable fluorescent signal with LbCas12a DETECTR.

The ratio of an exonuclease to the total DETECTR reaction volume can be 1 U exonuclease:0.5 μl total DETECTR reaction volume, 1 U exonuclease:0.25 μl total DETECTR reaction volume, 1 U exonuclease:0.125 μl total DETECTR reaction volume, 1 U exonuclease:0.0625 μl total DETECTR reaction volume, 1 U exonuclease:0.03125 μl total DETECTR reaction volume, 1 U exonuclease:0.015625 μl total DETECTR reaction volume, 1 U exonuclease:0.0078125 μl total DETECTR reaction volume, 1 U exonuclease:0.00390625 μl total DETECTR reaction volume, 1 U exonuclease:0.001953125 μl total DETECTR reaction volume, 1 U exonuclease:0.000976563 μl total DETECTR reaction volume, 1 U exonuclease:0.000488281 pi total DETECTR reaction volume, 1 U exonuclease:0.000244141 μl total DETECTR reaction volume, 1 U exonuclease:0.00012207 μl total DETECTR reaction volume, 1 U exonuclease:1 total DETECTR reaction volume, 1 U exonuclease:1.5 μl total DETECTR reaction volume, 1 U exonuclease:2 μl total DETECTR reaction volume, 1 U exonuclease:2.5 p,1 total DETECTR reaction volume, 1 U exonuclease:3 μl total DETECTR reaction volume, 1 U exonuclease:3.5 total DETECTR reaction volume, 1 U exonuclease:4 μl total DETECTR reaction volume, 1 U exonuclease:4.5 μl total DETECTR reaction volume, 1 U exonuclease:5 μl total DETECTR reaction volume, 1 U exonuclease:5.5 μl total DETECTR reaction volume, 1 U exonuclease:6 total DETECTR reaction volume, 1 U exonuclease:6.5 μl total DETECTR reaction volume, 1 U exonuclease:7 μl total DETECTR reaction volume, 1 U exonuclease:7.5 p,1 total DETECTR reaction volume, 1 U exonuclease:8 μl total DETECTR reaction volume, 1 U exonuclease:8.5 total DETECTR reaction volume, 1 U exonuclease:9 μl total DETECTR reaction volume, 1 U exonuclease:9.5 μl total DETECTR reaction volume, 1 U exonuclease:10 μl total DETECTR reaction volume, 1 U exonuclease:10.5 μl total DETECTR reaction volume, 1 U exonuclease:11 pi total DETECTR reaction volume, 1 U exonuclease:11.5 μl total DETECTR reaction volume, 1 U exonuclease:12 μl total DETECTR reaction volume, 1 U exonuclease:12.5 μl total DETECTR reaction volume, 1 U exonuclease:13 μl total DETECTR reaction volume, 1 U exonuclease:13.5 pi total DETECTR reaction volume, 1 U exonuclease:14 μl total DETECTR reaction volume, 1 U exonuclease:14.5 μl total DETECTR reaction volume, 1 U exonuclease:15 μl total DETECTR reaction volume, 1 U exonuclease:20 μl total DETECTR reaction volume, 1 U exonuclease:25 total DETECTR reaction volume, 1 U exonuclease:30 μl total DETECTR reaction volume, 1 U exonuclease:35 μl total DETECTR reaction volume, 1 U exonuclease:40 μl total DETECTR reaction volume, 1 U exonuclease:45 μl total DETECTR reaction volume, 1 U exonuclease:50 total DETECTR reaction volume, 1 U exonuclease:55 μl total DETECTR reaction volume, 1 U exonuclease:60 μl total DETECTR reaction volume, 1 U exonuclease:65 μl total DETECTR reaction volume, 1 U exonuclease:70 μl total DETECTR reaction volume, 1 U exonuclease:75 total DETECTR reaction volume, 1 U exonuclease:80 μl total DETECTR reaction volume, 1 U exonuclease:85 μl total DETECTR reaction volume, 1 U exonuclease:90 μl total DETECTR reaction volume, 1 U exonuclease:95 μl total DETECTR reaction volume or 1 U exonuclease:100 IA total DETECTR reaction volume. In some embodiments, the ratio of an exonuclease to the total DETECTR reaction volume is 1 U exonuclease:4 μL total DETECTR reaction volume.

In some embodiments, all steps of the process, including amplification of modified dsDNA, degradation of the unmodified strand, and detection of the phosphorothioated (PT′d) strand, can occur simultaneously in one reaction mix. In some embodiments, all steps of the process, including amplification of modified dsDNA, degradation of the unmodified strand, and detection of the phosphorothioated (PT′d) strand, can occur in a common reaction volume (e.g., a single reaction volume).

ssDNA Amplification Using Asymmetric Concentrations of Primers

Amplification by an asymmetric concentration of primers is another DETECTR-compatible method that generates ssDNA (FIG. 6B). FIG. 6B illustrates a schematic exemplifying ssDNA amplification with an asymmetric concentration of primers. As shown from left to right in FIG. 6B, is the target nucleic acid template (e.g., ssDNA, dsDNA, or RNA). If the template is RNA, the RNA is reverse transcribed. An excess amount of forward primer is added and a limiting amount of reverse primer is added. Amplification is carried out and an excess of the amplified ssDNA activator is generated, which can then be used in the DETECTR system.

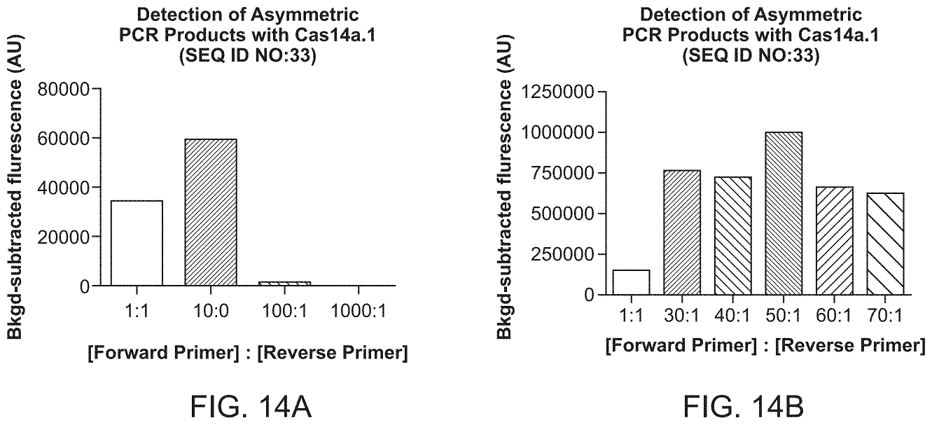

An initial nucleic acid template (e.g., cDNA, ssDNA, or dsDNA) is used as input for a primer-mediated nucleic acid amplification strategy such as PCR or isothermal techniques like recombinase polymerase amplification (RPA). Amplification occurs under standard conditions except for the concentration of primers, in which the primer whose direction matches that of the desired ssDNA amplicon is in excess over the other primer. An excess of one primer will lead to an excess of one DNA strand being amplified. As shown in FIG. 14B, the optimal ratio of primer concentrations is a 50:1 excess of the primer of interest. However, the ratio of primer concentrations is not limited to any particular ratio and may be varied to generate amplified ssDNA activator. For example, the ratio of primer concentrations can be 2:1 excess of the primer of interest, 4:1 excess of the primer of interest, 6:1 excess of the primer of interest, 8:1 excess of the primer of interest, 10:1 excess of the primer of interest, 12:1 excess of the primer of interest, 14:1 excess of the primer of interest, 16:1 excess of the primer of interest, 18:1 excess of the primer of interest, 20:1 excess of the primer of interest, 22:1 excess of the primer of interest, 24:1 excess of the primer of interest, 26:1 excess of the primer of interest, 28:1 excess of the primer of interest, 30:1 excess of the primer of interest, 32:1 excess of the primer of interest, 34:1 excess of the primer of interest, 36:1 excess of the primer of interest, 38:1 excess of the primer of interest, 40:1 excess of the primer of interest, 42:1 excess of the primer of interest, 44:1 excess of the primer of interest, 46:1 excess of the primer of interest, 48:1 excess of the primer of interest, 50:1 excess of the primer of interest, 52:1 excess of the primer of interest, 54:1 excess of the primer of interest, 56:1 excess of the primer of interest, 58:1 excess of the primer of interest, 60:1 excess of the primer of interest, 62:1 excess of the primer of interest, 64:1 excess of the primer of interest, 66:1 excess of the primer of interest, 68:1 excess of the primer of interest, 70:1 excess of the primer of interest, 72:1 excess of the primer of interest, 74:1 excess of the primer of interest, 76:1 excess of the primer of interest, 78:1 excess of the primer of interest, 80:1 excess of the primer of interest, 82:1 excess of the primer of interest, 84:1 excess of the primer of interest, 86:1 excess of the primer of interest, 88:1 excess of the primer of interest, 90:1 excess of the primer of interest, 92:1 excess of the primer of interest, 94:1 excess of the primer of interest, 96:1 excess of the primer of interest, 98:1 excess of the primer of interest, 100:1 excess of the primer of interest, 120:1 excess of the primer of interest, 140:1 excess of the primer of interest, 160:1 excess of the primer of interest, 180:1 excess of the primer of interest, 200:1 excess of the primer of interest, 300:1 excess of the primer of interest, 400:1 excess of the primer of interest, or 500:1 excess of the primer of interest.

2 uL of the amplified product, which largely consists of the ssDNA amplicon, is then transferred to the DETECTR reaction mix for detection. In some embodiments, amplification and detection can be separated into two steps. In other embodiments, amplification (e.g., isothermal amplification) of ssDNA and detection by Cas14a1 can be carried out in the same reaction.

The primers used in the present disclosure can be of any length For example primers consistent with the methods disclosed herein are 18 to 22 nucleotides, 4 to 100 nucleotides, 4 to 8 nucleotides, 8 to 12 nucleotides, 12 to 16 nucleotides, 16 to 20 nucleotides, 20 to 24 nucleotides, 24 to 28 nucleotides, 28 to 32 nucleotides, 32 to 36 nucleotides, 36 to 40 nucleotides, 40 to 44 nucleotides, 44 to 48 nucleotides, 48 to 52 nucleotides, 52 to 56 nucleotides, 56 to 60 nucleotides, 60 to 64 nucleotides, 64 to 68 nucleotides, 68 to 72 nucleotides, 72 to 76 nucleotides, 76 to 80 nucleotides, 80 to 84 nucleotides, 84 to 88 nucleotides, 88 to 92 nucleotides, 92 to 96 nucleotides, 96 to 100 nucleotides, 10 to 100 nucleotides, 20 to 90 nucleotides, 30 to 70 nucleotides, 50 to 70 nucleotides, 15 to 20 nucleotides, 10 to 30 nucleotides, or 15 to 25 nucleotides.

ssDNA Amplification Using Strand-Displacing Polymerase and Nested Primer Design

Another amplification method described herein implements a strand-displacing polymerase and nested primer design to enable Cas14a1 detection of an initial nucleic acid template (e.g., cDNA, ssDNA, dsDNA) (FIG. 6C) to generate ssDNA for use with DETECTR systems. FIG. 6C illustrates a schematic demonstrating ssDNA amplification with a strand displacing polymerase and nested forward primers. From left to right, FIG. 6C shows the target template (e.g., ssDNA, dsDNA, or RNA). If the template is RNA, the RNA is reverse transcribed. An outer forward primer an inner forward primer, and a reverse primer can be added. Amplification with strand displacing polymerase (SDP) can be carried out.

This strategy can involve three primers: two forward primers and one reverse primer. ssDNA amplification occurs when the strand displacing polymerase is replicating DNA simultaneously from both forward primer sites. In this case, the polymerase replicating DNA from the outermost primer site will displace the DNA strand generated by the polymerase replicating from the innermost primer site, creating two products: dsDNA generated by the outer/reverse primer combo, and the desired ssDNA amplicon (the displacement product). Several strand-displacing polymerases exist for isothermal amplification, such as the polymerase from the bacteriophage phi29. Following amplification with this polymerase and primer design, 2 μL of the amplified product is then transferred the DETECTR reaction mix.

The two forward primers and the one reverse primer used in this method can be of any length. For example primers consistent with the methods disclosed herein are 18 to 22 nucleotides, 4 to 100 nucleotides, 4 to 8 nucleotides, 8 to 12 nucleotides, 12 to 16 nucleotides, 16 to 20 nucleotides, 20 to 24 nucleotides, 24 to 28 nucleotides, 28 to 32 nucleotides, 32 to 36 nucleotides, 36 to 40 nucleotides, 40 to 44 nucleotides, 44 to 48 nucleotides, 48 to 52 nucleotides, 52 to 56 nucleotides, 56 to 60 nucleotides, 60 to 64 nucleotides, 64 to 68 nucleotides, 68 to 72 nucleotides, 72 to 76 nucleotides, 76 to 80 nucleotides, 80 to 84 nucleotides, 84 to 88 nucleotides, 88 to 92 nucleotides, 92 to 96 nucleotides, 96 to 100 nucleotides, 10 to 100 nucleotides, 20 to 90 nucleotides, 30 to 70 nucleotides, 50 to 70 nucleotides, 15 to 20 nucleotides, 10 to 30 nucleotides, or 15 to 25 nucleotides. In some embodiments, the outer forward primer is 4 to 6 nucleotides, 6 to 8 nucleotides, 8 to 10 nucleotides, 10 to 12 nucleotides, 12 to 14 nucleotides, 14 to 16 nucleotides, 16 to 18 nucleotides, 18 to 20 nucleotides, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides.

### Programmable Nuclease Reactions

The programmable nuclease reaction conditions can be the same for all three ssDNA generation/amplification strategies outlined above, with the exception of the aforementioned addition of T7 exonuclease in the PT′d primer strategy. 2 uL of the amplification product can be transferred to a 384 well plate and combined directly in the plate with the programmable reaction mix. The programmable nuclease reaction can be the same as the DETECTR reaction conditions in which the DETECTR reaction conditions can be the same for all three ssDNA generation/amplification strategies outlined above, with the exception of the aforementioned addition of T7 exonuclease in the PT′d primer strategy. 2 uL of the amplification product can be transferred to a 384 well plate and combined directly in the plate with the DETECTR reaction mix. The concentrations of the various reagents in the programmable nuclease DETECTR reaction mix can vary depending on the particular scale of the reaction.

For example, the final concentration of the programmable nuclease can vary from 1 pM to 1 nM, from 1 pM to 10 pM, from 10 pM to 100 pM, from 100 pM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM. The final concentration of the sgRNA complementary to the target nucleic acid can be from 1 pM to 1 nM, from 1 pM to 10 pM, from 10 pM to 100 pM, from 100 pM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM. The concentration of the ssDNA-FQ reporter can be from from 1 pM to 1 nM, from 1 pM to 10 pM, from 10 pM to 100 pM, from 100 pM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM.

An exemplary Cas14a1 DETECTR reaction consists of a final concentration of 100nM Cas14a1, 125nM sgRNA, and 50 nM ssDNA-FQ reporter in a total reaction volume of 20 μL. The LbCas12a DETECTR reaction consists of a final concentration of 50 nM LbCas12a, 50 nM sgRNA, and 50 nM ssDNA-FQ reporter in a total reaction volume of 20 μL. Reactions are incubated in a fluorescence plate reader (Tecan Infinite Pro 200 M Plex) for 2 hours at 37° C. with fluorescence measurements taken every 30 seconds (e.g., λex: 485 nm; λem: 535 nm). The fluorescence wavelength detected can vary depending on the reporter molecule.

Described herein are reagents comprising a single stranded detector nucleic acid comprising a detection moiety, wherein the detector nucleic acid (e.g., the ssDNA-FQ reporter described above) is capable of being cleaved by the ssDNA-activated programmable nuclease, upon generation and amplification of ssDNA from a nucleic acid template using the methods disclosed herein, thereby generating a first detectable signal.

The methods disclosed herein, thus, include generation and amplification of ssDNA from a target nucleic acid template (e.g., cDNA, ssDNA, or dsDNA) of interest in a sample, incubation of the ssDNA with an ssDNA activated programmable nuclease leading to indiscriminate, PAM-independent cleavage of detector nucleic acids (also referred to as ssDNA-FQ reporters) to generate a detectable signal, and quantification of the detectable signal to detect a target nucleic acid sequence of interest. In some embodiments, one or more steps of the methods disclosed herein may occur simultaneously in a common reaction volume (e.g., a single reaction mixture). For example, one or more of amplification of a nucleic acid, incubation of a sample with a programmable nuclease, and cleavage of a detector nucleic acid to produce a detectable signal may occur together in a common reaction volume.

As used herein, a detector nucleic acid is used interchangeably with reporter or reporter molecule. In some cases, the detector nucleic acid is a single-stranded nucleic acid sequence comprising deoxyribonucleotides. In other cases, the detector nucleic acid is a single-stranded nucleic acid sequence comprising ribonucleotides. The detector nucleic acid can be a single-stranded nucleic acid sequence comprising at least one deoxyribonucleotide and at least one ribonucleotide. In some cases, the detector nucleic acid is a single-stranded nucleic acid comprising at least one ribonucleotide residue at an internal position that functions as a cleavage site. In some cases, the detector nucleic acid comprises at least 2, 3, 4, 5, 6, 7, 8, 9, or 10 ribonucleotide residues at an internal position. Sometimes the ribonucleotide residues are continuous. Alternatively, the ribonucleotide residues are interspersed in between non-ribonucleotide residues. In some cases, the detector nucleic acid has only ribonucleotide residues. In some cases, the detector nucleic acid has only deoxyribonucleotide residues. In some cases, the detector nucleic acid comprises nucleotides resistant to cleavage by the programmable nuclease described herein. In some cases, the detector nucleic acid comprises synthetic nucleotides. In some cases, the detector nucleic acid comprises at least one ribonucleotide residue and at least one non-ribonucleotide residue. In some cases, detector nucleic acid is 5-20, 5-15, 5-10, 7-20, 7-15, or 7-10 nucleotides in length. In some cases, the detector nucleic acid comprises at least one uracil ribonucleotide. In some cases, the detector nucleic acid comprises at least two uracil ribonucleotides. Sometimes the detector nucleic acid has only uracil ribonucleotides. In some cases, the detector nucleic acid comprises at least one adenine ribonucleotide. In some cases, the detector nucleic acid comprises at least two adenine ribonucleotide. In some cases, the detector nucleic acid has only adenine ribonucleotides. In some cases, the detector nucleic acid comprises at least one cytosine ribonucleotide. In some cases, the detector nucleic acid comprises at least two cytosine ribonucleotide. In some cases, the detector nucleic acid comprises at least one guanine ribonucleotide. In some cases, the detector nucleic acid comprises at least two guanine ribonucleotide. A detector nucleic acid can comprise only unmodified ribonucleotides, only unmodified deoxyribonucleotides, or a combination thereof In some cases, the detector nucleic acid is from 5 to12 nucleotides in length. In some cases, the detector nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some cases, the detector nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by Cas13, a detector nucleic acid can be 5, 8, or 10 nucleotides in length. For cleavage by Cas12, a detector nucleic acid can be 10 nucleotides in length.

The single stranded detector nucleic acid comprises a detection moiety capable of generating a first detectable signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the detector nucleic acid. Sometimes the detection moiety is at the 3′ terminus of the detector nucleic acid. In some cases, the detection moiety is at the 5′ terminus of the detector nucleic acid. In some cases, the quenching moiety is at the 3′ terminus of the detector nucleic acid. In some cases, the single-stranded detector nucleic acid is at least one population of the single-stranded nucleic acid capable of generating a first detectable signal. In some cases, the single-stranded detector nucleic acid is a population of the single stranded nucleic acid capable of generating a first detectable signal. Optionally, there is more than one population of single-stranded detector nucleic acid. In some cases, there are 2, 3, 4, 5, 6, 7, 8, 9, 10, 11, 12, 13, 14, 15, 20, 30, 40, or 50 different populations of single-stranded detector nucleic acids capable of generating a detectable signal.

A detection moiety can be an infrared fluorophore. A detection moiety can be a fluorophore that emits fluorescence in the range of from 500 nm and 720 nm. A detection moiety can be a fluorophore that emits fluorescence in the range of from 500 nm and 720 nm. In some cases, the detection moiety emits fluorescence at a wavelength of 700 nm or higher. In other cases, the detection moiety emits fluorescence at about 660 nm or about 670 nm. In some cases, the detection moiety emits fluorescence at in the range of from 500 to 520, from 500 to 540, from 500 to 590, from 590 to 600, from 600 to 610, from 610 to 620, from 620 to 630, from 630 to 640, from 640 to 650, from 650 to 660, from 660 to 670, from 670 to 680, from 680 to 690, from 690 to 700, from 700 to 710, from 710 to 720, or from 720 to 730 nm. A detection moiety can be a fluorophore that emits fluorescence in the same range as 6-Fluorescein, IRDye 700, TYE 665, Alex Fluor, or ATTO TM 633 (NHS Ester). A detection moiety can be fluorescein amidite, 6-Fluorescein, IRDye 700, TYE 665, Alex Fluor 594, or ATTO TM 633 (NHS Ester). A detection moiety can be a fluorophore that emits fluorescence in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A detection moiety can be fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Any of the detection moieties described herein can be from any commercially available source, can be an alternative with a similar function, a generic, or a non-tradename of the detection moieties listed.

A quenching moiety can be chosen based on its ability to quench the detection moiety. A quenching moiety can be a non-fluorescent fluorescence quencher. A quenching moiety can quench a detection moiety that emits fluorescence in the range of from 500 nm and 720 nm. A quenching moiety can quench a detection moiety that emits fluorescence in the range of from 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety emits fluorescence at in the range of from 500 to 520, from 500 to 540, from 500 to 590, from 590 to 600, from 600 to 610, from 610 to 620, from 620 to 630, from 630 to 640, from 640 to 650, from 650 to 660, from 660 to 670, from 670 to 680, from 680 to 690, from 690 to 700, from 700 to 710, from 710 to 720, or from 720 to 730 nm. A quenching moiety can quench fluorescein amidite, 6-Fluorescein, IRDye 700, TYE 665, Alex Fluor 594, or ATTO TM 633 (NHS Ester). A quenching moiety can be Iowa Black RQ, Iowa Black FQ or IRDye QC-1 Quencher. A quenching moiety can quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety can be Iowa Black RQ (Integrated DNA Technologies), Iowa Black FQ (Integrated DNA Technologies) or IRDye QC-1 Quencher (LiCor). Any of the quenching moieties described herein can be from any commercially available source, can be an alternative with a similar function, a generic, or a non-tradename of the quenching moieties listed.

The generation of the detectable signal from the release of the detection moiety indicates that cleavage by the programmable nuclease has occurred and that the sample contains the target nucleic acid. In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively or in combination, the detection moiety comprises a polypeptide. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

The detectable signal can be a colorimetric signal or a signal visible by eye. In some instances, the detectable signal can be fluorescent, electrical, chemical, electrochemical, or magnetic. In some cases, the first detection signal can be generated by binding of the detection moiety to the capture molecule in the detection region, where the first detection signal indicates that the sample contained the target nucleic acid. Sometimes the system can be capable of detecting more than one type of target nucleic acid, wherein the system comprises more than one type of guide nucleic acid and more than one type of detector nucleic acid. In some cases, the detectable signal can be generated directly by the cleavage event. Alternatively or in combination, the detectable signal can be generated indirectly by the signal event. Sometimes the detectable signal is not a fluorescent signal. In some instances, the detectable signal can be a colorimetric or color-based signal. In some cases, the detected target nucleic acid can be identified based on its spatial location on the detection region of the support medium. In some cases, the second detectable signal can be generated in a spatially distinct location than the first generated signal.

In some cases, the threshold of detection, for a subject method of detecting a single stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to: 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 aM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, fom 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases, the minimum concentration at which a single stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a single stranded target nucleic acid can be detected in a sample is in a range of from 1 aM to 100 pM. In some cases, the minimum concentration at which a single stranded target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some cases, the devices, systems, fluidic devices, kits, and methods described herein detect a target single-stranded nucleic acid in a sample where the sample is contacted with the reagents for a predetermined length of time sufficient for the trans cleavage to occur or cleavage reaction to reach completion. In some cases, the devices, systems, fluidic devices, kits, and methods described herein detect a target single-stranded nucleic acid in a sample where the sample is contacted with the reagents for no greater than 60 minutes. Sometimes the sample is contacted with the reagents for no greater than: 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, or 1 minute. Sometimes the sample is contacted with the reagents for at least 120 minutes, at least 110 minutes, at least 100 minutes, at least 90 minutes, at least 80 minutes, at least 70 minutes, at least 60 minutes, at least 55 minutes, at least 50 minutes, at least 45 minutes, at least 40 minutes, at least 35 minutes, at least 30 minutes, at least 25 minutes, at least 20 minutes, at least 15 minutes, at least 10 minutes, or at least 5 minutes.

Some methods as described herein can be a method of detecting a target nucleic acid in a sample comprising contacting the sample comprising the target nucleic acid with a guide nucleic acid targeting a target sequence, a programmable nuclease capable of being activated when complexed with the guide nucleic acid and the target sequence, a single stranded detector nucleic acid comprising a detection moiety, wherein the detector nucleic acid is capable of being cleaved by the activated nuclease, thereby generating a first detectable signal, cleaving the single stranded detector nucleic acid using the programmable nuclease that cleaves as measured by a change in color, and measuring the first detectable signal on the support medium. The cleaving of the single stranded detector nucleic acid using the programmable nuclease may cleave with an efficiency of 50% as measured by a change in color. In some cases, the cleavage efficiency is at least 40%, at least 50%, at least 60%, at least 70%, at least 80%, at least 90%, or at least 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a guide nucleic acid targeting a target sequence, a programmable nuclease capable of being activated when complexed with the guide nucleic acid and the target sequence, and a single stranded detector nucleic acid comprising a detection moiety, wherein the detector nucleic acid is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample.

### Sample

A number of samples are consistent with the compositions and methods disclosed herein. Samples can comprise the target nucleic acid sequence for detection of an ailment, such as a disease, cancer, or genetic disorder, or genetic information, such as for phenotyping, genotyping, or determining ancestry. Generally, a sample from an individual or an animal or an environmental sample can be obtained to test for presence of a disease, cancer, or genetic disorder. A biological sample from the individual may be blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. A tissue sample may be dissociated, homogenized, or liquified prior to use with the compositions and methods of the present disclosure. A sample from an environment can be from soil, air, or water. In some instances, the environmental sample is taken as a swab from a surface of interest or taken directly from the surface of interest. In some instances, the sample is diluted with a buffer or a fluid or concentrated prior to application to the detection system or be applied neat to the detection system. In some instances, the sample is taken from single-cell eukaryotic organisms; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

The sample used for disease testing can comprise at least one target sequence that can bind to a guide nucleic acid of the reagents described herein. In some cases, the target sequence is a portion of a nucleic acid sequence. A portion of a nucleic acid sequence can be from a genomic locus, a transcribed mRNA, or a reverse transcribed cDNA. A portion of a nucleic acid sequence can be from 5 to 100, from 5 to 90, from 5 to 80, from 5 to 70, from 5 to 60, from 5 to 50, from 5 to 40, from 5 to 30, from 5 to 25, from 5 to 20, from 5 to 15, or from 5 to 10 nucleotides in length. A portion of a nucleic acid sequence can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target sequence can be reverse complementary to a guide nucleic acid sequence.

In some cases, the target sequence is a portion of a nucleic acid from a virus or a bacterium or other agents responsible for a disease in the sample. The target sequence, in some cases, is a portion of a nucleic acid from a sexually transmitted infection or a contagious disease, in the sample. These diseases can include but are not limited to human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens can include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses can include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens can include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M hyorhinis, M orale, M arginini, Acholeplasma laidlawii, M salivarium and M. pneumoniae. Often the target nucleic acid comprises a sequence from a virus or a bacterium or other agents responsible for a disease that can be found in the sample. In some cases, the target sequence is a portion of a nucleic acid from a genomic locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens can include viruses, fungi, helminths, protozoa, malarial parasites, Plasmodium parasites, Toxoplasma parasites, and Schistosoma parasites. Helminths can include roundworms, heartworms, and phytophagous nematodes, flukes, Acanthocephala, and tapeworms. Protozoan infections can include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens can include, but are not limited to: Plasmodium falciparum, P. vivax, Trypanosoma cruzi and Toxoplasma gondii. Fungal pathogens can include, but are not limited to Cryptococcus neoformans, Histoplasma capsulatum, Coccidioides immitis, Blastomyces dermatitidis, Chlamydia trachomatis, and Candida albicans. Pathogenic viruses can include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as antibiotic treatment.

The sample used for cancer testing may comprise at least one target sequence that can bind to a guide nucleic acid of the reagents described herein. The target sequence, in some cases, is a portion of a nucleic acid from a gene with a mutation associated with cancer, from a gene whose overexpression is associated with cancer, a tumor suppressor gene, an oncogene, a checkpoint inhibitor gene, a gene associated with cellular growth, a gene associated with cellular metabolism, or a gene associated with cell cycle. In some cases, the target sequence is a portion of a nucleic acid from a genomic locus, a transcribed mRNA, or a reverse transcribed cDNA from a locus. Non-limiting example include HER2, HERC2, ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1.

The sample used for genetic disorder testing may comprise at least one target sequence that can bind to a guide nucleic acid of the reagents described herein. The target sequence, in some cases, is a portion of a nucleic acid from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target sequence is a portion of a nucleic acid from a genomic locus, a transcribed mRNA, or a reverse transcribed cDNA from a locus. Non-limiting examples include CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUF S6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The sample used for phenotyping testing may comprise at least one target sequence that can bind to a guide nucleic acid of the reagents described herein. The target sequence, in some cases, is a portion of a nucleic acid from a gene associated with a phenotypic trait.

The sample used for genotyping testing may comprise at least one target sequence that can bind to a guide nucleic acid of the reagents described herein. The target sequence, in some cases, is a portion of a nucleic acid from a gene associated with a genotype.

The sample used for ancestral testing may comprise at least one target sequence that can bind to a guide nucleic acid of the reagents described herein. The target sequence, in some cases, is a portion of a nucleic acid from a gene associated with a geographic region of origin or ethnic group.

In some instances, the target nucleic acid is a single stranded nucleic acid. Alternatively or in combination, the target nucleic acid is a double stranded nucleic acid and is prepared into single stranded nucleic acids before or upon contacting the reagents. The target nucleic acid may be a RNA, DNA, synthetic nucleic acids, or nucleic acids found in biological or environmental samples. The target nucleic acids include but are not limited to mRNA, rRNA, tRNA, non-coding RNA, long non-coding RNA, and microRNA (miRNA). In some cases, the target nucleic acid is mRNA. In some cases, the target nucleic acid is from a virus, a parasite, or a bacterium described herein. In some cases, the target nucleic acid is transcribed from a gene as described herein.

A number of target nucleic acids are consistent with the methods and compositions disclosed herein. Some methods described herein can detect a target nucleic acid present in the sample in various concentrations or amounts as a target nucleic acid population. In some cases, the sample has at least 2 target nucleic acids. In some cases, the sample has at least 3, at least 5, at least 10, at least 20, at least 30, at least 40, at least 50, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, or at least 10000 target nucleic acids. In some cases, the method detects target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

A number of target nucleic acid populations are consistent with the methods and compositions disclosed herein. Some methods described herein can detect two or more target nucleic acid populations present in the sample in various concentrations or amounts. In some cases, the sample has at least 2 target nucleic acid populations. In some cases, the sample has at least 3, at least 4, at least 5, at least 6, at least 7, at least 8, at least 9, at least 10, at least 20, at least 30, at least 40, or at least 50 target nucleic acid populations. In some cases, the method detects target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

### Support Medium

Disclosed herein are methods of detecting a nucleic acid using a programmable nuclease system such as the CRISPR/Cas system as discussed above, which can be performed on a support medium. The methods of assaying or detecting may detect a status of a nucleic acid modification. A modified nucleic acid can be a modified DNA or modified RNA. For example, a modified DNA is a methylated DNA or a modified RNA is a methylated RNA. A method of detection can comprise contacting a programmable nuclease that is sensitive to the modification of a segment of the target nucleic acid to a sample comprising the modified nucleic acid. A method of assaying or detection can comprise contacting a sample comprising a modified target nucleic acid to an enzyme composition comprising a programmable nuclease, wherein the enzyme composition exhibits cleavage sensitivity to the modification of the target nucleic acid. A method of detection can comprise contacting a sample comprising a modified target nucleic acid to a reagent that differentially reacts to the modified bases of the target nucleic acid and to a programmable nuclease. A method of detection can comprise contacting a CRISPR enzyme that is sensitive to the modification of a nucleic acid to a sample comprising a modified nucleic acid on a support medium. A method of detection can comprise contacting a sample comprising a modified nucleic acid to an enzyme composition comprising a CRISPR enzyme, wherein the enzyme composition exhibits cleavage sensitivity to the modification of the nucleic acid on a support medium. A method of detection can comprise contacting a sample comprising a modified nucleic acid to a reagent that differentially reacts to modified bases and to a CRISPR enzyme on a support medium. Detection of nucleic acids having modifications on a support medium can be used to diagnose or identify diseases associated with the modification of target nucleic acid sequences. Detection of nucleic acids having modifications such as methylation or other modifications that interfere with endonuclease activity on a support medium are applicable to a number of fields, such as clinically, as a diagnostic, in laboratories as a research tool, and in agricultural applications. In some embodiments, one or more steps of detecting a nucleic acid having a modification may be performed in a common reaction volume (e.g., a single reaction mixture).

The methods of assaying for or detecting a nucleic acid using a programmable nuclease system which can be performed on a support medium may detect a ssDNA. The methods of detecting may detect an amplified ssDNA. Methods and reagents for amplifying and detecting a ssDNA are disclosed herein. A method of detection can comprise contacting a programmable nuclease that is capable of complexing with a ssDNA and a guide nucleic acid to activate trans cleavage activity. A method of detection can comprise contacting a sample comprising a target ssDNA to an enzyme composition comprising a programmable nuclease, wherein the enzyme composition exhibits cleavage sensitivity to the sequence of the ssDNA. A method of detection can comprise contacting a CRISPR enzyme that is sensitive to the sequence of a ssDNA to a sample comprising a ssDNA on a support medium. A method of detection can comprise contacting a sample comprising a modified nucleic acid to an enzyme composition comprising a CRISPR enzyme, wherein the enzyme composition exhibits cleavage sensitivity to the sequence of the ssDNA on a support medium. Detection of ssDNA on a support medium can be used to diagnose or identify diseases associated with the sequence of the ssDNA.

A number of support mediums are consistent with the methods disclosed herein. These support mediums are compatible with methods described herein for detection of a target nucleic acid or nucleic acid modification. A support medium described herein can provide a way to present the results from the activity between the target nucleic acid and the programmable nuclease system described herein. The support medium provides a medium to present the detectable signal in a detectable format. Optionally, the support medium concentrates the detectable signal to a detection spot in a detection region to increase the sensitivity, specificity, or accuracy of the assay. The support mediums can present the results of the assay and indicate the presence or absence of the target nucleic acid. The support mediums can present the results of the assay and provide the status of the modification of the target nucleic acid. The result on the support medium can be read by eye or using a machine. The support medium helps to stabilize the detectable signal generated by the cleaved detector molecule on the surface of the support medium. In some instances, the support medium is a lateral flow assay strip. In some instances, the support medium is a PCR plate. The PCR plate can have 96 wells or 384 wells. The PCR plate can have a subset number of wells of a 96 well plate or a 384 well plate. A subset number of wells of a 96 well PCR plate is, for example, 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, or 95 wells. For example, a PCR subset plate can have 4 wells wherein a well is the size of a well from a 96 well PCR plate (e.g., a 4 well PCR subset plate wherein the wells are the size of a well from a 96 well PCR plate). A subset number of wells of a 384 well PCR plate is, for example, 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, or 380 wells. For example, a PCR subset plate can have 20 wells wherein a well is the size of a well from a 384 well PCR plate (e.g., a 20 well PCR subset plate wherein the wells are the size of a well from a 384 well PCR plate). The PCR plate or PCR subset plate can be paired with a fluorescent light reader, a visible light reader, or other imaging device. Often, the imaging device is a digital camera, such a digital camera on a mobile device. The mobile device can have a software program or a mobile application that can capture an image of the PCR plate or PCR subset plate, identify the assay being performed, detect the individual wells and the sample therein, provide image properties of the individuals wells comprising the assayed sample, analyze the image properties of the contents of the individual wells, and provide a result.

The support medium can have at least one specialized zone or region to present the detectable signal. The regions can comprise at least one of a sample pad region, a nucleic acid amplification region, a conjugate pad region, a detection region, and a collection pad region. In some instances, the regions are overlapping completely, overlapping partially, or in series and in contact only at the edges of the regions, where the regions are in fluid communication with its adjacent regions. The support medium can have a sample pad located upstream of the other regions; a conjugate pad region having a means for specifically labeling the detector moiety; a detection region located downstream from sample pad; and at least one matrix which defines a flow path in fluid connection with the sample pad. In some instances, the support medium has an extended base layer on top of which the various zones or regions are placed. The extended base layer can provide a mechanical support for the zones.

Described herein are sample pad that can provide an area to apply the sample to the support medium. The sample can be applied to the support medium by a dropper or a pipette on top of the sample pad, by pouring or dispensing the sample on top of the sample pad region, or by dipping the sample pad into a reagent chamber holding the sample. The sample can be applied to the sample pad prior to reaction with the reagents when the reagents are placed on the support medium or be reacted with the reagents prior to application on the sample pad. The sample pad region can transfer the reacted reagents and sample into the other zones of the support medium. Transfer of the reacted reagents and sample can be by capillary action, diffusion, convection or active transport aided by a pump. The support medium can be integrated with or overlaid by microfluidic channels to facilitate the fluid transport.

The dropper or the pipette can dispense a predetermined volume. The predetermined volume can range from about 1 μl to about 1000 μl, about 1 μl to about 500μ1, about 1 μl to about 100 μl, or about 1 μl to about 50 μl. The predetermined volume can be at least 1 μl, 2 μl, 3 μl, 4 μl, 5 μl, 6 μl, 7 μl, 8 μl, 9 μl, 10 μl, 25 μl, 50 μl, 75 μl, 100 μl, 250 μl, 500 μl, 750 μl, or 1000 μl. The predetermined volume can be no more than 5 μl, 10 μl, 25 μl, 50 μl, 75 μl, 100 μl, 250 μl, 500 μl, 750 μl, or 1000 μl. The dropper or the pipette can be disposable or be single-use.

Optionally, a buffer or a fluid can also be applied to the sample pad to help drive the movement of the sample along the support medium. The volume of the buffer or the fluid can range from about 1 μl to about 1000 μl, about 1 μl to about 500 μl, about 1 μl to about 100 μl, or about 1 μl to about 50 μl. The volume of the buffer or the fluid can be at least 1 μl, 2 μl, 3 μl, 4 μl, 5 μl, 6 μl, 7 μl, 8 μl, 9 μl, 10 μl, 25 μl, 50 μl, 75 μl, 100 μl, 250 μl, 500 μl, 750 μl, or 1000 μl. The volume of the buffer or the fluid can be no more than 5 μl, 10 μl, 25 μl, 50 μl, 75 μl, 100 μl, 250 μl, 500 μl, 750 μl, or 1000 μl. The buffer or fluid can have a ratio of the sample to the buffer or fluid of at least 1:1, 1:2, 1:3, 1:4, 1:5, 1:6, 1:7, 1:8, 1:9, or 1:10.

The sample pad can be made from various materials that transfer most of the applied reacted reagents and samples to the subsequent regions. The sample pad may comprise cellulose fiber filters, woven meshes, porous plastic membranes, glass fiber filters, aluminum oxide coated membranes, nitrocellulose, paper, polyester filter, or polymer-based matrices. The material for the sample pad region may be hydrophilic and have low non-specific binding. The material for the sample pad may range from about 50 μm to about 1000 μm, about 50 μm to about 750 μm, about 50 μm to about 500 μm, or about 100 μm to about 500 μm.

The sample pad can be treated with chemicals to improve the presentation of the reaction results on the support medium. The sample pad can be treated to enhance extraction of nucleic acid in the sample, to control the transport of the reacted reagents and sample or the conjugate to other regions of the support medium, or to enhance the binding of the cleaved detection moiety to the conjugate binding molecule on the surface of the conjugate or to the capture molecule in the detection region. The chemicals may comprise detergents, surfactants, buffers, salts, viscosity enhancers, or polypeptides. In some instances, the chemical comprises bovine serum albumin.

Described herein are conjugate pads that provide a region on the support medium comprising conjugates coated on its surface by conjugate binding molecules that can bind to the detector moiety from the cleaved detector molecule or to the control molecule. The conjugate pad can be made from various materials that facilitate binding of the conjugate binding molecule to the detection moiety from cleaved detector molecule and transfer of most of the conjugate-bound detection moiety to the subsequent regions. The conjugate pad may comprise the same material as the sample pad or other zones or a different material than the sample pad. The conjugate pad may comprise glass fiber filters, porous plastic membranes, aluminum oxide coated membranes, paper, cellulose fiber filters, woven meshes, polyester filter, or polymer-based matrices. The material for the conjugate pad region may be hydrophilic, have low non-specific binding, or have consistent fluid flow properties across the conjugate pad. In some cases, the material for the conjugate pad may range from about 50 μm to about 1000 μm, about 50 μm to about 750 μm, about 50 μm to about 500 μm, or about 100 μm to about 500 μm.

Further described herein are conjugates that are placed on the conjugate pad and immobilized to the conjugate pad until the sample is applied to the support medium. The conjugates may comprise a nanoparticle, a gold nanoparticle, a latex nanoparticle, a quantum dot, a chemiluminescent nanoparticle, a carbon nanoparticle, a selenium nanoparticle, a fluorescent nanoparticle, a liposome, or a dendrimer. The surface of the conjugate may be coated by a conjugate binding molecule that binds to the detection moiety from the cleaved detector molecule.

The conjugate binding molecules described herein coat the surface of the conjugates and can bind to detection moiety. The conjugate binding molecule binds selectively to the detection moiety cleaved from the detector nucleic acid. Some suitable conjugate binding molecules comprise an antibody, a polypeptide, or a single stranded nucleic acid. In some cases, the conjugate binding molecule binds a dye and a fluorophore. Some such conjugate binding molecules that bind to a dye or a fluorophore can quench their signal. In some cases, the conjugate binding molecule is a monoclonal antibody. In some cases, an antibody, also referred to as an immunoglobulin, includes any isotype, variable regions, constant regions, Fc region, Fab fragments, F(ab′)2 fragments, and Fab′ fragments. Alternatively, the conjugate binding molecule is a non-antibody compound that specifically binds the detection moiety. Sometimes, the conjugate binding molecule is a polypeptide that can bind to the detection moiety. Sometimes, the conjugate binding molecule is avidin or a polypeptide that binds biotin. Sometimes, the conjugate binding molecule is a detector moiety binding nucleic acid.

The diameter of the conjugate may be selected to provide a desired surface to volume ratio. In some instances, a high surface area to volume ratio may allow for more conjugate binding molecules that are available to bind to the detection moiety per total volume of the conjugates. In some cases, the diameter of the conjugate may range from about 1 nm to about 1000 nm, about 1 nm to about 500 nm, about 1 nm to about 100 nm, or about 1 nm to about 50 nm. In some cases, the diameter of the conjugate may be at least 1 nm, 2 nm, 3 nm, 4 nm, 5 nm, 6 nm, 7 nm, 8 nm, 9 nm, 10 nm, 15 nm, 20 nm, 25 nm, 30 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 200 nm, 300 nm, 400 nm, 500 nm, 600 nm, 700 nm, 800 nm, 900 nm, or 1000 nm. In some cases, the diameter of the conjugate may be no more than 1 nm, 2 nm, 3 nm, 4 nm, 5 nm, 6 nm, 7 nm, 8 nm, 9 nm, 10 nm, 15 nm, 20 nm, 25 nm, 30 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 200 nm, 300 nm, 400 nm, 500 nm, 600 nm, 700 nm, 800 nm, 900 nm, or 1000 nm.

The ratio of conjugate binding molecules to the conjugates can be tailored to achieve desired binding properties between the conjugate binding molecules and the detection moiety. In some instances, the molar ratio of conjugate binding molecules to the conjugates is at least 1:1, 1:5, 1:10, 1:20, 1:30, 1:40, 1:50, 1:60, 1:70, 1:80, 1:90, 1:100, 1:110, 1:120, 1:130, 1:140, 1:150, 1:160, 1:170, 1:180, 1:190, 1:200, 1:250, 1:300, 1:350, 1:400, 1:450, or 1:500. In some instances, the mass ratio of conjugate binding molecules to the conjugates is at least 1:1, 1:5, 1:10, 1:20, 1:30, 1:40, 1:50, 1:60, 1:70, 1:80, 1:90, 1:100, 1:110, 1:120, 1:130, 1:140, 1:150, 1:160, 1:170, 1:180, 1:190, 1:200, 1:250, 1:300, 1:350, 1:400, 1:450, or 1:500. In some instances, the number of conjugate binding molecules per conjugate is at least 1, 10, 50, 100, 500, 1000, 5000, or 10000.

The conjugate binding molecules can be bound to the conjugates by various approached. Sometimes, the conjugate binding molecule can be bound to the conjugate by passive binding. Some such passive binding comprises adsorption, absorption, hydrophobic interaction, electrostatic interaction, ionic binding, or surface interactions. In some cases, the conjugate binding molecule can be bound to the conjugate covalently. Sometimes, the covalent bonding of the conjugate binding molecule to the conjugate is facilitated by EDC/NHS chemistry or thiol chemistry.

Described herein are detection region on the support medium that provide a region for presenting the assay results. The detection region can be made from various materials that facilitate binding of the conjugate-bound detection moiety from cleaved detector molecule to the capture molecule specific for the detection moiety. The detection pad may comprise the same material as other zones or a different material than the other zones. The detection region may comprise nitrocellulose, paper, cellulose, cellulose fiber filters, glass fiber filters, porous plastic membranes, aluminum oxide coated membranes, woven meshes, polyester filter, or polymer-based matrices. Often the detection region may comprise nitrocellulose. The material for the region pad region may be hydrophilic, have low non-specific binding, or have consistent fluid flow properties across the region pad. The material for the conjugate pad may range from about 10 μm to about 1000 μm, about 10 μm to about 750 μm, about 10 μm to about 500 μm, or about 10 μm to about 300 μm.

The detection region comprises at least one capture area with a high density of a capture molecule that can bind to the detection moiety from cleaved detection molecule and at least one area with a high density of a positive control capture molecule. The capture area with a high density of capture molecule or a positive control capture molecule may be a line, a circle, an oval, a rectangle, a triangle, a plus sign, or any other shapes. In some instances, the detection region comprise more than one capture area with high densities of more than one capture molecules, where each capture area comprises one type of capture molecule that specifically binds to one type of detection moiety from cleaved detection molecule and are different from the capture molecules in the other capture areas. The capture areas with different capture molecules may be overlapping completely, overlapping partially, or spatially separate from each other. In some instances, the capture areas may overlap and produce a combined detectable signal distinct from the detectable signals generated by the individual capture areas. Usually, the positive control spot is spatially distinct from any of the detection spot.

The capture molecule described herein bind to detection moiety and immobilized in the detection spot in the detect region. Some suitable capture molecules comprise an antibody, a polypeptide, or a single stranded nucleic acid. In some cases, the capture molecule binds a dye and a fluorophore. Some such capture molecules that bind to a dye or a fluorophore can quench their signal. Sometimes, the capture molecule is an antibody that that binds to a dye or a fluorophore can quench their signal. In some cases, the capture molecule is a monoclonal antibody. In some cases, an antibody, also referred to as an immunoglobulin, includes any isotype, variable regions, constant regions, Fc region, Fab fragments, F(ab′)2 fragments, and Fab′ fragments. Alternatively, the capture molecule is a non-antibody compound that specifically binds the detection moiety. Sometimes, the capture molecule is a polypeptide that can bind to the detection moiety. In some instances, the detection moiety from cleaved detection molecule has a conjugate bound to the detection moiety, and the conjugate-detection moiety complex may bind to the capture molecule specific to the detection moiety on the detection region. Sometimes, the capture molecule is a polypeptide that can bind to the detection moiety. Sometimes, the capture molecule is avidin or a polypeptide that binds biotin. Sometimes, the capture molecule is a detector moiety binding nucleic acid.

The detection region described herein comprises at least one area with a high density of a positive control capture molecule. The positive control spot in the detection region provides a validation of the assay and a confirmation of completion of the assay. If the positive control spot is not detectable by the visualization methods described herein, the assay is not valid and should be performed again with a new system or kit. The positive control capture molecule binds at least one of the conjugates, the conjugate binding molecule, or detection moiety and is immobilized in the positive control spot in the detect region. Some suitable positive control capture molecules comprise an antibody, a polypeptide, or a single stranded nucleic acid. In some cases, the positive control capture molecule binds to the conjugate binding molecule. Some such positive control capture molecules that bind to a dye or a fluorophore can quench their signal. Sometimes, the positive control capture molecule is an antibody that that binds to a dye or a fluorophore can quench their signal. In some cases, the positive control capture molecule is a monoclonal antibody. In some cases, an antibody includes any isotype, variable regions, constant regions, Fc region, Fab fragments, F(ab′)2 fragments, and Fab′ fragments. Alternatively, the positive control capture molecule is a non-antibody compound that specifically binds the detection moiety. Sometimes, the positive control capture molecule is a polypeptide that can bind to at least one of the conjugates, the conjugate binding molecule, or detection moiety. In some instances, the conjugate unbound to the detection moiety binds to the positive control capture molecule specific to at least one of the conjugates, the conjugate binding molecule.

The kit or method described herein may also comprise or use a positive control sample to determine the activity of at least one of programmable nuclease, a guide nucleic acid, or a single stranded detector nucleic acid. Often, the positive control sample comprises a target nucleic acid that binds to the guide nucleic acid. The positive control sample is contacted with the reagents in the same manner as the test sample and visualized using the support medium. The visualization of the positive control spot and the detection spot for the positive control sample provides a validation of the reagents and the assay.

The kit or method of detection of a target nucleic acid described herein can further comprise reagents for nucleic acid amplification of target nucleic acids in the sample. Isothermal nucleic acid amplification allows the use of the kit or method in remote regions or low resource settings without specialized equipment for amplification. Often, the reagents for nucleic acid amplification comprise a recombinase, a oligonucleotide primer, a single-stranded DNA binding (SSB) protein, and a polymerase. Sometimes, nucleic acid amplification of the sample improves at least one of sensitivity, specificity, or accuracy of the assay in detecting the target nucleic acid. In some cases, the nucleic acid amplification is performed in a nucleic acid amplification region on the support medium. Alternatively, or in combination, the nucleic acid amplification is performed in a reagent chamber, and the resulting sample is applied to the support medium. Sometimes, the nucleic acid amplification is isothermal nucleic acid amplification. In some cases, the nucleic acid amplification is transcription mediated amplification (TMA). Nucleic acid amplification is helicase dependent amplification (HDA) or circular helicase dependent amplification (cHDA) in other cases. In additional cases, nucleic acid amplification is strand displacement amplification (SDA). In some cases, nucleic acid amplification is by recombinase polymerase amplification (RPA). In some cases, nucleic acid amplification is by at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. In some cases, the nucleic acid amplification reaction is performed at a temperature no greater than 20° C., 25° C., 30° C., 35° C., 37° C., 40° C., 45° C. In some cases, the nucleic acid amplification reaction is performed at a temperature of at least 20° C., 25° C., 30° C., 35° C., 37° C., 40° C., or 45° C.

Described herein are collection pad region that provide a region to collect the sample that flows down the support medium. Often the collection pads are placed downstream of the detection region and comprise an absorbent material. The collection pad can increase the total volume of sample that enters the support medium by collecting and removing the sample from other regions of the support medium. This increased volume can be used to wash unbound conjugates away from the detection region to lower the background and enhance assay sensitivity. When the design of the support medium does not include a collection pad, the volume of sample analyzed in the support medium may be determined by the bed volume of the support medium. The collection pad may provide a reservoir for sample volume and may help to provide capillary force for the flow of the sample down the support medium.

The collection pad may be prepared from various materials that are highly absorbent and able to retain fluids. Often the collection pads comprise cellulose filters. In some instances, the collection pads comprise cellulose, cotton, woven meshes, polymer-based matrices. The dimension of the collection pad, usually the length of the collection pad, may be adjusted to change the overall volume absorbed by the support medium.

The support medium described herein may have a barrier around the edge of the support medium. Often the barrier is a hydrophobic barrier that facilitates the maintenance of the sample within the support medium or flow of the sample within the support medium. Usually, the transport rate of the sample in the hydrophobic barrier is much lower than through the regions of the support medium. In some cases, the hydrophobic barrier is prepared by contacting a hydrophobic material around the edge of the support medium. Sometimes, the hydrophobic barrier comprises at least one of wax, polydimethylsiloxane, rubber, or silicone.

Any of the regions on the support medium can be treated with chemicals to improve the visualization of the detection spot and positive control spot on the support medium. The regions can be treated to enhance extraction of nucleic acid in the sample, to control the transport of the reacted reagents and sample or the conjugate to other regions of the support medium, or to enhance the binding of the cleaved detection moiety to the conjugate binding molecule on the surface of the conjugate or to the capture molecule in the detection region. The chemicals may comprise detergents, surfactants, buffers, salts, viscosity enhancers, or polypeptides. In some instances, the chemical comprises bovine serum albumin. In some cases, the chemicals or physical agents enhance flow of the sample with a more even flow across the width of the region. In some cases, the chemicals or physical agents provide a more even mixing of the sample across the width of the region. In some cases, the chemicals or physical agents control flow rate to be faster or slower in order to improve performance of the assay. Sometimes, the performance of the assay is measured by at least one of shorter assay time, longer times during cleavage activity, longer or shorter binding time with the conjugate, sensitivity, specificity, or accuracy.

### Kits

Disclosed herein are kits for use to assay for or detect the segment of the target nucleic acid to determine modification status of the segment of the target nucleic acid using the methods as discuss above. In some embodiments, the kit comprises the programmable nuclease system, reagents, and the support medium. The reagents and programmable nuclease system can be provided in a reagent chamber or on the support medium. Alternatively, the reagent and programmable nuclease system can be placed into the reagent chamber or the support medium by the individual using the kit. Optionally, the kit further comprises a buffer and a dropper. The reagent chamber can be a test well or container. The opening of the reagent chamber can be large enough to accommodate the support medium. The buffer can be provided in a dropper bottle for ease of dispensing. The dropper can be disposable and transfer a fixed volume. The dropper can be used to place a sample into the reagent chamber or on the support medium.

In some embodiments, a kit for assaying for or detecting a segment of a target nucleic acid comprising a support medium; a guide nucleic acid targeting a target sequence; a programmable nuclease capable of being activated when complexed with the guide nucleic acid and the target sequence; and a detector nucleic acid comprising a detection moiety, wherein the detector nucleic acid is capable of being cleaved by the activated nuclease, thereby generating a first detectable signal.

In some embodiments, a kit for assaying for or detecting a segment of a target nucleic acid comprising a PCR plate; a guide nucleic acid targeting a target sequence; a programmable nuclease capable of being activated when complexed with the guide nucleic acid and the target sequence; and a single stranded detector nucleic acid comprising a detection moiety, wherein the detector nucleic acid is capable of being cleaved by the activated nuclease, thereby generating a first detectable signal. The wells of the PCR plate can be pre-aliquoted with the guide nucleic acid targeting a target sequence, a programmable nuclease capable of being activated when complexed with the guide nucleic acid and the target sequence, and at least one population of a single stranded detector nucleic acid comprising a detection moiety. A user can thus add the biological sample of interest to a well of the pre-aliquoted PCR plate and measure for the detectable signal with a fluorescent light reader or a visible light reader.

In some instances, such kits may include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers.

The kit or systems described herein contain packaging materials. Examples of packaging materials include, but are not limited to, pouches, blister packs, bottles, tubes, bags, containers, bottles, and any packaging material suitable for intended mode of use.

A kit typically includes labels listing contents and/or instructions for use, and package inserts with instructions for use. A set of instructions will also typically be included. In one embodiment, a label is on or associated with the container. In some instances, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

### Stability

Disclosed herein are stable compositions of the reagents and the programmable nuclease system for use in the methods as discussed above. The reagents and programmable nuclease system described herein may be stable in various storage conditions including refrigerated, ambient, and accelerated conditions. Disclosed herein are stable reagents and programmable nuclease system. The stability may be measured for the reagents and programmable nuclease system themselves or the reagents and programmable nuclease system present on the support medium.

In some instances, stable as used herein refers to a reagents having about 5% w/w or less total impurities at the end of a given storage period. Stability may be assessed by HPLC or any other known testing method. The stable reagents may have about 10% w/w, about 5% w/w, about 4% w/w, about 3% w/w, about 2% w/w, about 1% w/w, or about 0.5% w/w total impurities at the end of a given storage period.

In some embodiments, stable as used herein refers to a reagents and programmable nuclease system having about 10% or less loss of detection activity at the end of a given storage period and at a given storage condition. Detection activity can be assessed by known positive sample using a known method. Alternatively or combination, detection activity can be assessed by the sensitivity, accuracy, or specificity. In some embodiments, the stable reagents has about 10%, about 9%, about 8%, about 7%, about 6%, about 5%, about 4%, about 3%, about 2%, about 1%, or about 0.5% loss of detection activity at the end of a given storage period.

In some embodiments, the stable composition has zero loss of detection activity at the end of a given storage period and at a given storage condition. The given storage condition may comprise humidity of equal to or less than 10%, 20%, 30%, 40%, 50%, 60%, 70%, 80%, 90%, or 100% relative humidity. The controlled storage environment may comprise humidity between 0% and 50% relative humidity, 0% and 40% relative humidity, 0% and 30% relative humidity, 0% and 20% relative humidity, or 0% and 10% relative humidity. The controlled storage environment may comprise temperatures of-100° C., −80° C., −20° C., 4° C., about 25° C. (room temperature), or 40° C. The controlled storage environment may comprise temperatures between −80° C. and 25° C., or −100° C. and 40° C. The controlled storage environment may protect the system or kit from light or from mechanical damage. The controlled storage environment may be sterile or aseptic or maintain the sterility of the light conduit. The controlled storage environment may be aseptic or sterile.

The kit can be packaged to be stored for extended periods of time prior to use. The kit or system may be packaged to avoid degradation of the kit or system. The packaging may include desiccants or other agents to control the humidity within the packaging. The packaging may protect the kit or system from mechanical damage or thermal damage. The packaging may protect the kit or system from contamination of the reagents and programmable nuclease system. The kit or system may be transported under conditions similar to the storage conditions that result in high stability of the reagent or little loss of reagent activity. The packaging may be configured to provide and maintain sterility of the kit. The kit can be compatible with standard manufacturing and shipping operations.

While various embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### DETAILED FIGURE DESCRIPTIONS

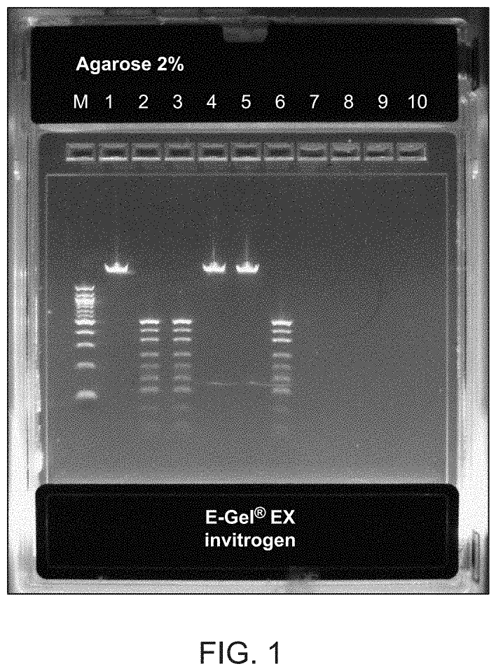

FIG. 1 depicts a 2% agarose gel confirming restriction digestion of unmethylated or methylated pUC19 with Thermo Scientific EpiJET DNA Methylation Analysis kit. Lane M is a ladder. Lane 1: Unmethylated pUC19+no enzyme. Lane 2: Unmethylated pUC19+Epi HpaII. Lane 3: Unmethylated pUC19+Epi Mpsl. Lane 4: Methylated pUC19+no enzyme. Lane 5: Methylated pUC19+Epi HpaII. Lane 6: Methylated pUC19+Epi MpsI. Lanes 1, 4, and 5 have bands that are approximately the same size about one third of the way down the gel from the top. Lanes 2, 3, and 6 have bands of multiple sizes with the first band approximately half way down the gel in each lane. Lanes 7, 8, 9, and 10 are empty.

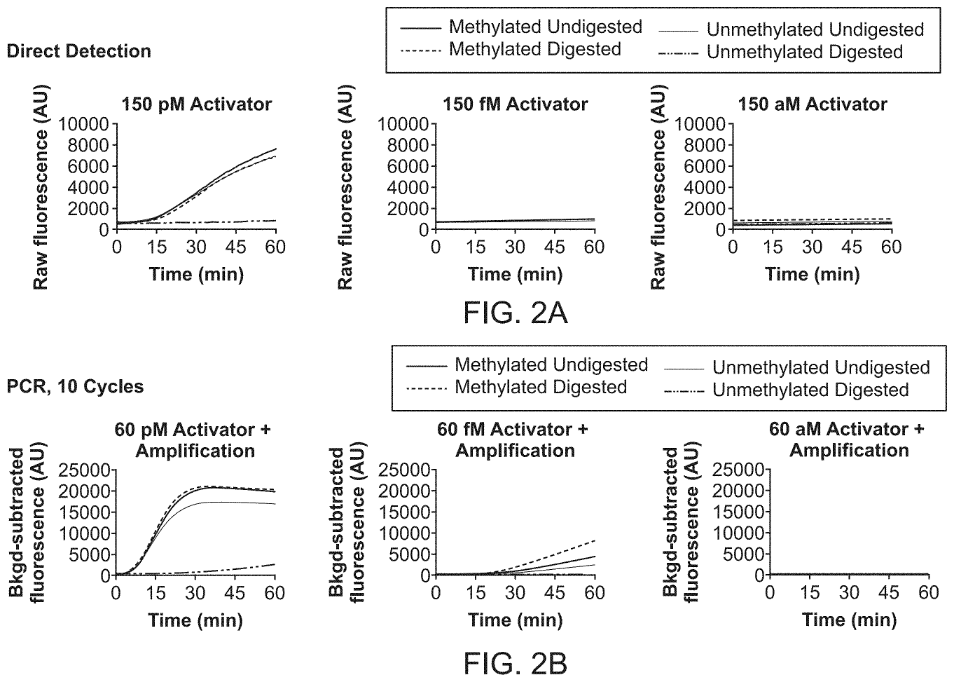

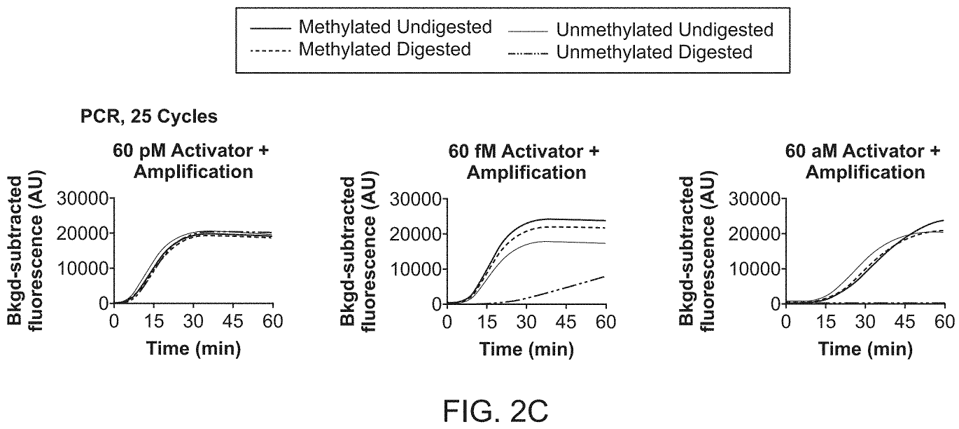

FIG. 2A depicts detection of Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) without intermediate amplification. The graph on the left side is for 150 pM activator, has a y-axis indicating Raw fluorescence (AU) from 0 to 10000 in intervals of 2000 and an x-axis indicating time (min) from 0 to 60 in intervals of 15. The bottom line indicates the unmethylated digest DNA. The top line indicates the methylated undigested DNA. The middle line indicates the unmethylated undigested DNA and overlaps with the methylated digested DNA. The graph in the middle is for 150 fM activator, has a y-axis indicating Raw fluorescence (AU) from 0 to 10000 in intervals of 2000 and an x-axis indicating time (min) from 0 to 60 in intervals of 15. The lines indicating the unmethylated digested DNA, the methylated digested DNA, the methylated digest DNA, and the unmethylated undigested DNA are all approximately overlapping. The graph on the right is for 150 aM activator, has a y-axis indicating Raw fluorescence (AU) from 0 to 10000 in intervals of 2000 and an x-axis indicating time (min) from 0 to 60 in intervals of 15. The lines indicating the unmethylated digested DNA, the methylated digested DNA, the methylated digest DNA, and the unmethylated undigested DNA are all approximately overlapping.

FIG. 2B depicts detection of Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) after 10 cycles of PCR amplification. The graph on the left side is for 60 pM activator+amplification, has a y-axis indicating background subtracted fluorescence (AU) from 0 to 25000 in intervals of 5000 and an x-axis indicating time (min) from 0 to 60 in intervals of 15. The bottom line indicates the unmethylated digested DNA. The top line indicates the unmethylated digested DNA, which also approximately overlaps with (but is slightly below) the line indicating the methylated digest DNA. The middle line indicates the unmethylated undigested DNA. The graph in the middle is for 60 fM activator+amplification, has a y-axis indicating background subtracted fluorescence (AU) from 0 to 25000 in intervals of 5000 and an x-axis indicating time (min) from 0 to 60 in intervals of 15. The line indicating the unmethylated digested DNA is overlapping with the x-axis. Above the unmethylated digested DNA line is the unmethylated undigested DNA. Above the unmethylated undigested DNA line is the methylated digested DNA line. The top line is the methylated digested DNA line. The graph on the right is for 600 aM activator, has a y-axis indicating background subtracted fluorescence (AU) from 0 to 25000 in intervals of 5000 and an x-axis indicating time (min) from 0 to 60 in intervals of 15. The lines indicating the unmethylated digested DNA, the methylated digested DNA, the methylated digested DNA, and the unmethylated undigested DNA are all approximately overlapping with the x-axis.

FIG. 2C depicts detection of Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) after 25 cycles of PCR amplification. The graph on the left side is for 60 pM activator+amplification, has a y-axis indicating background subtracted fluorescence (AU) from 0 to 30000 in intervals of 10000 and an x-axis indicating time (min) from 0 to 60 in intervals of 15. All lines are approximately overlapping for digested methylated DNA, undigested methylated DNA, digested unmethylated DNA, or undigested unmethylated DNA . The graph in the middle is for 60 fM activator+amplification, has a y-axis indicating background subtracted fluorescence (AU) from 0 to 30000 in intervals of 10000 and an x-axis indicating time (min) from 0 to 60 in intervals of 15. The bottom line indicates the unmethylated digested DNA. Above the unmethylated digested DNA is the line indicating the unmethylated undigested DNA. Above the unmethylated undigested DNA line is the line indicating the methylated digested DNA. Above the methylated digested DNA line is the line indicating the methylated undigested DNA. The graph on the right is for 60 aM+activator, has a y-axis indicating background subtracted fluorescence (AU) from 0 to 30000 in intervals of 10000 and an x-axis indicating time (min) from 0 to 60 in intervals of 15. The lines indicating the unmethylated digested DNA is approximately overlapping with the x-axis. The beginning of the line indicating the methylated digested DNA is overlapping with the methylated undigestd DNA, but is lower and approximately overlapping with the unmethylated undigested DNA at 60 min. The unmethylated undigest DNA line begins slightly above the methylated undigested DNA line but at approximately 45 minutes to 60 minutes, the unmethylated undigested line is lower than the methylated undigested line. The methylated undigest DNA line begins slightly below the unmethylated undigested DNA line but at approximately 45 minutes to 60 minutes, the methylated undigested DNA line is higher than the unmethylated undigested line.

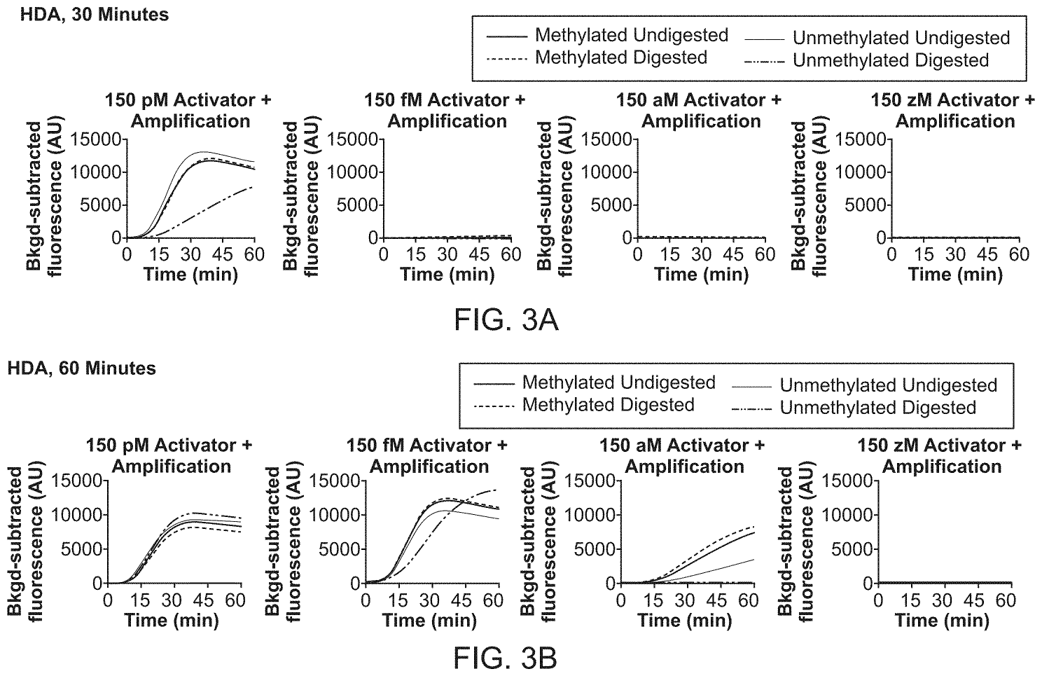

FIG. 3A depicts detection of helicase-dependent isothermal amplified Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) after 30 minutes of incubation. The graph on the left side is for 150 pM activator+amplification, has a y-axis indicating background subtracted fluorescence (AU) from 0 to 15000 in intervals of 5000 and an x-axis indicating time (min) from 0 to 60 in intervals of 15. The top most line indicates the methylated undigested DNA. The bottom line indicates the un methylated digested DNA. The methylated digested DNA and the methylated undigested DNA lines are approximately overlapping and are between the unmethylated undigested DNA line and methylated undigested DNA line. The graph on the middle left is for 150 fM activator+amplification, has a y-axis indicating background subtracted fluorescence (AU) from 0 to 15000 in intervals of 5000 and an x-axis indicating time (min) from 0 to 60 in intervals of 15. All lines are approximately overlapping with the x-axis. The graph on the middle right is for 150 aM activator+amplification, has a y-axis indicating background subtracted fluorescence (AU) from 0 to 15000 in intervals of 5000 and an x-axis indicating time (min) from 0 to 60 in intervals of 15. All lines are approximately overlapping with the x-axis. The graph on the right is for 150 zM activator+amplification, has a y-axis indicating background subtracted fluorescence (AU) from 0 to 15000 in intervals of 5000 and an x-axis indicating time (min) from 0 to 60 in intervals of 15. All lines are approximately overlapping with the x-axis.

FIG. 3B depicts detection of helicase-dependent isothermal amplified Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) after 60 minutes of incubation. The graph on the left side is for 150 pM activator+amplification, has a y-axis indicating background subtracted fluorescence (AU) from 0 to 15000 in intervals of 5000 and an x-axis indicating time (min) from 0 to 60 in intervals of 15. The top most line indicates the unmethylated digested DNA. The bottom line indicates the methylated digested DNA. The unmethylated undigested DNA and the methylated undigested DNA lines are approximately overlapping and are between the unmethylated undigested DNA line and methylated undigested DNA line. The graph on the middle left is for 150 fM activator+amplification, has a y-axis indicating background subtracted fluorescence (AU) from 0 to 15000 in intervals of 5000 and an x-axis indicating time (min) from 0 to 60 in intervals of 15. The unmethylated digested DNA line starts below the other lines, but at approximately 45 min is above all the lines. The methylated undigested DNA line and the methylated digested DNA line are approximately overlapping and are above the unmethylated undigested DNA line and below the unmethylated digested DNA line at 60 min. The unmethylated undigested DNA line is below all the other lines at 60 min. The graph on the middle right is for 150 aM activator+amplification, has a y-axis indicating background subtracted fluorescence (AU) from 0 to 15000 in intervals of 5000 and an x-axis indicating time (min) from 0 to 60 in intervals of 15. The unmethylated digested DNA line is approximately overlapping with the x-axis, the unmethlyated undigested DNA line is above the unmethylated undigested DNA line but below the methylated digested DNA line and the methylated undigested DNA line, the methylated undigested DNA line is between the unmethylated undigested DNA line and the methylated digested DNA line (but closer to the methylated digested DNA line), and the methylated digested DNA line is above all the lines. The graph on the right is for 150 zM activator+amplification, has a y-axis indicating background subtracted fluorescence (AU) from 0 to 15000 in intervals of 5000 and an x-axis indicating time (min) from 0 to 60 in intervals of 15. All lines are approximately overlapping with the x-axis.

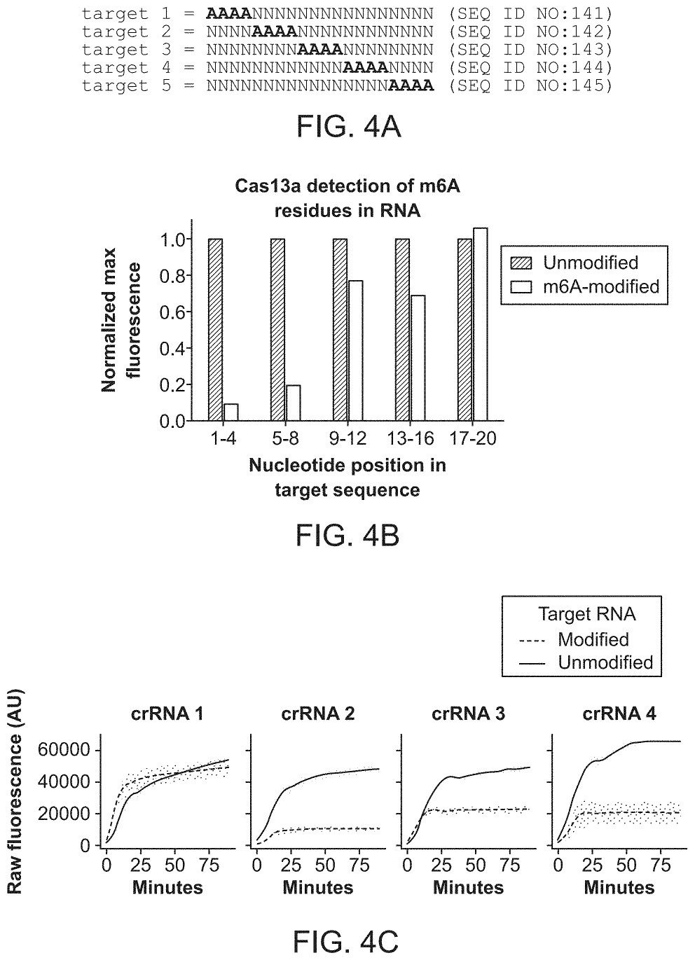

FIG. 4A depicts a schematic of various positions of adenosines (A) in target RNAs, wherein each target RNA contains identical surrounding sequence context (N). The adenosines can either be unmodified or modified (N6-methyladenosine). All the sequences are 20 nucleic acids in length. Target 1 comprises AAAANNNNNNNNNNNNNNNN (SEQ ID NO: 141); target 2 comprises NNNNAAAANNNNNNNNNNNN (SEQ ID NO: 142); target 3 comprise NNNNNNNNAAAANNNNNNNN (SEQ ID NO: 143); target 4 comprise NNNNNNNNNNNNAAAANNNN (SEQ ID NO: 144); and target 5 comprises NNNNNNNNNNNNNNNAAAA (SEQ ID NO: 145).

FIG. 4B depicts the normalized fluorescence readings from Cas13a detection assay using a LbuCas13a programmable nuclease (SEQ ID NO: 124) with unmodified adenosine or modified adenosine (N6-methyladenosine) target RNAs of FIG. 4A. The y-axis is the normalized fluorescence max from 0.0 to 1.0 in intervals of 0.2 and the x-axis indicates the nucleotide position of the four adenosine residues in the target nucleic acids from FIG. 4A. From left to right, the 1-4 corresponds to target 1; 5-8 corresponds to target 2; 9-12 corresponds to target 13-16 corresponds to target 4; and 17-20 corresponds to target 5 of FIG. 4A. For each target, there are two bars indicating the fluorescence of the target in a Cas13a detection assay when the target was unmodified (left bar) or N6-methyladenosine modified (right bar).

FIG. 4C depicts raw fluorescent results of four different crRNAs using a LbuCas13a programmable nuclease (SEQ ID NO: 124) along either an unmodified or modified (N6-methyladenosine) target RNA derived from a natural sequence. The y-axis for all the graphs is for raw fluorescence (AU) from 0 to 60000 in intervals of 20000, and the x-axis for all the graphs is minutes from 0 to 75 in intervals of 25. From left to right, the graphs are for crRNA 1, crRNA 2, crRNA 3, crRNA 4, and crRNA 5. For the crRNA 1 graph the line indicating the modified target RNA starts above the unmodified target RNA, but at approximately 50 minutes, the unmodified target RNA line is higher than the modified target RNA line. For the crRNA 2 graph, the unmodified target RNA line is above the modified target RNA line. For the crRNA3 graph, both lines are approximately overlapping until about 15 min, after which the unmodified target RNA line is higher than the modified target RNA line. For the crRNA 4 graph, the line indicating unmodified target RNA is higher than the line indicating modified target RNA.

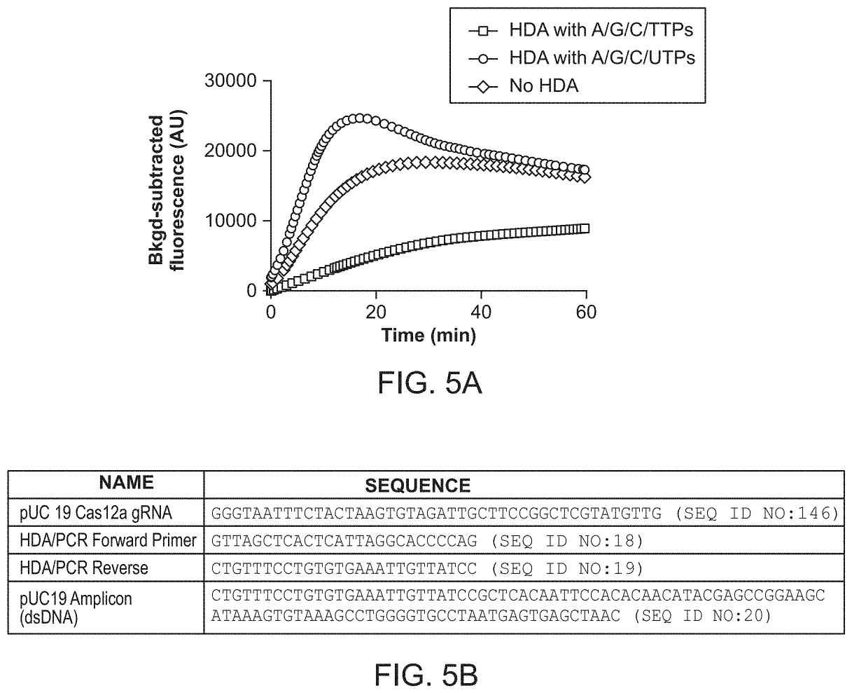

FIG. 5A depicts 10 nM DNA amplified by HDA with standard dNTPs (A/G/C/T) and with a dA/G/C/UTP mix (no thymines). LbCas12a (SEQ ID NO: 21) detection of 2 μL of these reactions is shown alongside a no amplification control, demonstrating that Cas12a can detect uracil-containing amplicons at a rate similar to that of thymine-containing amplicons. The y-axis is the background subtracted fluorescence (AU) from 0 to 30000 at intervals of 1000 and the x-axis time (minutes) from 0 to 60 in intervals of 20. The top line indicates HDA performed with no thymine mix dNTPs (A/G/C/U) , the middle line indicates HDA performed with standard dNTPs (A/G/C/T), and the bottom line indicates no HDA was performed.

FIG. 5B shows the sequences of the crRNA (pUC19 Cas12a gRNA), forward and reverse HDA/PCR primers, and the pUC19 amplicon used in FIG. 2, FIG. 3, and FIG. 5A.

FIG. 6A shows a schematic entitled “Amplification with PT′d primer+T7 exonuclease”. At the top left is a region of target ssDNA, dsDNA, or RNA. An arrow is shown leading to the bottom left of the schematic, indicating to reverse transcribe if the target is RNA. At the bottom left are two strands of DNA. At the top left is a note that dsDNA is generated using a PT′d forward (abbreviated as “fwd”) primer. At the bottom right is a note that the strands were generated using a non-PT′d reverse primer (“No PT rev primer”). An arrow is shown leading to the bottom right of the schematic indicating amplification. Four strands of amplified dsDNA is depicted, wherein the PT′d strand is on top and contains the PT′d nucleotides at the 5′ end as denoted by a solid square. An arrow is shown leading to the top right of the schematic indicating treatment with exonuclease. Four strands of amplified ssDNA activator are shown with PT′d nucleotides at the 5′ end denoted by a solid square.

FIG. 6B shows a schematic entitled “Asymmetric Concentration of Primers”. At the left most end of the schematic is a region of target ssDNA, dsDNA, or RNA. An arrow is shown leading to the right, indicating to reverse transcribe if the target is RNA. In the middle is a double stranded DNA. At the top left of the dsDNA is a large arrow noting that the forward primer is added in excess (“Fwd primer (excess)”). At the bottom right of the dsDNA is a small arrow noting that reverse primer is added in a limited quantity (“Rev primer (limiting)”). An arrow is shown leading to the right indicating amplification. Shown at the right is one dsDNA and four (excess) amplified ssDNA activator.

FIG. 6C shows a schematic entitled “Strand Displacing Amplification with Nested Primers.” At the left most end of the schematic if a region of target ssDNA, dsDNA, or RNA. An arrow is shown leading to the right, indicating to reverse transcribe if the target is RNA. In the middle is a double stranded DNA. At the top left of the dsDNA is an arrow at the outer edge noting an outer forward primer (“outer fwd primer”). An arrow is shown leading to the right indicating amplification with SDP. Shown at the right are four dsDNA and four of the amplified ssDNA activators.

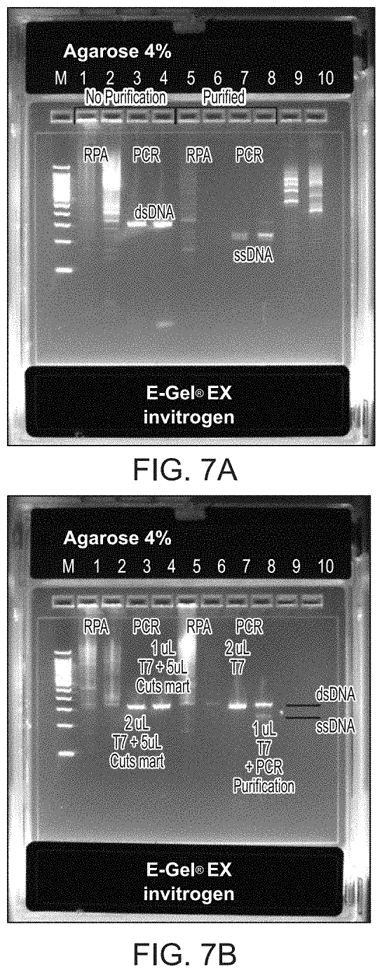

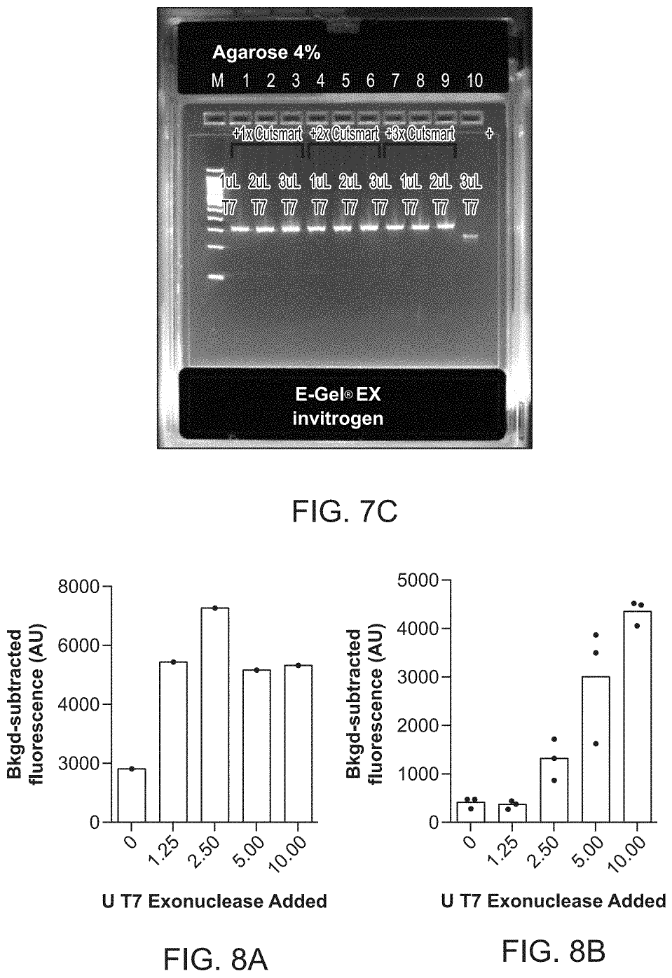

FIG. 7A shows a gel showing several lanes with a ladder in the left most lane titled “M”. Lanes 1-4 are samples with no purification. Lanes 5-8 show purified samples. Lanes 1 and 2 show recombinase polymerase amplification (RPA) with no purification. Lanes 5 and 6 show RPA with purification. The unpurified PCR lanes (lanes 3-4) show dsDNA, as evidenced by a band in each lane immediately adjacent each other. The purified PCR lanes (lanes 7-8) show ssDNA, as evidenced by a band in each lane immediately adjacent each other.

FIG. 7B shows a gel showing several lanes with a ladder in the left most lane titled “M”. Lanes 2 and 3 show RPA. Lane 3 shows PCR amplification with 2 μL T7+5 μL Cutsmart and Lane 4 shows PCR amplification with 1 μL T7+5 μL Cutsmart. Lanes 5 and 6 show RPA. Lane 7 shows results of PCR amplification with 2 μL T7. Lane 8 shows results of PCR amplification with 1 μL T7+PCR purification. Bands corresponding to dsDNA are indicated on the right and ssDNA are indicated to the right, below dsDNA. ssDNA was generated in Lane 8 (PCR amplification with 1 μL T7+purification).

FIG. 7C shows a gel showing several lanes with a ladder in the left most lane titled “M”. Lane 1 shows 1X Cutsmart with 1 μL T7, Lane 2 shows 1X Cutsmart with 2 μL T7, Lane 3 shows 1X Cutsmart with 3 μL T7, Lane 4 shows 2X Cutsmart with 1 μL T7, Lane 5 shows 2X Cutsmart with 2 μL T7, Lane 6 shows 2X Cutsmart with 3 μL T7, Lane 7 shows 3X Cutsmart with 1 μL T7, Lane 8 shows 3X Cutsmart with 3 μL T7. All lanes show unpurified PCR products.

FIG. 8A shows a graph of units (U) of T7 exonuclease added on the x-axis and background-subtracted fluorescence (absorbance units; AU) on the y-axis. The y-axis ranges from 0 to 8000 in increments of 2000. Groups shown on the x-axis include 0 U, 1.25 U, 2.5 U, 5 U, and 10 U.

FIG. 8B shows a graph of units (U) of T7 exonuclease added on the x-axis and background-subtracted fluorescence (absorbance units; AU) on the y-axis. The y-axis ranges from 0 to 5000 in increments of 1000. Groups shown on the x-axis include 0 U, 1.25 U, 2.5 U, 5 U, and 10 U.

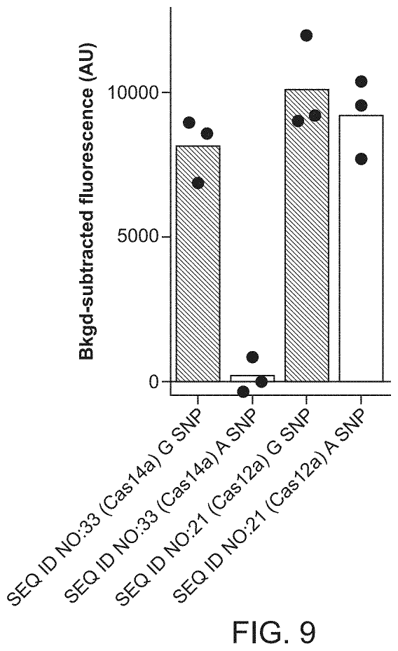

FIG. 9 shows a graph of various groups on the x-axis and background subtracted fluorescence (absorbance units; AU) on the y-axis. The y-axis ranges from 0 to 10000 in increments of 5000. The groups on the x-axis include, from left to right, Cas14a G SNP, Cas14a A SNP, Cas12a G SNP, and Cas12a A SNP.

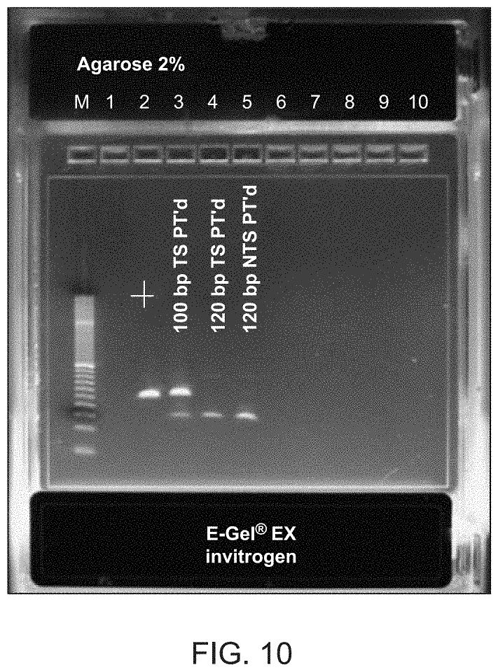

FIG. 10 shows a gel with several lanes. A ladder is in the left most lane titled “M”. Lane 3 shows 100 bp TS PT′d, Lane 4 shows 120 bp TS PT′d, and Lane 5 shows 120bp NTS PT′d. Bands seen in experimental lanes show the products of helicase-dependent amplification.

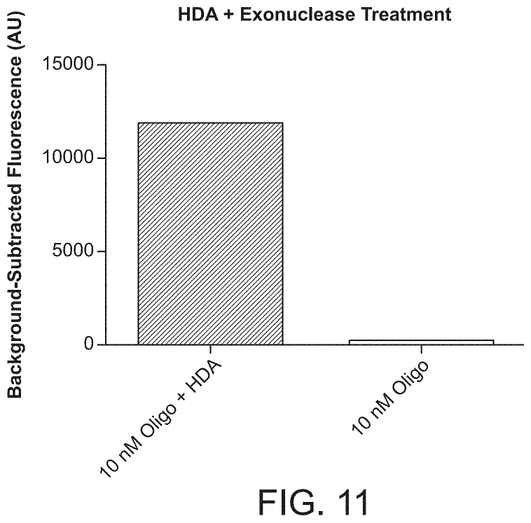

FIG. 11 shows a bar graph entitled “HDA+Exonuclease Treatment”. The x-axis shows two groups, which from left to right are 10 nm Oligo+HDA and 10 nM Oligo. The y-axis shows background subtracted fluorescence (absorbance units; AU). At the top of each figure is the concentration of T7 exonuclease in each reaction.

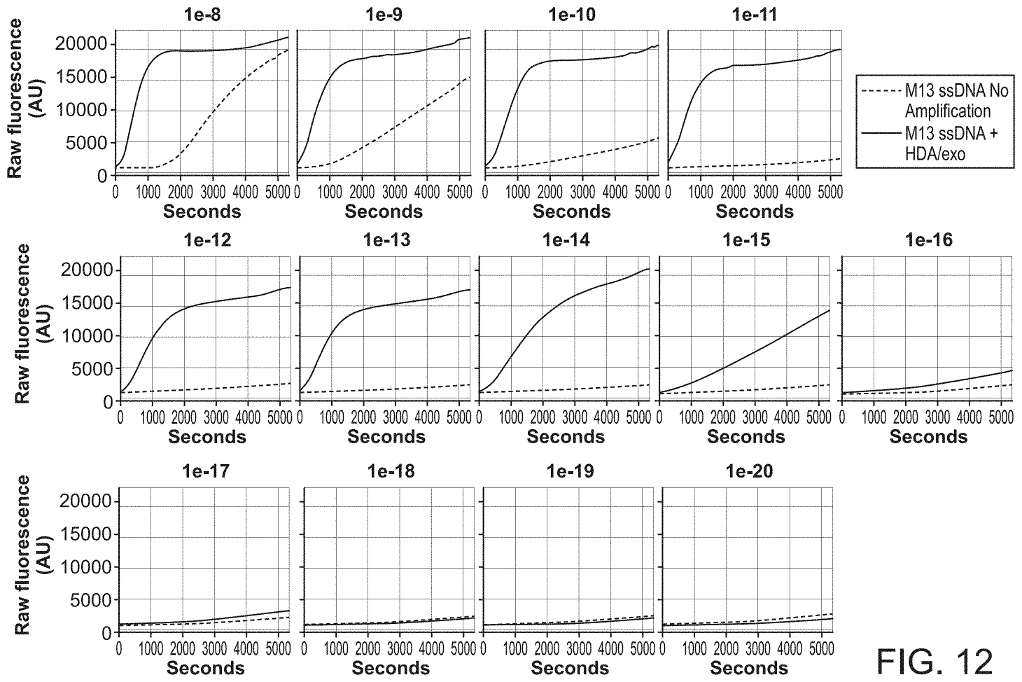

FIG. 12 shows 13 line graphs of seconds on the x-axis versus raw fluorescence (absorbance units; AU) on the y-axis. The x-axis ranges from 0 to 5000 in increments of 1000. The y-axis ranges from 0 to 20000 in increments of 5000. Higher raw fluorescence indicates detection of M13 ssDNA after treatment with a T7 exonuclease. The titles for each graph correspond to the amount of exonuclease. In the top row, from left to right, the concentrations are 1e-8, 1e-9, 1e-10, and 1e-11. In the middle row, from left to right, the concentrations are 1e-12, 1e-13, 1e-14, and 1e-15. In the bottom row, from left to right, the concentrations are 1e-16, 1e-17, 1e-18, 1e-19, and 1e-20. In most graphs, the M13 ssDNA+HDA/exonuclease is the line, corresponding to detection of ssDNA. M13 ssDNA with no amplification is the lower line.

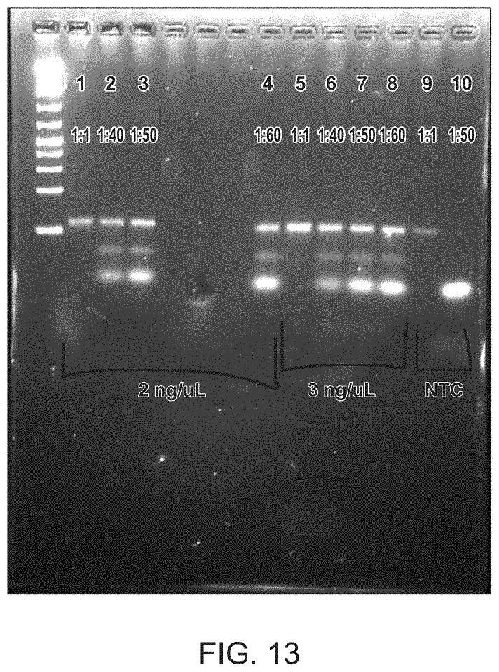

FIG. 13 shows a gel with a DNA ladder in the left most lane. Lanes vary primer ratios and the concentration of starting DNA. Lane 1 shows a 1:1 ratio of forward to reverse primers and a starting DNA concentration of 2 ng/μL. Lane 2 shows a 1:40 ratio of forward to reverse primers and a starting DNA concentration of 2 ng/μL. Lane 3 shows a 1:50 ratio of forward to reverse primers and a starting DNA concentration of 2 ng/μL. Lane 4 shows a 1:60 ratio of forward to reverse primers and a starting DNA concentration of 2 ng/μL. Lane 5 shows a 1:1 ratio of forward to reverse primers and a starting DNA concentration of 3 ng/μL. Lane 6 shows a 1:40 ratio of forward to reverse primers and a starting DNA concentration of 3 ng/μL. Lane 7 shows a 1:50 ratio of forward to reverse primers and a starting DNA concentration of 3 ng/μL. Lane 8 shows a 1:50 ratio of forward to reverse primers and a starting DNA concentration of 3 ng/μL. Lane 9 shows a 1:1 ratio of forward to reverse primers and NTC. Lane 10 shows a 1:50 ratio of forward to reverse primers and NTC.

FIG. 14A shows a bar graph entitled “Detection of Asymmetric PCR Products with Cas14a.1”. The x-axis shows experimental groups, where each experimental group is a particular forward primer concentration to reverse primer concentration ratio. The groups shown on the x-axis include 1:1, 10:1, 100:1, and 1000:1. The y-axis shows background subtracted fluorescence (absorbance units; AU) and ranges from 0 to 80000 in increments of 20000.

FIG. 14B shows a bar graph entitled “Detection of Asymmetric PCR Products with Cas14a.1”. The x-axis shows experimental groups, where each experimental group is a particular forward primer concentration to primer concentration ratio. The groups shown on the x-axis include 1:1, 30:1, 40:1, 50:1, 60:1, and 70:1. The y-axis shows background subtracted fluorescence (absorbance units; AU) and ranges from 0 to 1250000 in increments of 250000.

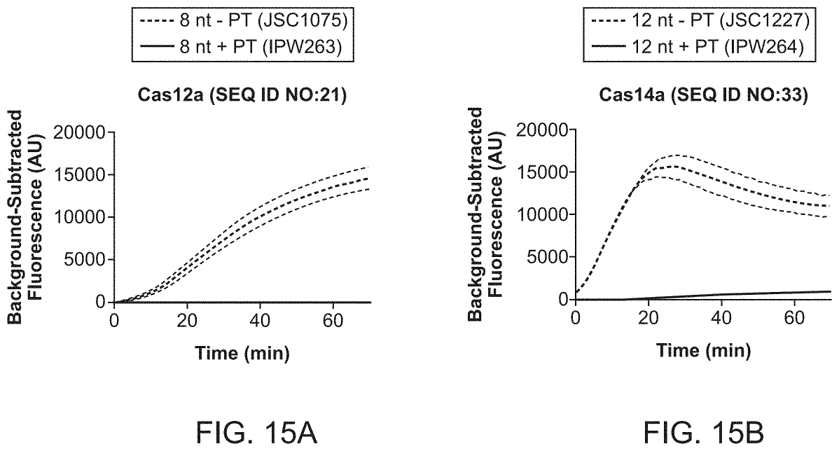

FIG. 15A is titled Cas12a and shows time (min) on the x-axis, ranging from 0 to 60 in increments of 20, versus background subtracted fluorescence (absorbance units; AU) on the y-axis, ranging from 0 to 20000 in increments of 5000. The top line shown in the graph is 8 nt−PT (JSC1075), as shown in the legend to the right. The lighter line, near 0 on the y-axis over the time period tested, is 8 nt+PT (IPW263).

FIG. 15B is titled Cas14a and shows time (min) on the x-axis, ranging from 0 to 60 in increments of 20, versus background subtracted fluorescence (absorbance units; AU) on the y-axis, ranging from 0 to 20000 in increments of 5000. The top line shown in the graph is 12 nt−PT (JSC1227). The bottom line shown in the graph is 12 nt+PT (IPW264).

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A method comprising contacting a methylation sensitive programmable nuclease to a sample comprising a methylated nucleic acid. 2. The method of embodiment 1, wherein the methylation sensitive programmable nuclease is a methylation sensitive CRISPR enzyme. 3. A method comprising contacting a sample comprising a methylated nucleic acid to an enzyme composition comprising a programmable nuclease, wherein the enzyme composition exhibits methylation sensitive cleavage. 4. The method of embodiment 3, wherein the enzyme composition comprises a CRISPR enzyme. 5. A method comprising contacting a sample comprising a methylated nucleic acid to a reagent and to a programmable nuclease, wherein the reagent differentially reacts to methylated bases. 6. The method of embodiment 5, wherein the programmable nuclease is a CRISPR enzyme. 7. A method of detecting a methylation state of a target nucleic acid, comprising: contacting a population comprising a detector nucleic acid and a programmable nuclease to the target nucleic acid, wherein the programmable nuclease comprises a guide nucleic acid molecule having a sequence that is reverse complementary to a methylation variable region in the target nucleic acid, wherein the detector nucleic acid comprises a detectable moiety, and wherein the programmable nuclease cleaves the detector nucleic acid when the guide nucleic acid hybridizes to the target nucleic acid; and determining the methylation state is unmethylated when the detectable moiety is detected by a detectable signal upon cleavage by the programmable nuclease. 8. The method of embodiment 7, wherein the guide nucleic acid comprises at least one uracil in a region from nucleic acid residue 5 to 20 of the reverse complementary region. 9. A method of detecting a methylation state of a target RNA, the method comprising: contacting a first sample comprising a target RNA to a Cas guide nucleic acid molecule having a sequence that is reverse complementary to a sequence of the target RNA, a detector nucleic acid, and a Cas protein that cleaves the detector nucleic acid; and contacting a second sample comprising an unmethylated target RNA to a Cas guide nucleic acid molecule having a sequence that is reverse complementary to a sequence of the unmethylated target RNA; the detector nucleic acid; and the Cas protein that cleaves the detector nucleic acid, wherein the target RNA has the same sequence as the unmethylated target RNA; assaying a first signal produced by cleavage of the detector nucleic acid in the first sample; assaying a second signal produced by cleavage of the detector nucleic acid in the second sample; and determining that the methylation state is unmethylated when the first signal is substantially the same as the second signal or determining that the methylation state is methylated when the first signal is less than the second signal. 10. A method of detecting a methylation state of a target RNA, the method comprising: contacting a sample comprising the target RNA to a Cas guide nucleic acid molecule having a sequence that is reverse complementary to a methylation variable region in a target RNA; a detector nucleic acid, and a Cas protein that cleaves the detector nucleic acid; and assaying a signal produced by the cleavage of the detector nucleic acid. 11. The method of embodiment 9 or embodiment 10, wherein the guide nucleic acid comprises at least one uracil in a region from nucleic acid residue 5 to 20 of the reverse complementary region. 12. The method of any one of embodiments 9-11, wherein the Cas guide nucleic acid molecule is a guide RNA molecule. 13. A method of designing a methylation sensitive CRISPR enzyme complex, comprising: a) providing a guide nucleic acid molecule having a sequence that is reverse complementary to a methylation variable region in a target nucleic acid, wherein the guide nucleic acid comprises at least one uracil in a region from nucleic acid residue 5 to 20 of the reverse complementary region; b) providing a Cas protein that cleaves a detector nucleic acid; and c) assembling the methylation sensitive CRISPR enzyme complex using the guide nucleic acid molecule and the Cas protein. 14. A method of designing a methylation sensitive CRISPR enzyme complex, comprising: a) identifying a methylation variable region in a target nucleic acid sequence; b) selecting a guide nucleic acid having a sequence that is reverse complementary to a methylation variable region in a target nucleic acid, wherein the guide nucleic acid comprises at least one uracil in a region from nucleic acid residue 5 to 20 of the reverse complementary region; and c) assembling the guide nucleic acid and a Cas protein to form the methylation sensitive CRISPR enzyme complex. 15. A method of detecting methylation of a nucleic acid, comprising: a) providing a methylation sensitive CRISPR enzyme comprising: i) a guide nucleic acid molecule having a sequence that is reverse complementary to a methylation variable region in a target nucleic acid, wherein the guide nucleic acid comprises at least one uracil in a region from nucleic acid residue 5 to 20 of the reverse complementary region, and ii) a Cas protein that cleaves a detector nucleic acid; b) assaying for methylation of the nucleic acid by contacting the nucleic acid to the methylation sensitive CRISPR enzyme; c) determining the nucleic acid is methylated when a signal is produced by the cleavage of the detector nucleic acid. 16. A method of detecting methylation of a target RNA, the method comprising: a) contacting (i) a first sample comprising a target RNA to (1) a Cas guide RNA molecule having a sequence that is reverse complementary to a sequence of the target RNA; (2) a detector nucleic acid; and (3) a Cas protein that cleaves the detector nucleic acid; and (ii) a second sample comprising an unmethylated target RNA to (1) a Cas guide RNA molecule having a sequence that is reverse complementary to a sequence of the unmethylated target RNA; (2) the detector nucleic acid; and (3) the Cas protein that cleaves the detector nucleic acid; wherein the target RNA has the same sequence as the unmethylated target RNA; b) measuring a first signal produced by cleavage of the detector nucleic acid in the first sample; c) measuring a second signal produced by cleavage of the detector nucleic acid in the second sample; and d) determining the first sample comprises methylated RNA when the first signal is less than the second signal. 17. A method of detecting methylation of a target RNA, the method comprising: a) contacting a sample comprising the target RNA to (1) a Cas guide RNA molecule having a sequence that is reverse complementary to a methylation variable region in a target RNA, wherein the guide nucleic acid comprises at least one uracil in a region from nucleic acid residue 5 to 20 of the reverse complementary region; (2) a detector nucleic acid; and (3) a Cas protein that cleaves the detector nucleic acid; and b) measuring a signal produced by cleavage of the detector nucleic acid. 18. A method of detecting methylation of a target DNA in a sample, the method comprising: contacting the sample to a methylation-specific restriction enzyme; contacting the sample to a composition comprising a CRISPR enzyme comprising a Cas protein that cleaves a reporter nucleic acid and a guide RNA molecule having a sequence that is reverse complementary to a sequence of the target DNA; and assaying for detector nucleic acid signal. 19. A method of detecting methylation of a target DNA in a sample, the method comprising contacting the sample to a reagent that differentially reacts to methylated bases; contacting the sample to a composition comprising a CRISPR enzyme comprising a Cas protein that cleaves a detector nucleic acid and a guide RNA molecule having a sequence that is reverse complementary to a sequence of the target DNA; and assaying for detector nucleic acid signal. 20. A method of detecting methylation of a target DNA of a sample, the method comprising: performing bisulfite conversion on the sample; contacting the sample to a Cas guide RNA molecule having a sequence that is complementary to a sequence of the target DNA, a detector nucleic acid, and a Cas protein that cleaves the reporter nucleic acid; and observing a signal produced by cleavage of the detector nucleic acid when the target DNA is unmethylated. 21. A method of detecting methylation of a target DNA in a sample, the method comprising: a) contacting the sample to a methylation-specific restriction enzyme; b) contacting the sample to a composition comprising: (i) a CRISPR enzyme comprising a Cas protein that cleaves a reporter nucleic acid and a guide RNA molecule having a sequence that is reverse complementary to a sequence of the target DNA; and c) assaying for detector nucleic acid signal. 22. A method of detecting methylation of a target DNA in a sample, the method comprising: a) contacting the sample to a reagent that differentially reacts to methylated bases; b) contacting the sample to a composition comprising (i) a CRISPR enzyme comprising a Cas protein that cleaves a detector nucleic acid and (ii) a guide RNA molecule having a sequence that is reverse complementary to a sequence of the target DNA; and c) assaying for detector nucleic acid signal. 23. A method of detecting methylation of a target DNA of a sample, the method comprising: a) performing bisulfite conversion on the sample; b) contacting the sample to (i) a Cas guide RNA molecule having a sequence that is complementary to a sequence of the target DNA; (2) a detector nucleic acid; and (3) a Cas protein that cleaves the detector nucleic acid; and c) observing a signal produced by cleavage of the detector nucleic acid when the target DNA is unmethylated. 24. The method of any one of embodiments 1-23, wherein the programmable nuclease comprises a Cas protein that cleaves a detector nucleic acid and a guide RNA molecule having a sequence that is reverse complementary to a sequence of the methylated nucleic acid. 25. The method of any one of embodiments 1-24, wherein the methylation sensitive CRISPR enzyme comprises a Cas protein that cleaves a detector nucleic acid and a guide RNA molecule having a sequence that is reverse complementary to a sequence of the methylated nucleic acid. 26. The method of any one of embodiments 1-25, wherein the methylated nucleic acid is methylated RNA or methylated DNA. 27. The method of any one of embodiments 1-26, further comprising a detector nucleic acid. 28. The method of any one of embodiments 7-27, wherein the detector nucleic acid comprises a fluorophore and fluorescence quencher. 29. The method of any one of embodiments 7-28, wherein the detector nucleic acid comprises from 5 nucleotides to 14 nucleotides. 30. The method of any one of embodiments 7-29, wherein the detector nucleic acid comprises 6 nucleotides. 31. The method of any one of embodiments 7-29, wherein the detector nucleic acid comprises 8 nucleotides. 32. The method of any one of embodiments 7-29, wherein the detector nucleic acid comprises 10 nucleotides. 33. The method of any one of embodiments 7-32, wherein a Cas protein cleaves the detector nucleic acid. 34. The method of any one of embodiments 7-33, further comprising measuring a signal produced by cleavage of the detector nucleic acid. 35. The method of any one of embodiments 1-34, further comprising a methylation-specific restriction enzyme. 36. The method of embodiment 35, wherein the methylation-specific restriction enzyme is Dpnl, DpnII, Mspl, MspJlAat II, Acc II, Aor13H I, Aor51H I, BspT104 I, BssH II, Cfr10 I, Cla I, Cpo I, Eco52, I, Hae II, Hha I, Mlu I, Nae I, Not I, Nru I, Nsb I, PmaC I, Psp1406 I, Pvu I, Sac II, Sal I, Sma I, SnaB I, or Epi HpaII. 37. The method of any one of embodiments 35-36, wherein the methylation-specific restriction enzyme is Epi HpaII. 38. The method of any one of embodiments 1-37, further comprising mutating unmethylated cytosine residues by bisulfite conversion. 39. The method of any one of embodiments 9-38, wherein the Cas protein is Cas12 protein, Cas13 protein, or Cas14 protein. 40. The method of embodiment 39, wherein the Cas12 protein is Cas12a, Cas12b, Cas12c, Cas12d, or Cas12e. 41. The method of embodiment 39, wherein the Cas13 protein is Cas13a, Cas13b, Cas13c, Cas13d, or Cas13e. 42. The method of embodiment 39, wherein the Cas14 protein is Cas14a, Cas14b, Cas14c, Cas14d, Cas14e, Cas14f, Cas14g, Cas14h, Cas14i, Cas14j, or Cas14k. 43. The method of any one of embodiments 1-42, wherein the methylated nucleic acid comprises a methylation variable region. 44. The method of embodiment 43, wherein a nucleic acid residue of the methylated nucleic acid is methylated in the methylation variable region. 45. The method of any one of embodiments 1-44, wherein the methylated nucleic acid comprises a CpG methylation. 46. The method of any one of embodiments 1-44, wherein the methylated nucleic acid comprises a N6-methyladenosine. 47. The method of any one of embodiments 7-46, wherein the guide nucleic acid molecule has a sequence comprising at least one uracil in a region from nucleic acid residue 5 to 20 that is reverse complementary to a methylation variable region in the methylated nucleic acid. 48. The method of any one of embodiments 1-47, further comprising amplifying the methylated nucleic acid. 49. The method of embodiment 48, wherein the amplifying is by PCR amplification. 50. The method of embodiment 48, wherein the amplifying is by isothermal amplification. 51. The method of embodiment 50, wherein the isothermal amplification is isothermal recombinase polymerase amplification (RPA), transcription mediated amplification (TMA), strand displacement amplification (SDA), helicase dependent amplification (HDA), loop mediated amplification (LAMP), rolling circle amplification (RCA), single primer isothermal amplification (SPIA), ligase chain reaction (LCR), simple method amplifying RNA targets (SMART), improved multiple displacement amplification (IMDA), or nicking enzyme amplification reaction (NEAR). 52. The method of any one of embodiments 50-51, wherein the amplification is helicase dependent amplification (HDA). 53. The method of any one of embodiments 50-51, wherein the amplification is loop mediated amplification (LAMP). 54. The method of any one of embodiments 50-51, wherein the amplification is recombinase polymerase amplification (RPA). 55. The method of any one of embodiments 1-54, wherein the sample is from a human subject. 56. The method of any one of embodiments 1-55, wherein the sample is a biological sample. 57. The method of any one of embodiments 1-56, wherein the sample is a tissue, blood, or urine sample. 58. The method of any one of embodiments 55-57, wherein the human subject has cancer. 59. A method comprising contacting a modification sensitive programmable nuclease to a sample comprising a modified nucleic acid. 60. The method of embodiment 59, wherein the modification sensitive programmable nuclease is a modification sensitive CRISPR enzyme. 61. A method comprising contacting a sample comprising a modified nucleic acid to an enzyme composition comprising a programmable nuclease, wherein the enzyme composition exhibits modification sensitive cleavage. 62. The method of embodiment 61, wherein the enzyme composition comprises a CRISPR enzyme, wherein the enzyme composition exhibits modification sensitive cleavage. 63. A method comprising contacting a sample comprising a modified nucleic acid to a reagent and to a programmable nuclease, wherein the reagent differentially reacts to modified bases. 64. The method of embodiment 63, wherein the programmable nuclease is a CRISPR enzyme. 65. The method of any one of embodiments 59-64, wherein the modified nucleic acid comprises an adenosine-to-inosine modification or methylation modification. 66. The method of any one of embodiments 59-64, wherein the modified nucleic acid comprises an 5-hydroxymethylcytosine (5hmC), 5-formylcytosine (5fC), 5-carboxylcytosine (5caC), 5-hydroxymethyluracil (5hmU), 5-methylcytosine (5mC), 3-methylcytosine (3mC), N6-methyladenine (m6A), N6, 2′-O-dimethyladenine (m6Am), N1-methyladenine (m 1A), N1-methylguanine (m1 G), 5-methylcytosine (m5C), or 5-hydroxymethylcytosine (hm5C). 67. The method of any one of embodiments 1-66, wherein the target nucleic acid comprises a segment of a gene encoding APC, p 16INK4A, DAPK11, NANOG, FOXM1, MYC, YAP, RASSF1A, p16INK4A, CDH1, or TFPI2. 68. The method of any one of embodiments 1-67, wherein the target nucleic acid comprises a segment of a gene encoding TFPI2. 69. The method of any one of embodiments 1-67, wherein the target nucleic acid comprises a segment of a gene encoding APC. 70. The method of any one of embodiments 1-67, wherein the target nucleic acid comprises a segment of a gene encoding p16INK4A. 71. The method of any one of embodiments 1-67, wherein the target nucleic acid comprises a segment of a gene encoding DAPK11. 72. The method of any one of embodiments 1-67, wherein the target nucleic acid comprises a segment of a gene encoding NANOG. 73. The method of any one of embodiments 1-67, wherein the target nucleic acid comprises a segment of a gene encoding FOXM1 . 74. The method of any one of embodiments 1-67, wherein the target nucleic acid comprises a segment of a gene encoding MYC. 75. The method of any one of embodiments 1-67, wherein the target nucleic acid comprises a segment of a gene encoding YAP. 76. The method of any one of embodiments 1-67, wherein the target nucleic acid comprises a segment of a gene encoding p16INK4A. 77. The method of any one of embodiments 1-67, wherein the target nucleic acid comprises a segment of a gene encoding CDH1. 78. A method of assaying for a target nucleic acid in a sample, comprising: selectively producing a target single stranded DNA (ssDNA) using amplification of the target nucleic acid of the sample; contacting the target ssDNA to a complex comprising a guide nucleic acid comprising a segment that is reverse complementary to a segment of the target ssDNA and a programmable nuclease, wherein the programmable nuclease exhibits sequence independent cleavage upon forming a complex comprising the segment of the guide nucleic acid binding to the segment of the target ssDNA; and assaying for cleavage of at least one detector nucleic acid molecule of a population of detector nucleic acid molecules, wherein the cleavage indicates a presence of the target nucleic acid in the sample and wherein the absence of the cleavage indicates an absence of the target nucleic acid in the sample. 79. The method of embodiment 78, wherein selectively producing the target ssDNA comprises amplifying a target double stranded DNA having the target ssDNA and a nontarget ssDNA and selectively degrading the nontarget ssDNA. 80. The method of any one of embodiments 78-79, wherein selectively producing the target ssDNA comprises amplifying the target ssDNA, wherein amplifying the target ssDNA comprises amplifying a target double stranded DNA having the target ssDNA and a nontarget ssDNA and selectively producing an amplified target ssDNA. 81. The method of any one of embodiments 78-80, wherein the amplifying comprises thermal cycling amplification. 82. The method of any one of embodiments 78-80, wherein the amplifying comprises isothermal amplification. 83. The method of embodiment 82, wherein the isothermal amplification is select from the group consisting of isothermal recombinase polymerase amplification (RPA), transcription mediated amplification (TMA), strand displacement amplification (SDA), helicase dependent amplification (HDA), loop mediated amplification (LAMP), rolling circle amplification (RCA), single primer isothermal amplification (SPIA), ligase chain reaction (LCR), simple method amplifying RNA targets (SMART), improved multiple displacement amplification (IMDA), nucleic acid sequence-based amplification (NASBA), and nicking enzyme amplification reaction (NEAR). 84. The method of any one of embodiments 82-83, wherein the isothermal amplification is helicase dependent amplification (HDA). 85. The method of any one of embodiments 82-83, wherein the isothermal amplification is loop mediated amplification (LAMP). 86. The method of any one of embodiments 78-85, wherein the producing, the contacting, and the assaying are performed in a common reaction volume. 87. The method of any one of embodiments 78-86, wherein the producing of the target ssDNA from the target nucleic acid comprises contacting the target nucleic acid with a forward primer and a reverse primer. 88. The method of embodiment 87, wherein the forward primer comprises at least 2, at least 3, at least 4, at least 5, at least 6, at least 7, at least 8, at least 9, or at least 10 phosphorothioated nucleotides at the 5′ end. 89. The method of any one of embodiments 87-88, wherein the forward primer comprises at least 4 phosphorothioated nucleotides at the 5′ end. 90. The method of any one of embodiments 87-89, wherein the forward primer comprises 4 phosphorothioated nucleotides at the 5′ end. 91. The method of any one of embodiments 87-90, wherein the reverse primer comprises at least 2, at least 3, at least 4, at least 5, at least 6, at least, 7, at least 8, at least 9, or at least 10 phosphorothioated nucleotides at the 5′ end. 92. The method of any one of embodiments 87-91, wherein the reverse primer comprises at least 4 phophorothioated nucleotides at the 5′ end. 93. The method of any one of embodiments 80-92, wherein the amplifying generates amplified double stranded DNA (dsDNA). 94. The method of embodiment 93, wherein the amplified dsDNA is treated with an exonuclease to produce the target ssDNA. 95. The method of embodiment 94, wherein the exonuclease comprises T7 exonuclease. 96. The method of any one of embodiments 87-95, wherein the forward primer is added in excess of the reverse primer. 97. The method of any one of embodiments 87-96, wherein the forward primer is between 10-fold and 100-fold in excess of the reverse primer. 98. The method of any one of embodiments 87-97, wherein the forward primer is 50-fold in excess of the reverse primer. 99. The method of any one of embodiments 87-98, wherein the reverse primer is added in excess of the forward primer. 100. The method of any one of embodiments 87-99, wherein the reverse primer is between 10-fold and 100-fold in excess of the forward primer. 101. The method of any one of embodiments 87-100, wherein the reverse primer is 50-fold in excess of the forward primer. 102. The method of any one of embodiments 78-101, wherein the producing of the target ssDNA from the target nucleic acid comprises contacting the target nucleic acid with an outer forward primer, an inner forward primer, and a reverse primer. 103. The method of any one of embodiments 78-101, wherein the producing of the target ssDNA from the target nucleic acid comprises contacting the target nucleic acid with an outer reverse primer, an inner reverse primer, and a forward primer. 104. The method of any one of embodiments 78-103, wherein the method further comprises amplification using a strand-displacing polymerase. 105. The method of any one of embodiments 78-104, wherein the target nucleic acid comprises cDNA, ssDNA, dsDNA, or RNA. 106. The method of any one of embodiments 78-105, wherein the target nucleic acid is RNA and wherein the method further comprises reverse transcribing the RNA prior to the producing. 107. The method of any one of embodiments 78-106, wherein the programmable nuclease comprises a Cas nuclease. 108. The method of embodiment 107, wherein the Cas nuclease comprises a Cas12 protein. 109. The method of embodiment 107, wherein the Cas nuclease comprises a Cas14 protein. 110. The method of embodiment 107, wherein the Cas nuclease comprises a Cas14a protein. 111. The method of embodiment 108, wherein the Cas12 protein comprises LbCas12a. 112. The method of any one of embodiments 78-111, wherein the guide nucleic acid comprises a crRNA. 113. The method of any one of embodiments 78-112, wherein the guide nucleic acid comprises a crRNA and a tracrRNA. 114. The method of any one of embodiments 78-113, wherein the programmable nuclease is an RNA guided nuclease. 115. The method of any one of embodiments 78-114, wherein the programmable nuclease is an ssDNA activated effector protein that exhibits sequence independent cleavage upon activation. 116. The method of any one of embodiments 78-115, wherein the sequence independent cleavage comprises PAM-independent sequence independent cleavage. 117. The method of any one of embodiments 78-116, wherein the detector nucleic acid comprises a nucleic acid comprising a detectable moiety. 118. The method of any one of embodiments 78-117, wherein the detector nucleic acid comprises a nucleic acid comprising at least two nucleotides, a fluorophore, and a fluorescence quencher, wherein the fluorophore and the fluorescence quencher are linked by the nucleic acid. 119. The method of any one of embodiments 78-118, wherein cleavage of at least one detector nucleic acid yields a signal. 120. The method of any one embodiments 78-119, wherein cleavage of at least one detector nucleic acid activates a photoexcitable fluorophore. 121. The method of any one of embodiments 78-120, wherein cleavage of at least one detector nucleic acid deactivates a photoexcitable fluorophore. 122. The method of any one of embodiments 119-121, wherein the signal is present prior to detector nucleic acid cleavage. 123. The method of any one of embodiments 119-121, wherein the signal is absent prior to detector nucleic acid cleavage. 124. The method of any one of embodiments 78-123, wherein the sample comprises blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. 125. The method of any one of embodiments 78-124, wherein the target nucleic acid comprises a sequence encoding a single nucleotide polymorphism (SNP). 126. The method of any one of embodiments 78-124, wherein the target nucleic acid comprises a sequence encoding a wild type sequence. 127. A method of assaying for a target nucleic acid in a sample, comprising: selectively amplifying a target single stranded DNA (ssDNA); contacting the target ssDNA to a complex comprising a guide nucleic acid comprising a segment that is reverse complementary to a segment of the target ssDNA and a programmable nuclease that exhibits sequence independent cleavage upon forming a complex comprising the segment of the guide nucleic acid binding to the segment of the target ssDNA; and assaying for cleavage of at least some detector nucleic acid molecules of a population of detector nucleic acid molecules, wherein the cleavage indicates a presence of the target nucleic acid in the sample and wherein the absence of the cleavage indicates an absence of the target nucleic acid in the sample. 128. The method of embodiment 127, wherein selectively amplifying the target ssDNA comprises amplifying a target double stranded DNA having the target ssDNA and a nontarget ssDNA and selectively degrading the nontarget ssDNA. 129. The method of any one of embodiments 127-128, wherein selectively amplifying the target ssDNA comprises amplifying a target double stranded DNA having the target ssDNA and a nontarget ssDNA and selectively producing an amplified target ssDNA. 130. The method of any one of embodiments 127-129, wherein the amplifying comprises thermal cycling amplification. 131. The method of any one of embodiments 127-129, wherein the amplifying comprises isothermal amplification. 132. The method of embodiment 131, wherein the isothermal amplification is select from the group consisting of isothermal recombinase polymerase amplification (RPA), transcription mediated amplification (TMA), strand displacement amplification (SDA), helicase dependent amplification (HDA), loop mediated amplification (LAMP), rolling circle amplification (RCA), single primer isothermal amplification (SPIA), ligase chain reaction (LCR), simple method amplifying RNA targets (SMART), improved multiple displacement amplification (IMDA), and nucleic acid sequence-based amplification (NASBA). 133. The method of any one of embodiments 127-132, wherein the producing, the contacting, and the assaying are performed in a common reaction volume. 134. The method of any one of embodiments 127-133, wherein the producing of the target ssDNA from the target nucleic acid comprises contacting the target nucleic acid with a forward primer and a reverse primer. 135. The method of embodiment 134, wherein the forward primer comprises at least 2, at least 3, at least 4, at least 5, at least 6, at least, 7, at least 8, at least 9, or at least 10 phosphorothioated nucleotides at the 5′ end. 136. The method of any one of embodiments 134-135, wherein the forward primer comprises at least 4 phosphorothioated nucleotides at the 5′ end. 137. The method of any one of embodiments 134-136, wherein the forward primer comprises 4 phosphorothioated nucleotides at the 5′ end. 138. The method of any one of embodiments 134-137, wherein the reverse primer comprises at least 2, at least 3, at least 4, at least 5, at least 6, at least, 7, at least 8, at least 9, or at least 10 phosphorothioated nucleotides at the 5′ end. 139. The method of any one of embodiments 134-138, wherein the reverse primer comprises at least 4 phophorothioated nucleotides at the 5′ end. 140. The method of any one of embodiments 127-139, wherein the amplifying generates amplified double stranded DNA (dsDNA). 141. The method of embodiment 140, wherein the amplified dsDNA is treated with an exonuclease to produce the target ssDNA. 142. The method of any one of embodiments 140-141, wherein the exonuclease comprises T7 exonuclease. 143. The method of any one of embodiments 134-142, wherein the forward primer is added in excess of the reverse primer. 144. The method of any one of embodiments 134-143, wherein the forward primer is between 10-fold and 100-fold in excess of the reverse primer. 145. The method of any one of embodiments 134-144, wherein the forward primer is 50-fold in excess of the reverse primer. 146. The method of any one of embodiments 134-145, wherein the reverse primer is added in excess of the forward primer. 147. The method of any one of embodiments 134-146, wherein the reverse primer is between 10-fold and 100-fold in excess of the forward primer. 148. The method of any one of embodiments 134-147, wherein the reverse primer is 50-fold in excess of the forward primer. 149. The method of any one of embodiments 129-148, wherein the producing of the target ssDNA from the target nucleic acid comprises contacting the target nucleic acid with an outer forward primer, an inner forward primer, and a reverse primer. 150. The method of any one of embodiments 129-148, wherein the producing of the target ssDNA from the target nucleic acid comprises contacting the target nucleic acid with an outer reverse primer, an inner reverse primer, and a forward primer. 151. The method of any one of embodiments 127-150, wherein the method further comprises amplification using a strand-displacing polymerase. 152. The method of any one of embodiments 127-151, wherein the target nucleic acid comprises cDNA, ssDNA, dsDNA, or RNA. 153. The method of any one of embodiments 127-152, wherein the target nucleic acid is RNA and wherein the method further comprises reverse transcribing the RNA prior to the producing. 154. The method of any one of embodiments 127-153, wherein the programmable nuclease comprises a Cas nuclease. 155. The method of embodiment 154, wherein the Cas nuclease comprises a Cas12 protein. 156. The method of embodiment 154, wherein the Cas nuclease comprises a Cas14 protein. 157. The method of embodiment 154, wherein the Cas nuclease comprises a Cas14a protein. 158. The method of embodiment 155, wherein the Cas12 protein comprises LbCas12a. 159. The method of any one of embodiments 127-158, wherein the guide nucleic acid comprises a crRNA. 160. The method of any one of embodiments 127-159, wherein the guide nucleic acid comprises a crRNA and a tracrRNA. 161. The method of any one of embodiments 127-160, wherein the programmable nuclease is an RNA guided nuclease. 162. The method of any one of embodiments 127-161, wherein the programmable nuclease is an ssDNA activated effector protein that exhibits sequence independent cleavage upon activation. 163. The method of any one of embodiments 127-162, wherein the sequence independent cleavage comprises PAM-independent sequence independent cleavage. 164. The method of any one of embodiments 127-163, wherein the detector nucleic acid comprises a nucleic acid comprising a detectable moiety. 165. The method of any one of embodiments 127-164, wherein the detector nucleic acid comprises a nucleic acid comprising at least two nucleotides, a fluorophore, and a fluorescence quencher, wherein the fluorophore and the fluorescence quencher are linked by the nucleic acid. 166. The method of any one of embodiments 127-165, wherein cleavage of at least one detector nucleic acid yields a signal. 167. The method of any one embodiments 127-166, wherein cleavage of at least one detector nucleic acid activates a photoexcitable fluorophore. 168. The method of any one of embodiments 127-166, wherein cleavage of at least one detector nucleic acid deactivates a photoexcitable fluorophore. 169. The method of any one of embodiments 166-168, wherein the signal is present prior to detector nucleic acid cleavage. 170. The method of any one of embodiments 166-168, wherein the signal is absent prior to detector nucleic acid cleavage. 171. The method of any one of embodiments 127-170, wherein the sample comprises blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. 172. The method of any one of embodiments 127-171, wherein the target nucleic acid comprises a sequence encoding a single nucleotide polymorphism (SNP). 173. The method of any one of embodiments 127-172, wherein the target nucleic acid comprises a sequence encoding a wild type sequence.

### EXAMPLES

The following examples are included to further describe some aspects of the present disclosure, and should not be used to limit the scope of the invention.

### Example 1

### Detection of Methylated DNA Using Methylation-Specific Restriction Enzymes

This example shows the detection of a methylated target DNA. Methylated and unmethylated pUC19 dsDNA from the Thermo Scientific EpiJET DNA Methylation Analysis Kit were used as target DNA.

15 nM (final concentration) of methylated and unmethylated pUC19 dsDNA underwent a restriction digest by Epi HPAII. The success of the digest was confirmed by gel electrophoresis, where the Epi HpaII digested products were run alongside no enzyme controls and Epi MspI digested products (Epi MspI is an endonuclease with 5′-CCGG-3′ specificity that is not methylation specific). The results are shown in FIG. 1, which depicts the 2% agarose gel confirming restriction digest with Thermo Scientific EpiJET DNA Methylation Analysis kit. Lane 1: Unmethylated pUC19+no enzyme. Lane 2: Unmethylated pUC19+Epi HpaII. Lane 3: Unmethylated pUC19+Epi MpsI. Lane 4: Methylated pUC19+no enzyme. Lane 5: Methylated pUC19+Epi HpaII. Lane 6: Methylated pUC19+Epi MpsI.

To assess detection of DNA methylation without amplification, digested DNA and untreated control DNA were serially diluted and then added to DETECTR reactions to cover a concentration range of 150 pM to 150 aM (final concentration in the DETECTR reaction). For this DETECTR reaction, 2 μl of digested DNA or control DNA were transferred to a 384 well plate and combined directly to the DETECTR reaction mix for a final concentration of 50 nM LbCas12a effector protein, 62.5 nM crRNA, and 50 nm ssDNA-FQ reporter comprising/5′ 6-Fluorescein/TTATTATT/3′ Iowa Black FQ/(SEQ ID NO: 9) in a total reaction volume of 20 μL. Reactions were incubated in a fluorescence plate reader (Tecan Infinite Pro 200 M Plex) for 2 hours at 37° C. with fluorescence measurements taken every 30 seconds (λex: 485 nm; λem: 535 nm). Results are shown in FIG. 2A, which depicts detection of Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) without intermediate amplification. Without amplification, the assay's limit of detection is approximately 100 pM.

To increase sensitivity of the assay, both polymerase chain reaction (PCR) and helicase-dependent amplification (HDA) were tested as intermediate amplification steps following Epi HpaII digestion and preceding detection using the same DETECTR protocol as above.

To assess the sensitivity of detection of PCR-amplified DNA, a serial dilution of Epi HpaII digested DNA underwent both 10 and 25 cycles of amplification. PCRs amplified a 100 bp region containing the CCGG restriction site and the protospacer complementary to the crRNA, and each reaction consisted of 1 μL template DNA, 10 μL 5× Q5 Buffer, 0.48 μM forward/reverse primers, 200 μM (each) dNTPs, and 1 U Q5 DNA Polymerase in a final volume of 50 μL. 2 μL of PCR product was added to the DETECTR reaction for a final concentration of DNA (pre-amplification) ranging from 60 pM to 60 aM. Results are shown in FIG. 2B and FIG. 2C. FIG. 2B depicts the detection of Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) after 10 cycles of PCR amplification. FIG. 2C depicts the detection of Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) after 25 cycles of PCR amplification.

To assess the impact of HDA on sensitivity of the assay, a serial dilution of Epi HpaII digested DNA underwent amplification for 30 minutes and 60 minutes. HDA amplified a 100bp region containing the CCGG restriction site and the protospacer complementary to the crRNA, and each reaction consisted of 1 μL template DNA, 1 μL 10X IsoAmp annealing buffer II, 4 mM MgSO4, 40 mM NaCl, 0.5 μL IsoAmp dNTP solution, 0.075 μM forward/reverse primers, and 0.4 μL IsoAmp Enzyme Mix III in a final volume of 10 μL. HDA reactions were incubated at 65° C. for either 30 or 60 minutes and heat inactivated at 80C for 10 minutes. 2 μL of HDA product was added to the DETECTR reaction for a final concentration of DNA (pre-amplification) ranging from 150 pM to 150 zM. Results are shown in FIG. 3A and FIG. 3B. FIG. 3A depicts the detection of helicase-dependent isothermal amplified Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) after 30 minutes of incubation. FIG. 3B depicts the detection of helicase-dependent isothermal amplified Epi HpaII digested methylated DNA, undigested methylated DNA, Epi HpaII digested unmethylated DNA, or undigested unmethylated DNA serial dilutions using a LbCas12a programmable nuclease (SEQ ID NO: 21) after 60 minutes of incubation.

Both PCR and HDA preserved methylation-specificity in the DETECTR reaction and improved the sensitivity of the assay. 10 cycles of PCR allowed for adequate detection of methylated DNA at a concentration of 60 fM, and 25 cycles of PCR allowed for detection of methylated DNA at 60 aM. However, 25 cycles of PCR amplified background DNA present at higher concentrations of template DNA, as shown in FIG. 2C with a template concentration of 60 pM activator. While 30 minutes of HDA does not dramatically improve the limit of detection for the assay, amplification enables faster detection compared to the same concentration without amplification (FIG. 2A & FIG. 3A). 60 minutes of HDA enables detection of methylated DNA at 150 aM, though it amplifies background significantly at template concentrations of 150 pM and 150 fM.

### Example 2

### Detection of Methylated DNA Using Bisulfite Conversion

This example shows the detection of a methylated target DNA. Methylated and unmethylated pUC19 dsDNA from the Thermo Scientific EpiJET DNA Methylation Analysis Kit are used as target DNA.

Methylated pUC19 dsDNA and unmethylated pUC19 dsDNA are treated with sodium bisulfite using the Thermo Scientific EpiJET Bisulfite Conversion kit. Briefly, 20 ul of molecular grade water containing 200-500 ng of purified methylated pUC19 dsDNA or purified unmethylated pUC19 dsDNA is added to a PCR tube. 120 ul of prepared Modification Reagent solution is added to the methylated pUC19 dsDNA PCR tube and the unmethylated pUC19 dsDNA PCR tube, which are then mixed by pipetting and then centrifuged so that the liquid is at the bottom of the tube. The methylated pUC19 dsDNA PCR tube and the unmethylated pUC19 dsDNA PCR tube are placed in a thermal cycler and proceed with Protocol A: 1) 98 C/10 min, 2) 60 C/150 min, and 3) optionally store overnight at 4 C; or proceed with Protocol B: 1) 98 C/30 min, and 2) optionally store overnight at 4 C, which perform the denaturation and bisulfite conversion of the samples in the PCR tubes. 400 ul of Binding Buffer is added to DNA Purification Micro columns and are placed into a collection tubes. The methylated pUC19 dsDNA PCR tube and the unmethylated pUC19 dsDNA PCR tube after Protocol A or B are then loaded into the Binding Buffer in the columns and are mixed completely by pipetting. The micro columns are placed into the collection tubes and are centrifuged at 12,000 rpm for 30 seconds. The flow through is discarded and the micro columns are placed in the same collection tubes. 120 ul of Desulfonated buffer prepared with ethanol is added to the micro columns, which are then allowed to stand at room temperature for 20 min. The micro columns in the collection tubes are centrifuged for 30 seconds at 12,000 rpm. The flow through is discarded, and the micro columns are placed in the same collection tubes. 200 ul of Wash buffer, prepared with ethanol, is added to the micro column and centrifuged for 30 seconds at 12,000 rpm. The flow through is discarded, and the micro columns are placed in the same collection tubes. 200 ul of Wash buffer, prepared with ethanol, is added to the micro column and centrifuged for 30 seconds at 12,000 rpm. The flow through is discarded, and the micro columns are placed in the same collection tubes. The columns are then placed in clean 1.5 mL microcentrifuge tubes, 10 ul of Elution Buffer is added to each micro column, and then the micro columns are centrifuged at 12,000 rpm for 60 sec. The elution from this last step comprises the bisulfite conversion treated sample. The bisulfite conversion treated methylated pUC19 dsDNA and bisulfite conversion treated unmethylated pUC19 dsDNA are added to DETECTR reactions. For the DETECTR reactions, 2 ul of bisulfite conversion treated methylated pUC19 dsDNA and bisulfite conversion treated unmethylated pUC19 dsDNA are transferred to a 384 well plate and combined directly to the DETECTR reaction mix for a final concentration of 50 nM Cas12a effector protein, 62.5 nM guide RNA, and 50 nm ssDNA-FQ reporter in a total reaction volume of 20 μL. Reactions are incubated in a fluorescence plate reader (Tecan Infinite Pro 200 M Plex) for 2 hours at 37° C. with fluorescence measurements taken every 30 seconds (λex: 485 nm; λem: 535 nm). No fluorescent signal is detected for the methylated pUC19 dsDNA, indicating that the target DNA is methylated. Fluorescent signal is detected for the unmethylated pUC19 dsDNA, indicating that the target DNA is unmethylated.

### Example 3

### Cas13a Sensitivity to Methylation Location on the Target RNA

This example demonstrates the Cas13a sensitivity to the methylation location on target RNA. Unmodified and N6-methyladenosine-modified target RNAs were generated with sets of four consecutive adenosines at different positions along the 20 nucleotide long targeting region. A schematic of the methylation target sites is shown in FIG. 4A. FIG. 4A depicts a schematic of various positions of adenosines (A) in target RNAs, wherein each target RNA contains identical surrounding sequence context (N). The adenosines can either be unmodified or modified (N6-methyladenosine).

A Cas13a assay was then performed on the unmodified target RNA and the m6A-modified (comprising an N6-methyladenine (m6A) nitrogenous base) target RNA. For these Cas13a assays, Cas13a and crRNA were first incubated at 37° C. for 30 min in lx Cas13a reaction buffer to generate RNA-protein complexes. 15 ul of the RNA-protein complexes were combined with 5 ul of RNA-FQ detector nucleic acids comprising/5′ 6-Fluorescein/rUrUrUrUrU/3′ Iowa Black FQ/(SEQ ID NO: 1) and 10 pM (final concentration) of unmodified target RNA or m6A-modified target RNA. The components of the final reaction contained lx Cas13a reaction buffer, 40 nM crRNA, 40 nM LbuCas13a, 170 nM RNA-FQ detector nucleic acids, and 10 pM of unmodified target RNA or m6A-modified target RNA. The reactions were incubated for 1.5 hours in a fluorescent plate reader (Tecan Infinite Pro 200 M Plex) at 37 C with fluorescence measurements taken every 30 seconds (λex: 490 nm; λem: 525 nm). Generally, since m6A disrupts RNA:RNA interactions, the base pairing between the m6A-modified target RNA was disrupted so that the Cas13a was unable to recognize the target RNA to initiate non-specific cleavage of the RNA-FQ detector nucleic acid, resulting in decreased detection of the RNA-FQ detector nucleic acid, as read by the fluorescence plate reader as compared to the detection of the RNA-FQ in the sample with the unmodified target RNA. Results are shown in FIG. 4B, which depicts the normalized fluorescence readings from Cas13a detection assay with unmodified adenosine or modified adenosine (N6-methyladenosine) target RNAs of FIG. 4A. Results indicate that Cas13a was most sensitive to m6A modifications in the 5′ end of the target RNA, particularly in the regions 1-4 and 5-8. Partial sensitivity was seen in regions 9-12 and 13-16. No sensitivity to N6-methyladenosine modified RNAs was observed in the region 17-20 of the target RNA.

### Example 4

### Detection of Methylated RNA

This example shows the detection of methylated RNA using Cas13a. Additionally, this example demonstrates that Cas13a can detect N6-methyladenine (m6A) on sequences with increased complexity. First, a 115 nucleotide long target RNA was synthesized by in vitro transcription using either ATP or m6ATP. This target RNA was derived from a naturally occurring RNA sequence and had adenosine residues throughout. To determine whether Cas13a is sensitive to this more diverse substrate, crRNAs were designed based on the observations seen in EXAMPLE 2 and EXAMPLE 3 to differentiate between unmodified and m6A-modified RNAs with one or two adenosines in the methylation variable region.

Unmodified and m6A-modified target RNAs were added to a Cas13a detection assay for each of the crRNAs at a final concentration of 10 pM. The results are shown in FIG. 4C, which depicts raw fluorescent results of four different crRNAs along either an unmodified or modified (N6-methyladenosine) target RNA derived from a natural sequence.

### Example 5

### Diagnosis of Cancer Due to Methylation Status of a Gene

This example shows the diagnosis of cancer due to the methylation status of a gene. A biological sample is taken from a subject and DNA is extracted from the biological sample. The extracted sample DNA and a control DNA comprising the unmodified DNA of the cancer gene are digested using the restriction endonuclease Epi HPAII. The digested sample DNA and control DNA are added to DETECTR reactions comprising a guide nucleic acid that targets the cancer gene, and fluorescent signal is assessed. For these DETECTR reactions, 2 ul of the sample DNA or control DNA are transferred to a 384 well plate and are combined directly with the DETECTR reaction mix for a final concentration of 50 nM Cas12a effector protein, 62.5 nM crRNA, and 50 nm ssDNA-FQ reporter in a total reaction volume of 20 μL. The reactions are incubated in a fluorescence plate reader (Tecan Infinite Pro 200 M Plex) for 2 hours at 37° C. with fluorescence measurements taken every 30 seconds (λex: 485 nm; λem: 535 nm). If a fluorescent signal is detected, the cancer gene DNA is methylated and indicates the subject is diagnosed with cancer. If no fluorescent signal is detected, the cancer gene DNA is unmethylated and the subject is not diagnosed with cancer.

### Example 6

### Diagnosis of a Genetic Disorder Due to RNA Methylation Status

This example shows the diagnosis of genetic disorder due to the methylation status of a RNA associated with a genetic disorder. A biological sample is taken from a subject and RNA is extracted from the biological sample. The extracted RNA is added to a Cas13a assay comprising a guide nucleic acid that binds to variable methylation region of the RNA, and fluorescent signal is assessed. If a fluorescent signal is detected, the RNA is not methylated and indicates the subject does not have the genetic disorder. If no fluorescent signal is detected, the RNA is unmethylated and the subject is diagnosed the genetic disorder.

### Example 7

### Diagnosis of Colorectal Cancer Due to Methylation Status of the promoter of TFPI2

This example shows the diagnosis of colorectal cancer due to increased detection of methylation of the promoter of the TFPI2 gene. A biological sample is taken from a subject and DNA is extracted from the biological sample. The extracted sample DNA and a control DNA comprising the unmodified DNA of the TFPI2 gene are digested using the restriction endonulcease Epi HPAII. A serial dilution of Epi HpaII digested sample DNA and control DNA will then undergo amplification for 30 minutes and 60 minutes. HDA amplified a 100 bp region containing the CCGG restriction site and the protospacer complementary to the crRNA, and each reaction consisted of 1 μL template DNA, 1 μL 10× IsoAmp annealing buffer II, 4 mM MgSO₄, 40 mM NaCl, 0.5 μL IsoAmp dNTP solution, 0.075 μM forward/reverse primers, and 0.4 μL IsoAmp Enzyme Mix III in a final volume of 10 μL. HDA reactions are then incubated at 65° C. for either 30 or 60 minutes and heat inactivated at 80° C. for 10 minutes. 2 μL of HDA products of the sample DNA reaction or the control DNA reaction are added to a DETECTR reaction, in which they are transferred to a 384 well plate and are combined directly with the DETECTR reaction mix for a final concentration of 50 nM Cas12a effector protein, 62.5 nM crRNA, and 50 nm ssDNA-FQ reporter in a total reaction volume of 20 μL. The reactions are incubated in a fluorescence plate reader (Tecan Infinite Pro 200 M Plex) for 2 hours at 37° C. with fluorescence measurements taken every 30 seconds (λex: 485 nm; λem: 535 nm). Fluorescent signal from the sample DNA is compared with a standard curve determined by detection of fluorescence in the control DNA to determine whether methylation is more frequent in the sample DNA than the control DNA. If the fluorescent signal is more frequent in the sample DNA, the subject is diagnosed with colorectal cancer.

### Example 8

### Diagnosis of a Parkinson's Disease Due to Hypermethylation of SNCA

This example shows the diagnosis of Parkinson's disease due to the detection of hypermethylation of the SNCA gene. A biological sample is taken from a subject and DNA is extracted from the biological sample. The extracted sample DNA and a control DNA comprising the unmethylated DNA of the SNCA gene are digested using the restriction endonuclease Epi HPAII. A serial dilution of Epi HpaII digested sample DNA and control DNA will then undergo amplification for 30 minutes and 60 minutes. HDA amplified a 100bp region containing the CCGG restriction site and the protospacer complementary to the crRNA, and each reaction consisted of 1 template DNA, 1 μL 10× IsoAmp annealing buffer II, 4 mM MgSO₄, 40 mM NaCl, 0.5 IsoAmp dNTP solution, 0.075 ᵢ.tM forward/reverse primers, and 0.4 μL IsoAmp Enzyme Mix III in a final volume of 10 μL. HDA reactions are then incubated at 65° C. for either 30 or 60 minutes and heat inactivated at 80° C. for 10 minutes. 2 μL of HDA products of the sample DNA reaction or the control DNA reaction are added to a DETECTR reaction, in which they are transferred to a 384 well plate and are combined directly with the DETECTR reaction mix for a final concentration of 50 nM Cas12a effector protein, 62.5 nM crRNA, and 50 nm ssDNA-FQ reporter in a total reaction volume of 20 μL. The reactions are incubated in a fluorescence plate reader (Tecan Infinite Pro 200 M Plex) for 2 hours at 37° C. with fluorescence measurements taken every 30 seconds (λex: 485 nm; λem: 535 nm). Fluorescent signal from the sample DNA is compared with a standard curve determined by detection of fluorescence in the control DNA to determine whether the population of methylated nucleic acid is higher in the sample DNA than the control DNA. Fluorescent signal increases as a function of increasing methylated nucleic acid population. If the fluorescent signal is higher in the sample DNA, the subject is diagnosed with Parkinson's Disease.

### Example 9

### Cas12a Detection of Uracil-Containing Amplicons

This example shows that Cas12a detects uracil-containing amplicons. 10 nM DNA was amplified by HDA with standard dNTPs (A/G/C/T) or with a dA/G/C/UTP mix (no thymines). Cas12a detection of 2 μL of these reactions is shown in FIG. 5A alongside a no amplification control, demonstrating that Cas12a can detect uracil-containing amplicons at a rate similar to that of thymine-containing amplicons. FIG. 5B shows the sequences of the crRNA (pUC19 Cas12a gRNA), forward and reverse HDA/PCR primers, and the pUC19 amplicon of FIG. 5A.

### Example 10

### Cas14a1 and LbCas12a Detection of the HERC2 Gene in Human Saliva Via PCR with a Phosphorothioated (PT′d) Primer and Treatment by T7 Exonuclease

This example illustrates Cas14a1 and LbCas12a detection of the HERC2 gene in human saliva via PCR with a phosphorothioated (PT′d) primer and treatment by T7 exonuclease.

PCR with a PT′d/unmodified primer pair and treatment with T7 exonuclease enabled Cas14a1 detection of the HERC2 A/G SNP in the human genome (dsDNA) and PAM-independent detection of the HERC2 A/G SNP in the human genome (dsDNA) by LbCas12a.

Saliva samples were taken at three independent times from brown and blue-eyed individuals. For crude DNA extraction, saliva was pelleted and washed twice in phosphate buffered saline (PBS) (1 x PBS), incubated for 5 min at 100° C., and centrifuged for 5 min at 10000×g.

50 μL PCR reactions consisting of 1 μL template DNA, 10 μL 5× Q5 Buffer, 0.48μM forward/reverse primers, 200 μM (each) dNTPs, and 1 U Q5 DNA Polymerase underwent 25 cycles of amplification. The first four 5′ nucleotides of the forward primer were phosphorothioated to protect from degradation by T7 exonuclease in the subsequent detection step, while the 5′ end of the reverse primer was unmodified. DETECTR assays by LbCas12a and Cas14a1 were conducted as described above.

The graph of FIG. 9 illustrates the background subtracted fluorescence (absorbance units; AU) on the y-axis and each of the test group on the x-axis. A high fluorescence indicated Cas14a and Cas12a detection of a target nucleic acid encoding a segment of the HERC2 gene. illustrates SNP ssDNA detection using Cas14a-DETECTR with a blue-eye targeting guide for saliva samples from blue-eyed and brown-eyed individuals compared with ssDNA detection using Cas12a. Amplification of the HERC2 gene from human genomic DNA was conducted with a PT′d primer followed by T7 exonuclease treatment, enabling Cas14a1 (SEQ ID NO: 33) detection of an originally dsDNA target and PAM-independent detection by LbCas12a (SEQ ID NO: 21). As shown in FIG. 9, this method of amplification generated ssDNA amplicons as the target ssDNA for Cas14a1 wherein the original target nucleic acid was dsDNA and also allowed for PAM-independent detection by LbCas12a using the ssDNA amplicons as the target ssDNA. The Cas14a A SNP group shows little fluorescence because a region of the blue-eyed targeting guide RNA is sensitive to the A SNP mismatch of target ssDNA and therefore did not activate the Cas14a1 trans cleavage activity in the presence of the target ssDNA encoding the A SNP of brown eyed individuals.

### Example 11

### Cas14a1 and LbCas12a Detection of ssDNA Amplicons Via Helicase-Dependent Amplification (HDA) with a Phosphorothioated (PT′d) Primer and Treatment by T7 Exonuclease

This example illustrates Cas14a1 and LbCas12a detection of ssDNA amplicons via helicase-dependent amplification (HDA) with a phosphorothioated (PT′d) primer and treatment by T7 exonuclease.

HDA reactions were conducted with the Qudiel IsoAmp III kit, consisting of 1 μL template DNA, 1 μL 10× IsoAmp annealing buffer II, 4 mM MgSO₄, 40 mM NaCl, 0.5 μL IsoAmp dNTP solution, 0.075 μM forward/reverse primers, and 0.4 μL IsoAmp Enzyme Mix III in a final volume of 10 μL. HDA reactions were incubated at 65 C for 60 minutes and heat inactivated at 80 C for 10 minutes. 2 μL of HDA product was added to the DETECTR reaction for T7 exonuclease treatment and detection by Cas14a1.

Results comparing Cas14a1 detection of amplified and unamplified ssDNA oligonucleotides are shown in FIG. 11. FIG. 11 illustrates Cas14a1 (SEQ ID NO: 33) DETECTR detection of ssDNA amplicons generated by HDA with a PT′d primer followed by treatment with an exonuclease compared to Cas14a1 DETECTR detection of ssDNA oligonucleotides without HDA. This figure shows a graph of background-subtracted fluorescence (absorbance units; AU) for the 10 nM oligo+HDA group (amplified) and the 10 nM oligo group (unamplified). A high fluorescence indicated successful detection of ssDNA.

Results demonstrating the boost in sensitivity of PAM-less detection of M13 ssDNA by LbCas12a are shown in FIG. 12. FIG. 12 illustrates LbCas12a (SEQ ID NO: 21) DETECTR detection of HDA amplified M13 ssDNA plasmid following treatment with a T7 exonuclease compared to detection of M13 ssDNA without HDA.

HDA with a PT′d/unmodified primer pair and treatment with T7 exonuclease improved Cas14a1 detection of ssDNA oligonucleotides containing the human HERC2 gene. This strategy also enabled high-sensitivity PAM-less detection of M13 ssDNA plasmid by LbCas12a.

### Example 12

### Cas14a1 Detection of Oligonucleotides Via Asymmetric PCR

This example illustrates Cas14a1 detection of oligonucleotides via asymmetric PCR.

Amplification with PCR with an asymmetric concentration of primers enabled ssDNA amplification from an ssDNA oligonucleotide template. The ssDNA oligonucleotide template corresponded to the non-targeted strand by the Cas14a1 crRNA, thus only amplified ssDNA by the asymmetric PCR would be detectable in the DETECTR assay.

50 μL PCR reactions consisting of 1 μL template DNA, 10 μL 5× Q5 Buffer, forward primer, 0.04 μM reverse primer, 200 μM (each) dNTPs, and 1 U Q5 DNA Polymerase underwent 25 cycles of amplification. 2 μL of amplified product were subsequently either ran on 2% agarose gel electrophoresis or used as input for a Cas14a1 DETECTR reaction. Primer ratios were adjusted by increasing the amount of forward primer and keeping the reverse primer concentration fixed at 0.04 μM. For example, a forward:reverse ratio of 100:1 would entail a forward primer concentration of 4 μM and a reverse primer concentration.

A gel of PCR amplified products with forward:reverse primer concentration ratios of 1:1, 40:1, 50:1, and 60:1 are shown in FIG. 13. FIG. 13 illustrates gel electrophoresis of amplicons generated by PCR with an asymmetric concentration of primers, demonstrating the effect of varying primer ratios and starting DNA concentration to maximize ssDNA amplification.

Cas14a1 DETECTR results of both a broad and refined screen of primer ratios are shown in FIG. 14. FIG. 14 illustrates Cas14a1 (SEQ ID NO: 33) DETECTR assays on PCR amplified oligonucleotides. NTS ssDNA oligonucleotides were the template for the PCR reaction, and TS ssDNA amplicons were generated by an asymmetric concentration of primers. FIG. 14A illustrates the effect of wide range of forward:reverse primer concentration ratio. FIG. 14B illustrates refining forward:reverse primer concentration ratio to optimize ssDNA amplification. ssDNA amplification is maximal when the forward primer is in 50:1 excess over the reverse primer.

### Example 13

### LbCas12a Detection of pUC19 dsDNA

This example illustrates LbCas12a detection of pUC19 dsDNA.

20 μL RPA/DETECTR reactions contained 10 μL 2× TwistAmp Reaction Buffer, 450 μM dNTPs (each), 2 μL 10X TwistAmp Basic E-mix, 0.48 μM forward/reverse primer (PT-modified or unmodified), 1 μL of TwistAmp Core Reaction Mix, 50 nM LbCas12a:crRNA complex, 50 nM FQ-reporter ssDNA, 14 mM MgOAc, and 2 μL of template DNA. All RPA components were mixed with precomplexed LbCas12a RNP and FQ-reporter ssDNA for a final volume of 18 and 2 μL of template DNA was added to initiate the simultaneous amplification/detection.

20 μL DETECTR reactions (no amplification) contained 50 nM FQ-reporter ssDNA and 50 nM precomplexed LbCas12a:crRNA. 2 μL of template DNA was added to 18 μL DETECTR master mix to initiate detection.

All reactions were incubated in a fluorescence plate reader (Tecan Infinite Pro 200 M Plex) for 1.5 hours at 37° C. with fluorescence measurements taken every 30 seconds (λex: 485 nm; λem: 535 nm). TABLE 5 shows relevant DNA sequences.

TABLE 5
DNA Sequences

| Name | Sequence |
|---|---|
| Forward Primer | GTTAGCTCACTCATTAGGCACCCCAG |
| (+/− PT) | (SEQ ID NO: 18) |
| Reverse Primer | CTGTTTCCTGTGTGAAATTGTTATCC |
| (+/− PT) | (SEQ ID NO: 19) |
| pUC19 Amplicon | CTGTTTCCTGTGTGAAATTGTTATCCGC |
| | TCACAATTCCACACAACATACGAGCCGG |
| | AAGCATAAAGTGTAAAGCCTGGGGTGCC |
| | TAATGAGTGAGCTAAC |
| | (SEQ ID NO: 20) |
| 8 nt ssDNA FQ | /56-FAM/TTTTTTTT/3IABkFQ |
| (+/− PT) | (SEQ ID NO: 11) |
| 12 nt ssDNA FQ | /56-FAM/TTTTTTTTTTTT/3IABkFQ |
| (+/− PT) | (SEQ ID NO: 13) |

Since Cas12a and Cas14a become indiscriminate ssDNases after activation by target DNA, one challenge to a one-pot amplification/detection step performed in a common reaction volume is the potential degradation of ssDNA primers by Cas12a or Cas14a. Previous results (FIG. 15A-FIG. 15B) have shown that phosphorothioated (“PT′d”) DNA inhibits cleavage by Cas12a and Cas14a1. FIG. 15A illustrates Cas12a (SEQ ID NO: 21) trans cleavage of an unmodified ssDNA FQ Reporter (8 nt−PT) and a fully phosphorothioated ssDNA FQ reporter (8 nt+PT). Full phosphorothioation appears to inhibit trans cleavage by SEQ ID NO: 21. FIG. 15B illustrates Cas14a1 (SEQ ID NO: 33) trans cleavage of an unmodified ssDNA FQ Reporter (12 nt−PT) and a fully phosphorothioated ssDNA FQ reporter (12 nt+PT). Full phosphorothioation appears to inhibit trans cleavage by SEQ ID NO: 33.

### Example 14

### ssDNA Amplification with PT′d Forward Primers

This example illustrates ssDNA amplification with PT′d forward primers.

The minimal amount of T7 exonuclease necessary for reliable degradation of the unmodified DNA strand in the DETECTR reaction mix was evaluated. PT-modified dsDNA fragments were amplified via PCR or isothermal techniques.

100 nM Cas14a1, 125 nM sgRNA, and 50 nM ssDNA-FQ reporter in a total reaction volume of 20 μL. The LbCas12a DETECTR reaction consists of a final concentration of 50 nM LbCas12a, 50 nM sgRNA, and 50 nM ssDNA-FQ reporter in a total reaction volume of 20 μL. Reactions are incubated in a fluorescence plate reader (Tecan Infinite Pro 200 M Plex) for 2 hours at 37° C. with fluorescence measurements taken every 30 seconds (e.g., λex: 485 nm; λem: 535 nm).

As shown in FIG. 8B, the addition of 5 U of NEB T7 exonuclease to the DETECTR reaction (total volume of 20 μL) is enough to achieve viable detection of the ssDNA activator by Cas14a1. FIG. 8B illustrates the minimum amount of NEB T7 exonuclease added to a 20 DETECTR reaction required to achieve a viable fluorescent signal with Cas14a1 DETECTR.

As shown in FIG. 8A, 2.5 U of NEB T7 exonuclease allows for PAM-independent detection of ssDNA by LbCas12a. FIG. 8A illustrates the minimum amount of NEB T7 exonuclease added to a 20 μL DETECTR reaction required to achieve a viable fluorescent signal with LbCas12a DETECTR.

FIG. 10 illustrates gel electrophoresis of helicase-dependent amplification (HDA) products demonstrating that HDA prefers amplicons less than 120 bp (lane 3, 4), and that HDA tolerates amplification with PT′d primers making it a compatible amplification platform for use with the PT′d primer/exonuclease ssDNA amplification strategy.

### Example 15

### Detection of a Target Nucleic Acid with ssDNA-Activated Programmable Nucleases and DETECTR by Amplification with Asymmetric Concentrations of Primers

This example illustrates detection of a target nucleic acid with ssDNA activated programmable nuclease and DETECTR by amplification with asymmetric concentrations of primers.

A biological sample containing a target nucleic acid is provided. The target nucleic acid is cDNA, ssDNA, dsDNA, or RNA. If the target nucleic acid is RNA, the RNA is reverse transcribed. The target nucleic acid is contacted with an excess of forward primer as compared to the reverse primer or with an excess of reverse primer as compared to the forward primer. Amplification is carried out by PCR, isothermal techniques, recombinase polymerase amplification (RPA), or helicase dependent amplification (HDA), generating amplified ssDNA activator. The amplified ssDNA activator activates a programmable nuclease in a PAM-independent manner. The programmable nuclease is a Cas nuclease, such as Cas12a or Cas14a. The sample is contacted with a reporter, such as an ssDNA fluorescence-quenching (FQ) reporter molecule. The activated programmable nuclease indiscriminately cleaves the reporter, generating a detectable signal. The detectable signal is colorimetric or fluorescent. The detectable signal is captured by an instrument, thereby detecting the presence of the target nucleic acid.

### Example 16

### Detection of a Target Nucleic Acid with ssDNA-Activated Programmable Nucleases and DETECTR by Strand Displacing Amplification of ssDNA with Nested Primer

This example illustrates detection of a target nucleic acid with ssDNA activated programmable nuclease and DETECTR by strand displacing amplification of ssDNA with nested primers.

A biological sample containing a target nucleic acid is provided. The target nucleic acid is cDNA, ssDNA, dsDNA, or RNA. If the target nucleic acid is RNA, the RNA is reverse transcribed. The target nucleic acid is contacted with an outer forward primer, an inner forward primer, and a reverse primer or an outer reverse primer, an inner reverse primer, and a forward primer. Amplification is carried out using a strand-displacing polymerase, generating amplified ssDNA activator. The amplified ssDNA activator activates a programmable nuclease in a PAM-independent manner. The programmable nuclease is a Cas nuclease, such as Cas12a or Cas14a. The sample is contacted with a reporter, such as an ssDNA fluorescence-quenching (FQ) reporter molecule. The activated programmable nuclease indiscriminately cleaves the reporter, generating a detectable signal. The detectable signal is colorimetric or fluorescent. The detectable signal is captured by an instrument, thereby detecting the presence of the target nucleic acid.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of assaying for a modification state of a segment of a target nucleic acid, the method comprising:
contacting a sample comprising the target nucleic acid to:
a guide nucleic acid that hybridizes to the segment of the target nucleic acid;
a detector nucleic acid; and
a programmable nuclease that cleaves the detector nucleic acid upon hybridization of the guide nucleic acid to the segment of the target nucleic acid; and
assaying for a first signal produced by cleavage of the detector nucleic acid to determine the modification state of the segment of the target nucleic acid.

2. The method of claim 1, further comprising:
contacting a second sample comprising a nucleic acid having an unmodified segment comprising the same sequence as the segment of the target nucleic acid to:
the guide nucleic acid;
the detector nucleic acid; and
the programmable nuclease that cleaves the detector nucleic acid upon hybridization of the guide nucleic acid to the segment of the unmodified nucleic acid;
assaying for a second signal produced by cleavage of the detector nucleic acid in the second sample; and
determining the modification state of the target nucleic acid based on a comparison of the first signal to the second signal.

3. The method of claim 1, wherein the target nucleic acid is target RNA,

4. The method of claim 1, wherein the target nucleic acid is target DNA.

5. The method of claim 2, wherein the modification state of the segment is modified when the first signal is less than the second signal,

6. The method of claim 2, wherein the modification state of the segment is unmodified when the first signal is substantially the same as the second signal.

7. The method of claim 1, wherein the modification state is modified when the segment of the target nucleic acid comprises at least one base with a modification.

8. The method of claim 7, wherein the at least one base with the modification is present on a nucleic acid in a region 5′ to 3′ from nucleic acid 1 to nucleic acid 16 of the segment.

9. The method of claim 7, wherein the at least one base with the modification is present on a nucleic acid in the region 5′ to 3′ from nucleic acid 1 to nucleic acid 8 of the segment.

10. The method of claim 3, further comprising reverse transcribing the target RNA into DNA, amplifying the DNA, and in vitro transcribing the DNA into the target RNA.

11. The method of claim 4, further comprising:
contacting the target DNA to:
a DNA modification reagent.

12. The method of claim 11, wherein the DNA modification reagent is a modification-specific restriction enzyme or sodium bisulfite.

13. The method of claim 12, wherein the
modification-specific restriction enzyme cleaves the segment of the target DNA when the segment of the target DNA is unmodified.

14. The method of claim 4, wherein detection of the first signal indicates the segment of the target DNA is modified.

15. The method of claim 13, wherein the contacting the sample to the guide nucleic acid, the detector nucleic acid, the programmable nuclease, or any combination thereof occurs after the contacting the sample to the modification-specific restriction enzyme.

16. The method of claim 13, wherein the modification-specific restriction enzyme is DpnI, DpnII, MspI, MspJIAat II, Acc II, Aor13H I, Aor51H I, BspT104 I, BssH II, Cfr10 I, Cla I, Cpo I, Eco52, I, Hae II, Hha I, Mlu I, Nae I, Not I, Nru I, Nsb I, PmaC I, Psp1406 I, Pvu I, Sac II, Sal I, Sma I, SnaB I, or Epi HpaII.

17. (canceled)

18. (canceled)

19. The method of claim 4, wherein detection of the first signal indicates the modification state of the segment of the target DNA is unmodified.

20. The method of claim 12, wherein the
guide nucleic acid hybridizes to a sodium bisulfate converted segment of the target DNA.

21. (canceled)

22. The method ofclaim 12, the method further comprising deaminating an unmethylated cytosine into a uracil in the segment of the target DNA upon contacting the sample to the sodium bisulfite, thereby producing a sodium bisulfite converted segment of the target DNA.

23. The method of claim 1, further comprising amplifying the target nucleic acid.

24. (canceled)

25. (canceled)

26. (canceled)

27. The method of claim 7, wherein the modification comprises methylation.

28. (canceled)

29. (canceled)

30. The method of claim 7, wherein the modification comprises an 5-hydroxymethylcytosine (5hmC), 5-formylcytosine (5fC), 5-carboxylcytosine (5caC), 5-hydroxymethyluracil (5hmU), 5-methylcytosine (5mC), 3-methylcytosine (3mC), N6-methyladenosine (m6A), N6, 2′-O-dimethyladenosine (m6Am), N1-methyladenosine (m1A), N1-methylguanosine (m1G), 5-methylcytosine (m5C), or 5-hydroxymethylcytosine (hm5C).

31. The method of claim 7, wherein the modification comprises acetylation.

32. The method of claim 1, wherein the programmable nuclease is a Type VI programmable nuclease or a Type V programmable nuclease.

33. The method of claim 32, wherein the Type VI programmable nuclease is a Cas13 protein.

34. (canceled)

35. (canceled)

36. The method of claim 32, wherein the Type V programmable nuclease is a Cas12 protein or a Cas14 protein.

37. (canceled)

38. (canceled)

39. The method of claim 23, wherein the amplifying comprises thermal cycling amplification or isothermal amplification.

40.-47. (canceled)

48. A method of assaying for a target nucleic acid in a sample, the method comprising:
selectively producing a target single stranded DNA (ssDNA) by isothermal amplification of the target nucleic acid of the sample with a forward primer and a reverse primer to produce a target double stranded DNA having the target ssDNA and a nontarget ssDNA; and
contacting the sample to:
an exonuclease that selectively degrades the nontarget ssDNA;
a guide nucleic acid that hybridizes to a segment of the target ssDNA;
a detector nucleic acid; and
a programmable nuclease that cleaves the detector nucleic acid upon hybridization of the guide nucleic acid to the segment of the target ssDNA; and
assaying for a signal produced by cleavage of the detector nucleic acid to determine a presence of the target nucleic acid.

49.-55. (canceled)

56. A method of assaying for a target nucleic acid in a sample, the method comprising:
selectively producing a target single stranded DNA (ssDNA) by amplifying the target nucleic acid lacking a PAM sequence with:
a strand displacing polymerase; and
an outer forward primer, an inner forward primer, and a reverse primer or
an outer reverse primer, an inner reverse primer, and a forward primer; and
contacting the target ssDNA to:
a guide nucleic acid that hybridizes to a segment of the target ssDNA;
a detector nucleic acid; and
a programmable nuclease that cleaves the detector nucleic acid upon hybridization of the guide nucleic acid to the segment of the target ssDNA; and
assaying for a signal produced by cleavage of the detector nucleic acid to determine a presence of the target nucleic acid.

57.-63. (canceled)

64. The method of claim 1, wherein cleaving by the programmable nuclease comprises PAM-independent cleavage.

65.-73. (canceled)
